SEQUENCE LISTING (88 sequences)

SEQ ID NO: 1 - DNA, 19424 bp, Homo sapiens
  source 1..19424: mol_type = genomic DNA
gacagagccacagagggctgtgagcttgcccggccccaggtaacgctggcggtgggtgggcctccagcttggagcagagaccccccgaggcatctgcagacagaactggatggacccatgaatacggatttagctgctggaaaggtgagccctactaggtccccagggagagcgggagtctaggctgggaaagcctacccaagctgccagagctggaaggaccccatctccacccctattgtacagatgggaagactgaggcccagagaaaggatgcaatgagcaaaactgtgtgcctttgccccggggctgcccttgtctttcttccaggtccccctgcctcctaggagcgggcacttggggaggctgtgaaggcctgagaggcaagcggcatctctagctagggcctctctccagcactttttattttattttattttatttttatttttattttttgagatggagtctccctctgtcacccaggctagagtgcaatggcacgatctcagctcactgcaacttccacctcctgggttcaagagatcttcctgcctcagcctcccaagtagttgggattacaggtgtgcaccaccacgcccggctaatttttgtacttttcctagagacagggtttcaccatgttggctaggctggtctcaaactcctgacttcaggtgagctgcccgcctcggcttcccagagtgctgggattacagatgtgagccaccgtgcctgccatacttttttgttttttgaaacaaggtcttgctctgtgacccagggtggagtgcagtggggtggccatagctcactgcagcctcaacctcctgggttcaagggatcctcctacctcagcctccccagtagctgggacgacaagtgtgtgccaccgcagccagctaatttttttaaattatttgcagtagggataggggtctctatgttgcgcaggctggtcttgaactcctgggctcaagtgatcctcctgccccggcctcccaaagtgctgagattacaggcatgagccaccacatccagcctctccagcactttaagagaaattggctctcacactgtggaattctctctatcagcctcaaagtccagatttggaaagggagtctcagcgaggggcagcagctggcccaacccggaggcagagcggcaactgaactctagccggaaagagccagggttatgtgcacatgggaggtggggaggacaggggctgtatgtgaccctcacatctgttcctcgcgccccagatggcttctgctgcctgctccatggaccccatcgacagctttgagctcctggatctcctgtttgaccggcaggacggcatcctgagacacgtggagctgggcgagggctggggtcacgtcaaggaccaggtgaggagtccactgcagttgccccgggaccacagagctccaggcgggccaactgaggccccacgaaggtgctagacccgccagagcccagccagagctctgctcagctctacgatgcactaatgggtcacggtgcttgattttagccaagcacatcatttagcgggtagcatcagctccttcttacaggtggggaatggaggcccagagcagggtggaaactcatccaaagtcacacagcaatttggagttaggttcaaactcaaacttggggcatctccctggctaggttcggttctcttctccttcctctccttcctttctctctttcttcttcttttttttttttttgaaggagtctcgctctgttgcccaggctggagtgcagtggtgccatctcggctcactgcaacctctgcctcccgggttcaagcgattctcttgcctcagcctcccaagtagctgggattacaggcgcccgccactgcacctggctaatttttgtatttttagtagagatgggtttcactatcctggccaggctggtcttgaactcctgaccttgtgatccacccacctcagcctcccaaagtgctgagattacaggcgtgtgccaccgcgcccggcccactttcttttttttctttcttttctctctctttttctctttccttccttccttccttccttttttccttcctttctccctctttcttttactctctttttttttttttgagatagagtttcactcttgtcacccaggctggagcgcaatggcgcgatcttggctcactgcaacctccgcatcccaggttcaagtgattctcctacctcaacttcccgagtagctgggattacaggcagccgccaccacgctcggctaatttcttgtgtttttagtagagatggggtttcaccatgttggccaggctggtcttgaacttctgacctcgggtgatccacctgccttggcctcccaaagtgctgggattataggcgtgagccaccacacccggcccttttttctctttctttctctctctctctcgtttctctctctctctctctctctctctctctctctctctccccccctctctctctgtctctctctctctccccctccctcccttcctcccttcctcccttccttcctctctctctctctttcttagacagagtttcgctgttgttttgcccaggctggagtgcaatggtgtgatcttggcataccgcaacccccacctcccaggttcaagtgattctcctgcctcagcttcccgagtagctgagattacaggcatgtgccaccatgcctggctaattttgaatttttagtagagacggggtttctccctgttggtcaggctggtctcaaactcccgacctcaggtgatctgcccgccttggcctcccaaagtgctgggattacaggcctgagccaccgtgcctggctcttttgtttttctttttttcttttttttttttttttgagatggagtcttgctctgttgcctagggtggagtgcggtggcgtgatctcggctcactgcagcctccgtcttacgggttcagaatctccttcctcagactcccgagtagctgggactacagatgcccgccaccacgcccagctactttttgtatttttagaagaaatggggtttcatcatgttggccaggcggatctcgaactcctgacctcaagtgattcgccctcctcggcctcccaaagtgctgggattacaggcaggagccactgcacctggcactggctgggatttttatgctatgttactgcgtttgagcctcagcagccccgggaggaagaagcagtaactcatcttggagaagggaaggacacctaaggcccaaagagggtcactggtttccctggggccacacactaatctaaaaagagtgagcacttcctaattcctactacccctccctgtccaccccagcaggtcctgccaaaccccgactctgacgacttcctcagctccatcctgggctctggagactcactgcccagctccccactctggtcccccgaaggcagtgatagtggcatctccgaagacctcccctccgacccccaggacacccctccacgcagcggaccagccacctcccccgccggctgccatcctgcccagcctggcaaggggccctgcctctcctatcatcctggcaactcttgctccaccacaaccccagggccagtgatccaagtacctgaagcctctgtgaccatagacctgggtgagtcctgctgtgtctctgcccaccgccctcaccttgttccagggcccttcctgtaagtgaggaaatggaggcccagagagggctggcatcttgtccaaggtcacacagcaatttggagctgggcccagactcaaactcaggacacgtgtctcccttggctaggtgtttccatgctttcacgtggatgctattgggagcccattttccagatcgtgaaacagtcctgtcaggttgagcaatgagctaaaaccacagagctaggatgcaaccccggcaggtggctcaggcaccaggcatggtgctgacaagcacagcccaaagtcagggtcttgccttctacgtgtctgtaccccgtgcctggttcctagtagatttttttttattttttatttatttttattttttgagacagagtttcactctgtcacccaggctggagtaaagtggcgcgatctcggctcactgcaacctctgccttccaggttcaagcaattcacctgcctcagcctcctgagtagctgggatcacaagcatatgccaccatgcccagctaatatttattttttagtagagacgaggtttcaccatgttggccaggctggtcttgaactcctgagctcaggtgatctgcccacctcagcctcccaaagtgctgagattacaggtgtgagccgctccacccagcctgtagatgctcaataaaagtgcatgaggttaccaggtcctaggttccagggtgctcctcctagacgtggggaacagcagatcagtaatgatttactctattgattaagcattcccggcgggccaggcagagccctggctggagatacttggcctctgggtggcctttgtcccttgacccttgctatgagctcttcttggtcccattggagagttgacaaaactgaggctcaggcccggagcggtgactcatgcctgtaatcccagcactttgggaggctgaggcaggcggatcacttgaggttaggacttcaagaccagcctggccaacatggtgaaaccccatctctactaaaaatacaaaattagccgggcatggtgggggccacctgtagtcccagctactcaggagcctgaggcaggagaatcacctgaacccgggaggcggaggttgcagtgagctgagatcatgccactgcactccatcctggctgacagagccagactctgtctcgaaaaaaagccaaaaacaaaaaatgctgggcacagtggttcacgcctgtaatcccagcactttgggaggccgaagcgagcggatcacgaggtcaagagatcgagaccatcctggccaacatggtgaaacctctgtctctactaaaaatacaaaaattagctgggtgtggtggcaggcgcctgtagtctcagctactcgggaggctaaggcaggagaatctcttgaacctgggaggtggaggttgcggtgagccaagattgccacactgctctccagcctggcgacagagcgagactccgtctcaaaaaaaaaaaaaaaaaaaaatctgaggctcagagaggtgaagtcacttactcaaggtcacacagccctgcccaaccaggattagaaccaagggttacctgacccaaagtttgtgtgctgggacagcatgcctactgcatgccggctaccaggaggggacaaccagaggtgaatcagacctcgtccctgccccttgggtgagataacgcctggctggctgcaaatctagcctgtaattctgtgtgataacagccactcccttttgaatcagccggtgtctcctgacaatgtcctgtgtcttcctcagccttgtgggacggacagaaccaagccctgttgaatttgggtccccatcttttttttttttttgagatggagtctcgctgtgtcacccaggctggagggcagtgccacattctcagctcactgtaacgtccaccacccgggttcaagtgattctcctgcctcagcctcccgagcagctgggattacaggtgcacgccaccaggcccggctaatttttgtatttttaatagagatgggatttcaccatgttggccaggctggtcttgaactcctaacctcagatgatccgcctgccttggcctcccaaagtgctgggaatacaggcgtgagccaccatgactggctgggttcccatcttatagcagaggggagaccgagtcactagtgagggctgcccccttcaactcaggccttggggactccaaactctggcagaagtcatatgaatgttgggattaatcatgggagacttggtggaggaggcggttagaggctggaagctcaggactccccccgtctgacactctccctgtctccgtccccacctgccctggtcagaaatgtggagcccaggaggaaggatctgtgctgagaagccggctgatccggtggacctgtccccacgatgcaatctcaccgtgaaagacctcctcctttcgggcagcagtggggacctggtgagcacccccacaccctcccatggggcgttggagctgggagggctgctcgggttcgaggagcctaaatatccccgtttcagagactggaaaactgagtcacagaaaggcgtggaatttgcccacagttagagaccattccagtcctgggctcactgtcgcctgggctggggtgtcttcctctgtagtacttagtagtaacaatagcaacaacaactaaaagctaaaactggctgagtgtggtggttcacacctgtaatcccagcactttgggaggccaaggcaggcagatcgcttgagctcaggagtttgagaccagcctgggcaacatggtgaaaccccatctatacaaaaaatacaaaaattagcctggcatggtggcacatgcctgtggtcccagctactcgggaggctgaggtggaaggatcgcttgagcccaggaggtcaaggctgcagtgagctgtgattgtaccactgcactccagcctggccaacagaccaggactgtctcaacaataacaacaacaaaaagaacaaagctaaaactgaggctgcacctgctatatgccaggcgctctttatttatcatcttaaaaaagttttttttgagacaggatctcactctatcgcccaggctggagtgcagtggcgcaatcctagctcactgcagcctcaaactcctggcctcaagcgatcctcctgcctcagactcccaagtagctgggactacaggcgtgcaccaccacacccagctaatttttcttgtcttttttattttattttatttttctgagacagaatcttgctctgttgcccaggctggagtgcagtggtgcaatctcggatcactgccacctctgcctcccgggttcaagtgattctcctgcctcagcctcccaagtagctgagattacaggcatgcaccaccaggcctggctaattttgtttttcttttttctttttttttttgagatggcgtcttgctctgtcacctaggctggagtgcagtggcgtgatctcggctcactgcaagctccgcctaccgggttcacaccattctcctgcctcagcctccccagcagctgggactacaggagcatgccgcgccacctgtctaatttttcttttcttttttttttttttttttgagacggagtcttgctctgtcacctaggctggagtgcagtggcgtgatctcggctcactgcaagctccgcctaccgggttcacaccattctcctgcctcagcctcccgagtagctgggactacaggcgcccgccaccacacctggctgattttttgtatctttttagtagagatggggtttaactgtattagccaggattttttttgtatttttagtggagacagggtttcatcatgttagccaggatggtctcgatctcctgacctcgtgatctgcccacctcggcctcccaaagtgctgggattacaggcgtgagccaccgtgccccgtttagacccagctgatttttcttttaatttttttctgtagatatgggtcttgctatgtttcccaggctggtcttgaactcgtagcctcaagtgatcctcctgctttgtcctctcacagtgctggattataggcatgagctactgcgccccgcctggggcttttattatcttaattttacataggagaaaaagggcacagagaggttcagtcatttgcccaatgctacacagctgatgcaggcttgatccctgggatggtctattgagcttatggtgcccattgcacaccaattgaaggccatctaccctgccaagggcttcacgctcccataagccaggtttgacaacccaaacccattttgcagatgagaaggtaggtcaaggcccagagaggttaagccatgagccctaagtcacacagcatgcaagtggcagactcagggttttgaaatcctgtttctccaatgccaaaggttatcctgataatctctagtgaaggccagggtgcccctggcaactagagggatatgcagtgagcaccagctgtgtgctcagcctctaggatctgatggtcaggatggggcgatgtactgttcattaaggtgtacagaaaagaaagagagcctgcagggtcactgcagatccaggaaggcttcccggaggaggcagtacttcagcaggccatgaaggaagggaaaggagaatgaacgactgctttggcaatgataactggcttattttgttttattttatttgtttattttttgagacagagtcatggtccgtcgtccaggctaagtgcagtgccacagtctcggctcactgtagcctccacctcctgggctcaagtgattctcatacctcagcccccaggtagctaggactacaggtgcctgccaccatgcccgactaatttttgtatttttagtagagacggggtttcaccatgttggtcaggctggtctcgatctcctgacatcaggtgatccacccgcctcagcctcccaaagtgctgggattacaggcatgagccactgtgcctggccttattttattttatttaaagtcaaggtttccctctgtcaccacaactgcctcaaattcctaagctcaagtgatcctcctgcctcgacctcccattttttttgtatagctgacatacagaacaacgatacaaaaaaaaatgatgcatttataaatgctagttatcagcagttgaagaccagcctgggcaacatagcaagacctcgtctctacaaaaaaaaaaaaaaaaaattagctgagtgtggtggcgcacacctgtagtcccagctactcaggaggcagaggcaggaggatcacttgagctcaggaggtcaaggctgcagttagctatgatcacacgactgcactccagcctgggtgacacagtgacatcttgcatcttaaaagaagaaaagatgggtgtggtggctcatgcctgtaatcccaggaatttgggaggctgaagtggacagattgcttgagcccaggagtttgagaccagcctgggcaacagagtgaaaccccatctctacaaaaaattcaaaattagccaagcatggcggtacgctctgtggttccagctactcgggaagctgaagctggagaatcgcttgagcccgggaggcggaggttgcagtgagccaagagagcaccacggcactccaacccgggtgaagtagcaagaccctatttcaaaaataataatagtaataatacaatgaaaagaagaaagctgtcaaaagggccgggcgcggtggctcacgcctgtgatcccagcactttgggaggccgaggcgggcagatcacgaggtcaggagatcgagaccttcctggctaacacggtgaaaccccgtctctactaaaaataacaaaaaattagccgggcgtggtggcgggcacctgtagtcccagctactcaggaggctgaggcaggagaatggcgtgaacccgggaagcagagcttgcagtgagccgagatcgcgccactgcactccagcctgggcgacagagcgagactcgtctaagaaagaaagaaagaagaaagaaaagaaagaaagaaagaaagaaagaaggaaggaaggaaggaaggaaagaaagaaagaaccctgtccatatgccattttgcgcgtgtgtgaggatatctgtaggactgatttctgaaggtagaatgacccggtcaaaggaccgtgtgttggttatttcgagccctgctgccaagggagattctgtggtggcaggagtgatagtttttgttgttgctgttttgagacagaatttctgtcgcccaggctggatctcagatcactgcaacccctgcctcccgggttcaagcggttcttctgcctcagcctcccgagtagctgggattgcaggcacgtgccaccacaccaggctgatttttgtatttttaatggagacagggtttcaccatgttggccaggctagtctcgaactcctgacctcaagtgatccacctgcctcggcctcccaaagtgctgggactacagacgtgagccaccttttctttttttttttttttgagacagagtcttactctgtctctcaggctggagtgcagtggtgtgatctgggctcactggaacctctgcttcccgggttcaagcagttctcctgcctcagcctcctgagtagctgggattacaggcgccgccaccatggtcgggtaatttttttttttttttttttttgagacggagtctcatactgtcactagggctggagtgcagtggcacaatctcggctcactgcaacctctgacttctgggttcaagcgattctcctgcctcagcctcgcaagtagctggtattacaggcacccgccactacgcccagctaactttttgtatttttagtagagacggggtttcaccatgttgaccaggatggtctcaaactcctgacctagtgatttgcccacctcggcctcccaaagtgctgggattacaggcatgagccaccgcacctgggcaatttttgtatttttagtagagacgaggtttcatcatgttacccaggctggtctcgatctcctgagctcaagtgatccacatgcctcagcctctctaagtgctgggatgacaggcgtgagccaccacacccagcctggggtgatagtgttttcgatctgatacctctttcatttcctgaaggcagttggcgtccctgcacccgccgtcattcctctgattttttccgtagcaacagcatcacctgggggcctcctacctcctgcgacctggggctgggcactgtcaggagctggtgctcaccgaggatgagaagaagctgctggctaaagaaggcatcaccctgcccactcagctgcccctcactaaggtgagtctggggggacttccagccctggatccatctccccttcgtgcacttacctgcatgtgtgtcccacctttatttatttatttagagtagagtctttctctgtcactgaggctgggatgcagtggcacgatctcagcttgctgcaacctctgcctctcagattccagcgattctcccgcctcagcctcctgagtagctgggattacagccatgcgccaccacgcccagctaatttttgtatctttagtagagacagggtttcaccatgttggccaggctggtctcgagctcctgacctcatgtgatcctcctgcctcggtctcccaaagtgctgggattccaggcgtgagccaccgcactggctatgtgtctcacctttagaacacccgtactagaatgctatgctgtgtctgtcaccagagccaaaacaatcagtcagaagccccattgtaaattcaactcagagggtaattatcaacatccaggaatgaatggcggactccaggtggcagcaatttgggtgcagcaaacttttttttttttttggagatagggtcttgctgtgtcccgctggagtgcagtggtgccatcatagctcacttcagccttgacctcctgggctcaagagatcctcccgcctcagcctctcaagtagctaggatgacaggtgcacgccactacacttggctaatttaattttttttttttttttgtagaaatggggtcttgttatgttgcccaggctgggctgggactcctgggttcaagtgattctcccaccttggcctcccaaagtgttgggattacaggcatgagccatcatgcctggccctgcagacatgttttgattggactgtactgcattttatacatttttgaattgcttgataatgtttttctgaccggtagattttacaagagaactcagtgacttgaggctgggcacggtggctcacacctgtaatcccagcactgtgggaggctgaggcagaggatcacttgaggtcaggagttcgagactagcctggccaacatggtgaaaccccgtctctactaaaattacaaaaattagctgtgcctggtggtgggcaactgtaaggtgcttctcagcctcccagcttctcaggaggctgaggcaggaggatctcccttgaacccgggaggtggaggttgcagtgagccgagatggtaccactgcactccagcctgggtgacaagagcgaaactctgtcaaaacaaaaacaaaaacaaaaaaactaaaaacataaacaaaacaaaatccaaaaaaccaactcaattacttgaatgtcctctgtttcatattctcaaacctccatatgcaccagtcaatggctgggtagctgtttgggttctctagctaaccacagtccccacccagcctgctgcactcagtatcaatgcctgagtttgatattctcatctgttttttttttttttctattgagacaggttctgactctgttgcccaggctggagtggagtgaggtgatcttggctcactccaacttccccctctggagtacaagtgattctcccacatcagcctccctagcagctgggactacaggtgtatgccattatgcctagctaatttttttttttttcctgagacggagtttcgcttttgttgcccaggctggagtgcagtggcgcgatctcggctcactgcaacctccgcttcccaggttcaagtgattctcctgcctcagcctcctaagtagctgggattacaggcacgcgccaccacgcctggctaatttttttttttttttttttttttgagtagagacagagtttcaccatgttggccaggatggtctcgatctcttgaccttgtgatctgcccagctcagcctcccaaagtgttgggattacaggtgtgagccactgcgcccagcatttttttttttccttagagacagggtcttgctatgttgcccaggctggtctcaaactcctggcctcaagcagtcctctgaccttggcctcccaaagtgttaagattacaggtgtgagccaccgtgcctggccccatctgtttctattgttgttgttgtttgttttgagataggatctcactttgtcgctcaggctggagtgcagtggtacaatctgcaggtagcagctaactgcaaccttgaactcctggtctcaagggatcctcctgcctcagcctcccaagtagctgggactataggcatgcaccaccacacccagctgattttgtggttgttgttttgttttagtagagacagggtctttactaaatgttgcccagtctagtcttgaactcctggacttatgcgatcctcttgccttggcctcccaaagtgctgggattacagcactgtgaaccaccgcgcccagcctgacattcccatttgaagacacttattggccgggtgtggtgtctcatgcctgtaatcccagctctttgggaggcaaagatgggcggatcacctgaggtcaggagtttgagaccagcctggccaacatggtgaaaccctgtctctactaaaaatacaaaaattagccaggcatggtgttgtgcacctgtaatcccagctacttgggaagctggggtgggagaatcacttgaaccctgggggtggaggttgcagtgagctgagatcttgccactgcactccagcctgggtgacaagagcgaaacaagaaagaaagaaagaaagagagagagagagagagagagaaaaggaaggaaagaaaagaaagaaagaaagaaaggaaaagaaagaaaagaaagaaagaaaggaaagaaagaaaggaagaaaagaaggaaagaaaggaagaaaaggaaagaaagagagaaagagaaagagaaagagagaaagagagaaagggagaaaggaaggaagggagggagggagggagggaggggaaaagaaaggaaagaaaagaaagaaacgtaagaaagaagaaaaaaattattaattagtcttaaaacacttgctgtcgccagggaaaggtgatgtttccagggatggccacaaggtggtggagtggtacccatgcccaaagtctggtgggcagttgcccattttacagtattgggagctgggaaaagaccctcagaacccaggcacctcccagagactctgtgagggtttgggcaaagggtagcttcaagtcattaggaatctgcctctgggtccttggctccgggatgtcttgtatgcaaagaggatggggtgaagttgtggggtcgaggttaggtgaagtggggtcaggacagtggggactgtcgcaccatccccaaagtgaagactttaattttttaattttaattattttatttatttatttatttatttatttatctatttatttattgagatggagtttcgctcttgttgcccaggctggattgcaatggcacgatcttggctcactgcaacctccgcctcccgggttcatgccattctcctgcctcagcctcccgagtagctgggattacaggcacccgccaccatgcctggctaatttttgtatttttagtagagacggtgtttcaccatgttggccaggctggtctcgaactcctgacctcaggtgatacgcctgcctcggcctcccaaagtgctgggattacaggcgagagctactgcccccggactccaagtggggactttctggcctgaaaccctcctccccatttcacttggcagtacgaggagcgagtgctgaaaaaaatccgccggaaaatccggaacaagcagtcggcgcaagaaagcaggaagaagaagaaggaatatatcgatggcctggagactcggtgggtagtgctggacccagactctacactcgtggaggaaactgaggcccagagagaaggagatgtggctaaggccacacagcttagagtctgacaaaattggaggtgattctcctctctgggagagatcatgcttcttactgattgcagggatggaaagatgcctagtgatggtgtaggacaagggtttgggggaccctttgccccctagaaggtgtgcggaaaaaggacaaagtgtgacccatcctattctgaccctctgcctgaagatagacacttcctttttgtgtgtgtggaagggtctccttctatcacccaggctggagtgcagtggtgcgatcttggctcactgcaacctccacctcccgggttcaagcgattctcctgcctcagcctctccagaagctgggattacaggtgcatggccaccacgcccagctaatttctttgtatttttggtagagatggggtttcactgtgttgcccaggctagtctcaaactcctgacctcaggtgatctggccgcctcagcctcccaaagtgctgggattacaggcgtgagccactgcgcctggtctctcccctttcttaaatgcaatctgctaagctcctgatcatctttatccagggtcattccccccaccaccctatgtcctgggtaattgtatcccattttacagacacaaaagctgagatcagagagtggagtggcccaaggtcatggggagagggagagatgaaacttgaatctcggccgggcgccgtggctcacacttgtaatcccagcactttgggaggctgaggcaggcggatcacgagttcaggagatcgagaccacggtgaaactctgtctttacaaaaaatccaaaaaaattaactgggcttggtggcgggtgcctgtagtcccagctactcgggaggctgaggcaggagaatggcgtgaacccagaaggcagagcttgcagtgagtcgagattgtgccactgcattccagcctgggcgacagagcgagactccatctcaaaaaacaaacaaacaaaaaaagaaacttgagtctcccaggatgcctggttgtcctgatatttcatcttctggaaacatcactgcctgaatctaggaggctcagcttttttttttttttttttttttttgacacagagtttcattcttgttgcccaggctggagtgcaatggtgcgatcttgactcactgcaacctccacttcccgggttcaatcgattctcctgccttagcctcccaagttcccaagtagctggaattataggcgcccaccaccacacctggctaattttgcatttttagtacaggcagggtttcaccatgttggccaggctggtcttgaactcctgacctcaggtgatccgcccaccacggcctcccaaagtgctgggattacaggtgtgaaccaccacacccatccagggggctcagctttgaatgaagccccctccatattaatgcagaaatgggacctaggaaccaggcagatgtgggttcaaatcccagccctgtctgacctgccgagtaaccttggatgaatgacttcccctctctggccttgggttctcttggcttgtaacgtgaggcctctgggggagatgtcagtggggccagctggtgactggcatatgctgaatgtcgatgcgtcttcctcctttcaggatgtcagcttgcactgctcagaatcaggagttacagaggaaagtcttgcatctcgagaagcaaaacctgtgagtctgggtccagctggggcagaggacaggggaagcagccccagagtccctttgcaggaagagcagagagcccatgtgatggcagaatgacatagggaataagagttttacctatgggccgcgtgcagtggctcacgcctgtaatctcagcactttgggaggccgaggcgggtggatcacctgaggtcaggagttcaaaaccagcctggccaacatggtgaaaccccgtctctactaaaaatacaaaaaaattagccaggcgtggtggcgggcacctgcagtcccagctactcgagaggctgaggcaggagaattgcttgaactcgggaggcggaggttgccgtgagccgagatcgcaccactgcactccagcctgggtgacagagcaaggctctgtctcaaaaataaataaataaggcctggtgtggtggctcacgcctgtaatcccagcactttgggaggctgaggtgggcggatcacaaggtcgggagatcaagaccatcctggctaacacggtgaaaccccgtctctactaaaaatacaaaaaaaattagctgggtgtggtggtgggcgcctgtagtcccagctactcgggaggctgaggcaggagaatggtgtgaacctgggaggcggagcttgcagtgagctgccatcccaccactcactccagcctgggcgatggagcaagactctgtctcaaaataaataaataaataaataaacaaataaataaataaataaaataaataataaagagccagaggcccttgaagaatggatggaatttggactttagcggggctgggggaccccggaaatggacgagaagcagaaccgaggccctttagggctcagcggaggcctgcctgtctctctaaggtccctcttggagcaactgaagaaactccaggccattgtggtgcagtccaccagcaagtcagcccagacaggcacctgtgtcgcagtgagtcctggtgcccccaggcaagccggggacctaggcttctgtagaggggcccatagggaggtgacaatgagtccaagctctccttgtgccccagctcaagtatgatccagtctggtctttggggcctcagtttccctgcctgtgggatggagatgcttgcaggggaggggagggagggggtgactctgccgctgtctccaccaggtcctgttgctgtcctttgccctcatcatcctcccctccatcagcccttttggccccaacaaaaccgagagccctggggactttgcgcctgtacgaggtaggggatccccacctttgaaacccttgtctggtctccccaagtccccgtcctgggcctctgggggagggggagaagacccctgtgcccttgttccctgaggctgggggccagggaagggagcataggaccccacctccaccttgtcccctgtgatgccccccttccccaatcagtgttctccagaactttgcacaacgatgctgcctcccgcgtggctgctgatgctgtgccaggctccgaggccccaggaccccgacccgaggctgacacaacccgagaagagtctccaggaagccccggggcagactggggcttccaggacaccgcgaacctgaccaattcgacggaggagctggacaacgccaccctggtcctgaggaatgcaacagaggggctgggccaggtcgccctgctggactgggtggcgcctgggccgagcactggctcaggacgtgcagggctggaggcggcgggagacgagctgtgagccccgccaggactatgctcccaggcccctctgcccaggggtgccttggggatgctgcactgggcagctacccacctggggatgggacgtgaggccaagaccccagcagagatgccagaatgggggaggcacagctcatagccacacacccagggcctgactgaggcccacgcaggaaccgacactcagacacaaggcaaagagggccacaggacccgggaaatacacacagagccaggagcagaagcaaagagcagacacacatacagcctgaaacagacctggacagacagacacagcctgaaacagacccggacagacagacacagcctgaaacagacccagacaaacagacagacagacacagcctgaaacagacccagacagacagacagacagcctgaaacagacccagacacagcctgaaacagatccggacagacagacagaaacagcctgaaacagacccagacagacagacagacacagcctgaaacagacccggacagacagacagacacagcctgaaacagacccggacagacagacagacacagcctgaaacagacccggacagacagacagacacagcctgaaacagacctagacagacagacacagattgaaacagacccagacaaacagacagacacagcctgaaacagacccagacacagcctgaaacagacccggacagacagacagacacagcctgaaacagacccagacagacagacagacacagcctgaaacagacccagacagacagaccgacgcagcctgaaagagacccagacagagagacaggcagacacagcctaaaacagacctggacagacaggcagacgtagtctgaaacagacctgaacagacagacagacgcacacacacaacagatgcgcagcaactccccgcccagggacccctcccggcctccctcgcacactgggaggaggaagccgccgagactgcagggagcctggccccggagccccgggtgcgccctggtctttggagcagccacggcccacaatcaccccccttttctaagactgcctgatccgaaataaagtattttgacagaa

SEQ ID NO: 2 - DNA, 19424 bp, Homo sapiens
  source 1..19424: mol_type = genomic DNA
gacagagccacagagggctgtgagcttgcccggccccaggtaacgctggcggtgggtgggcctccagcttggagcagagaccccccgaggcatctgcagacagaactggatggacccatgaatacggatttagctgctggaaaggtgagccctactaggtccccagggagagcgggagtctaggctgggaaagcctacccaagctgccagagctggaaggaccccatctccacccctattgtacagatgggaagactgaggcccagagaaaggatgcaatgagcaaaactgtgtgcctttgccccggggctgcccttgtctttcttccaggtccccctgcctcctaggagcgggcacttggggaggctgtgaaggcctgagaggcaagcggcatctctagctagggcctctctccagcactttttattttattttattttatttttatttttattttttgagatggagtctccctctgtcacccaggctagagtgcaatggcacgatctcagctcactgcaacttccacctcctgggttcaagagatcttcctgcctcagcctcccaagtagttgggattacaggtgtgcaccaccacgcccggctaatttttgtacttttcctagagacagggtttcaccatgttggctaggctggtctcaaactcctgacttcaggtgagctgcccgcctcggcttcccagagtgctgggattacagatgtgagccaccgtgcctgccatacttttttgttttttgaaacaaggtcttgctctgtgacccagggtggagtgcagtggggtggccatagctcactgcagcctcaacctcctgggttcaagggatcctcctacctcagcctccccagtagctgggacgacaagtgtgtgccaccgcagccagctaatttttttaaattatttgcagtagggataggggtctctatgttgcgcaggctggtcttgaactcctgggctcaagtgatcctcctgccccggcctcccaaagtgctgagattacaggcatgagccaccacatccagcctctccagcactttaagagaaattggctctcacactgtggaattctctctatcagcctcaaagtccagatttggaaagggagtctcagcgaggggcagcagctggcccaacccggaggcagagcggcaactgaactctagccggaaagagccagggttatgtgcacatgggaggtggggaggacaggggctgtatgtgaccctcacatctgttcctcgcgccccagatggcttctgctgcctgctccatggaccccatcgacagctttgagctcctggatctcctgtttgaccggcaggacggcatcctgagacacgtggagctgggcgagggctggggtcacgtcaaggaccaggtgaggagtccactgcagttgccccgggaccacagagctccaggcgggccaactgaggccccacgaaggtgctagacccgccagagcccagccagagctctgctcagctctacgatgcactaatgggtcacggtgcttgattttagccaagcacatcatttagcgggtagcatcagctccttcttacaggtggggaatggaggcccagagcagggtggaaactcatccaaagtcacacagcaatttggagttaggttcaaactcaaacttggggcatctccctggctaggttcggttctcttctccttcctctccttcctttctctctttcttcttcttttttttttttttgaaggagtctcgctctgttgcccaggctggagtgcagtggtgccatctcggctcactgcaacctctgcctcccgggttcaagcgattctcttgcctcagcctcccaagtagctgggattacaggcgcccgccactgcacctggctaatttttgtatttttagtagagatgggtttcactatcctggccaggctggtcttgaactcctgaccttgtgatccacccacctcagcctcccaaagtgctgagattacaggcgtgtgccaccgcgcccggcccactttcttttttttctttcttttctctctctttttctctttccttccttccttccttccttttttccttcctttctccctctttcttttactctctttttttttttttgagatagagtttcactcttgtcacccaggctggagcgcaatggcgcgatcttggctcactgcaacctccgcatcccaggttcaagtgattctcctacctcaacttcccgagtagctgggattacaggcagccgccaccacgctcggctaatttcttgtgtttttagtagagatggggtttcaccatgttggccaggctggtcttgaacttctgacctcgggtgatccacctgccttggcctcccaaagtgctgggattataggcgtgagccaccacacccggcccttttttctctttctttctctctctctctcgtttctctctctctctctctctctctctctctctctctctccccccctctctctctgtctctctctctctccccctccctcccttcctcccttcctcccttccttcctctctctctctctttcttagacagagtttcgctgttgttttgcccaggctggagtgcaatggtgtgatcttggcataccgcaacccccacctcccaggttcaagtgattctcctgcctcagcttcccgagtagctgagattacaggcatgtgccaccatgcctggctaattttgaatttttagtagagacggggtttctccctgttggtcaggctggtctcaaactcccgacctcaggtgatctgcccgccttggcctcccaaagtgctgggattacaggcctgagccaccgtgcctggctcttttgtttttctttttttcttttttttttttttttgagatggagtcttgctctgttgcctagggtggagtgcggtggcgtgatctcggctcactgcagcctccgtcttacgggttcagaatctccttcctcagactcccgagtagctgggactacagatgcccgccaccacgcccagctactttttgtatttttagaagaaatggggtttcatcatgttggccaggcggatctcgaactcctgacctcaagtgattcgccctcctcggcctcccaaagtgctgggattacaggcaggagccactgcacctggcactggctgggatttttatgctatgttactgcgtttgagcctcagcagccccgggaggaagaagcagtaactcatcttggagaagggaaggacacctaaggcccaaagagggtcactggtttccctggggccacacactaatctaaaaagagtgagcacttcctaattcctactacccctccctgtccaccccagcaggtcctgccaaaccccgactctgacgacttcctcagctccatcctgggctctggagactcactgcccagctccccactctggtcccccgaaggcagtgatagtggcatctccgaagacctcccctccgacccccaggacacccctccacgcagcggaccagccacctcccccgccggctgccatcctgcccagcctggcaaggggccctgcctctcctatcatcctggcaactcttgctccaccacaaccccagggccagtgatccaagtacctgaagcctctgtgaccatagacctgggtgagtcctgctgtgtctctgcccaccgccctcaccttgttccagggcccttcctgtaagtgaggaaatggaggcccagagagggctggcatcttgtccaaggtcacacagcaatttggagctgggcccagactcaaactcaggacacgtgtctcccttggctaggtgtttccatgctttcacgtggatgctattgggagcccattttccagatcgtgaaacagtcctgtcaggttgagcaatgagctaaaaccacagagctaggatgcaaccccggcaggtggctcaggcaccaggcatggtgctgacaagcacagcccaaagtcagggtcttgccttctacgtgtctgtaccccgtgcctggttcctagtagatttttttttattttttatttatttttattttttgagacagagtttcactctgtcacccaggctggagtaaagtggcgcgatctcggctcactgcaacctctgccttccaggttcaagcaattcacctgcctcagcctcctgagtagctgggatcacaagcatatgccaccatgcccagctaatatttattttttagtagagacgaggtttcaccatgttggccaggctggtcttgaactcctgagctcaggtgatctgcccacctcagcctcccaaagtgctgagattacaggtgtgagccgctccacccagcctgtagatgctcaataaaagtgcatgaggttaccaggtcctaggttccagggtgctcctcctagacgtggggaacagcagatcagtaatgatttactctattgattaagcattcccggcgggccaggcagagccctggctggagatacttggcctctgggtggcctttgtcccttgacccttgctatgagctcttcttggtcccattggagagttgacaaaactgaggctcaggcccggagcggtgactcatgcctgtaatcccagcactttgggaggctgaggcaggcggatcacttgaggttaggacttcaagaccagcctggccaacatggtgaaaccccatctctactaaaaatacaaaattagccgggcatggtgggggccacctgtagtcccagctactcaggagcctgaggcaggagaatcacctgaacccgggaggcggaggttgcagtgagctgagatcatgccactgcactccatcctggctgacagagccagactctgtctcgaaaaaaagccaaaaacaaaaaatgctgggcacagtggttcacgcctgtaatcccagcactttgggaggccgaagcgagcggatcacgaggtcaagagatcgagaccatcctggccaacatggtgaaacctctgtctctactaaaaatacaaaaattagctgggtgtggtggcaggcgcctgtagtctcagctactcgggaggctaaggcaggagaatctcttgaacctgggaggtggaggttgcggtgagccaagattgccacactgctctccagcctggcgacagagcgagactccgtctcaaaaaaaaaaaaaaaaaaaaatctgaggctcagagaggtgaagtcacttactcaaggtcacacagccctgcccaaccaggattagaaccaagggttacctgacccaaagtttgtgtgctgggacagcatgcctactgcatgccggctaccaggaggggacaaccagaggtgaatcagacctcgtccctgccccttgggtgagataacgcctggctggctgcaaatctagcctgtaattctgtgtgataacagccactcccttttgaatcagccggtgtctcctgacaatgtcctgtgtcttcctcagccttgtgggacggacagaaccaagccctgttgaatttgggtccccatcttttttttttttttgagatggagtctcgctgtgtcacccaggctggagggcagtgccacattctcagctcactgtaacgtccaccacccgggttcaagtgattctcctgcctcagcctcccgagcagctgggattacaggtgcacgccaccaggcccggctaatttttgtatttttaatagagatgggatttcaccatgttggccaggctggtcttgaactcctaacctcagatgatccgcctgccttggcctcccaaagtgctgggaatacaggcgtgagccaccatgactggctgggttcccatcttatagcagaggggagaccgagtcactagtgagggctgcccccttcaactcaggccttggggactccaaactctggcagaagtcatatgaatgttgggattaatcatgggagacttggtggaggaggcggttagaggctggaagctcaggactccccccgtctgacactctccctgtctccgtccccacctgccctggtcagaaatgtggagcccaggaggaaggatctgtgctgagaagccggctgatccggtggacctgtccccacgatgcaatctcaccgtgaaaaacctcctcctttcgggcagcagtggggacctggtgagcacccccacaccctcccatggggcgttggagctgggagggctgctcgggttcgaggagcctaaatatccccgtttcagagactggaaaactgagtcacagaaaggcgtggaatttgcccacagttagagaccattccagtcctgggctcactgtcgcctgggctggggtgtcttcctctgtagtacttagtagtaacaatagcaacaacaactaaaagctaaaactggctgagtgtggtggttcacacctgtaatcccagcactttgggaggccaaggcaggcagatcgcttgagctcaggagtttgagaccagcctgggcaacatggtgaaaccccatctatacaaaaaatacaaaaattagcctggcatggtggcacatgcctgtggtcccagctactcgggaggctgaggtggaaggatcgcttgagcccaggaggtcaaggctgcagtgagctgtgattgtaccactgcactccagcctggccaacagaccaggactgtctcaacaataacaacaacaaaaagaacaaagctaaaactgaggctgcacctgctatatgccaggcgctctttatttatcatcttaaaaaagttttttttgagacaggatctcactctatcgcccaggctggagtgcagtggcgcaatcctagctcactgcagcctcaaactcctggcctcaagcgatcctcctgcctcagactcccaagtagctgggactacaggcgtgcaccaccacacccagctaatttttcttgtcttttttattttattttatttttctgagacagaatcttgctctgttgcccaggctggagtgcagtggtgcaatctcggatcactgccacctctgcctcccgggttcaagtgattctcctgcctcagcctcccaagtagctgagattacaggcatgcaccaccaggcctggctaattttgtttttcttttttctttttttttttgagatggcgtcttgctctgtcacctaggctggagtgcagtggcgtgatctcggctcactgcaagctccgcctaccgggttcacaccattctcctgcctcagcctccccagcagctgggactacaggagcatgccgcgccacctgtctaatttttcttttcttttttttttttttttttgagacggagtcttgctctgtcacctaggctggagtgcagtggcgtgatctcggctcactgcaagctccgcctaccgggttcacaccattctcctgcctcagcctcccgagtagctgggactacaggcgcccgccaccacacctggctgattttttgtatctttttagtagagatggggtttaactgtattagccaggattttttttgtatttttagtggagacagggtttcatcatgttagccaggatggtctcgatctcctgacctcgtgatctgcccacctcggcctcccaaagtgctgggattacaggcgtgagccaccgtgccccgtttagacccagctgatttttcttttaatttttttctgtagatatgggtcttgctatgtttcccaggctggtcttgaactcgtagcctcaagtgatcctcctgctttgtcctctcacagtgctggattataggcatgagctactgcgccccgcctggggcttttattatcttaattttacataggagaaaaagggcacagagaggttcagtcatttgcccaatgctacacagctgatgcaggcttgatccctgggatggtctattgagcttatggtgcccattgcacaccaattgaaggccatctaccctgccaagggcttcacgctcccataagccaggtttgacaacccaaacccattttgcagatgagaaggtaggtcaaggcccagagaggttaagccatgagccctaagtcacacagcatgcaagtggcagactcagggttttgaaatcctgtttctccaatgccaaaggttatcctgataatctctagtgaaggccagggtgcccctggcaactagagggatatgcagtgagcaccagctgtgtgctcagcctctaggatctgatggtcaggatggggcgatgtactgttcattaaggtgtacagaaaagaaagagagcctgcagggtcactgcagatccaggaaggcttcccggaggaggcagtacttcagcaggccatgaaggaagggaaaggagaatgaacgactgctttggcaatgataactggcttattttgttttattttatttgtttattttttgagacagagtcatggtccgtcgtccaggctaagtgcagtgccacagtctcggctcactgtagcctccacctcctgggctcaagtgattctcatacctcagcccccaggtagctaggactacaggtgcctgccaccatgcccgactaatttttgtatttttagtagagacggggtttcaccatgttggtcaggctggtctcgatctcctgacatcaggtgatccacccgcctcagcctcccaaagtgctgggattacaggcatgagccactgtgcctggccttattttattttatttaaagtcaaggtttccctctgtcaccacaactgcctcaaattcctaagctcaagtgatcctcctgcctcgacctcccattttttttgtatagctgacatacagaacaacgatacaaaaaaaaatgatgcatttataaatgctagttatcagcagttgaagaccagcctgggcaacatagcaagacctcgtctctacaaaaaaaaaaaaaaaaaattagctgagtgtggtggcgcacacctgtagtcccagctactcaggaggcagaggcaggaggatcacttgagctcaggaggtcaaggctgcagttagctatgatcacacgactgcactccagcctgggtgacacagtgacatcttgcatcttaaaagaagaaaagatgggtgtggtggctcatgcctgtaatcccaggaatttgggaggctgaagtggacagattgcttgagcccaggagtttgagaccagcctgggcaacagagtgaaaccccatctctacaaaaaattcaaaattagccaagcatggcggtacgctctgtggttccagctactcgggaagctgaagctggagaatcgcttgagcccgggaggcggaggttgcagtgagccaagagagcaccacggcactccaacccgggtgaagtagcaagaccctatttcaaaaataataatagtaataatacaatgaaaagaagaaagctgtcaaaagggccgggcgcggtggctcacgcctgtgatcccagcactttgggaggccgaggcgggcagatcacgaggtcaggagatcgagaccttcctggctaacacggtgaaaccccgtctctactaaaaataacaaaaaattagccgggcgtggtggcgggcacctgtagtcccagctactcaggaggctgaggcaggagaatggcgtgaacccgggaagcagagcttgcagtgagccgagatcgcgccactgcactccagcctgggcgacagagcgagactcgtctaagaaagaaagaaagaagaaagaaaagaaagaaagaaagaaagaaagaaggaaggaaggaaggaaggaaagaaagaaagaaccctgtccatatgccattttgcgcgtgtgtgaggatatctgtaggactgatttctgaaggtagaatgacccggtcaaaggaccgtgtgttggttatttcgagccctgctgccaagggagattctgtggtggcaggagtgatagtttttgttgttgctgttttgagacagaatttctgtcgcccaggctggatctcagatcactgcaacccctgcctcccgggttcaagcggttcttctgcctcagcctcccgagtagctgggattgcaggcacgtgccaccacaccaggctgatttttgtatttttaatggagacagggtttcaccatgttggccaggctagtctcgaactcctgacctcaagtgatccacctgcctcggcctcccaaagtgctgggactacagacgtgagccaccttttctttttttttttttttgagacagagtcttactctgtctctcaggctggagtgcagtggtgtgatctgggctcactggaacctctgcttcccgggttcaagcagttctcctgcctcagcctcctgagtagctgggattacaggcgccgccaccatggtcgggtaatttttttttttttttttttttgagacggagtctcatactgtcactagggctggagtgcagtggcacaatctcggctcactgcaacctctgacttctgggttcaagcgattctcctgcctcagcctcgcaagtagctggtattacaggcacccgccactacgcccagctaactttttgtatttttagtagagacggggtttcaccatgttgaccaggatggtctcaaactcctgacctagtgatttgcccacctcggcctcccaaagtgctgggattacaggcatgagccaccgcacctgggcaatttttgtatttttagtagagacgaggtttcatcatgttacccaggctggtctcgatctcctgagctcaagtgatccacatgcctcagcctctctaagtgctgggatgacaggcgtgagccaccacacccagcctggggtgatagtgttttcgatctgatacctctttcatttcctgaaggcagttggcgtccctgcacccgccgtcattcctctgattttttccgtagcaacagcatcacctgggggcctcctacctcctgcgacctggggctgggcactgtcaggagctggtgctcaccgaggatgagaagaagctgctggctaaagaaggcatcaccctgcccactcagctgcccctcactaaggtgagtctggggggacttccagccctggatccatctccccttcgtgcacttacctgcatgtgtgtcccacctttatttatttatttagagtagagtctttctctgtcactgaggctgggatgcagtggcacgatctcagcttgctgcaacctctgcctctcagattccagcgattctcccgcctcagcctcctgagtagctgggattacagccatgcgccaccacgcccagctaatttttgtatctttagtagagacagggtttcaccatgttggccaggctggtctcgagctcctgacctcatgtgatcctcctgcctcggtctcccaaagtgctgggattccaggcgtgagccaccgcactggctatgtgtctcacctttagaacacccgtactagaatgctatgctgtgtctgtcaccagagccaaaacaatcagtcagaagccccattgtaaattcaactcagagggtaattatcaacatccaggaatgaatggcggactccaggtggcagcaatttgggtgcagcaaacttttttttttttttggagatagggtcttgctgtgtcccgctggagtgcagtggtgccatcatagctcacttcagccttgacctcctgggctcaagagatcctcccgcctcagcctctcaagtagctaggatgacaggtgcacgccactacacttggctaatttaattttttttttttttttgtagaaatggggtcttgttatgttgcccaggctgggctgggactcctgggttcaagtgattctcccaccttggcctcccaaagtgttgggattacaggcatgagccatcatgcctggccctgcagacatgttttgattggactgtactgcattttatacatttttgaattgcttgataatgtttttctgaccggtagattttacaagagaactcagtgacttgaggctgggcacggtggctcacacctgtaatcccagcactgtgggaggctgaggcagaggatcacttgaggtcaggagttcgagactagcctggccaacatggtgaaaccccgtctctactaaaattacaaaaattagctgtgcctggtggtgggcaactgtaaggtgcttctcagcctcccagcttctcaggaggctgaggcaggaggatctcccttgaacccgggaggtggaggttgcagtgagccgagatggtaccactgcactccagcctgggtgacaagagcgaaactctgtcaaaacaaaaacaaaaacaaaaaaactaaaaacataaacaaaacaaaatccaaaaaaccaactcaattacttgaatgtcctctgtttcatattctcaaacctccatatgcaccagtcaatggctgggtagctgtttgggttctctagctaaccacagtccccacccagcctgctgcactcagtatcaatgcctgagtttgatattctcatctgttttttttttttttctattgagacaggttctgactctgttgcccaggctggagtggagtgaggtgatcttggctcactccaacttccccctctggagtacaagtgattctcccacatcagcctccctagcagctgggactacaggtgtatgccattatgcctagctaatttttttttttttcctgagacggagtttcgcttttgttgcccaggctggagtgcagtggcgcgatctcggctcactgcaacctccgcttcccaggttcaagtgattctcctgcctcagcctcctaagtagctgggattacaggcacgcgccaccacgcctggctaatttttttttttttttttttttttgagtagagacagagtttcaccatgttggccaggatggtctcgatctcttgaccttgtgatctgcccagctcagcctcccaaagtgttgggattacaggtgtgagccactgcgcccagcatttttttttttccttagagacagggtcttgctatgttgcccaggctggtctcaaactcctggcctcaagcagtcctctgaccttggcctcccaaagtgttaagattacaggtgtgagccaccgtgcctggccccatctgtttctattgttgttgttgtttgttttgagataggatctcactttgtcgctcaggctggagtgcagtggtacaatctgcaggtagcagctaactgcaaccttgaactcctggtctcaagggatcctcctgcctcagcctcccaagtagctgggactataggcatgcaccaccacacccagctgattttgtggttgttgttttgttttagtagagacagggtctttactaaatgttgcccagtctagtcttgaactcctggacttatgcgatcctcttgccttggcctcccaaagtgctgggattacagcactgtgaaccaccgcgcccagcctgacattcccatttgaagacacttattggccgggtgtggtgtctcatgcctgtaatcccagctctttgggaggcaaagatgggcggatcacctgaggtcaggagtttgagaccagcctggccaacatggtgaaaccctgtctctactaaaaatacaaaaattagccaggcatggtgttgtgcacctgtaatcccagctacttgggaagctggggtgggagaatcacttgaaccctgggggtggaggttgcagtgagctgagatcttgccactgcactccagcctgggtgacaagagcgaaacaagaaagaaagaaagaaagagagagagagagagagagagaaaaggaaggaaagaaaagaaagaaagaaagaaaggaaaagaaagaaaagaaagaaagaaaggaaagaaagaaaggaagaaaagaaggaaagaaaggaagaaaaggaaagaaagagagaaagagaaagagaaagagagaaagagagaaagggagaaaggaaggaagggagggagggagggagggaggggaaaagaaaggaaagaaaagaaagaaacgtaagaaagaagaaaaaaattattaattagtcttaaaacacttgctgtcgccagggaaaggtgatgtttccagggatggccacaaggtggtggagtggtacccatgcccaaagtctggtgggcagttgcccattttacagtattgggagctgggaaaagaccctcagaacccaggcacctcccagagactctgtgagggtttgggcaaagggtagcttcaagtcattaggaatctgcctctgggtccttggctccgggatgtcttgtatgcaaagaggatggggtgaagttgtggggtcgaggttaggtgaagtggggtcaggacagtggggactgtcgcaccatccccaaagtgaagactttaattttttaattttaattattttatttatttatttatttatttatttatctatttatttattgagatggagtttcgctcttgttgcccaggctggattgcaatggcacgatcttggctcactgcaacctccgcctcccgggttcatgccattctcctgcctcagcctcccgagtagctgggattacaggcacccgccaccatgcctggctaatttttgtatttttagtagagacggtgtttcaccatgttggccaggctggtctcgaactcctgacctcaggtgatacgcctgcctcggcctcccaaagtgctgggattacaggcgagagctactgcccccggactccaagtggggactttctggcctgaaaccctcctccccatttcacttggcagtacgaggagcgagtgctgaaaaaaatccgccggaaaatccggaacaagcagtcggcgcaagaaagcaggaagaagaagaaggaatatatcgatggcctggagactcggtgggtagtgctggacccagactctacactcgtggaggaaactgaggcccagagagaaggagatgtggctaaggccacacagcttagagtctgacaaaattggaggtgattctcctctctgggagagatcatgcttcttactgattgcagggatggaaagatgcctagtgatggtgtaggacaagggtttgggggaccctttgccccctagaaggtgtgcggaaaaaggacaaagtgtgacccatcctattctgaccctctgcctgaagatagacacttcctttttgtgtgtgtggaagggtctccttctatcacccaggctggagtgcagtggtgcgatcttggctcactgcaacctccacctcccgggttcaagcgattctcctgcctcagcctctccagaagctgggattacaggtgcatggccaccacgcccagctaatttctttgtatttttggtagagatggggtttcactgtgttgcccaggctagtctcaaactcctgacctcaggtgatctggccgcctcagcctcccaaagtgctgggattacaggcgtgagccactgcgcctggtctctcccctttcttaaatgcaatctgctaagctcctgatcatctttatccagggtcattccccccaccaccctatgtcctgggtaattgtatcccattttacagacacaaaagctgagatcagagagtggagtggcccaaggtcatggggagagggagagatgaaacttgaatctcggccgggcgccgtggctcacacttgtaatcccagcactttgggaggctgaggcaggcggatcacgagttcaggagatcgagaccacggtgaaactctgtctttacaaaaaatccaaaaaaattaactgggcttggtggcgggtgcctgtagtcccagctactcgggaggctgaggcaggagaatggcgtgaacccagaaggcagagcttgcagtgagtcgagattgtgccactgcattccagcctgggcgacagagcgagactccatctcaaaaaacaaacaaacaaaaaaagaaacttgagtctcccaggatgcctggttgtcctgatatttcatcttctggaaacatcactgcctgaatctaggaggctcagcttttttttttttttttttttttttgacacagagtttcattcttgttgcccaggctggagtgcaatggtgcgatcttgactcactgcaacctccacttcccgggttcaatcgattctcctgccttagcctcccaagttcccaagtagctggaattataggcgcccaccaccacacctggctaattttgcatttttagtacaggcagggtttcaccatgttggccaggctggtcttgaactcctgacctcaggtgatccgcccaccacggcctcccaaagtgctgggattacaggtgtgaaccaccacacccatccagggggctcagctttgaatgaagccccctccatattaatgcagaaatgggacctaggaaccaggcagatgtgggttcaaatcccagccctgtctgacctgccgagtaaccttggatgaatgacttcccctctctggccttgggttctcttggcttgtaacgtgaggcctctgggggagatgtcagtggggccagctggtgactggcatatgctgaatgtcgatgcgtcttcctcctttcaggatgtcagcttgcactgctcagaatcaggagttacagaggaaagtcttgcatctcgagaagcaaaacctgtgagtctgggtccagctggggcagaggacaggggaagcagccccagagtccctttgcaggaagagcagagagcccatgtgatggcagaatgacatagggaataagagttttacctatgggccgcgtgcagtggctcacgcctgtaatctcagcactttgggaggccgaggcgggtggatcacctgaggtcaggagttcaaaaccagcctggccaacatggtgaaaccccgtctctactaaaaatacaaaaaaattagccaggcgtggtggcgggcacctgcagtcccagctactcgagaggctgaggcaggagaattgcttgaactcgggaggcggaggttgccgtgagccgagatcgcaccactgcactccagcctgggtgacagagcaaggctctgtctcaaaaataaataaataaggcctggtgtggtggctcacgcctgtaatcccagcactttgggaggctgaggtgggcggatcacaaggtcgggagatcaagaccatcctggctaacacggtgaaaccccgtctctactaaaaatacaaaaaaaattagctgggtgtggtggtgggcgcctgtagtcccagctactcgggaggctgaggcaggagaatggtgtgaacctgggaggcggagcttgcagtgagctgccatcccaccactcactccagcctgggcgatggagcaagactctgtctcaaaataaataaataaataaataaacaaataaataaataaataaaataaataataaagagccagaggcccttgaagaatggatggaatttggactttagcggggctgggggaccccggaaatggacgagaagcagaaccgaggccctttagggctcagcggaggcctgcctgtctctctaaggtccctcttggagcaactgaagaaactccaggccattgtggtgcagtccaccagcaagtcagcccagacaggcacctgtgtcgcagtgagtcctggtgcccccaggcaagccggggacctaggcttctgtagaggggcccatagggaggtgacaatgagtccaagctctccttgtgccccagctcaagtatgatccagtctggtctttggggcctcagtttccctgcctgtgggatggagatgcttgcaggggaggggagggagggggtgactctgccgctgtctccaccaggtcctgttgctgtcctttgccctcatcatcctcccctccatcagcccttttggccccaacaaaaccgagagccctggggactttgcgcctgtacgaggtaggggatccccacctttgaaacccttgtctggtctccccaagtccccgtcctgggcctctgggggagggggagaagacccctgtgcccttgttccctgaggctgggggccagggaagggagcataggaccccacctccaccttgtcccctgtgatgccccccttccccaatcagtgttctccagaactttgcacaacgatgctgcctcccgcgtggctgctgatgctgtgccaggctccgaggccccaggaccccgacccgaggctgacacaacccgagaagagtctccaggaagccccggggcagactggggcttccaggacaccgcgaacctgaccaattcgacggaggagctggacaacgccaccctggtcctgaggaatgcaacagaggggctgggccaggtcgccctgctggactgggtggcgcctgggccgagcactggctcaggacgtgcagggctggaggcggcgggagacgagctgtgagccccgccaggactatgctcccaggcccctctgcccaggggtgccttggggatgctgcactgggcagctacccacctggggatgggacgtgaggccaagaccccagcagagatgccagaatgggggaggcacagctcatagccacacacccagggcctgactgaggcccacgcaggaaccgacactcagacacaaggcaaagagggccacaggacccgggaaatacacacagagccaggagcagaagcaaagagcagacacacatacagcctgaaacagacctggacagacagacacagcctgaaacagacccggacagacagacacagcctgaaacagacccagacaaacagacagacagacacagcctgaaacagacccagacagacagacagacagcctgaaacagacccagacacagcctgaaacagatccggacagacagacagaaacagcctgaaacagacccagacagacagacagacacagcctgaaacagacccggacagacagacagacacagcctgaaacagacccggacagacagacagacacagcctgaaacagacccggacagacagacagacacagcctgaaacagacctagacagacagacacagattgaaacagacccagacaaacagacagacacagcctgaaacagacccagacacagcctgaaacagacccggacagacagacagacacagcctgaaacagacccagacagacagacagacacagcctgaaacagacccagacagacagaccgacgcagcctgaaagagacccagacagagagacaggcagacacagcctaaaacagacctggacagacaggcagacgtagtctgaaacagacctgaacagacagacagacgcacacacacaacagatgcgcagcaactccccgcccagggacccctcccggcctccctcgcacactgggaggaggaagccgccgagactgcagggagcctggccccggagccccgggtgcgccctggtctttggagcagccacggcccacaatcaccccccttttctaagactgcctgatccgaaataaagtattttgacagaa

SEQ ID NO: 3 - RNA, 2588 nt, Homo sapiens
  source 1..2588: mol_type = genomic RNA
gacagagccacagagggctgtgagcttgcccggccccaggtaacgctggcggtgggtgggcctccagcttggagcagagaccccccgaggcatctgcagacagaactggatggacccatgaatacggatttagctgctggaaagatggcttctgctgcctgctccatggaccccatcgacagctttgagctcctggatctcctgtttgaccggcaggacggcatcctgagacacgtggagctgggcgagggctggggtcacgtcaaggaccagcaggtcctgccaaaccccgactctgacgacttcctcagctccatcctgggctctggagactcactgcccagctccccactctggtcccccgaaggcagtgatagtggcatctccgaagacctcccctccgacccccaggacacccctccacgcagcggaccagccacctcccccgccggctgccatcctgcccagcctggcaaggggccctgcctctcctatcatcctggcaactcttgctccaccacaaccccagggccagtgatccaagtacctgaagcctctgtgaccatagacctggaaatgtggagcccaggaggaaggatctgtgctgagaagccggctgatccggtggacctgtccccacgatgcaatctcaccgtgaaagacctcctcctttcgggcagcagtggggacctgcaacagcatcacctgggggcctcctacctcctgcgacctggggctgggcactgtcaggagctggtgctcaccgaggatgagaagaagctgctggctaaagaaggcatcaccctgcccactcagctgcccctcactaagtacgaggagcgagtgctgaaaaaaatccgccggaaaatccggaacaagcagtcggcgcaagaaagcaggaagaagaagaaggaatatatcgatggcctggagactcggatgtcagcttgcactgctcagaatcaggagttacagaggaaagtcttgcatctcgagaagcaaaacctgtccctcttggagcaactgaagaaactccaggccattgtggtgcagtccaccagcaagtcagcccagacaggcacctgtgtcgcagtcctgttgctgtcctttgccctcatcatcctcccctccatcagcccttttggccccaacaaaaccgagagccctggggactttgcgcctgtacgagtgttctccagaactttgcacaacgatgctgcctcccgcgtggctgctgatgctgtgccaggctccgaggccccaggaccccgacccgaggctgacacaacccgagaagagtctccaggaagccccggggcagactggggcttccaggacaccgcgaacctgaccaattcgacggaggagctggacaacgccaccctggtcctgaggaatgcaacagaggggctgggccaggtcgccctgctggactgggtggcgcctgggccgagcactggctcaggacgtgcagggctggaggcggcgggagacgagctgtgagccccgccaggactatgctcccaggcccctctgcccaggggtgccttggggatgctgcactgggcagctacccacctggggatgggacgtgaggccaagaccccagcagagatgccagaatgggggaggcacagctcatagccacacacccagggcctgactgaggcccacgcaggaaccgacactcagacacaaggcaaagagggccacaggacccgggaaatacacacagagccaggagcagaagcaaagagcagacacacatacagcctgaaacagacctggacagacagacacagcctgaaacagacccggacagacagacacagcctgaaacagacccagacaaacagacagacagacacagcctgaaacagacccagacagacagacagacagcctgaaacagacccagacacagcctgaaacagatccggacagacagacagaaacagcctgaaacagacccagacagacagacagacacagcctgaaacagacccggacagacagacagacacagcctgaaacagacccggacagacagacagacacagcctgaaacagacccggacagacagacagacacagcctgaaacagacctagacagacagacacagattgaaacagacccagacaaacagacagacacagcctgaaacagacccagacacagcctgaaacagacccggacagacagacagacacagcctgaaacagacccagacagacagacagacacagcctgaaacagacccagacagacagaccgacgcagcctgaaagagacccagacagagagacaggcagacacagcctaaaacagacctggacagacaggcagacgtagtctgaaacagacctgaacagacagacagacgcacacacacaacagatgcgcagcaactccccgcccagggacccctcccggcctccctcgcacactgggaggaggaagccgccgagactgcagggagcctggccccggagccccgggtgcgccctggtctttggagcagccacggcccacaatcaccccccttttctaagactgcctgatccgaaataaagtattttgacagaa

SEQ ID NO: 4 - RNA, 1491 nt, Homo sapiens
  source 1..1491: mol_type = genomic RNA
acagagggctgtgagcttgcccggccccaggtaacgctggcggtgggtgggcctccagcttggagcagagaccccccgaggcatctgcagacagaactggatggacccatgaatacggatttagctgctggaaagatggcttctgctgcctgctccatggaccccatcgacagctttgagctcctggatctcctgtttgaccggcaggacggcatcctgagacacgtggagctgggcgagggctggggtcacgtcaaggaccaggtcctgccaaaccccgactctgacgacttcctcagctccatcctgggctctggagactcactgcccagctccccactctggtcccccgaaggcagtgatagtggcatctccgaagacctcccctccgacccccaggacacccctccacgcagcggaccagccacctcccccgccggctgccatcctgcccagcctggcaaggggccctgcctctcctatcatcctggcaactcttgctccaccacaaccccagggccagtgatccaagtacctgaagcctctgtgaccatagacctggaaatgtggagcccaggaggaaggatctgtgctgagaagccggctgatccggtggacctgtccccacgatgcaatctcaccgtgaaagacctcctcctttcgggcagcagtggggacctgcaacagcatcacctgggggcctcctacctcctgcgacctggggctgggcactgtcaggagctggtgctcaccgaggatgagaagaagctgctggctaaagaaggcatcaccctgcccactcagctgcccctcactaagtacgaggagcgagtgctgaaaaaaatccgccggaaaatccggaacaagcagtcggcgcaagaaagcaggaagaagaagaaggaatatatcgatggcctggagactcggatgtcagcttgcactgctcagaatcaggagttacagaggaaagtcttgcatctcgagaagcaaaacctgtccctcttggagcaactgaagaaactccaggccattgtggtgcagtccaccagcaagtcagcccagacaggcacctgtgtcgcagtcctgttgctgtcctttgccctcatcatcctcccctccatcagcccttttggccccaacaaaaccgagagccctggggactttgcgcctgtacgagtgttctccagaactttgcacaacgatgctgcctcccgcgtggctgctgatgctgtgccaggctccgaggccccaggaccccgacccgaggctgacacaacccgagaagagtctccaggaagccccggggcagactggggcttccaggacaccgcgaacctgaccaattcgacggaggagctggacaacgccaccctggtcctgaggaatgcaacagaggggctgggccaggtcgccctgctggactgggtggcgcctgggccgagcactggctcaggacgtgcagggctggaggcggcgggagacgagctgtga

SEQ ID NO: 5 - RNA, 1475 nt, Homo sapiens
  source 1..1475: mol_type = genomic RNA
aggtaacgctggcggtgggtgggcctccagcttggagcagagaccccccgaggcatctgcagacagaactggatggacccatgaatacggatttagctgctggaaagatggcttctgctgcctgctccatggaccccatcgacagctttgagctcctggatctcctgtttgaccggcaggacggcatcctgagacacgtggagctgggcgagggctggggtcacgtcaaggaccagcaggtcctgccaaaccccgactctgacgacttcctcagctccatcctgggctctggagactcactgcccagctccccactctggtcccccgaaggcagtgatagtggcatctccgaagacctcccctccgacccccaggacacccctccacgcagcggaccagccacctcccccgccggctgccatcctgcccagcctggcaaggggccctgcctctcctatcatcctggcaactcttgctccaccacaaccccagggccagtgatccaagtacctgaagcctctgtgaccatagacctggaaatgtggagcccaggaggaaggatctgtgctgagaagccggctgatccggtggacctgtccccacgatgcaatctcaccgtgaaagacctcctcctttcgggcagcagtggggacctgcatcacctgggggcctcctacctcctgcgacctggggctgggcactgtcaggagctggtgctcaccgaggatgagaagaagctgctggctaaagaaggcatcaccctgcccactcagctgcccctcactaagtacgaggagcgagtgctgaaaaaaatccgccggaaaatccggaacaagcagtcggcgcaagaaagcaggaagaagaagaaggaatatatcgatggcctggagactcggatgtcagcttgcactgctcagaatcaggagttacagaggaaagtcttgcatctcgagaagcaaaacctgtccctcttggagcaactgaagaaactccaggccattgtggtgcagtccaccagcaagtcagcccagacaggcacctgtgtcgcagtcctgttgctgtcctttgccctcatcatcctcccctccatcagcccttttggccccaacaaaaccgagagccctggggactttgcgcctgtacgagtgttctccagaactttgcacaacgatgctgcctcccgcgtggctgctgatgctgtgccaggctccgaggccccaggaccccgacccgaggctgacacaacccgagaagagtctccaggaagccccggggcagactggggcttccaggacaccgcgaacctgaccaattcgacggaggagctggacaacgccaccctggtcctgaggaatgcaacagaggggctgggccaggtcgccctgctggactgggtggcgcctgggccgagcactggctcaggacgtgcagggctggaggcggcgggagacgagctgtgagccccgccaggacta

SEQ ID NO: 6 - RNA, 1382 nt, Homo sapiens
  source 1..1382: mol_type = genomic RNA
aggtaacgctggcggtgggtgggcctccagcttggagcagagaccccccgaggcatctgcagacagaactggatggacccatgaatacggatttagctgctggaaagatggcttctgctgcctgctccatggaccccatcgacagctttgagctcctggatctcctgtttgaccggcaggacggcatcctgagacacgtggagctgggcgagggctggggtcacgtcaaggaccagcaggtcctgccaaaccccgactctgacgacttcctcagctccatcctgggctctggagactcactgcccagctccccactctggtcccccgaaggcagtgatagtggcatctccgaagacctcccctccgacccccaggacacccctccacgcagcggaccagccacctcccccgccggctgccatcctgcccagcctggcaaggggccctgcctctcctatcatcctggcaactcttgctccaccacaaccccagggccagtgatccaagtacctgaagcctctgtgaccatagacctggaaatgtggagcccaggaggaaggatctgtgctgagaagccggctgatccggtggacctgtccccacgatgcaatctcaccgtgaaagacctcctcctttcgggcagcagtggggacctgcaacagcatcacctgggggcctcctacctcctgcgacctggggctgggcactgtcaggagctggtgctcaccgaggatgagaagaagctgctggctaaagaaggcatcaccctgcccactcagctgcccctcactaaggatgtcagcttgcactgctcagaatcaggagttacagaggaaagtcttgcatctcgagaagcaaaacctgtccctcttggagcaactgaagaaactccaggccattgtggtgcagtccaccagcaagtcagcccagacaggcacctgtgtcgcagtcctgttgctgtcctttgccctcatcatcctcccctccatcagcccttttggccccaacaaaaccgagagccctggggactttgcgcctgtacgagtgttctccagaactttgcacaacgatgctgcctcccgcgtggctgctgatgctgtgccaggctccgaggccccaggaccccgacccgaggctgacacaacccgagaagagtctccaggaagccccggggcagactggggcttccaggacaccgcgaacctgaccaattcgacggaggagctggacaacgccaccctggtcctgaggaatgcaacagaggggctgggccaggtcgccctgctggactgggtggcgcctgggccgagcactggctcaggacgtgcagggctggaggcggcgggagacgagctgtgagccccgccaggactatgctccca

SEQ ID NO: 7 - RNA, 2585 nt, Homo sapiens
  source 1..2585: mol_type = genomic RNA
gacagagccacagagggctgtgagcttgcccggccccaggtaacgctggcggtgggtgggcctccagcttggagcagagaccccccgaggcatctgcagacagaactggatggacccatgaatacggatttagctgctggaaagatggcttctgctgcctgctccatggaccccatcgacagctttgagctcctggatctcctgtttgaccggcaggacggcatcctgagacacgtggagctgggcgagggctggggtcacgtcaaggaccaggtcctgccaaaccccgactctgacgacttcctcagctccatcctgggctctggagactcactgcccagctccccactctggtcccccgaaggcagtgatagtggcatctccgaagacctcccctccgacccccaggacacccctccacgcagcggaccagccacctcccccgccggctgccatcctgcccagcctggcaaggggccctgcctctcctatcatcctggcaactcttgctccaccacaaccccagggccagtgatccaagtacctgaagcctctgtgaccatagacctggaaatgtggagcccaggaggaaggatctgtgctgagaagccggctgatccggtggacctgtccccacgatgcaatctcaccgtgaaagacctcctcctttcgggcagcagtggggacctgcaacagcatcacctgggggcctcctacctcctgcgacctggggctgggcactgtcaggagctggtgctcaccgaggatgagaagaagctgctggctaaagaaggcatcaccctgcccactcagctgcccctcactaagtacgaggagcgagtgctgaaaaaaatccgccggaaaatccggaacaagcagtcggcgcaagaaagcaggaagaagaagaaggaatatatcgatggcctggagactcggatgtcagcttgcactgctcagaatcaggagttacagaggaaagtcttgcatctcgagaagcaaaacctgtccctcttggagcaactgaagaaactccaggccattgtggtgcagtccaccagcaagtcagcccagacaggcacctgtgtcgcagtcctgttgctgtcctttgccctcatcatcctcccctccatcagcccttttggccccaacaaaaccgagagccctggggactttgcgcctgtacgagtgttctccagaactttgcacaacgatgctgcctcccgcgtggctgctgatgctgtgccaggctccgaggccccaggaccccgacccgaggctgacacaacccgagaagagtctccaggaagccccggggcagactggggcttccaggacaccgcgaacctgaccaattcgacggaggagctggacaacgccaccctggtcctgaggaatgcaacagaggggctgggccaggtcgccctgctggactgggtggcgcctgggccgagcactggctcaggacgtgcagggctggaggcggcgggagacgagctgtgagccccgccaggactatgctcccaggcccctctgcccaggggtgccttggggatgctgcactgggcagctacccacctggggatgggacgtgaggccaagaccccagcagagatgccagaatgggggaggcacagctcatagccacacacccagggcctgactgaggcccacgcaggaaccgacactcagacacaaggcaaagagggccacaggacccgggaaatacacacagagccaggagcagaagcaaagagcagacacacatacagcctgaaacagacctggacagacagacacagcctgaaacagacccggacagacagacacagcctgaaacagacccagacaaacagacagacagacacagcctgaaacagacccagacagacagacagacagcctgaaacagacccagacacagcctgaaacagatccggacagacagacagaaacagcctgaaacagacccagacagacagacagacacagcctgaaacagacccggacagacagacagacacagcctgaaacagacccggacagacagacagacacagcctgaaacagacccggacagacagacagacacagcctgaaacagacctagacagacagacacagattgaaacagacccagacaaacagacagacacagcctgaaacagacccagacacagcctgaaacagacccggacagacagacagacacagcctgaaacagacccagacagacagacagacacagcctgaaacagacccagacagacagaccgacgcagcctgaaagagacccagacagagagacaggcagacacagcctaaaacagacctggacagacaggcagacgtagtctgaaacagacctgaacagacagacagacgcacacacacaacagatgcgcagcaactccccgcccagggacccctcccggcctccctcgcacactgggaggaggaagccgccgagactgcagggagcctggccccggagccccgggtgcgccctggtctttggagcagccacggcccacaatcaccccccttttctaagactgcctgatccgaaataaagtattttgacagaa

SEQ ID NO: 8 - RNA, 2582 nt, Homo sapiens
  source 1..2582: mol_type = genomic RNA
gacagagccacagagggctgtgagcttgcccggccccaggtaacgctggcggtgggtgggcctccagcttggagcagagaccccccgaggcatctgcagacagaactggatggacccatgaatacggatttagctgctggaaagatggcttctgctgcctgctccatggaccccatcgacagctttgagctcctggatctcctgtttgaccggcaggacggcatcctgagacacgtggagctgggcgagggctggggtcacgtcaaggaccagcaggtcctgccaaaccccgactctgacgacttcctcagctccatcctgggctctggagactcactgcccagctccccactctggtcccccgaaggcagtgatagtggcatctccgaagacctcccctccgacccccaggacacccctccacgcagcggaccagccacctcccccgccggctgccatcctgcccagcctggcaaggggccctgcctctcctatcatcctggcaactcttgctccaccacaaccccagggccagtgatccaagtacctgaagcctctgtgaccatagacctggaaatgtggagcccaggaggaaggatctgtgctgagaagccggctgatccggtggacctgtccccacgatgcaatctcaccgtgaaagacctcctcctttcgggcagcagtggggacctgcatcacctgggggcctcctacctcctgcgacctggggctgggcactgtcaggagctggtgctcaccgaggatgagaagaagctgctggctaaagaaggcatcaccctgcccactcagctgcccctcactaagtacgaggagcgagtgctgaaaaaaatccgccggaaaatccggaacaagcagtcggcgcaagaaagcaggaagaagaagaaggaatatatcgatggcctggagactcggatgtcagcttgcactgctcagaatcaggagttacagaggaaagtcttgcatctcgagaagcaaaacctgtccctcttggagcaactgaagaaactccaggccattgtggtgcagtccaccagcaagtcagcccagacaggcacctgtgtcgcagtcctgttgctgtcctttgccctcatcatcctcccctccatcagcccttttggccccaacaaaaccgagagccctggggactttgcgcctgtacgagtgttctccagaactttgcacaacgatgctgcctcccgcgtggctgctgatgctgtgccaggctccgaggccccaggaccccgacccgaggctgacacaacccgagaagagtctccaggaagccccggggcagactggggcttccaggacaccgcgaacctgaccaattcgacggaggagctggacaacgccaccctggtcctgaggaatgcaacagaggggctgggccaggtcgccctgctggactgggtggcgcctgggccgagcactggctcaggacgtgcagggctggaggcggcgggagacgagctgtgagccccgccaggactatgctcccaggcccctctgcccaggggtgccttggggatgctgcactgggcagctacccacctggggatgggacgtgaggccaagaccccagcagagatgccagaatgggggaggcacagctcatagccacacacccagggcctgactgaggcccacgcaggaaccgacactcagacacaaggcaaagagggccacaggacccgggaaatacacacagagccaggagcagaagcaaagagcagacacacatacagcctgaaacagacctggacagacagacacagcctgaaacagacccggacagacagacacagcctgaaacagacccagacaaacagacagacagacacagcctgaaacagacccagacagacagacagacagcctgaaacagacccagacacagcctgaaacagatccggacagacagacagaaacagcctgaaacagacccagacagacagacagacacagcctgaaacagacccggacagacagacagacacagcctgaaacagacccggacagacagacagacacagcctgaaacagacccggacagacagacagacacagcctgaaacagacctagacagacagacacagattgaaacagacccagacaaacagacagacacagcctgaaacagacccagacacagcctgaaacagacccggacagacagacagacacagcctgaaacagacccagacagacagacagacacagcctgaaacagacccagacagacagaccgacgcagcctgaaagagacccagacagagagacaggcagacacagcctaaaacagacctggacagacaggcagacgtagtctgaaacagacctgaacagacagacagacgcacacacacaacagatgcgcagcaactccccgcccagggacccctcccggcctccctcgcacactgggaggaggaagccgccgagactgcagggagcctggccccggagccccgggtgcgccctggtctttggagcagccacggcccacaatcaccccccttttctaagactgcctgatccgaaataaagtattttgacagaa

SEQ ID NO: 9 - RNA, 2481 nt, Homo sapiens
  source 1..2481: mol_type = genomic RNA
gacagagccacagagggctgtgagcttgcccggccccaggtaacgctggcggtgggtgggcctccagcttggagcagagaccccccgaggcatctgcagacagaactggatggacccatgaatacggatttagctgctggaaagatggcttctgctgcctgctccatggaccccatcgacagctttgagctcctggatctcctgtttgaccggcaggacggcatcctgagacacgtggagctgggcgagggctggggtcacgtcaaggaccagcaggtcctgccaaaccccgactctgacgacttcctcagctccatcctgggctctggagactcactgcccagctccccactctggtcccccgaaggcagtgatagtggcatctccgaagacctcccctccgacccccaggacacccctccacgcagcggaccagccacctcccccgccggctgccatcctgcccagcctggcaaggggccctgcctctcctatcatcctggcaactcttgctccaccacaaccccagggccagtgatccaagtacctgaagcctctgtgaccatagacctggaaatgtggagcccaggaggaaggatctgtgctgagaagccggctgatccggtggacctgtccccacgatgcaatctcaccgtgaaagacctcctcctttcgggcagcagtggggacctgcaacagcatcacctgggggcctcctacctcctgcgacctggggctgggcactgtcaggagctggtgctcaccgaggatgagaagaagctgctggctaaagaaggcatcaccctgcccactcagctgcccctcactaaggatgtcagcttgcactgctcagaatcaggagttacagaggaaagtcttgcatctcgagaagcaaaacctgtccctcttggagcaactgaagaaactccaggccattgtggtgcagtccaccagcaagtcagcccagacaggcacctgtgtcgcagtcctgttgctgtcctttgccctcatcatcctcccctccatcagcccttttggccccaacaaaaccgagagccctggggactttgcgcctgtacgagtgttctccagaactttgcacaacgatgctgcctcccgcgtggctgctgatgctgtgccaggctccgaggccccaggaccccgacccgaggctgacacaacccgagaagagtctccaggaagccccggggcagactggggcttccaggacaccgcgaacctgaccaattcgacggaggagctggacaacgccaccctggtcctgaggaatgcaacagaggggctgggccaggtcgccctgctggactgggtggcgcctgggccgagcactggctcaggacgtgcagggctggaggcggcgggagacgagctgtgagccccgccaggactatgctcccaggcccctctgcccaggggtgccttggggatgctgcactgggcagctacccacctggggatgggacgtgaggccaagaccccagcagagatgccagaatgggggaggcacagctcatagccacacacccagggcctgactgaggcccacgcaggaaccgacactcagacacaaggcaaagagggccacaggacccgggaaatacacacagagccaggagcagaagcaaagagcagacacacatacagcctgaaacagacctggacagacagacacagcctgaaacagacccggacagacagacacagcctgaaacagacccagacaaacagacagacagacacagcctgaaacagacccagacagacagacagacagcctgaaacagacccagacacagcctgaaacagatccggacagacagacagaaacagcctgaaacagacccagacagacagacagacacagcctgaaacagacccggacagacagacagacacagcctgaaacagacccggacagacagacagacacagcctgaaacagacccggacagacagacagacacagcctgaaacagacctagacagacagacacagattgaaacagacccagacaaacagacagacacagcctgaaacagacccagacacagcctgaaacagacccggacagacagacagacacagcctgaaacagacccagacagacagacagacacagcctgaaacagacccagacagacagaccgacgcagcctgaaagagacccagacagagagacaggcagacacagcctaaaacagacctggacagacaggcagacgtagtctgaaacagacctgaacagacagacagacgcacacacacaacagatgcgcagcaactccccgcccagggacccctcccggcctccctcgcacactgggaggaggaagccgccgagactgcagggagcctggccccggagccccgggtgcgccctggtctttggagcagccacggcccacaatcaccccccttttctaagactgcctgatccgaaataaagtattttgacagaa

SEQ ID NO: 10 - RNA, 1505 nt, Homo sapiens
  source 1..1505: mol_type = genomic RNA
gtgacagagccacagagggctgtgagcttgcccagccccaggtaacgctggcggtgggtgggcctccagcttggagcagagaccccccgaggcatctgcagacagaactggatggacccatgaatacggatttagctgctggaaagatggcttctgctgcctgctccatggaccccatcgacagctttgagctcctggatctcctgtttgaccggcaggacggcatcctgagacacgtggagctgggcgagggctggggtcacgtcaaggaccagcaggtcctgccaaaccccgactctgacgacttcctcagctccatcctgggctctggagactcactgcccagctccccactctggtcccccgaaggcagtgatagtggcatctccgaagacctcccctccgacccccaggacacccctccacgcagcggaccagccacctcccccgccggctgccatcctgcccagcctggcaaggggccctgcctctcctatcatcctggcaactcttgctccaccacaaccccagggccagtgatccaagtacctgaagcctctgtgaccatagacctggaaatgtggagcccaggaggaaggatctgtgctgagaagccggctgatccggtggacctgtccccacgatgcaatctcaccgtgaaagacctcctcctttcgggcagcagtggggacctgcaacagcatcacctgggggcctcctacctcctgcgacctggggctgggcactgtcaggagctggtgctcaccgaggatgagaagaagctgctggctaaagaaggcatcaccctgcccactcagctgcccctcactaagtacgaggagcgagtgctgaaaaaaatccgccggaaaatccggaacaagcagtcggcgcaagaaagcaggaagaagaagaaggaatatatcgatggcctggagactcggatgtcagcttgcactgctcagaatcaggagttacagaggaaagtcttgcacctcgagaagcaaaacctgtccctcttggagcaactgaagaaactccaggccattgtggtgcagtccaccagcaagtcagcccagacaggcacctgtgtcgcagtcctgttgctgtcctttgccctcatcatcctcccctccatcagcccttttggccccaacaaaaccgagagccctggggactttgcgcctgtacgagtgttctccagaactttgcacaacgatgctgcctcccgcgtggctgctgatgctgtgccaggctccgaggccccaggaccccgacccgaggctgacacaacccgagaagagtctccaggaagccccggggcagactggggcttccaggacaccgcgaacctgaccaattcgacggaggagctggacaacgccaccctggtcctgaggaatgcaacagaggggctgggccaggtcgccctgctggactgggtggcgcctgggccgagcactggctcaggacgtgcagggctggaggcggcgggagacgagctgtga

SEQ ID NO: 11 - RNA, 2206 nt, Homo sapiens
  source 1..2206: mol_type = genomic RNA
acagagccacagagggctgtgagcttgcccagccccaggtaacgctggcggtgggtgggccaccagcttggagcagagaccccccgaggcatctgcagacagaactggatggacccatgaatacggatttagctgctggaaagatggcttctgctgcctgctccatggaccccatcgacagctttgagctcctggatctcctgtttgaccggcaggacggcatcctgagacacgtggagctgggcgagggctggggtcacgtcaaggaccagcaggtcctgccaaaccccgactctgacgacttcctcagctccatcctgggctctggagactcactgcccagctccccactctggtcccccgaaggcagtgatagtggcatctccgaagacctcccctccgacccccaggacacccctccacgcagcggaccagccacctcccccgccggctgccatcctgcccagcctggcaaggggccctgcctctcctatcatcctggcaactcttgctccaccacaaccccagggccagtgatccaagtacctgaagcctctgtgaccatagacctggaaatgtggagcccaggaggaaggatctgtgctgagaagccggctgatccggtggacctgtccccacgatgcaatctcaccgtgaaagacctcctcctttcgggcagcagtggggacctgcaacagcatcacctgggggcctcctacctcctgcgacctggggctgggcactgtcaggagctggtgctcaccgaggatgagaagaagctgctggctaaagaaggcatcaccctgcccactcagctgcccctcactaagtacgaggagcgagtgctgaaaaaaatccgccggaaaatccggaacaagcagtcggcgcaagaaagcaggaagaagaagaaggccattgtggtgcagtccaccagcaagtcagcccagacaggcacctgtgtcgcagtcctgttgctgtcctttgccctcatcatcctcccctccatcagcccttttggccccaacaaaaccgagagccctggggactttgcgcctgtacgagtgttctccagaactttgcacaacgatgctgcctcccgcgtggctgctgatgctgtgccaggctccgaggccccaggaccccgacccgaggctgacacaacccgagaagagtctccaggaagccccggggcagactggggcttccaggacaccgcgaacctgaccaattcgacggaggagctggacaacgccaccctggtcctgaggaatgcaacagaggggctgggccaggtcgccccgctggactgggtggcgcctgggcccgagcactggctcaggacgtgcagggctggaggcggcgggagacgagcttgtgagccccgccaggactatgctcccaggcccctctgcccaggggtgccttggggatgctgcactgggcagctacccacctggggatgggacgtgaggccaagaccccagcagagatgccagaatgggggaggcacagctcatagccacacacccagggcctgactgaggcccacgcaggaaccgacactcagacacaaggcaaagagggccacaggacccgggaaatacacacagagccaggagcagaagcaaagagcagacacacatacagcctgaaacagacctggacagacagacacagcctgaaacagacccggacagacagacacagcctgaaacagacccagacaaacagacagacagacacagcctgaaacagacccagacagacagacagacagcctgaaacagacccagacacagcctgaaacagatccggacagacagacagaaacagcctgaaacagacccagacagacagacagacacagcctgaaacagacccggacagacagacagacacagcctgaaagagacccagacagagagacaggcagacacagcctaaaacagacctggacagacaggcagacgtagtctgaaacagacctgaacagacagacagacgcacacacacaacagatgcgcagcaactccccgcccagggacccctcccggcctccctcgcacactgggaggaggaagccgccgagactgcagggagcctggccccggagccccgggtgcgccctggtctttggagcagccacggcccacaatcaccccccttttctaagactgcctgatccgaaataaagtattttgacaaa

SEQ ID NO: 12 - RNA, 2582 nt, Homo sapiens
  source 1..2582: mol_type = genomic RNA
acagagggctgtgagcttgcccggccccaggtaacgctggcggtgggtgggcctccagcttggagcagagaccccccgaggcatctgcagacagaactggatggacccatgaatacggatttagctgctggaaagatggcttctgctgcctgctccatggaccccatcgacagctttgagctcctggatctcctgtttgaccggcaggacggcatcctgagacacgtggagctgggcgagggctggggtcacgtcaaggaccaggtcctgccaaaccccgactctgacgacttcctcagctccatcctgggctctggagactcactgcccagctccccactctggtcccccgaaggcagtgatagtggcatctccgaagacctcccctccgacccccaggacacccctccacgcagcggaccagccacctcccccgccggctgccatcctgcccagcctggcaaggggccctgcctctcctatcatcctggcaactcttgctccaccacaaccccagggccagtgatccaagtacctgaagcctctgtgaccatagacctggaaatgtggagcccaggaggaaggatctgtgctgagaagccggctgatccggtggacctgtccccacgatgcaatctcaccgtgaaagacctcctcctttcgggcagcagtggggacctgcaacagcatcacctgggggcctcctacctcctgcgacctggggctgggcactgtcaggagctggtgctcaccgaggatgagaagaagctgctggctaaagaaggcatcaccctgcccactcagctgcccctcactaagtacgaggagcgagtgctgaaaaaaatccgccggaaaatccggaacaagcagtcggcgcaagaaagcaggaagaagaagaaggaatatatcgatggcctggagactcggatgtcagcttgcactgctcagaatcaggagttacagaggaaagtcttgcatctcgagaagcaaaacctgtccctcttggagcaactgaagaaactccaggccattgtggtgcagtccaccagcaagtcagcccagacaggcacctgtgtcgcagtcctgttgctgtcctttgccctcatcatcctcccctccatcagcccttttggccccaacaaaaccgagagccctggggactttgcgcctgtacgagtgttctccagaactttgcacaacgatgctgcctcccgcgtggctgctgatgctgtgccaggctccgaggccccaggaccccgacccgaggctgacacaacccgagaagagtctccaggaagccccggggcagactggggcttccaggacaccgcgaacctgaccaattcgacggaggagctggacaacgccaccctggtcctgaggaatgcaacagaggggctgggccaggtcgccctgctggactgggtggcgcctgggccgagcactggctcaggacgtgcagggctggaggcggcgggagacgagctgtgagccccgccaggactatgctcccaggcccctctgcccaggggtgccttggggatgctgcactgggcagctacccacctggggatgggacgtgaggccaagaccccagcagagatgccagaatgggggaggcacagctcatagccacacacccagggcctgactgaggcccacgcaggaaccgacactcagacacaaggcaaagagggccacaggacccgggaaatacacacagagccaggagcagaagcaaagagcagacacacatacagcctgaaacagacctggacagacagacacagcctgaaacagacccggacagacagacacagcctgaaacagacccagacaaacagacagacagacacagcctgaaacagacccagacagacagacagacagcctgaaacagacccagacacagcctgaaacagatccggacagacagacagaaacagcctgaaacagacccagacagacagacagacacagcctgaaacagacccggacagacagacagacacagcctgaaacagacccggacagacagacagacacagcctgaaacagacccggacagacagacagacacagcctgaaacagacctagacagacagacacagattgaaacagacccagacaaacagacagacacagcctgaaacagacccagacacagcctgaaacagacccggacagacagacagacacagcctgaaacagacccagacagacagacagacacagcctgaaacagacccagacagacagaccgacgcagcctgaaagagacccagacagagagacaggcagacacagcctaaaacagacctggacagacaggcagacgtagtctgaaacagacctgaacagacagacagacgcacacacacaacagatgcgcagcaactccccgcccagggacccctcccggcctccctcgcacactgggaggaggaagccgccgagactgcagggagcctggccccggagccccgggtgcgccctggtctttggagcagccacggcccacaatcaccccccttttctaagactgcctgatccgaaataaagtattttgacagaaaaaaaa

SEQ ID NO: 13 - RNA, 1489 nt, Homo sapiens
  source 1..1489: mol_type = genomic RNA
aggtaacgctggcggtgggtgggcctccagcttggagcagagaccccccgaggcatctgcagacagaactggatggacccatgaatacggatttagctgctggaaagatggcttctgctgcctgctccatggaccccatcgacagctttgagctcctggatctcctgtttgaccggcaggacggcatcctgagacacgtggagctgggcgagggctggggtcacgtcaaggaccagcaggtcctgccaaaccccgactctgacgacttcctcagctccatcctgggctctggagactcactgcccagctccccactctggtcccccgaaggcagtgatagtggcatctccgaagacctcccctccgacccccaggacacccctccacgcagcggaccagccacctcccccgccggctgccatcctgcccagcctggcaaggggccctgcctctcctatcatcctggcaactcttgctccaccacaaccccagggccagtgatccaagtacctgaagcctctgtgaccatagacctggaaatgtggagcccaggaggaaggatctgtgctgagaagccggctgatccggtggacctgtccccacgatgcaatctcaccgtgaaagacctcctcctttcgggcagcagtggggacctgcaacagcatcacctgggggcctcctacctcctgcgacctggggctgggcactgtcaggagctggtgctcaccgaggatgagaagaagctgctggctaaagaaggcatcaccctgcccactcagctgcccctcactaagtacgaggagcgagtgctgaaaaaaatccgccggaaaatccggaacaagcagtcggcgcaagaaagcaggaagaagaagaaggaatatatcgatggcctggagactcggatgtcagcttgcactgctcagaatcaggagttacagaggaaagtcttgcatctcgagaagcaaaacctgtccctcttggagcaactgaagaaactccaggccattgtggtgcagtccaccagcaagtcagcccagacaggcacctgtgtcgcagtcctgttgctgtcctttgccctcatcatcctcccctccatcagcccttttggccccaacaaaaccgagagccctggggactttgcgcctgtacgagtgttctccagaactttgcacaacgatgctgcctcccgcgtggctgctgatgctgtgccaggctccgaggccccaggaccccgacccgaggctgacacaacccgagaagagtctccaggaagccccggggcagactggggcttccaggacaccgcgaacctgaccaattcgacggaggagctggacaacgccaccctggtcctgaggaatgcaacagaggggctgggccaggtcgccctgctggactgggtggcgcctgggccgagcactggctcaggacgtgcagggctggaggcggcgggagacgagctgtgagccccgccaggactatgctccca

SEQ ID NO: 14 - RNA, 2618 nt, Homo sapiens
  source 1..2618: mol_type = genomic RNA
gtgaccgcttcagcatcttttgggagtggtgacagagccacagagggctgtgagcttgcccggccccaggtaacgctggcggtgggtgggcctccagcttggagcagagaccccccgaggcatctgcagacagaactggatggacccatgaatacggatttagctgctggaaagatggcttctgctgcctgctccatggaccccatcgacagctttgagctcctggatctcctgtttgaccggcaggacggcatcctgagacacgtggagctgggcgagggctggggtcacgtcaaggaccagcaggtcctgccaaaccccgactctgacgacttcctcagctccatcctgggctctggagactcactgcccagctccccactctggtcccccgaaggcagtgatagtggcatctccgaagacctcccctccgacccccaggacacccctccacgcagcggaccagccacctcccccgccggctgccatcctgcccagcctggcaaggggccctgcctctcctatcatcctggcaactcttgctccaccacaaccccagggccagtgatccaagtacctgaagcctctgtgaccatagacctggaaatgtggagcccaggaggaaggatctgtgctgagaagccggctgatccggtggacctgtccccacgatgcaatctcaccgtgaaagacctcctcctttcgggcagcagtggggacctgcaacagcatcacctgggggcctcctacctcctgcgacctggggctgggcactgtcaggagctggtgctcaccgaggatgagaagaagctgctggctaaagaaggcatcaccctgcccactcagctgcccctcactaagtacgaggagcgagtgctgaaaaaaatccgccggaaaatccggaacaagcagtcggcgcaagaaagcaggaagaagaagaaggaatatatcgatggcctggagactcggatgtcagcttgcactgctcagaatcaggagttacagaggaaagtcttgcatctcgagaagcaaaacctgtccctcttggagcaactgaagaaactccaggccattgtggtgcagtccaccagcaagtcagcccagacaggcacctgtgtcgcagtcctgttgctgtcctttgccctcatcatcctcccctccatcagcccttttggccccaacaaaaccgagagccctggggactttgcgcctgtacgagtgttctccagaactttgcacaacgatgctgcctcccgcgtggctgctgatgctgtgccaggctccgaggccccaggaccccgacccgaggctgacacaacccgagaagagtctccaggaagccccggggcagactggggcttccaggacaccgcgaacctgaccaattcgacggaggagctggacaacgccaccctggtcctgaggaatgcaacagaggggctgggccaggtcgccctgctggactgggtggcgcctgggccgagcactggctcaggacgtgcagggctggaggcggcgggagacgagctgtgagccccgccaggactatgctcccaggcccctctgcccaggggtgccttggggatgctgcactgggcagctacccacctggggatgggacgtgaggccaagaccccagcagagatgccagaatgggggaggcacagctcatagccacacacccagggcctgactgaggcccacgcaggaaccgacactcagacacaaggcaaagagggccacaggacccgggaaatacacacagagccaggagcagaagcaaagagcagacacacatacagcctgaaacagacctggacagacagacacagcctgaaacagacccggacagacagacacagcctgaaacagacccagacaaacagacagacagacacagcctgaaacagacccagacagacagacagacagcctgaaacagacccagacacagcctgaaacagatccggacagacagacagaaacagcctgaaacagacccagacagacagacagacacagcctgaaacagacccggacagacagacagacacagcctgaaacagacccggacagacagacagacacagcctgaaacagacccggacagacagacagacacagcctgaaacagacctagacagacagacacagattgaaacagacccagacaaacagacagacacagcctgaaacagacccagacacagcctgaaacagacccggacagacagacagacacagcctgaaacagacccagacagacagacagacacagcctgaaacagacccagacagacagaccgacgcagcctgaaagagacccagacagagagacaggcagacacagcctaaaacagacctggacagacaggcagacgtagtctgaaacagacctgaacagacagacagacgcacacacacaacagatgcgcagcaactccccgcccagggacccctcccggcctccctcgcacactgggaggaggaagccgccgagactgcagggagcctggccccggagccccgggtgcgccctggtctttggagcagccacggcccacaatcaccccccttttctaagactgcctgatccgaaataaagtattttgacagaa

SEQ ID NO: 15 - RNA, 2586 nt, Homo sapiens
  source 1..2586: mol_type = genomic RNA
acagagccacagagggctgtgagcttgcccagccccaggtaacgctggcggtgggtgggcctccagcttggagcagagaccccccgaggcatctgcagacagaactggatggacccatgaatacggatttagctgctggaaagatggcttctgctgcctgctccatggaccccatcgacagctttgagctcctggatctcctgtttgaccggcaggacggcatcctgagacacgtggagctgggcgagggctggggtcacgtcaaggaccagcaggtcctgccaaaccccgactctgacgacttcctcagctccatcctgggctctggagactcactgcccagctccccactctggtcccccgaaggcagtgatagtggcatctccgaagacctcccctccgacccccaggacacccctccacgcagcggaccagccacctcccccgccggctgccatcctgcccagcctggcaaggggccctgcctctcctatcatcctggcaactcttgctccaccacaaccccagggccagtgatccaagtacctgaagcctctgtgaccatagacctggaaatgtggagcccaggaggaaggatctgtgctgagaagccggctgatccggtggacctgtccccacgatgcaatctcaccgtgaaagacctcctcctttcgggcagcagtggggacctgcaacagcatcacctgggggcctcctacctcctgcgacctggggctgggcactgtcaggagctggtgctcaccgaggatgagaagaagctgctggctaaagaaggcatcaccctgcccactcagctgcccctcactaagtacgaggagcgagtgctgaaaaaaatccgccggaaaatccggaacaagcagtcggcgcaagaaagcaggaagaagaagaaggaatatatcgatggcctggagactcggatgtcagcttgcactgctcagaatcaggagttacagaggaaagtcttgcatctcgagaagcaaaacctgtccctcttggagcaactgaagaaactccaggccattgtggtgcagtccaccagcaagtcagcccagacaggcacctgtgtcgcagtcctgttgctgtcctttgccctcatcatcctcccctccatcagcccttttggccccaacaaaaccgagagccctggggactttgcgcctgtacgagtgttctccagaactttgcacaacgatgctgcctcccgcgtggctgctgatgctgtgccaggctccgaggccccaggaccccgacccgaggctgacacaacccgagaagagtctccaggaagccccggggcagactggggcttccaggacaccgcgaacctgaccaattcgacggaggagctggacaacgccaccctggtcctgaggaatgcaacagaggggctgggccaggtcgccctgctggactgggtggcgcctgggccgagcactggctcaggacgtgcagggctggaggcggcgggagacgagctgtgagccccgccaggactatgctcccaggcccctctgcccaggggtgccttggggatgctgcactgggcagctacccacctggggatgggacgtgaggccaagaccccagcagagatgccagaatgggggaggcacagctcatagccacacacccagggcctgactgaggcccacgcaggaaccgacactcagacacaaggcaaagagggccacaggacccgggaaatacacacagagccaggagcagaagcaaagagcagacacacatacagcctgaaacagacctggacagacagacacagcctgaaacagacccggacagacagacacagcctgaaacagacccagacaaacagacagacagacacagcctgaaacagacccagacagacagacagacagcctgaaacagacccagacacagcctgaaacagatccggacagacagacagaaacagcctgaaacagacccagacagacagacagacacagcctgaaacagacccggacagacagacagacacagcctgaaacagacccggacagacagacagacacagcctgaaacagacccggacagacagacagacacagcctgaaacagacctagacagacagacacagattgaaacagacccagacaaacagacagacacagcctgaaacagacccagacacagcctgaaacagacccggacagacagacagacacagcctgaaacagacccagacagacagacagacacagcctgaaacagacccagacagacagaccgacgcagcctgaaagagacccagacagagagacaggcagacacagcctaaaacagacctggacagacaggcagacgtagtctgaaacagacctgaacagacagacagacgcacacacacaacagatgcgcagcaactccccgcccagggacccctcccggcctccctcgcacactgggaggaggaagccgccgagactgcagggagcctggccccggagccccgggtgcgccctggtctttggagcagccacggcccacaatcaccccccttttctaagactgcctgatccgaaataaagtattttgacaaa

SEQ ID NO: 16 - RNA, 1485 nt, Homo sapiens
  source 1..1485: mol_type = genomic RNA
aacgctggcggtgggtgggcctccagcttggagcagagaccccccgaggcatctgcagacagaactggatggacccatgaatacggatttagctgctggaaagatggcttctgctgcctgctccatggaccccatcgacagctttgagctcctggatctcctgtttgaccggcaggacggcatcctgagacacgtggagctgggcgagggctggggtcacgtcaaggaccagcaggtcctgccaaaccccgactctgacgacttcctcagctccatcctgggctctggagactcactgcccagctccccactctggtcccccgaaggcagtgatagtggcatctccgaagacctcccctccgacccccaggacacccctccacgcagcggaccagccacctcccccgccggctgccatcctgcccagcctggcaaggggccctgcctctcctatcatcctggcaactcttgctccaccacaaccccagggccagtgatccaagtacctgaagcctctgtgaccatagacctggaaatgtggagcccaggaggaaggatctgtgctgagaagccggctgatccggtggacctgtccccacgatgcaatctcaccgtgaaagacctcctcctttcgggcagcagtggggacctgcaacagcatcacctgggggcctcctacctcctgcgacctggggctgggcactgtcaggagctggtgctcaccgaggatgagaagaagctgctggctaaagaaggcatcaccctgcccactcagctgcccctcactaagtacgaggagcgagtgctgaaaaaaatccgccggaaaatccggaacaagcagtcggcgcaagaaagcaggaagaagaagaaggaatatatcgatggcctggagactcggatgtcagcttgcactgctcagaatcaggagttacagaggaaagtcttgcatctcgagaagcaaaacctgtccctcttggagcaactgaagaaactccaggccattgtggtgcagtccaccagcaagtcagcccagacaggcacctgtgtcgcagtcctgttgctgtcctttgccctcatcatcctcccctccatcagcccttttggccccaacaaaaccgagagccctggggactttgcgcctgtacgagtgttctccagaactttgcacaacgatgctgcctcccgcgtggctgctgatgctgtgccaggctccgaggccccaggaccccgacccgaggctgacacaacccgagaagagtctccaggaagccccggggcagactggggcttccaggacaccgcgaacctgaccaattcgacggaggagctggacaacgccaccctggtcctgaggaatgcaacagaggggctgggccaggtcgccctgctggactgggtggcgcctgggccgagcactggctcaggacgtgcagggctggaggcggcgggagacgagctgtgagccccgccaggactatgctccca

SEQ ID NO: 17 - RNA, 2588 nt, Homo sapiens
  source 1..2588: mol_type = genomic RNA
gacagagccacagagggctgtgagcttgcccggccccaggtaacgctggcggtgggtgggcctccagcttggagcagagaccccccgaggcatctgcagacagaactggatggacccatgaatacggatttagctgctggaaagatggcttctgctgcctgctccatggaccccatcgacagctttgagctcctggatctcctgtttgaccggcaggacggcatcctgagacacgtggagctgggcgagggctggggtcacgtcaaggaccagcaggtcctgccaaaccccgactctgacgacttcctcagctccatcctgggctctggagactcactgcccagctccccactctggtcccccgaaggcagtgatagtggcatctccgaagacctcccctccgacccccaggacacccctccacgcagcggaccagccacctcccccgccggctgccatcctgcccagcctggcaaggggccctgcctctcctatcatcctggcaactcttgctccaccacaaccccagggccagtgatccaagtacctgaagcctctgtgaccatagacctggaaatgtggagcccaggaggaaggatctgtgctgagaagccggctgatccggtggacctgtccccacgatgcaatctcaccgtgaaaaacctcctcctttcgggcagcagtggggacctgcaacagcatcacctgggggcctcctacctcctgcgacctggggctgggcactgtcaggagctggtgctcaccgaggatgagaagaagctgctggctaaagaaggcatcaccctgcccactcagctgcccctcactaagtacgaggagcgagtgctgaaaaaaatccgccggaaaatccggaacaagcagtcggcgcaagaaagcaggaagaagaagaaggaatatatcgatggcctggagactcggatgtcagcttgcactgctcagaatcaggagttacagaggaaagtcttgcatctcgagaagcaaaacctgtccctcttggagcaactgaagaaactccaggccattgtggtgcagtccaccagcaagtcagcccagacaggcacctgtgtcgcagtcctgttgctgtcctttgccctcatcatcctcccctccatcagcccttttggccccaacaaaaccgagagccctggggactttgcgcctgtacgagtgttctccagaactttgcacaacgatgctgcctcccgcgtggctgctgatgctgtgccaggctccgaggccccaggaccccgacccgaggctgacacaacccgagaagagtctccaggaagccccggggcagactggggcttccaggacaccgcgaacctgaccaattcgacggaggagctggacaacgccaccctggtcctgaggaatgcaacagaggggctgggccaggtcgccctgctggactgggtggcgcctgggccgagcactggctcaggacgtgcagggctggaggcggcgggagacgagctgtgagccccgccaggactatgctcccaggcccctctgcccaggggtgccttggggatgctgcactgggcagctacccacctggggatgggacgtgaggccaagaccccagcagagatgccagaatgggggaggcacagctcatagccacacacccagggcctgactgaggcccacgcaggaaccgacactcagacacaaggcaaagagggccacaggacccgggaaatacacacagagccaggagcagaagcaaagagcagacacacatacagcctgaaacagacctggacagacagacacagcctgaaacagacccggacagacagacacagcctgaaacagacccagacaaacagacagacagacacagcctgaaacagacccagacagacagacagacagcctgaaacagacccagacacagcctgaaacagatccggacagacagacagaaacagcctgaaacagacccagacagacagacagacacagcctgaaacagacccggacagacagacagacacagcctgaaacagacccggacagacagacagacacagcctgaaacagacccggacagacagacagacacagcctgaaacagacctagacagacagacacagattgaaacagacccagacaaacagacagacacagcctgaaacagacccagacacagcctgaaacagacccggacagacagacagacacagcctgaaacagacccagacagacagacagacacagcctgaaacagacccagacagacagaccgacgcagcctgaaagagacccagacagagagacaggcagacacagcctaaaacagacctggacagacaggcagacgtagtctgaaacagacctgaacagacagacagacgcacacacacaacagatgcgcagcaactccccgcccagggacccctcccggcctccctcgcacactgggaggaggaagccgccgagactgcagggagcctggccccggagccccgggtgcgccctggtctttggagcagccacggcccacaatcaccccccttttctaagactgcctgatccgaaataaagtattttgacagaa

SEQ ID NO: 18 - RNA, 1491 nt, Homo sapiens
  source 1..1491: mol_type = genomic RNA
acagagggctgtgagcttgcccggccccaggtaacgctggcggtgggtgggcctccagcttggagcagagaccccccgaggcatctgcagacagaactggatggacccatgaatacggatttagctgctggaaagatggcttctgctgcctgctccatggaccccatcgacagctttgagctcctggatctcctgtttgaccggcaggacggcatcctgagacacgtggagctgggcgagggctggggtcacgtcaaggaccaggtcctgccaaaccccgactctgacgacttcctcagctccatcctgggctctggagactcactgcccagctccccactctggtcccccgaaggcagtgatagtggcatctccgaagacctcccctccgacccccaggacacccctccacgcagcggaccagccacctcccccgccggctgccatcctgcccagcctggcaaggggccctgcctctcctatcatcctggcaactcttgctccaccacaaccccagggccagtgatccaagtacctgaagcctctgtgaccatagacctggaaatgtggagcccaggaggaaggatctgtgctgagaagccggctgatccggtggacctgtccccacgatgcaatctcaccgtgaaaaacctcctcctttcgggcagcagtggggacctgcaacagcatcacctgggggcctcctacctcctgcgacctggggctgggcactgtcaggagctggtgctcaccgaggatgagaagaagctgctggctaaagaaggcatcaccctgcccactcagctgcccctcactaagtacgaggagcgagtgctgaaaaaaatccgccggaaaatccggaacaagcagtcggcgcaagaaagcaggaagaagaagaaggaatatatcgatggcctggagactcggatgtcagcttgcactgctcagaatcaggagttacagaggaaagtcttgcatctcgagaagcaaaacctgtccctcttggagcaactgaagaaactccaggccattgtggtgcagtccaccagcaagtcagcccagacaggcacctgtgtcgcagtcctgttgctgtcctttgccctcatcatcctcccctccatcagcccttttggccccaacaaaaccgagagccctggggactttgcgcctgtacgagtgttctccagaactttgcacaacgatgctgcctcccgcgtggctgctgatgctgtgccaggctccgaggccccaggaccccgacccgaggctgacacaacccgagaagagtctccaggaagccccggggcagactggggcttccaggacaccgcgaacctgaccaattcgacggaggagctggacaacgccaccctggtcctgaggaatgcaacagaggggctgggccaggtcgccctgctggactgggtggcgcctgggccgagcactggctcaggacgtgcagggctggaggcggcgggagacgagctgtga

SEQ ID NO: 19 - RNA, 1475 nt, Homo sapiens
  source 1..1475: mol_type = genomic RNA
aggtaacgctggcggtgggtgggcctccagcttggagcagagaccccccgaggcatctgcagacagaactggatggacccatgaatacggatttagctgctggaaagatggcttctgctgcctgctccatggaccccatcgacagctttgagctcctggatctcctgtttgaccggcaggacggcatcctgagacacgtggagctgggcgagggctggggtcacgtcaaggaccagcaggtcctgccaaaccccgactctgacgacttcctcagctccatcctgggctctggagactcactgcccagctccccactctggtcccccgaaggcagtgatagtggcatctccgaagacctcccctccgacccccaggacacccctccacgcagcggaccagccacctcccccgccggctgccatcctgcccagcctggcaaggggccctgcctctcctatcatcctggcaactcttgctccaccacaaccccagggccagtgatccaagtacctgaagcctctgtgaccatagacctggaaatgtggagcccaggaggaaggatctgtgctgagaagccggctgatccggtggacctgtccccacgatgcaatctcaccgtgaaaaacctcctcctttcgggcagcagtggggacctgcatcacctgggggcctcctacctcctgcgacctggggctgggcactgtcaggagctggtgctcaccgaggatgagaagaagctgctggctaaagaaggcatcaccctgcccactcagctgcccctcactaagtacgaggagcgagtgctgaaaaaaatccgccggaaaatccggaacaagcagtcggcgcaagaaagcaggaagaagaagaaggaatatatcgatggcctggagactcggatgtcagcttgcactgctcagaatcaggagttacagaggaaagtcttgcatctcgagaagcaaaacctgtccctcttggagcaactgaagaaactccaggccattgtggtgcagtccaccagcaagtcagcccagacaggcacctgtgtcgcagtcctgttgctgtcctttgccctcatcatcctcccctccatcagcccttttggccccaacaaaaccgagagccctggggactttgcgcctgtacgagtgttctccagaactttgcacaacgatgctgcctcccgcgtggctgctgatgctgtgccaggctccgaggccccaggaccccgacccgaggctgacacaacccgagaagagtctccaggaagccccggggcagactggggcttccaggacaccgcgaacctgaccaattcgacggaggagctggacaacgccaccctggtcctgaggaatgcaacagaggggctgggccaggtcgccctgctggactgggtggcgcctgggccgagcactggctcaggacgtgcagggctggaggcggcgggagacgagctgtgagccccgccaggacta

SEQ ID NO: 20 - RNA, 1382 nt, Homo sapiens
  source 1..1382: mol_type = genomic RNA
aggtaacgctggcggtgggtgggcctccagcttggagcagagaccccccgaggcatctgcagacagaactggatggacccatgaatacggatttagctgctggaaagatggcttctgctgcctgctccatggaccccatcgacagctttgagctcctggatctcctgtttgaccggcaggacggcatcctgagacacgtggagctgggcgagggctggggtcacgtcaaggaccagcaggtcctgccaaaccccgactctgacgacttcctcagctccatcctgggctctggagactcactgcccagctccccactctggtcccccgaaggcagtgatagtggcatctccgaagacctcccctccgacccccaggacacccctccacgcagcggaccagccacctcccccgccggctgccatcctgcccagcctggcaaggggccctgcctctcctatcatcctggcaactcttgctccaccacaaccccagggccagtgatccaagtacctgaagcctctgtgaccatagacctggaaatgtggagcccaggaggaaggatctgtgctgagaagccggctgatccggtggacctgtccccacgatgcaatctcaccgtgaaaaacctcctcctttcgggcagcagtggggacctgcaacagcatcacctgggggcctcctacctcctgcgacctggggctgggcactgtcaggagctggtgctcaccgaggatgagaagaagctgctggctaaagaaggcatcaccctgcccactcagctgcccctcactaaggatgtcagcttgcactgctcagaatcaggagttacagaggaaagtcttgcatctcgagaagcaaaacctgtccctcttggagcaactgaagaaactccaggccattgtggtgcagtccaccagcaagtcagcccagacaggcacctgtgtcgcagtcctgttgctgtcctttgccctcatcatcctcccctccatcagcccttttggccccaacaaaaccgagagccctggggactttgcgcctgtacgagtgttctccagaactttgcacaacgatgctgcctcccgcgtggctgctgatgctgtgccaggctccgaggccccaggaccccgacccgaggctgacacaacccgagaagagtctccaggaagccccggggcagactggggcttccaggacaccgcgaacctgaccaattcgacggaggagctggacaacgccaccctggtcctgaggaatgcaacagaggggctgggccaggtcgccctgctggactgggtggcgcctgggccgagcactggctcaggacgtgcagggctggaggcggcgggagacgagctgtgagccccgccaggactatgctccca

SEQ ID NO: 21 - RNA, 2585 nt, Homo sapiens
  source 1..2585: mol_type = genomic RNA
gacagagccacagagggctgtgagcttgcccggccccaggtaacgctggcggtgggtgggcctccagcttggagcagagaccccccgaggcatctgcagacagaactggatggacccatgaatacggatttagctgctggaaagatggcttctgctgcctgctccatggaccccatcgacagctttgagctcctggatctcctgtttgaccggcaggacggcatcctgagacacgtggagctgggcgagggctggggtcacgtcaaggaccaggtcctgccaaaccccgactctgacgacttcctcagctccatcctgggctctggagactcactgcccagctccccactctggtcccccgaaggcagtgatagtggcatctccgaagacctcccctccgacccccaggacacccctccacgcagcggaccagccacctcccccgccggctgccatcctgcccagcctggcaaggggccctgcctctcctatcatcctggcaactcttgctccaccacaaccccagggccagtgatccaagtacctgaagcctctgtgaccatagacctggaaatgtggagcccaggaggaaggatctgtgctgagaagccggctgatccggtggacctgtccccacgatgcaatctcaccgtgaaaaacctcctcctttcgggcagcagtggggacctgcaacagcatcacctgggggcctcctacctcctgcgacctggggctgggcactgtcaggagctggtgctcaccgaggatgagaagaagctgctggctaaagaaggcatcaccctgcccactcagctgcccctcactaagtacgaggagcgagtgctgaaaaaaatccgccggaaaatccggaacaagcagtcggcgcaagaaagcaggaagaagaagaaggaatatatcgatggcctggagactcggatgtcagcttgcactgctcagaatcaggagttacagaggaaagtcttgcatctcgagaagcaaaacctgtccctcttggagcaactgaagaaactccaggccattgtggtgcagtccaccagcaagtcagcccagacaggcacctgtgtcgcagtcctgttgctgtcctttgccctcatcatcctcccctccatcagcccttttggccccaacaaaaccgagagccctggggactttgcgcctgtacgagtgttctccagaactttgcacaacgatgctgcctcccgcgtggctgctgatgctgtgccaggctccgaggccccaggaccccgacccgaggctgacacaacccgagaagagtctccaggaagccccggggcagactggggcttccaggacaccgcgaacctgaccaattcgacggaggagctggacaacgccaccctggtcctgaggaatgcaacagaggggctgggccaggtcgccctgctggactgggtggcgcctgggccgagcactggctcaggacgtgcagggctggaggcggcgggagacgagctgtgagccccgccaggactatgctcccaggcccctctgcccaggggtgccttggggatgctgcactgggcagctacccacctggggatgggacgtgaggccaagaccccagcagagatgccagaatgggggaggcacagctcatagccacacacccagggcctgactgaggcccacgcaggaaccgacactcagacacaaggcaaagagggccacaggacccgggaaatacacacagagccaggagcagaagcaaagagcagacacacatacagcctgaaacagacctggacagacagacacagcctgaaacagacccggacagacagacacagcctgaaacagacccagacaaacagacagacagacacagcctgaaacagacccagacagacagacagacagcctgaaacagacccagacacagcctgaaacagatccggacagacagacagaaacagcctgaaacagacccagacagacagacagacacagcctgaaacagacccggacagacagacagacacagcctgaaacagacccggacagacagacagacacagcctgaaacagacccggacagacagacagacacagcctgaaacagacctagacagacagacacagattgaaacagacccagacaaacagacagacacagcctgaaacagacccagacacagcctgaaacagacccggacagacagacagacacagcctgaaacagacccagacagacagacagacacagcctgaaacagacccagacagacagaccgacgcagcctgaaagagacccagacagagagacaggcagacacagcctaaaacagacctggacagacaggcagacgtagtctgaaacagacctgaacagacagacagacgcacacacacaacagatgcgcagcaactccccgcccagggacccctcccggcctccctcgcacactgggaggaggaagccgccgagactgcagggagcctggccccggagccccgggtgcgccctggtctttggagcagccacggcccacaatcaccccccttttctaagactgcctgatccgaaataaagtattttgacagaa

SEQ ID NO: 22 - RNA, 2582 nt, Homo sapiens
  source 1..2582: mol_type = genomic RNA
gacagagccacagagggctgtgagcttgcccggccccaggtaacgctggcggtgggtgggcctccagcttggagcagagaccccccgaggcatctgcagacagaactggatggacccatgaatacggatttagctgctggaaagatggcttctgctgcctgctccatggaccccatcgacagctttgagctcctggatctcctgtttgaccggcaggacggcatcctgagacacgtggagctgggcgagggctggggtcacgtcaaggaccagcaggtcctgccaaaccccgactctgacgacttcctcagctccatcctgggctctggagactcactgcccagctccccactctggtcccccgaaggcagtgatagtggcatctccgaagacctcccctccgacccccaggacacccctccacgcagcggaccagccacctcccccgccggctgccatcctgcccagcctggcaaggggccctgcctctcctatcatcctggcaactcttgctccaccacaaccccagggccagtgatccaagtacctgaagcctctgtgaccatagacctggaaatgtggagcccaggaggaaggatctgtgctgagaagccggctgatccggtggacctgtccccacgatgcaatctcaccgtgaaaaacctcctcctttcgggcagcagtggggacctgcatcacctgggggcctcctacctcctgcgacctggggctgggcactgtcaggagctggtgctcaccgaggatgagaagaagctgctggctaaagaaggcatcaccctgcccactcagctgcccctcactaagtacgaggagcgagtgctgaaaaaaatccgccggaaaatccggaacaagcagtcggcgcaagaaagcaggaagaagaagaaggaatatatcgatggcctggagactcggatgtcagcttgcactgctcagaatcaggagttacagaggaaagtcttgcatctcgagaagcaaaacctgtccctcttggagcaactgaagaaactccaggccattgtggtgcagtccaccagcaagtcagcccagacaggcacctgtgtcgcagtcctgttgctgtcctttgccctcatcatcctcccctccatcagcccttttggccccaacaaaaccgagagccctggggactttgcgcctgtacgagtgttctccagaactttgcacaacgatgctgcctcccgcgtggctgctgatgctgtgccaggctccgaggccccaggaccccgacccgaggctgacacaacccgagaagagtctccaggaagccccggggcagactggggcttccaggacaccgcgaacctgaccaattcgacggaggagctggacaacgccaccctggtcctgaggaatgcaacagaggggctgggccaggtcgccctgctggactgggtggcgcctgggccgagcactggctcaggacgtgcagggctggaggcggcgggagacgagctgtgagccccgccaggactatgctcccaggcccctctgcccaggggtgccttggggatgctgcactgggcagctacccacctggggatgggacgtgaggccaagaccccagcagagatgccagaatgggggaggcacagctcatagccacacacccagggcctgactgaggcccacgcaggaaccgacactcagacacaaggcaaagagggccacaggacccgggaaatacacacagagccaggagcagaagcaaagagcagacacacatacagcctgaaacagacctggacagacagacacagcctgaaacagacccggacagacagacacagcctgaaacagacccagacaaacagacagacagacacagcctgaaacagacccagacagacagacagacagcctgaaacagacccagacacagcctgaaacagatccggacagacagacagaaacagcctgaaacagacccagacagacagacagacacagcctgaaacagacccggacagacagacagacacagcctgaaacagacccggacagacagacagacacagcctgaaacagacccggacagacagacagacacagcctgaaacagacctagacagacagacacagattgaaacagacccagacaaacagacagacacagcctgaaacagacccagacacagcctgaaacagacccggacagacagacagacacagcctgaaacagacccagacagacagacagacacagcctgaaacagacccagacagacagaccgacgcagcctgaaagagacccagacagagagacaggcagacacagcctaaaacagacctggacagacaggcagacgtagtctgaaacagacctgaacagacagacagacgcacacacacaacagatgcgcagcaactccccgcccagggacccctcccggcctccctcgcacactgggaggaggaagccgccgagactgcagggagcctggccccggagccccgggtgcgccctggtctttggagcagccacggcccacaatcaccccccttttctaagactgcctgatccgaaataaagtattttgacagaa

SEQ ID NO: 23 - RNA, 2481 nt, Homo sapiens
  source 1..2481: mol_type = genomic RNA
gacagagccacagagggctgtgagcttgcccggccccaggtaacgctggcggtgggtgggcctccagcttggagcagagaccccccgaggcatctgcagacagaactggatggacccatgaatacggatttagctgctggaaagatggcttctgctgcctgctccatggaccccatcgacagctttgagctcctggatctcctgtttgaccggcaggacggcatcctgagacacgtggagctgggcgagggctggggtcacgtcaaggaccagcaggtcctgccaaaccccgactctgacgacttcctcagctccatcctgggctctggagactcactgcccagctccccactctggtcccccgaaggcagtgatagtggcatctccgaagacctcccctccgacccccaggacacccctccacgcagcggaccagccacctcccccgccggctgccatcctgcccagcctggcaaggggccctgcctctcctatcatcctggcaactcttgctccaccacaaccccagggccagtgatccaagtacctgaagcctctgtgaccatagacctggaaatgtggagcccaggaggaaggatctgtgctgagaagccggctgatccggtggacctgtccccacgatgcaatctcaccgtgaaaaacctcctcctttcgggcagcagtggggacctgcaacagcatcacctgggggcctcctacctcctgcgacctggggctgggcactgtcaggagctggtgctcaccgaggatgagaagaagctgctggctaaagaaggcatcaccctgcccactcagctgcccctcactaaggatgtcagcttgcactgctcagaatcaggagttacagaggaaagtcttgcatctcgagaagcaaaacctgtccctcttggagcaactgaagaaactccaggccattgtggtgcagtccaccagcaagtcagcccagacaggcacctgtgtcgcagtcctgttgctgtcctttgccctcatcatcctcccctccatcagcccttttggccccaacaaaaccgagagccctggggactttgcgcctgtacgagtgttctccagaactttgcacaacgatgctgcctcccgcgtggctgctgatgctgtgccaggctccgaggccccaggaccccgacccgaggctgacacaacccgagaagagtctccaggaagccccggggcagactggggcttccaggacaccgcgaacctgaccaattcgacggaggagctggacaacgccaccctggtcctgaggaatgcaacagaggggctgggccaggtcgccctgctggactgggtggcgcctgggccgagcactggctcaggacgtgcagggctggaggcggcgggagacgagctgtgagccccgccaggactatgctcccaggcccctctgcccaggggtgccttggggatgctgcactgggcagctacccacctggggatgggacgtgaggccaagaccccagcagagatgccagaatgggggaggcacagctcatagccacacacccagggcctgactgaggcccacgcaggaaccgacactcagacacaaggcaaagagggccacaggacccgggaaatacacacagagccaggagcagaagcaaagagcagacacacatacagcctgaaacagacctggacagacagacacagcctgaaacagacccggacagacagacacagcctgaaacagacccagacaaacagacagacagacacagcctgaaacagacccagacagacagacagacagcctgaaacagacccagacacagcctgaaacagatccggacagacagacagaaacagcctgaaacagacccagacagacagacagacacagcctgaaacagacccggacagacagacagacacagcctgaaacagacccggacagacagacagacacagcctgaaacagacccggacagacagacagacacagcctgaaacagacctagacagacagacacagattgaaacagacccagacaaacagacagacacagcctgaaacagacccagacacagcctgaaacagacccggacagacagacagacacagcctgaaacagacccagacagacagacagacacagcctgaaacagacccagacagacagaccgacgcagcctgaaagagacccagacagagagacaggcagacacagcctaaaacagacctggacagacaggcagacgtagtctgaaacagacctgaacagacagacagacgcacacacacaacagatgcgcagcaactccccgcccagggacccctcccggcctccctcgcacactgggaggaggaagccgccgagactgcagggagcctggccccggagccccgggtgcgccctggtctttggagcagccacggcccacaatcaccccccttttctaagactgcctgatccgaaataaagtattttgacagaa

SEQ ID NO: 24 - RNA, 1505 nt, Homo sapiens
  source 1..1505: mol_type = genomic RNA
gtgacagagccacagagggctgtgagcttgcccagccccaggtaacgctggcggtgggtgggcctccagcttggagcagagaccccccgaggcatctgcagacagaactggatggacccatgaatacggatttagctgctggaaagatggcttctgctgcctgctccatggaccccatcgacagctttgagctcctggatctcctgtttgaccggcaggacggcatcctgagacacgtggagctgggcgagggctggggtcacgtcaaggaccagcaggtcctgccaaaccccgactctgacgacttcctcagctccatcctgggctctggagactcactgcccagctccccactctggtcccccgaaggcagtgatagtggcatctccgaagacctcccctccgacccccaggacacccctccacgcagcggaccagccacctcccccgccggctgccatcctgcccagcctggcaaggggccctgcctctcctatcatcctggcaactcttgctccaccacaaccccagggccagtgatccaagtacctgaagcctctgtgaccatagacctggaaatgtggagcccaggaggaaggatctgtgctgagaagccggctgatccggtggacctgtccccacgatgcaatctcaccgtgaaaaacctcctcctttcgggcagcagtggggacctgcaacagcatcacctgggggcctcctacctcctgcgacctggggctgggcactgtcaggagctggtgctcaccgaggatgagaagaagctgctggctaaagaaggcatcaccctgcccactcagctgcccctcactaagtacgaggagcgagtgctgaaaaaaatccgccggaaaatccggaacaagcagtcggcgcaagaaagcaggaagaagaagaaggaatatatcgatggcctggagactcggatgtcagcttgcactgctcagaatcaggagttacagaggaaagtcttgcacctcgagaagcaaaacctgtccctcttggagcaactgaagaaactccaggccattgtggtgcagtccaccagcaagtcagcccagacaggcacctgtgtcgcagtcctgttgctgtcctttgccctcatcatcctcccctccatcagcccttttggccccaacaaaaccgagagccctggggactttgcgcctgtacgagtgttctccagaactttgcacaacgatgctgcctcccgcgtggctgctgatgctgtgccaggctccgaggccccaggaccccgacccgaggctgacacaacccgagaagagtctccaggaagccccggggcagactggggcttccaggacaccgcgaacctgaccaattcgacggaggagctggacaacgccaccctggtcctgaggaatgcaacagaggggctgggccaggtcgccctgctggactgggtggcgcctgggccgagcactggctcaggacgtgcagggctggaggcggcgggagacgagctgtga

SEQ ID NO: 25 - RNA, 2206 nt, Homo sapiens
  source 1..2206: mol_type = genomic RNA
acagagccacagagggctgtgagcttgcccagccccaggtaacgctggcggtgggtgggccaccagcttggagcagagaccccccgaggcatctgcagacagaactggatggacccatgaatacggatttagctgctggaaagatggcttctgctgcctgctccatggaccccatcgacagctttgagctcctggatctcctgtttgaccggcaggacggcatcctgagacacgtggagctgggcgagggctggggtcacgtcaaggaccagcaggtcctgccaaaccccgactctgacgacttcctcagctccatcctgggctctggagactcactgcccagctccccactctggtcccccgaaggcagtgatagtggcatctccgaagacctcccctccgacccccaggacacccctccacgcagcggaccagccacctcccccgccggctgccatcctgcccagcctggcaaggggccctgcctctcctatcatcctggcaactcttgctccaccacaaccccagggccagtgatccaagtacctgaagcctctgtgaccatagacctggaaatgtggagcccaggaggaaggatctgtgctgagaagccggctgatccggtggacctgtccccacgatgcaatctcaccgtgaaaaacctcctcctttcgggcagcagtggggacctgcaacagcatcacctgggggcctcctacctcctgcgacctggggctgggcactgtcaggagctggtgctcaccgaggatgagaagaagctgctggctaaagaaggcatcaccctgcccactcagctgcccctcactaagtacgaggagcgagtgctgaaaaaaatccgccggaaaatccggaacaagcagtcggcgcaagaaagcaggaagaagaagaaggccattgtggtgcagtccaccagcaagtcagcccagacaggcacctgtgtcgcagtcctgttgctgtcctttgccctcatcatcctcccctccatcagcccttttggccccaacaaaaccgagagccctggggactttgcgcctgtacgagtgttctccagaactttgcacaacgatgctgcctcccgcgtggctgctgatgctgtgccaggctccgaggccccaggaccccgacccgaggctgacacaacccgagaagagtctccaggaagccccggggcagactggggcttccaggacaccgcgaacctgaccaattcgacggaggagctggacaacgccaccctggtcctgaggaatgcaacagaggggctgggccaggtcgccccgctggactgggtggcgcctgggcccgagcactggctcaggacgtgcagggctggaggcggcgggagacgagcttgtgagccccgccaggactatgctcccaggcccctctgcccaggggtgccttggggatgctgcactgggcagctacccacctggggatgggacgtgaggccaagaccccagcagagatgccagaatgggggaggcacagctcatagccacacacccagggcctgactgaggcccacgcaggaaccgacactcagacacaaggcaaagagggccacaggacccgggaaatacacacagagccaggagcagaagcaaagagcagacacacatacagcctgaaacagacctggacagacagacacagcctgaaacagacccggacagacagacacagcctgaaacagacccagacaaacagacagacagacacagcctgaaacagacccagacagacagacagacagcctgaaacagacccagacacagcctgaaacagatccggacagacagacagaaacagcctgaaacagacccagacagacagacagacacagcctgaaacagacccggacagacagacagacacagcctgaaagagacccagacagagagacaggcagacacagcctaaaacagacctggacagacaggcagacgtagtctgaaacagacctgaacagacagacagacgcacacacacaacagatgcgcagcaactccccgcccagggacccctcccggcctccctcgcacactgggaggaggaagccgccgagactgcagggagcctggccccggagccccgggtgcgccctggtctttggagcagccacggcccacaatcaccccccttttctaagactgcctgatccgaaataaagtattttgacaaa

SEQ ID NO: 26 - RNA, 2582 nt, Homo sapiens
  source 1..2582: mol_type = genomic RNA
acagagggctgtgagcttgcccggccccaggtaacgctggcggtgggtgggcctccagcttggagcagagaccccccgaggcatctgcagacagaactggatggacccatgaatacggatttagctgctggaaagatggcttctgctgcctgctccatggaccccatcgacagctttgagctcctggatctcctgtttgaccggcaggacggcatcctgagacacgtggagctgggcgagggctggggtcacgtcaaggaccaggtcctgccaaaccccgactctgacgacttcctcagctccatcctgggctctggagactcactgcccagctccccactctggtcccccgaaggcagtgatagtggcatctccgaagacctcccctccgacccccaggacacccctccacgcagcggaccagccacctcccccgccggctgccatcctgcccagcctggcaaggggccctgcctctcctatcatcctggcaactcttgctccaccacaaccccagggccagtgatccaagtacctgaagcctctgtgaccatagacctggaaatgtggagcccaggaggaaggatctgtgctgagaagccggctgatccggtggacctgtccccacgatgcaatctcaccgtgaaaaacctcctcctttcgggcagcagtggggacctgcaacagcatcacctgggggcctcctacctcctgcgacctggggctgggcactgtcaggagctggtgctcaccgaggatgagaagaagctgctggctaaagaaggcatcaccctgcccactcagctgcccctcactaagtacgaggagcgagtgctgaaaaaaatccgccggaaaatccggaacaagcagtcggcgcaagaaagcaggaagaagaagaaggaatatatcgatggcctggagactcggatgtcagcttgcactgctcagaatcaggagttacagaggaaagtcttgcatctcgagaagcaaaacctgtccctcttggagcaactgaagaaactccaggccattgtggtgcagtccaccagcaagtcagcccagacaggcacctgtgtcgcagtcctgttgctgtcctttgccctcatcatcctcccctccatcagcccttttggccccaacaaaaccgagagccctggggactttgcgcctgtacgagtgttctccagaactttgcacaacgatgctgcctcccgcgtggctgctgatgctgtgccaggctccgaggccccaggaccccgacccgaggctgacacaacccgagaagagtctccaggaagccccggggcagactggggcttccaggacaccgcgaacctgaccaattcgacggaggagctggacaacgccaccctggtcctgaggaatgcaacagaggggctgggccaggtcgccctgctggactgggtggcgcctgggccgagcactggctcaggacgtgcagggctggaggcggcgggagacgagctgtgagccccgccaggactatgctcccaggcccctctgcccaggggtgccttggggatgctgcactgggcagctacccacctggggatgggacgtgaggccaagaccccagcagagatgccagaatgggggaggcacagctcatagccacacacccagggcctgactgaggcccacgcaggaaccgacactcagacacaaggcaaagagggccacaggacccgggaaatacacacagagccaggagcagaagcaaagagcagacacacatacagcctgaaacagacctggacagacagacacagcctgaaacagacccggacagacagacacagcctgaaacagacccagacaaacagacagacagacacagcctgaaacagacccagacagacagacagacagcctgaaacagacccagacacagcctgaaacagatccggacagacagacagaaacagcctgaaacagacccagacagacagacagacacagcctgaaacagacccggacagacagacagacacagcctgaaacagacccggacagacagacagacacagcctgaaacagacccggacagacagacagacacagcctgaaacagacctagacagacagacacagattgaaacagacccagacaaacagacagacacagcctgaaacagacccagacacagcctgaaacagacccggacagacagacagacacagcctgaaacagacccagacagacagacagacacagcctgaaacagacccagacagacagaccgacgcagcctgaaagagacccagacagagagacaggcagacacagcctaaaacagacctggacagacaggcagacgtagtctgaaacagacctgaacagacagacagacgcacacacacaacagatgcgcagcaactccccgcccagggacccctcccggcctccctcgcacactgggaggaggaagccgccgagactgcagggagcctggccccggagccccgggtgcgccctggtctttggagcagccacggcccacaatcaccccccttttctaagactgcctgatccgaaataaagtattttgacagaaaaaaaa

SEQ ID NO: 27 - RNA, 1489 nt, Homo sapiens
  source 1..1489: mol_type = genomic RNA
aggtaacgctggcggtgggtgggcctccagcttggagcagagaccccccgaggcatctgcagacagaactggatggacccatgaatacggatttagctgctggaaagatggcttctgctgcctgctccatggaccccatcgacagctttgagctcctggatctcctgtttgaccggcaggacggcatcctgagacacgtggagctgggcgagggctggggtcacgtcaaggaccagcaggtcctgccaaaccccgactctgacgacttcctcagctccatcctgggctctggagactcactgcccagctccccactctggtcccccgaaggcagtgatagtggcatctccgaagacctcccctccgacccccaggacacccctccacgcagcggaccagccacctcccccgccggctgccatcctgcccagcctggcaaggggccctgcctctcctatcatcctggcaactcttgctccaccacaaccccagggccagtgatccaagtacctgaagcctctgtgaccatagacctggaaatgtggagcccaggaggaaggatctgtgctgagaagccggctgatccggtggacctgtccccacgatgcaatctcaccgtgaaaaacctcctcctttcgggcagcagtggggacctgcaacagcatcacctgggggcctcctacctcctgcgacctggggctgggcactgtcaggagctggtgctcaccgaggatgagaagaagctgctggctaaagaaggcatcaccctgcccactcagctgcccctcactaagtacgaggagcgagtgctgaaaaaaatccgccggaaaatccggaacaagcagtcggcgcaagaaagcaggaagaagaagaaggaatatatcgatggcctggagactcggatgtcagcttgcactgctcagaatcaggagttacagaggaaagtcttgcatctcgagaagcaaaacctgtccctcttggagcaactgaagaaactccaggccattgtggtgcagtccaccagcaagtcagcccagacaggcacctgtgtcgcagtcctgttgctgtcctttgccctcatcatcctcccctccatcagcccttttggccccaacaaaaccgagagccctggggactttgcgcctgtacgagtgttctccagaactttgcacaacgatgctgcctcccgcgtggctgctgatgctgtgccaggctccgaggccccaggaccccgacccgaggctgacacaacccgagaagagtctccaggaagccccggggcagactggggcttccaggacaccgcgaacctgaccaattcgacggaggagctggacaacgccaccctggtcctgaggaatgcaacagaggggctgggccaggtcgccctgctggactgggtggcgcctgggccgagcactggctcaggacgtgcagggctggaggcggcgggagacgagctgtgagccccgccaggactatgctccca

SEQ ID NO: 28 - RNA, 2618 nt, Homo sapiens
  source 1..2618: mol_type = genomic RNA
gtgaccgcttcagcatcttttgggagtggtgacagagccacagagggctgtgagcttgcccggccccaggtaacgctggcggtgggtgggcctccagcttggagcagagaccccccgaggcatctgcagacagaactggatggacccatgaatacggatttagctgctggaaagatggcttctgctgcctgctccatggaccccatcgacagctttgagctcctggatctcctgtttgaccggcaggacggcatcctgagacacgtggagctgggcgagggctggggtcacgtcaaggaccagcaggtcctgccaaaccccgactctgacgacttcctcagctccatcctgggctctggagactcactgcccagctccccactctggtcccccgaaggcagtgatagtggcatctccgaagacctcccctccgacccccaggacacccctccacgcagcggaccagccacctcccccgccggctgccatcctgcccagcctggcaaggggccctgcctctcctatcatcctggcaactcttgctccaccacaaccccagggccagtgatccaagtacctgaagcctctgtgaccatagacctggaaatgtggagcccaggaggaaggatctgtgctgagaagccggctgatccggtggacctgtccccacgatgcaatctcaccgtgaaaaacctcctcctttcgggcagcagtggggacctgcaacagcatcacctgggggcctcctacctcctgcgacctggggctgggcactgtcaggagctggtgctcaccgaggatgagaagaagctgctggctaaagaaggcatcaccctgcccactcagctgcccctcactaagtacgaggagcgagtgctgaaaaaaatccgccggaaaatccggaacaagcagtcggcgcaagaaagcaggaagaagaagaaggaatatatcgatggcctggagactcggatgtcagcttgcactgctcagaatcaggagttacagaggaaagtcttgcatctcgagaagcaaaacctgtccctcttggagcaactgaagaaactccaggccattgtggtgcagtccaccagcaagtcagcccagacaggcacctgtgtcgcagtcctgttgctgtcctttgccctcatcatcctcccctccatcagcccttttggccccaacaaaaccgagagccctggggactttgcgcctgtacgagtgttctccagaactttgcacaacgatgctgcctcccgcgtggctgctgatgctgtgccaggctccgaggccccaggaccccgacccgaggctgacacaacccgagaagagtctccaggaagccccggggcagactggggcttccaggacaccgcgaacctgaccaattcgacggaggagctggacaacgccaccctggtcctgaggaatgcaacagaggggctgggccaggtcgccctgctggactgggtggcgcctgggccgagcactggctcaggacgtgcagggctggaggcggcgggagacgagctgtgagccccgccaggactatgctcccaggcccctctgcccaggggtgccttggggatgctgcactgggcagctacccacctggggatgggacgtgaggccaagaccccagcagagatgccagaatgggggaggcacagctcatagccacacacccagggcctgactgaggcccacgcaggaaccgacactcagacacaaggcaaagagggccacaggacccgggaaatacacacagagccaggagcagaagcaaagagcagacacacatacagcctgaaacagacctggacagacagacacagcctgaaacagacccggacagacagacacagcctgaaacagacccagacaaacagacagacagacacagcctgaaacagacccagacagacagacagacagcctgaaacagacccagacacagcctgaaacagatccggacagacagacagaaacagcctgaaacagacccagacagacagacagacacagcctgaaacagacccggacagacagacagacacagcctgaaacagacccggacagacagacagacacagcctgaaacagacccggacagacagacagacacagcctgaaacagacctagacagacagacacagattgaaacagacccagacaaacagacagacacagcctgaaacagacccagacacagcctgaaacagacccggacagacagacagacacagcctgaaacagacccagacagacagacagacacagcctgaaacagacccagacagacagaccgacgcagcctgaaagagacccagacagagagacaggcagacacagcctaaaacagacctggacagacaggcagacgtagtctgaaacagacctgaacagacagacagacgcacacacacaacagatgcgcagcaactccccgcccagggacccctcccggcctccctcgcacactgggaggaggaagccgccgagactgcagggagcctggccccggagccccgggtgcgccctggtctttggagcagccacggcccacaatcaccccccttttctaagactgcctgatccgaaataaagtattttgacagaa

SEQ ID NO: 29 - RNA, 2586 nt, Homo sapiens
  source 1..2586: mol_type = genomic RNA
acagagccacagagggctgtgagcttgcccagccccaggtaacgctggcggtgggtgggcctccagcttggagcagagaccccccgaggcatctgcagacagaactggatggacccatgaatacggatttagctgctggaaagatggcttctgctgcctgctccatggaccccatcgacagctttgagctcctggatctcctgtttgaccggcaggacggcatcctgagacacgtggagctgggcgagggctggggtcacgtcaaggaccagcaggtcctgccaaaccccgactctgacgacttcctcagctccatcctgggctctggagactcactgcccagctccccactctggtcccccgaaggcagtgatagtggcatctccgaagacctcccctccgacccccaggacacccctccacgcagcggaccagccacctcccccgccggctgccatcctgcccagcctggcaaggggccctgcctctcctatcatcctggcaactcttgctccaccacaaccccagggccagtgatccaagtacctgaagcctctgtgaccatagacctggaaatgtggagcccaggaggaaggatctgtgctgagaagccggctgatccggtggacctgtccccacgatgcaatctcaccgtgaaaaacctcctcctttcgggcagcagtggggacctgcaacagcatcacctgggggcctcctacctcctgcgacctggggctgggcactgtcaggagctggtgctcaccgaggatgagaagaagctgctggctaaagaaggcatcaccctgcccactcagctgcccctcactaagtacgaggagcgagtgctgaaaaaaatccgccggaaaatccggaacaagcagtcggcgcaagaaagcaggaagaagaagaaggaatatatcgatggcctggagactcggatgtcagcttgcactgctcagaatcaggagttacagaggaaagtcttgcatctcgagaagcaaaacctgtccctcttggagcaactgaagaaactccaggccattgtggtgcagtccaccagcaagtcagcccagacaggcacctgtgtcgcagtcctgttgctgtcctttgccctcatcatcctcccctccatcagcccttttggccccaacaaaaccgagagccctggggactttgcgcctgtacgagtgttctccagaactttgcacaacgatgctgcctcccgcgtggctgctgatgctgtgccaggctccgaggccccaggaccccgacccgaggctgacacaacccgagaagagtctccaggaagccccggggcagactggggcttccaggacaccgcgaacctgaccaattcgacggaggagctggacaacgccaccctggtcctgaggaatgcaacagaggggctgggccaggtcgccctgctggactgggtggcgcctgggccgagcactggctcaggacgtgcagggctggaggcggcgggagacgagctgtgagccccgccaggactatgctcccaggcccctctgcccaggggtgccttggggatgctgcactgggcagctacccacctggggatgggacgtgaggccaagaccccagcagagatgccagaatgggggaggcacagctcatagccacacacccagggcctgactgaggcccacgcaggaaccgacactcagacacaaggcaaagagggccacaggacccgggaaatacacacagagccaggagcagaagcaaagagcagacacacatacagcctgaaacagacctggacagacagacacagcctgaaacagacccggacagacagacacagcctgaaacagacccagacaaacagacagacagacacagcctgaaacagacccagacagacagacagacagcctgaaacagacccagacacagcctgaaacagatccggacagacagacagaaacagcctgaaacagacccagacagacagacagacacagcctgaaacagacccggacagacagacagacacagcctgaaacagacccggacagacagacagacacagcctgaaacagacccggacagacagacagacacagcctgaaacagacctagacagacagacacagattgaaacagacccagacaaacagacagacacagcctgaaacagacccagacacagcctgaaacagacccggacagacagacagacacagcctgaaacagacccagacagacagacagacacagcctgaaacagacccagacagacagaccgacgcagcctgaaagagacccagacagagagacaggcagacacagcctaaaacagacctggacagacaggcagacgtagtctgaaacagacctgaacagacagacagacgcacacacacaacagatgcgcagcaactccccgcccagggacccctcccggcctccctcgcacactgggaggaggaagccgccgagactgcagggagcctggccccggagccccgggtgcgccctggtctttggagcagccacggcccacaatcaccccccttttctaagactgcctgatccgaaataaagtattttgacaaa

SEQ ID NO: 30 - RNA, 1485 nt, Homo sapiens
  source 1..1485: mol_type = genomic RNA
aacgctggcggtgggtgggcctccagcttggagcagagaccccccgaggcatctgcagacagaactggatggacccatgaatacggatttagctgctggaaagatggcttctgctgcctgctccatggaccccatcgacagctttgagctcctggatctcctgtttgaccggcaggacggcatcctgagacacgtggagctgggcgagggctggggtcacgtcaaggaccagcaggtcctgccaaaccccgactctgacgacttcctcagctccatcctgggctctggagactcactgcccagctccccactctggtcccccgaaggcagtgatagtggcatctccgaagacctcccctccgacccccaggacacccctccacgcagcggaccagccacctcccccgccggctgccatcctgcccagcctggcaaggggccctgcctctcctatcatcctggcaactcttgctccaccacaaccccagggccagtgatccaagtacctgaagcctctgtgaccatagacctggaaatgtggagcccaggaggaaggatctgtgctgagaagccggctgatccggtggacctgtccccacgatgcaatctcaccgtgaaaaacctcctcctttcgggcagcagtggggacctgcaacagcatcacctgggggcctcctacctcctgcgacctggggctgggcactgtcaggagctggtgctcaccgaggatgagaagaagctgctggctaaagaaggcatcaccctgcccactcagctgcccctcactaagtacgaggagcgagtgctgaaaaaaatccgccggaaaatccggaacaagcagtcggcgcaagaaagcaggaagaagaagaaggaatatatcgatggcctggagactcggatgtcagcttgcactgctcagaatcaggagttacagaggaaagtcttgcatctcgagaagcaaaacctgtccctcttggagcaactgaagaaactccaggccattgtggtgcagtccaccagcaagtcagcccagacaggcacctgtgtcgcagtcctgttgctgtcctttgccctcatcatcctcccctccatcagcccttttggccccaacaaaaccgagagccctggggactttgcgcctgtacgagtgttctccagaactttgcacaacgatgctgcctcccgcgtggctgctgatgctgtgccaggctccgaggccccaggaccccgacccgaggctgacacaacccgagaagagtctccaggaagccccggggcagactggggcttccaggacaccgcgaacctgaccaattcgacggaggagctggacaacgccaccctggtcctgaggaatgcaacagaggggctgggccaggtcgccctgctggactgggtggcgcctgggccgagcactggctcaggacgtgcagggctggaggcggcgggagacgagctgtgagccccgccaggactatgctccca

SEQ ID NO: 31 - DNA, 2588 bp, Homo sapiens
  source 1..2588: mol_type = other DNA
gacagagccacagagggctgtgagcttgcccggccccaggtaacgctggcggtgggtgggcctccagcttggagcagagaccccccgaggcatctgcagacagaactggatggacccatgaatacggatttagctgctggaaagatggcttctgctgcctgctccatggaccccatcgacagctttgagctcctggatctcctgtttgaccggcaggacggcatcctgagacacgtggagctgggcgagggctggggtcacgtcaaggaccagcaggtcctgccaaaccccgactctgacgacttcctcagctccatcctgggctctggagactcactgcccagctccccactctggtcccccgaaggcagtgatagtggcatctccgaagacctcccctccgacccccaggacacccctccacgcagcggaccagccacctcccccgccggctgccatcctgcccagcctggcaaggggccctgcctctcctatcatcctggcaactcttgctccaccacaaccccagggccagtgatccaagtacctgaagcctctgtgaccatagacctggaaatgtggagcccaggaggaaggatctgtgctgagaagccggctgatccggtggacctgtccccacgatgcaatctcaccgtgaaagacctcctcctttcgggcagcagtggggacctgcaacagcatcacctgggggcctcctacctcctgcgacctggggctgggcactgtcaggagctggtgctcaccgaggatgagaagaagctgctggctaaagaaggcatcaccctgcccactcagctgcccctcactaagtacgaggagcgagtgctgaaaaaaatccgccggaaaatccggaacaagcagtcggcgcaagaaagcaggaagaagaagaaggaatatatcgatggcctggagactcggatgtcagcttgcactgctcagaatcaggagttacagaggaaagtcttgcatctcgagaagcaaaacctgtccctcttggagcaactgaagaaactccaggccattgtggtgcagtccaccagcaagtcagcccagacaggcacctgtgtcgcagtcctgttgctgtcctttgccctcatcatcctcccctccatcagcccttttggccccaacaaaaccgagagccctggggactttgcgcctgtacgagtgttctccagaactttgcacaacgatgctgcctcccgcgtggctgctgatgctgtgccaggctccgaggccccaggaccccgacccgaggctgacacaacccgagaagagtctccaggaagccccggggcagactggggcttccaggacaccgcgaacctgaccaattcgacggaggagctggacaacgccaccctggtcctgaggaatgcaacagaggggctgggccaggtcgccctgctggactgggtggcgcctgggccgagcactggctcaggacgtgcagggctggaggcggcgggagacgagctgtgagccccgccaggactatgctcccaggcccctctgcccaggggtgccttggggatgctgcactgggcagctacccacctggggatgggacgtgaggccaagaccccagcagagatgccagaatgggggaggcacagctcatagccacacacccagggcctgactgaggcccacgcaggaaccgacactcagacacaaggcaaagagggccacaggacccgggaaatacacacagagccaggagcagaagcaaagagcagacacacatacagcctgaaacagacctggacagacagacacagcctgaaacagacccggacagacagacacagcctgaaacagacccagacaaacagacagacagacacagcctgaaacagacccagacagacagacagacagcctgaaacagacccagacacagcctgaaacagatccggacagacagacagaaacagcctgaaacagacccagacagacagacagacacagcctgaaacagacccggacagacagacagacacagcctgaaacagacccggacagacagacagacacagcctgaaacagacccggacagacagacagacacagcctgaaacagacctagacagacagacacagattgaaacagacccagacaaacagacagacacagcctgaaacagacccagacacagcctgaaacagacccggacagacagacagacacagcctgaaacagacccagacagacagacagacacagcctgaaacagacccagacagacagaccgacgcagcctgaaagagacccagacagagagacaggcagacacagcctaaaacagacctggacagacaggcagacgtagtctgaaacagacctgaacagacagacagacgcacacacacaacagatgcgcagcaactccccgcccagggacccctcccggcctccctcgcacactgggaggaggaagccgccgagactgcagggagcctggccccggagccccgggtgcgccctggtctttggagcagccacggcccacaatcaccccccttttctaagactgcctgatccgaaataaagtattttgacagaa

SEQ ID NO: 32 - DNA, 1491 bp, Homo sapiens
  source 1..1491: mol_type = other DNA
acagagggctgtgagcttgcccggccccaggtaacgctggcggtgggtgggcctccagcttggagcagagaccccccgaggcatctgcagacagaactggatggacccatgaatacggatttagctgctggaaagatggcttctgctgcctgctccatggaccccatcgacagctttgagctcctggatctcctgtttgaccggcaggacggcatcctgagacacgtggagctgggcgagggctggggtcacgtcaaggaccaggtcctgccaaaccccgactctgacgacttcctcagctccatcctgggctctggagactcactgcccagctccccactctggtcccccgaaggcagtgatagtggcatctccgaagacctcccctccgacccccaggacacccctccacgcagcggaccagccacctcccccgccggctgccatcctgcccagcctggcaaggggccctgcctctcctatcatcctggcaactcttgctccaccacaaccccagggccagtgatccaagtacctgaagcctctgtgaccatagacctggaaatgtggagcccaggaggaaggatctgtgctgagaagccggctgatccggtggacctgtccccacgatgcaatctcaccgtgaaagacctcctcctttcgggcagcagtggggacctgcaacagcatcacctgggggcctcctacctcctgcgacctggggctgggcactgtcaggagctggtgctcaccgaggatgagaagaagctgctggctaaagaaggcatcaccctgcccactcagctgcccctcactaagtacgaggagcgagtgctgaaaaaaatccgccggaaaatccggaacaagcagtcggcgcaagaaagcaggaagaagaagaaggaatatatcgatggcctggagactcggatgtcagcttgcactgctcagaatcaggagttacagaggaaagtcttgcatctcgagaagcaaaacctgtccctcttggagcaactgaagaaactccaggccattgtggtgcagtccaccagcaagtcagcccagacaggcacctgtgtcgcagtcctgttgctgtcctttgccctcatcatcctcccctccatcagcccttttggccccaacaaaaccgagagccctggggactttgcgcctgtacgagtgttctccagaactttgcacaacgatgctgcctcccgcgtggctgctgatgctgtgccaggctccgaggccccaggaccccgacccgaggctgacacaacccgagaagagtctccaggaagccccggggcagactggggcttccaggacaccgcgaacctgaccaattcgacggaggagctggacaacgccaccctggtcctgaggaatgcaacagaggggctgggccaggtcgccctgctggactgggtggcgcctgggccgagcactggctcaggacgtgcagggctggaggcggcgggagacgagctgtga

SEQ ID NO: 33 - DNA, 1475 bp, Homo sapiens
  source 1..1475: mol_type = other DNA
aggtaacgctggcggtgggtgggcctccagcttggagcagagaccccccgaggcatctgcagacagaactggatggacccatgaatacggatttagctgctggaaagatggcttctgctgcctgctccatggaccccatcgacagctttgagctcctggatctcctgtttgaccggcaggacggcatcctgagacacgtggagctgggcgagggctggggtcacgtcaaggaccagcaggtcctgccaaaccccgactctgacgacttcctcagctccatcctgggctctggagactcactgcccagctccccactctggtcccccgaaggcagtgatagtggcatctccgaagacctcccctccgacccccaggacacccctccacgcagcggaccagccacctcccccgccggctgccatcctgcccagcctggcaaggggccctgcctctcctatcatcctggcaactcttgctccaccacaaccccagggccagtgatccaagtacctgaagcctctgtgaccatagacctggaaatgtggagcccaggaggaaggatctgtgctgagaagccggctgatccggtggacctgtccccacgatgcaatctcaccgtgaaagacctcctcctttcgggcagcagtggggacctgcatcacctgggggcctcctacctcctgcgacctggggctgggcactgtcaggagctggtgctcaccgaggatgagaagaagctgctggctaaagaaggcatcaccctgcccactcagctgcccctcactaagtacgaggagcgagtgctgaaaaaaatccgccggaaaatccggaacaagcagtcggcgcaagaaagcaggaagaagaagaaggaatatatcgatggcctggagactcggatgtcagcttgcactgctcagaatcaggagttacagaggaaagtcttgcatctcgagaagcaaaacctgtccctcttggagcaactgaagaaactccaggccattgtggtgcagtccaccagcaagtcagcccagacaggcacctgtgtcgcagtcctgttgctgtcctttgccctcatcatcctcccctccatcagcccttttggccccaacaaaaccgagagccctggggactttgcgcctgtacgagtgttctccagaactttgcacaacgatgctgcctcccgcgtggctgctgatgctgtgccaggctccgaggccccaggaccccgacccgaggctgacacaacccgagaagagtctccaggaagccccggggcagactggggcttccaggacaccgcgaacctgaccaattcgacggaggagctggacaacgccaccctggtcctgaggaatgcaacagaggggctgggccaggtcgccctgctggactgggtggcgcctgggccgagcactggctcaggacgtgcagggctggaggcggcgggagacgagctgtgagccccgccaggacta

SEQ ID NO: 34 - DNA, 1382 bp, Homo sapiens
  source 1..1382: mol_type = other DNA
aggtaacgctggcggtgggtgggcctccagcttggagcagagaccccccgaggcatctgcagacagaactggatggacccatgaatacggatttagctgctggaaagatggcttctgctgcctgctccatggaccccatcgacagctttgagctcctggatctcctgtttgaccggcaggacggcatcctgagacacgtggagctgggcgagggctggggtcacgtcaaggaccagcaggtcctgccaaaccccgactctgacgacttcctcagctccatcctgggctctggagactcactgcccagctccccactctggtcccccgaaggcagtgatagtggcatctccgaagacctcccctccgacccccaggacacccctccacgcagcggaccagccacctcccccgccggctgccatcctgcccagcctggcaaggggccctgcctctcctatcatcctggcaactcttgctccaccacaaccccagggccagtgatccaagtacctgaagcctctgtgaccatagacctggaaatgtggagcccaggaggaaggatctgtgctgagaagccggctgatccggtggacctgtccccacgatgcaatctcaccgtgaaagacctcctcctttcgggcagcagtggggacctgcaacagcatcacctgggggcctcctacctcctgcgacctggggctgggcactgtcaggagctggtgctcaccgaggatgagaagaagctgctggctaaagaaggcatcaccctgcccactcagctgcccctcactaaggatgtcagcttgcactgctcagaatcaggagttacagaggaaagtcttgcatctcgagaagcaaaacctgtccctcttggagcaactgaagaaactccaggccattgtggtgcagtccaccagcaagtcagcccagacaggcacctgtgtcgcagtcctgttgctgtcctttgccctcatcatcctcccctccatcagcccttttggccccaacaaaaccgagagccctggggactttgcgcctgtacgagtgttctccagaactttgcacaacgatgctgcctcccgcgtggctgctgatgctgtgccaggctccgaggccccaggaccccgacccgaggctgacacaacccgagaagagtctccaggaagccccggggcagactggggcttccaggacaccgcgaacctgaccaattcgacggaggagctggacaacgccaccctggtcctgaggaatgcaacagaggggctgggccaggtcgccctgctggactgggtggcgcctgggccgagcactggctcaggacgtgcagggctggaggcggcgggagacgagctgtgagccccgccaggactatgctccca

SEQ ID NO: 35 - DNA, 2585 bp, Homo sapiens
  source 1..2585: mol_type = other DNA
gacagagccacagagggctgtgagcttgcccggccccaggtaacgctggcggtgggtgggcctccagcttggagcagagaccccccgaggcatctgcagacagaactggatggacccatgaatacggatttagctgctggaaagatggcttctgctgcctgctccatggaccccatcgacagctttgagctcctggatctcctgtttgaccggcaggacggcatcctgagacacgtggagctgggcgagggctggggtcacgtcaaggaccaggtcctgccaaaccccgactctgacgacttcctcagctccatcctgggctctggagactcactgcccagctccccactctggtcccccgaaggcagtgatagtggcatctccgaagacctcccctccgacccccaggacacccctccacgcagcggaccagccacctcccccgccggctgccatcctgcccagcctggcaaggggccctgcctctcctatcatcctggcaactcttgctccaccacaaccccagggccagtgatccaagtacctgaagcctctgtgaccatagacctggaaatgtggagcccaggaggaaggatctgtgctgagaagccggctgatccggtggacctgtccccacgatgcaatctcaccgtgaaagacctcctcctttcgggcagcagtggggacctgcaacagcatcacctgggggcctcctacctcctgcgacctggggctgggcactgtcaggagctggtgctcaccgaggatgagaagaagctgctggctaaagaaggcatcaccctgcccactcagctgcccctcactaagtacgaggagcgagtgctgaaaaaaatccgccggaaaatccggaacaagcagtcggcgcaagaaagcaggaagaagaagaaggaatatatcgatggcctggagactcggatgtcagcttgcactgctcagaatcaggagttacagaggaaagtcttgcatctcgagaagcaaaacctgtccctcttggagcaactgaagaaactccaggccattgtggtgcagtccaccagcaagtcagcccagacaggcacctgtgtcgcagtcctgttgctgtcctttgccctcatcatcctcccctccatcagcccttttggccccaacaaaaccgagagccctggggactttgcgcctgtacgagtgttctccagaactttgcacaacgatgctgcctcccgcgtggctgctgatgctgtgccaggctccgaggccccaggaccccgacccgaggctgacacaacccgagaagagtctccaggaagccccggggcagactggggcttccaggacaccgcgaacctgaccaattcgacggaggagctggacaacgccaccctggtcctgaggaatgcaacagaggggctgggccaggtcgccctgctggactgggtggcgcctgggccgagcactggctcaggacgtgcagggctggaggcggcgggagacgagctgtgagccccgccaggactatgctcccaggcccctctgcccaggggtgccttggggatgctgcactgggcagctacccacctggggatgggacgtgaggccaagaccccagcagagatgccagaatgggggaggcacagctcatagccacacacccagggcctgactgaggcccacgcaggaaccgacactcagacacaaggcaaagagggccacaggacccgggaaatacacacagagccaggagcagaagcaaagagcagacacacatacagcctgaaacagacctggacagacagacacagcctgaaacagacccggacagacagacacagcctgaaacagacccagacaaacagacagacagacacagcctgaaacagacccagacagacagacagacagcctgaaacagacccagacacagcctgaaacagatccggacagacagacagaaacagcctgaaacagacccagacagacagacagacacagcctgaaacagacccggacagacagacagacacagcctgaaacagacccggacagacagacagacacagcctgaaacagacccggacagacagacagacacagcctgaaacagacctagacagacagacacagattgaaacagacccagacaaacagacagacacagcctgaaacagacccagacacagcctgaaacagacccggacagacagacagacacagcctgaaacagacccagacagacagacagacacagcctgaaacagacccagacagacagaccgacgcagcctgaaagagacccagacagagagacaggcagacacagcctaaaacagacctggacagacaggcagacgtagtctgaaacagacctgaacagacagacagacgcacacacacaacagatgcgcagcaactccccgcccagggacccctcccggcctccctcgcacactgggaggaggaagccgccgagactgcagggagcctggccccggagccccgggtgcgccctggtctttggagcagccacggcccacaatcaccccccttttctaagactgcctgatccgaaataaagtattttgacagaa

SEQ ID NO: 36 - DNA, 2582 bp, Homo sapiens
  source 1..2582: mol_type = other DNA
gacagagccacagagggctgtgagcttgcccggccccaggtaacgctggcggtgggtgggcctccagcttggagcagagaccccccgaggcatctgcagacagaactggatggacccatgaatacggatttagctgctggaaagatggcttctgctgcctgctccatggaccccatcgacagctttgagctcctggatctcctgtttgaccggcaggacggcatcctgagacacgtggagctgggcgagggctggggtcacgtcaaggaccagcaggtcctgccaaaccccgactctgacgacttcctcagctccatcctgggctctggagactcactgcccagctccccactctggtcccccgaaggcagtgatagtggcatctccgaagacctcccctccgacccccaggacacccctccacgcagcggaccagccacctcccccgccggctgccatcctgcccagcctggcaaggggccctgcctctcctatcatcctggcaactcttgctccaccacaaccccagggccagtgatccaagtacctgaagcctctgtgaccatagacctggaaatgtggagcccaggaggaaggatctgtgctgagaagccggctgatccggtggacctgtccccacgatgcaatctcaccgtgaaagacctcctcctttcgggcagcagtggggacctgcatcacctgggggcctcctacctcctgcgacctggggctgggcactgtcaggagctggtgctcaccgaggatgagaagaagctgctggctaaagaaggcatcaccctgcccactcagctgcccctcactaagtacgaggagcgagtgctgaaaaaaatccgccggaaaatccggaacaagcagtcggcgcaagaaagcaggaagaagaagaaggaatatatcgatggcctggagactcggatgtcagcttgcactgctcagaatcaggagttacagaggaaagtcttgcatctcgagaagcaaaacctgtccctcttggagcaactgaagaaactccaggccattgtggtgcagtccaccagcaagtcagcccagacaggcacctgtgtcgcagtcctgttgctgtcctttgccctcatcatcctcccctccatcagcccttttggccccaacaaaaccgagagccctggggactttgcgcctgtacgagtgttctccagaactttgcacaacgatgctgcctcccgcgtggctgctgatgctgtgccaggctccgaggccccaggaccccgacccgaggctgacacaacccgagaagagtctccaggaagccccggggcagactggggcttccaggacaccgcgaacctgaccaattcgacggaggagctggacaacgccaccctggtcctgaggaatgcaacagaggggctgggccaggtcgccctgctggactgggtggcgcctgggccgagcactggctcaggacgtgcagggctggaggcggcgggagacgagctgtgagccccgccaggactatgctcccaggcccctctgcccaggggtgccttggggatgctgcactgggcagctacccacctggggatgggacgtgaggccaagaccccagcagagatgccagaatgggggaggcacagctcatagccacacacccagggcctgactgaggcccacgcaggaaccgacactcagacacaaggcaaagagggccacaggacccgggaaatacacacagagccaggagcagaagcaaagagcagacacacatacagcctgaaacagacctggacagacagacacagcctgaaacagacccggacagacagacacagcctgaaacagacccagacaaacagacagacagacacagcctgaaacagacccagacagacagacagacagcctgaaacagacccagacacagcctgaaacagatccggacagacagacagaaacagcctgaaacagacccagacagacagacagacacagcctgaaacagacccggacagacagacagacacagcctgaaacagacccggacagacagacagacacagcctgaaacagacccggacagacagacagacacagcctgaaacagacctagacagacagacacagattgaaacagacccagacaaacagacagacacagcctgaaacagacccagacacagcctgaaacagacccggacagacagacagacacagcctgaaacagacccagacagacagacagacacagcctgaaacagacccagacagacagaccgacgcagcctgaaagagacccagacagagagacaggcagacacagcctaaaacagacctggacagacaggcagacgtagtctgaaacagacctgaacagacagacagacgcacacacacaacagatgcgcagcaactccccgcccagggacccctcccggcctccctcgcacactgggaggaggaagccgccgagactgcagggagcctggccccggagccccgggtgcgccctggtctttggagcagccacggcccacaatcaccccccttttctaagactgcctgatccgaaataaagtattttgacagaa

SEQ ID NO: 37 - DNA, 2481 bp, Homo sapiens
  source 1..2481: mol_type = other DNA
gacagagccacagagggctgtgagcttgcccggccccaggtaacgctggcggtgggtgggcctccagcttggagcagagaccccccgaggcatctgcagacagaactggatggacccatgaatacggatttagctgctggaaagatggcttctgctgcctgctccatggaccccatcgacagctttgagctcctggatctcctgtttgaccggcaggacggcatcctgagacacgtggagctgggcgagggctggggtcacgtcaaggaccagcaggtcctgccaaaccccgactctgacgacttcctcagctccatcctgggctctggagactcactgcccagctccccactctggtcccccgaaggcagtgatagtggcatctccgaagacctcccctccgacccccaggacacccctccacgcagcggaccagccacctcccccgccggctgccatcctgcccagcctggcaaggggccctgcctctcctatcatcctggcaactcttgctccaccacaaccccagggccagtgatccaagtacctgaagcctctgtgaccatagacctggaaatgtggagcccaggaggaaggatctgtgctgagaagccggctgatccggtggacctgtccccacgatgcaatctcaccgtgaaagacctcctcctttcgggcagcagtggggacctgcaacagcatcacctgggggcctcctacctcctgcgacctggggctgggcactgtcaggagctggtgctcaccgaggatgagaagaagctgctggctaaagaaggcatcaccctgcccactcagctgcccctcactaaggatgtcagcttgcactgctcagaatcaggagttacagaggaaagtcttgcatctcgagaagcaaaacctgtccctcttggagcaactgaagaaactccaggccattgtggtgcagtccaccagcaagtcagcccagacaggcacctgtgtcgcagtcctgttgctgtcctttgccctcatcatcctcccctccatcagcccttttggccccaacaaaaccgagagccctggggactttgcgcctgtacgagtgttctccagaactttgcacaacgatgctgcctcccgcgtggctgctgatgctgtgccaggctccgaggccccaggaccccgacccgaggctgacacaacccgagaagagtctccaggaagccccggggcagactggggcttccaggacaccgcgaacctgaccaattcgacggaggagctggacaacgccaccctggtcctgaggaatgcaacagaggggctgggccaggtcgccctgctggactgggtggcgcctgggccgagcactggctcaggacgtgcagggctggaggcggcgggagacgagctgtgagccccgccaggactatgctcccaggcccctctgcccaggggtgccttggggatgctgcactgggcagctacccacctggggatgggacgtgaggccaagaccccagcagagatgccagaatgggggaggcacagctcatagccacacacccagggcctgactgaggcccacgcaggaaccgacactcagacacaaggcaaagagggccacaggacccgggaaatacacacagagccaggagcagaagcaaagagcagacacacatacagcctgaaacagacctggacagacagacacagcctgaaacagacccggacagacagacacagcctgaaacagacccagacaaacagacagacagacacagcctgaaacagacccagacagacagacagacagcctgaaacagacccagacacagcctgaaacagatccggacagacagacagaaacagcctgaaacagacccagacagacagacagacacagcctgaaacagacccggacagacagacagacacagcctgaaacagacccggacagacagacagacacagcctgaaacagacccggacagacagacagacacagcctgaaacagacctagacagacagacacagattgaaacagacccagacaaacagacagacacagcctgaaacagacccagacacagcctgaaacagacccggacagacagacagacacagcctgaaacagacccagacagacagacagacacagcctgaaacagacccagacagacagaccgacgcagcctgaaagagacccagacagagagacaggcagacacagcctaaaacagacctggacagacaggcagacgtagtctgaaacagacctgaacagacagacagacgcacacacacaacagatgcgcagcaactccccgcccagggacccctcccggcctccctcgcacactgggaggaggaagccgccgagactgcagggagcctggccccggagccccgggtgcgccctggtctttggagcagccacggcccacaatcaccccccttttctaagactgcctgatccgaaataaagtattttgacagaa

SEQ ID NO: 38 - DNA, 1505 bp, Homo sapiens
  source 1..1505: mol_type = other DNA
gtgacagagccacagagggctgtgagcttgcccagccccaggtaacgctggcggtgggtgggcctccagcttggagcagagaccccccgaggcatctgcagacagaactggatggacccatgaatacggatttagctgctggaaagatggcttctgctgcctgctccatggaccccatcgacagctttgagctcctggatctcctgtttgaccggcaggacggcatcctgagacacgtggagctgggcgagggctggggtcacgtcaaggaccagcaggtcctgccaaaccccgactctgacgacttcctcagctccatcctgggctctggagactcactgcccagctccccactctggtcccccgaaggcagtgatagtggcatctccgaagacctcccctccgacccccaggacacccctccacgcagcggaccagccacctcccccgccggctgccatcctgcccagcctggcaaggggccctgcctctcctatcatcctggcaactcttgctccaccacaaccccagggccagtgatccaagtacctgaagcctctgtgaccatagacctggaaatgtggagcccaggaggaaggatctgtgctgagaagccggctgatccggtggacctgtccccacgatgcaatctcaccgtgaaagacctcctcctttcgggcagcagtggggacctgcaacagcatcacctgggggcctcctacctcctgcgacctggggctgggcactgtcaggagctggtgctcaccgaggatgagaagaagctgctggctaaagaaggcatcaccctgcccactcagctgcccctcactaagtacgaggagcgagtgctgaaaaaaatccgccggaaaatccggaacaagcagtcggcgcaagaaagcaggaagaagaagaaggaatatatcgatggcctggagactcggatgtcagcttgcactgctcagaatcaggagttacagaggaaagtcttgcacctcgagaagcaaaacctgtccctcttggagcaactgaagaaactccaggccattgtggtgcagtccaccagcaagtcagcccagacaggcacctgtgtcgcagtcctgttgctgtcctttgccctcatcatcctcccctccatcagcccttttggccccaacaaaaccgagagccctggggactttgcgcctgtacgagtgttctccagaactttgcacaacgatgctgcctcccgcgtggctgctgatgctgtgccaggctccgaggccccaggaccccgacccgaggctgacacaacccgagaagagtctccaggaagccccggggcagactggggcttccaggacaccgcgaacctgaccaattcgacggaggagctggacaacgccaccctggtcctgaggaatgcaacagaggggctgggccaggtcgccctgctggactgggtggcgcctgggccgagcactggctcaggacgtgcagggctggaggcggcgggagacgagctgtga

SEQ ID NO: 39 - DNA, 2206 bp, Homo sapiens
  source 1..2206: mol_type = other DNA
acagagccacagagggctgtgagcttgcccagccccaggtaacgctggcggtgggtgggccaccagcttggagcagagaccccccgaggcatctgcagacagaactggatggacccatgaatacggatttagctgctggaaagatggcttctgctgcctgctccatggaccccatcgacagctttgagctcctggatctcctgtttgaccggcaggacggcatcctgagacacgtggagctgggcgagggctggggtcacgtcaaggaccagcaggtcctgccaaaccccgactctgacgacttcctcagctccatcctgggctctggagactcactgcccagctccccactctggtcccccgaaggcagtgatagtggcatctccgaagacctcccctccgacccccaggacacccctccacgcagcggaccagccacctcccccgccggctgccatcctgcccagcctggcaaggggccctgcctctcctatcatcctggcaactcttgctccaccacaaccccagggccagtgatccaagtacctgaagcctctgtgaccatagacctggaaatgtggagcccaggaggaaggatctgtgctgagaagccggctgatccggtggacctgtccccacgatgcaatctcaccgtgaaagacctcctcctttcgggcagcagtggggacctgcaacagcatcacctgggggcctcctacctcctgcgacctggggctgggcactgtcaggagctggtgctcaccgaggatgagaagaagctgctggctaaagaaggcatcaccctgcccactcagctgcccctcactaagtacgaggagcgagtgctgaaaaaaatccgccggaaaatccggaacaagcagtcggcgcaagaaagcaggaagaagaagaaggccattgtggtgcagtccaccagcaagtcagcccagacaggcacctgtgtcgcagtcctgttgctgtcctttgccctcatcatcctcccctccatcagcccttttggccccaacaaaaccgagagccctggggactttgcgcctgtacgagtgttctccagaactttgcacaacgatgctgcctcccgcgtggctgctgatgctgtgccaggctccgaggccccaggaccccgacccgaggctgacacaacccgagaagagtctccaggaagccccggggcagactggggcttccaggacaccgcgaacctgaccaattcgacggaggagctggacaacgccaccctggtcctgaggaatgcaacagaggggctgggccaggtcgccccgctggactgggtggcgcctgggcccgagcactggctcaggacgtgcagggctggaggcggcgggagacgagcttgtgagccccgccaggactatgctcccaggcccctctgcccaggggtgccttggggatgctgcactgggcagctacccacctggggatgggacgtgaggccaagaccccagcagagatgccagaatgggggaggcacagctcatagccacacacccagggcctgactgaggcccacgcaggaaccgacactcagacacaaggcaaagagggccacaggacccgggaaatacacacagagccaggagcagaagcaaagagcagacacacatacagcctgaaacagacctggacagacagacacagcctgaaacagacccggacagacagacacagcctgaaacagacccagacaaacagacagacagacacagcctgaaacagacccagacagacagacagacagcctgaaacagacccagacacagcctgaaacagatccggacagacagacagaaacagcctgaaacagacccagacagacagacagacacagcctgaaacagacccggacagacagacagacacagcctgaaagagacccagacagagagacaggcagacacagcctaaaacagacctggacagacaggcagacgtagtctgaaacagacctgaacagacagacagacgcacacacacaacagatgcgcagcaactccccgcccagggacccctcccggcctccctcgcacactgggaggaggaagccgccgagactgcagggagcctggccccggagccccgggtgcgccctggtctttggagcagccacggcccacaatcaccccccttttctaagactgcctgatccgaaataaagtattttgacaaa

SEQ ID NO: 40 - DNA, 2582 bp, Homo sapiens
  source 1..2582: mol_type = other DNA
acagagggctgtgagcttgcccggccccaggtaacgctggcggtgggtgggcctccagcttggagcagagaccccccgaggcatctgcagacagaactggatggacccatgaatacggatttagctgctggaaagatggcttctgctgcctgctccatggaccccatcgacagctttgagctcctggatctcctgtttgaccggcaggacggcatcctgagacacgtggagctgggcgagggctggggtcacgtcaaggaccaggtcctgccaaaccccgactctgacgacttcctcagctccatcctgggctctggagactcactgcccagctccccactctggtcccccgaaggcagtgatagtggcatctccgaagacctcccctccgacccccaggacacccctccacgcagcggaccagccacctcccccgccggctgccatcctgcccagcctggcaaggggccctgcctctcctatcatcctggcaactcttgctccaccacaaccccagggccagtgatccaagtacctgaagcctctgtgaccatagacctggaaatgtggagcccaggaggaaggatctgtgctgagaagccggctgatccggtggacctgtccccacgatgcaatctcaccgtgaaagacctcctcctttcgggcagcagtggggacctgcaacagcatcacctgggggcctcctacctcctgcgacctggggctgggcactgtcaggagctggtgctcaccgaggatgagaagaagctgctggctaaagaaggcatcaccctgcccactcagctgcccctcactaagtacgaggagcgagtgctgaaaaaaatccgccggaaaatccggaacaagcagtcggcgcaagaaagcaggaagaagaagaaggaatatatcgatggcctggagactcggatgtcagcttgcactgctcagaatcaggagttacagaggaaagtcttgcatctcgagaagcaaaacctgtccctcttggagcaactgaagaaactccaggccattgtggtgcagtccaccagcaagtcagcccagacaggcacctgtgtcgcagtcctgttgctgtcctttgccctcatcatcctcccctccatcagcccttttggccccaacaaaaccgagagccctggggactttgcgcctgtacgagtgttctccagaactttgcacaacgatgctgcctcccgcgtggctgctgatgctgtgccaggctccgaggccccaggaccccgacccgaggctgacacaacccgagaagagtctccaggaagccccggggcagactggggcttccaggacaccgcgaacctgaccaattcgacggaggagctggacaacgccaccctggtcctgaggaatgcaacagaggggctgggccaggtcgccctgctggactgggtggcgcctgggccgagcactggctcaggacgtgcagggctggaggcggcgggagacgagctgtgagccccgccaggactatgctcccaggcccctctgcccaggggtgccttggggatgctgcactgggcagctacccacctggggatgggacgtgaggccaagaccccagcagagatgccagaatgggggaggcacagctcatagccacacacccagggcctgactgaggcccacgcaggaaccgacactcagacacaaggcaaagagggccacaggacccgggaaatacacacagagccaggagcagaagcaaagagcagacacacatacagcctgaaacagacctggacagacagacacagcctgaaacagacccggacagacagacacagcctgaaacagacccagacaaacagacagacagacacagcctgaaacagacccagacagacagacagacagcctgaaacagacccagacacagcctgaaacagatccggacagacagacagaaacagcctgaaacagacccagacagacagacagacacagcctgaaacagacccggacagacagacagacacagcctgaaacagacccggacagacagacagacacagcctgaaacagacccggacagacagacagacacagcctgaaacagacctagacagacagacacagattgaaacagacccagacaaacagacagacacagcctgaaacagacccagacacagcctgaaacagacccggacagacagacagacacagcctgaaacagacccagacagacagacagacacagcctgaaacagacccagacagacagaccgacgcagcctgaaagagacccagacagagagacaggcagacacagcctaaaacagacctggacagacaggcagacgtagtctgaaacagacctgaacagacagacagacgcacacacacaacagatgcgcagcaactccccgcccagggacccctcccggcctccctcgcacactgggaggaggaagccgccgagactgcagggagcctggccccggagccccgggtgcgccctggtctttggagcagccacggcccacaatcaccccccttttctaagactgcctgatccgaaataaagtattttgacagaaaaaaaa

SEQ ID NO: 41 - DNA, 1489 bp, Homo sapiens
  source 1..1489: mol_type = other DNA
aggtaacgctggcggtgggtgggcctccagcttggagcagagaccccccgaggcatctgcagacagaactggatggacccatgaatacggatttagctgctggaaagatggcttctgctgcctgctccatggaccccatcgacagctttgagctcctggatctcctgtttgaccggcaggacggcatcctgagacacgtggagctgggcgagggctggggtcacgtcaaggaccagcaggtcctgccaaaccccgactctgacgacttcctcagctccatcctgggctctggagactcactgcccagctccccactctggtcccccgaaggcagtgatagtggcatctccgaagacctcccctccgacccccaggacacccctccacgcagcggaccagccacctcccccgccggctgccatcctgcccagcctggcaaggggccctgcctctcctatcatcctggcaactcttgctccaccacaaccccagggccagtgatccaagtacctgaagcctctgtgaccatagacctggaaatgtggagcccaggaggaaggatctgtgctgagaagccggctgatccggtggacctgtccccacgatgcaatctcaccgtgaaagacctcctcctttcgggcagcagtggggacctgcaacagcatcacctgggggcctcctacctcctgcgacctggggctgggcactgtcaggagctggtgctcaccgaggatgagaagaagctgctggctaaagaaggcatcaccctgcccactcagctgcccctcactaagtacgaggagcgagtgctgaaaaaaatccgccggaaaatccggaacaagcagtcggcgcaagaaagcaggaagaagaagaaggaatatatcgatggcctggagactcggatgtcagcttgcactgctcagaatcaggagttacagaggaaagtcttgcatctcgagaagcaaaacctgtccctcttggagcaactgaagaaactccaggccattgtggtgcagtccaccagcaagtcagcccagacaggcacctgtgtcgcagtcctgttgctgtcctttgccctcatcatcctcccctccatcagcccttttggccccaacaaaaccgagagccctggggactttgcgcctgtacgagtgttctccagaactttgcacaacgatgctgcctcccgcgtggctgctgatgctgtgccaggctccgaggccccaggaccccgacccgaggctgacacaacccgagaagagtctccaggaagccccggggcagactggggcttccaggacaccgcgaacctgaccaattcgacggaggagctggacaacgccaccctggtcctgaggaatgcaacagaggggctgggccaggtcgccctgctggactgggtggcgcctgggccgagcactggctcaggacgtgcagggctggaggcggcgggagacgagctgtgagccccgccaggactatgctccca

SEQ ID NO: 42 - DNA, 2618 bp, Homo sapiens
  source 1..2618: mol_type = other DNA
gtgaccgcttcagcatcttttgggagtggtgacagagccacagagggctgtgagcttgcccggccccaggtaacgctggcggtgggtgggcctccagcttggagcagagaccccccgaggcatctgcagacagaactggatggacccatgaatacggatttagctgctggaaagatggcttctgctgcctgctccatggaccccatcgacagctttgagctcctggatctcctgtttgaccggcaggacggcatcctgagacacgtggagctgggcgagggctggggtcacgtcaaggaccagcaggtcctgccaaaccccgactctgacgacttcctcagctccatcctgggctctggagactcactgcccagctccccactctggtcccccgaaggcagtgatagtggcatctccgaagacctcccctccgacccccaggacacccctccacgcagcggaccagccacctcccccgccggctgccatcctgcccagcctggcaaggggccctgcctctcctatcatcctggcaactcttgctccaccacaaccccagggccagtgatccaagtacctgaagcctctgtgaccatagacctggaaatgtggagcccaggaggaaggatctgtgctgagaagccggctgatccggtggacctgtccccacgatgcaatctcaccgtgaaagacctcctcctttcgggcagcagtggggacctgcaacagcatcacctgggggcctcctacctcctgcgacctggggctgggcactgtcaggagctggtgctcaccgaggatgagaagaagctgctggctaaagaaggcatcaccctgcccactcagctgcccctcactaagtacgaggagcgagtgctgaaaaaaatccgccggaaaatccggaacaagcagtcggcgcaagaaagcaggaagaagaagaaggaatatatcgatggcctggagactcggatgtcagcttgcactgctcagaatcaggagttacagaggaaagtcttgcatctcgagaagcaaaacctgtccctcttggagcaactgaagaaactccaggccattgtggtgcagtccaccagcaagtcagcccagacaggcacctgtgtcgcagtcctgttgctgtcctttgccctcatcatcctcccctccatcagcccttttggccccaacaaaaccgagagccctggggactttgcgcctgtacgagtgttctccagaactttgcacaacgatgctgcctcccgcgtggctgctgatgctgtgccaggctccgaggccccaggaccccgacccgaggctgacacaacccgagaagagtctccaggaagccccggggcagactggggcttccaggacaccgcgaacctgaccaattcgacggaggagctggacaacgccaccctggtcctgaggaatgcaacagaggggctgggccaggtcgccctgctggactgggtggcgcctgggccgagcactggctcaggacgtgcagggctggaggcggcgggagacgagctgtgagccccgccaggactatgctcccaggcccctctgcccaggggtgccttggggatgctgcactgggcagctacccacctggggatgggacgtgaggccaagaccccagcagagatgccagaatgggggaggcacagctcatagccacacacccagggcctgactgaggcccacgcaggaaccgacactcagacacaaggcaaagagggccacaggacccgggaaatacacacagagccaggagcagaagcaaagagcagacacacatacagcctgaaacagacctggacagacagacacagcctgaaacagacccggacagacagacacagcctgaaacagacccagacaaacagacagacagacacagcctgaaacagacccagacagacagacagacagcctgaaacagacccagacacagcctgaaacagatccggacagacagacagaaacagcctgaaacagacccagacagacagacagacacagcctgaaacagacccggacagacagacagacacagcctgaaacagacccggacagacagacagacacagcctgaaacagacccggacagacagacagacacagcctgaaacagacctagacagacagacacagattgaaacagacccagacaaacagacagacacagcctgaaacagacccagacacagcctgaaacagacccggacagacagacagacacagcctgaaacagacccagacagacagacagacacagcctgaaacagacccagacagacagaccgacgcagcctgaaagagacccagacagagagacaggcagacacagcctaaaacagacctggacagacaggcagacgtagtctgaaacagacctgaacagacagacagacgcacacacacaacagatgcgcagcaactccccgcccagggacccctcccggcctccctcgcacactgggaggaggaagccgccgagactgcagggagcctggccccggagccccgggtgcgccctggtctttggagcagccacggcccacaatcaccccccttttctaagactgcctgatccgaaataaagtattttgacagaa

SEQ ID NO: 43 - DNA, 2586 bp, Homo sapiens
  source 1..2586: mol_type = other DNA
acagagccacagagggctgtgagcttgcccagccccaggtaacgctggcggtgggtgggcctccagcttggagcagagaccccccgaggcatctgcagacagaactggatggacccatgaatacggatttagctgctggaaagatggcttctgctgcctgctccatggaccccatcgacagctttgagctcctggatctcctgtttgaccggcaggacggcatcctgagacacgtggagctgggcgagggctggggtcacgtcaaggaccagcaggtcctgccaaaccccgactctgacgacttcctcagctccatcctgggctctggagactcactgcccagctccccactctggtcccccgaaggcagtgatagtggcatctccgaagacctcccctccgacccccaggacacccctccacgcagcggaccagccacctcccccgccggctgccatcctgcccagcctggcaaggggccctgcctctcctatcatcctggcaactcttgctccaccacaaccccagggccagtgatccaagtacctgaagcctctgtgaccatagacctggaaatgtggagcccaggaggaaggatctgtgctgagaagccggctgatccggtggacctgtccccacgatgcaatctcaccgtgaaagacctcctcctttcgggcagcagtggggacctgcaacagcatcacctgggggcctcctacctcctgcgacctggggctgggcactgtcaggagctggtgctcaccgaggatgagaagaagctgctggctaaagaaggcatcaccctgcccactcagctgcccctcactaagtacgaggagcgagtgctgaaaaaaatccgccggaaaatccggaacaagcagtcggcgcaagaaagcaggaagaagaagaaggaatatatcgatggcctggagactcggatgtcagcttgcactgctcagaatcaggagttacagaggaaagtcttgcatctcgagaagcaaaacctgtccctcttggagcaactgaagaaactccaggccattgtggtgcagtccaccagcaagtcagcccagacaggcacctgtgtcgcagtcctgttgctgtcctttgccctcatcatcctcccctccatcagcccttttggccccaacaaaaccgagagccctggggactttgcgcctgtacgagtgttctccagaactttgcacaacgatgctgcctcccgcgtggctgctgatgctgtgccaggctccgaggccccaggaccccgacccgaggctgacacaacccgagaagagtctccaggaagccccggggcagactggggcttccaggacaccgcgaacctgaccaattcgacggaggagctggacaacgccaccctggtcctgaggaatgcaacagaggggctgggccaggtcgccctgctggactgggtggcgcctgggccgagcactggctcaggacgtgcagggctggaggcggcgggagacgagctgtgagccccgccaggactatgctcccaggcccctctgcccaggggtgccttggggatgctgcactgggcagctacccacctggggatgggacgtgaggccaagaccccagcagagatgccagaatgggggaggcacagctcatagccacacacccagggcctgactgaggcccacgcaggaaccgacactcagacacaaggcaaagagggccacaggacccgggaaatacacacagagccaggagcagaagcaaagagcagacacacatacagcctgaaacagacctggacagacagacacagcctgaaacagacccggacagacagacacagcctgaaacagacccagacaaacagacagacagacacagcctgaaacagacccagacagacagacagacagcctgaaacagacccagacacagcctgaaacagatccggacagacagacagaaacagcctgaaacagacccagacagacagacagacacagcctgaaacagacccggacagacagacagacacagcctgaaacagacccggacagacagacagacacagcctgaaacagacccggacagacagacagacacagcctgaaacagacctagacagacagacacagattgaaacagacccagacaaacagacagacacagcctgaaacagacccagacacagcctgaaacagacccggacagacagacagacacagcctgaaacagacccagacagacagacagacacagcctgaaacagacccagacagacagaccgacgcagcctgaaagagacccagacagagagacaggcagacacagcctaaaacagacctggacagacaggcagacgtagtctgaaacagacctgaacagacagacagacgcacacacacaacagatgcgcagcaactccccgcccagggacccctcccggcctccctcgcacactgggaggaggaagccgccgagactgcagggagcctggccccggagccccgggtgcgccctggtctttggagcagccacggcccacaatcaccccccttttctaagactgcctgatccgaaataaagtattttgacaaa

SEQ ID NO: 44 - DNA, 1485 bp, Homo sapiens
  source 1..1485: mol_type = other DNA
aacgctggcggtgggtgggcctccagcttggagcagagaccccccgaggcatctgcagacagaactggatggacccatgaatacggatttagctgctggaaagatggcttctgctgcctgctccatggaccccatcgacagctttgagctcctggatctcctgtttgaccggcaggacggcatcctgagacacgtggagctgggcgagggctggggtcacgtcaaggaccagcaggtcctgccaaaccccgactctgacgacttcctcagctccatcctgggctctggagactcactgcccagctccccactctggtcccccgaaggcagtgatagtggcatctccgaagacctcccctccgacccccaggacacccctccacgcagcggaccagccacctcccccgccggctgccatcctgcccagcctggcaaggggccctgcctctcctatcatcctggcaactcttgctccaccacaaccccagggccagtgatccaagtacctgaagcctctgtgaccatagacctggaaatgtggagcccaggaggaaggatctgtgctgagaagccggctgatccggtggacctgtccccacgatgcaatctcaccgtgaaagacctcctcctttcgggcagcagtggggacctgcaacagcatcacctgggggcctcctacctcctgcgacctggggctgggcactgtcaggagctggtgctcaccgaggatgagaagaagctgctggctaaagaaggcatcaccctgcccactcagctgcccctcactaagtacgaggagcgagtgctgaaaaaaatccgccggaaaatccggaacaagcagtcggcgcaagaaagcaggaagaagaagaaggaatatatcgatggcctggagactcggatgtcagcttgcactgctcagaatcaggagttacagaggaaagtcttgcatctcgagaagcaaaacctgtccctcttggagcaactgaagaaactccaggccattgtggtgcagtccaccagcaagtcagcccagacaggcacctgtgtcgcagtcctgttgctgtcctttgccctcatcatcctcccctccatcagcccttttggccccaacaaaaccgagagccctggggactttgcgcctgtacgagtgttctccagaactttgcacaacgatgctgcctcccgcgtggctgctgatgctgtgccaggctccgaggccccaggaccccgacccgaggctgacacaacccgagaagagtctccaggaagccccggggcagactggggcttccaggacaccgcgaacctgaccaattcgacggaggagctggacaacgccaccctggtcctgaggaatgcaacagaggggctgggccaggtcgccctgctggactgggtggcgcctgggccgagcactggctcaggacgtgcagggctggaggcggcgggagacgagctgtgagccccgccaggactatgctccca

SEQ ID NO: 45 - DNA, 2588 bp, Homo sapiens
  source 1..2588: mol_type = other DNA
gacagagccacagagggctgtgagcttgcccggccccaggtaacgctggcggtgggtgggcctccagcttggagcagagaccccccgaggcatctgcagacagaactggatggacccatgaatacggatttagctgctggaaagatggcttctgctgcctgctccatggaccccatcgacagctttgagctcctggatctcctgtttgaccggcaggacggcatcctgagacacgtggagctgggcgagggctggggtcacgtcaaggaccagcaggtcctgccaaaccccgactctgacgacttcctcagctccatcctgggctctggagactcactgcccagctccccactctggtcccccgaaggcagtgatagtggcatctccgaagacctcccctccgacccccaggacacccctccacgcagcggaccagccacctcccccgccggctgccatcctgcccagcctggcaaggggccctgcctctcctatcatcctggcaactcttgctccaccacaaccccagggccagtgatccaagtacctgaagcctctgtgaccatagacctggaaatgtggagcccaggaggaaggatctgtgctgagaagccggctgatccggtggacctgtccccacgatgcaatctcaccgtgaaaaacctcctcctttcgggcagcagtggggacctgcaacagcatcacctgggggcctcctacctcctgcgacctggggctgggcactgtcaggagctggtgctcaccgaggatgagaagaagctgctggctaaagaaggcatcaccctgcccactcagctgcccctcactaagtacgaggagcgagtgctgaaaaaaatccgccggaaaatccggaacaagcagtcggcgcaagaaagcaggaagaagaagaaggaatatatcgatggcctggagactcggatgtcagcttgcactgctcagaatcaggagttacagaggaaagtcttgcatctcgagaagcaaaacctgtccctcttggagcaactgaagaaactccaggccattgtggtgcagtccaccagcaagtcagcccagacaggcacctgtgtcgcagtcctgttgctgtcctttgccctcatcatcctcccctccatcagcccttttggccccaacaaaaccgagagccctggggactttgcgcctgtacgagtgttctccagaactttgcacaacgatgctgcctcccgcgtggctgctgatgctgtgccaggctccgaggccccaggaccccgacccgaggctgacacaacccgagaagagtctccaggaagccccggggcagactggggcttccaggacaccgcgaacctgaccaattcgacggaggagctggacaacgccaccctggtcctgaggaatgcaacagaggggctgggccaggtcgccctgctggactgggtggcgcctgggccgagcactggctcaggacgtgcagggctggaggcggcgggagacgagctgtgagccccgccaggactatgctcccaggcccctctgcccaggggtgccttggggatgctgcactgggcagctacccacctggggatgggacgtgaggccaagaccccagcagagatgccagaatgggggaggcacagctcatagccacacacccagggcctgactgaggcccacgcaggaaccgacactcagacacaaggcaaagagggccacaggacccgggaaatacacacagagccaggagcagaagcaaagagcagacacacatacagcctgaaacagacctggacagacagacacagcctgaaacagacccggacagacagacacagcctgaaacagacccagacaaacagacagacagacacagcctgaaacagacccagacagacagacagacagcctgaaacagacccagacacagcctgaaacagatccggacagacagacagaaacagcctgaaacagacccagacagacagacagacacagcctgaaacagacccggacagacagacagacacagcctgaaacagacccggacagacagacagacacagcctgaaacagacccggacagacagacagacacagcctgaaacagacctagacagacagacacagattgaaacagacccagacaaacagacagacacagcctgaaacagacccagacacagcctgaaacagacccggacagacagacagacacagcctgaaacagacccagacagacagacagacacagcctgaaacagacccagacagacagaccgacgcagcctgaaagagacccagacagagagacaggcagacacagcctaaaacagacctggacagacaggcagacgtagtctgaaacagacctgaacagacagacagacgcacacacacaacagatgcgcagcaactccccgcccagggacccctcccggcctccctcgcacactgggaggaggaagccgccgagactgcagggagcctggccccggagccccgggtgcgccctggtctttggagcagccacggcccacaatcaccccccttttctaagactgcctgatccgaaataaagtattttgacagaa

SEQ ID NO: 46 - DNA, 1491 bp, Homo sapiens
  source 1..1491: mol_type = other DNA
acagagggctgtgagcttgcccggccccaggtaacgctggcggtgggtgggcctccagcttggagcagagaccccccgaggcatctgcagacagaactggatggacccatgaatacggatttagctgctggaaagatggcttctgctgcctgctccatggaccccatcgacagctttgagctcctggatctcctgtttgaccggcaggacggcatcctgagacacgtggagctgggcgagggctggggtcacgtcaaggaccaggtcctgccaaaccccgactctgacgacttcctcagctccatcctgggctctggagactcactgcccagctccccactctggtcccccgaaggcagtgatagtggcatctccgaagacctcccctccgacccccaggacacccctccacgcagcggaccagccacctcccccgccggctgccatcctgcccagcctggcaaggggccctgcctctcctatcatcctggcaactcttgctccaccacaaccccagggccagtgatccaagtacctgaagcctctgtgaccatagacctggaaatgtggagcccaggaggaaggatctgtgctgagaagccggctgatccggtggacctgtccccacgatgcaatctcaccgtgaaaaacctcctcctttcgggcagcagtggggacctgcaacagcatcacctgggggcctcctacctcctgcgacctggggctgggcactgtcaggagctggtgctcaccgaggatgagaagaagctgctggctaaagaaggcatcaccctgcccactcagctgcccctcactaagtacgaggagcgagtgctgaaaaaaatccgccggaaaatccggaacaagcagtcggcgcaagaaagcaggaagaagaagaaggaatatatcgatggcctggagactcggatgtcagcttgcactgctcagaatcaggagttacagaggaaagtcttgcatctcgagaagcaaaacctgtccctcttggagcaactgaagaaactccaggccattgtggtgcagtccaccagcaagtcagcccagacaggcacctgtgtcgcagtcctgttgctgtcctttgccctcatcatcctcccctccatcagcccttttggccccaacaaaaccgagagccctggggactttgcgcctgtacgagtgttctccagaactttgcacaacgatgctgcctcccgcgtggctgctgatgctgtgccaggctccgaggccccaggaccccgacccgaggctgacacaacccgagaagagtctccaggaagccccggggcagactggggcttccaggacaccgcgaacctgaccaattcgacggaggagctggacaacgccaccctggtcctgaggaatgcaacagaggggctgggccaggtcgccctgctggactgggtggcgcctgggccgagcactggctcaggacgtgcagggctggaggcggcgggagacgagctgtga

SEQ ID NO: 47 - DNA, 1475 bp, Homo sapiens
  source 1..1475: mol_type = other DNA
aggtaacgctggcggtgggtgggcctccagcttggagcagagaccccccgaggcatctgcagacagaactggatggacccatgaatacggatttagctgctggaaagatggcttctgctgcctgctccatggaccccatcgacagctttgagctcctggatctcctgtttgaccggcaggacggcatcctgagacacgtggagctgggcgagggctggggtcacgtcaaggaccagcaggtcctgccaaaccccgactctgacgacttcctcagctccatcctgggctctggagactcactgcccagctccccactctggtcccccgaaggcagtgatagtggcatctccgaagacctcccctccgacccccaggacacccctccacgcagcggaccagccacctcccccgccggctgccatcctgcccagcctggcaaggggccctgcctctcctatcatcctggcaactcttgctccaccacaaccccagggccagtgatccaagtacctgaagcctctgtgaccatagacctggaaatgtggagcccaggaggaaggatctgtgctgagaagccggctgatccggtggacctgtccccacgatgcaatctcaccgtgaaaaacctcctcctttcgggcagcagtggggacctgcatcacctgggggcctcctacctcctgcgacctggggctgggcactgtcaggagctggtgctcaccgaggatgagaagaagctgctggctaaagaaggcatcaccctgcccactcagctgcccctcactaagtacgaggagcgagtgctgaaaaaaatccgccggaaaatccggaacaagcagtcggcgcaagaaagcaggaagaagaagaaggaatatatcgatggcctggagactcggatgtcagcttgcactgctcagaatcaggagttacagaggaaagtcttgcatctcgagaagcaaaacctgtccctcttggagcaactgaagaaactccaggccattgtggtgcagtccaccagcaagtcagcccagacaggcacctgtgtcgcagtcctgttgctgtcctttgccctcatcatcctcccctccatcagcccttttggccccaacaaaaccgagagccctggggactttgcgcctgtacgagtgttctccagaactttgcacaacgatgctgcctcccgcgtggctgctgatgctgtgccaggctccgaggccccaggaccccgacccgaggctgacacaacccgagaagagtctccaggaagccccggggcagactggggcttccaggacaccgcgaacctgaccaattcgacggaggagctggacaacgccaccctggtcctgaggaatgcaacagaggggctgggccaggtcgccctgctggactgggtggcgcctgggccgagcactggctcaggacgtgcagggctggaggcggcgggagacgagctgtgagccccgccaggacta

SEQ ID NO: 48 - DNA, 1382 bp, Homo sapiens
  source 1..1382: mol_type = other DNA
aggtaacgctggcggtgggtgggcctccagcttggagcagagaccccccgaggcatctgcagacagaactggatggacccatgaatacggatttagctgctggaaagatggcttctgctgcctgctccatggaccccatcgacagctttgagctcctggatctcctgtttgaccggcaggacggcatcctgagacacgtggagctgggcgagggctggggtcacgtcaaggaccagcaggtcctgccaaaccccgactctgacgacttcctcagctccatcctgggctctggagactcactgcccagctccccactctggtcccccgaaggcagtgatagtggcatctccgaagacctcccctccgacccccaggacacccctccacgcagcggaccagccacctcccccgccggctgccatcctgcccagcctggcaaggggccctgcctctcctatcatcctggcaactcttgctccaccacaaccccagggccagtgatccaagtacctgaagcctctgtgaccatagacctggaaatgtggagcccaggaggaaggatctgtgctgagaagccggctgatccggtggacctgtccccacgatgcaatctcaccgtgaaaaacctcctcctttcgggcagcagtggggacctgcaacagcatcacctgggggcctcctacctcctgcgacctggggctgggcactgtcaggagctggtgctcaccgaggatgagaagaagctgctggctaaagaaggcatcaccctgcccactcagctgcccctcactaaggatgtcagcttgcactgctcagaatcaggagttacagaggaaagtcttgcatctcgagaagcaaaacctgtccctcttggagcaactgaagaaactccaggccattgtggtgcagtccaccagcaagtcagcccagacaggcacctgtgtcgcagtcctgttgctgtcctttgccctcatcatcctcccctccatcagcccttttggccccaacaaaaccgagagccctggggactttgcgcctgtacgagtgttctccagaactttgcacaacgatgctgcctcccgcgtggctgctgatgctgtgccaggctccgaggccccaggaccccgacccgaggctgacacaacccgagaagagtctccaggaagccccggggcagactggggcttccaggacaccgcgaacctgaccaattcgacggaggagctggacaacgccaccctggtcctgaggaatgcaacagaggggctgggccaggtcgccctgctggactgggtggcgcctgggccgagcactggctcaggacgtgcagggctggaggcggcgggagacgagctgtgagccccgccaggactatgctccca

SEQ ID NO: 49 - DNA, 2585 bp, Homo sapiens
  source 1..2585: mol_type = other DNA
gacagagccacagagggctgtgagcttgcccggccccaggtaacgctggcggtgggtgggcctccagcttggagcagagaccccccgaggcatctgcagacagaactggatggacccatgaatacggatttagctgctggaaagatggcttctgctgcctgctccatggaccccatcgacagctttgagctcctggatctcctgtttgaccggcaggacggcatcctgagacacgtggagctgggcgagggctggggtcacgtcaaggaccaggtcctgccaaaccccgactctgacgacttcctcagctccatcctgggctctggagactcactgcccagctccccactctggtcccccgaaggcagtgatagtggcatctccgaagacctcccctccgacccccaggacacccctccacgcagcggaccagccacctcccccgccggctgccatcctgcccagcctggcaaggggccctgcctctcctatcatcctggcaactcttgctccaccacaaccccagggccagtgatccaagtacctgaagcctctgtgaccatagacctggaaatgtggagcccaggaggaaggatctgtgctgagaagccggctgatccggtggacctgtccccacgatgcaatctcaccgtgaaaaacctcctcctttcgggcagcagtggggacctgcaacagcatcacctgggggcctcctacctcctgcgacctggggctgggcactgtcaggagctggtgctcaccgaggatgagaagaagctgctggctaaagaaggcatcaccctgcccactcagctgcccctcactaagtacgaggagcgagtgctgaaaaaaatccgccggaaaatccggaacaagcagtcggcgcaagaaagcaggaagaagaagaaggaatatatcgatggcctggagactcggatgtcagcttgcactgctcagaatcaggagttacagaggaaagtcttgcatctcgagaagcaaaacctgtccctcttggagcaactgaagaaactccaggccattgtggtgcagtccaccagcaagtcagcccagacaggcacctgtgtcgcagtcctgttgctgtcctttgccctcatcatcctcccctccatcagcccttttggccccaacaaaaccgagagccctggggactttgcgcctgtacgagtgttctccagaactttgcacaacgatgctgcctcccgcgtggctgctgatgctgtgccaggctccgaggccccaggaccccgacccgaggctgacacaacccgagaagagtctccaggaagccccggggcagactggggcttccaggacaccgcgaacctgaccaattcgacggaggagctggacaacgccaccctggtcctgaggaatgcaacagaggggctgggccaggtcgccctgctggactgggtggcgcctgggccgagcactggctcaggacgtgcagggctggaggcggcgggagacgagctgtgagccccgccaggactatgctcccaggcccctctgcccaggggtgccttggggatgctgcactgggcagctacccacctggggatgggacgtgaggccaagaccccagcagagatgccagaatgggggaggcacagctcatagccacacacccagggcctgactgaggcccacgcaggaaccgacactcagacacaaggcaaagagggccacaggacccgggaaatacacacagagccaggagcagaagcaaagagcagacacacatacagcctgaaacagacctggacagacagacacagcctgaaacagacccggacagacagacacagcctgaaacagacccagacaaacagacagacagacacagcctgaaacagacccagacagacagacagacagcctgaaacagacccagacacagcctgaaacagatccggacagacagacagaaacagcctgaaacagacccagacagacagacagacacagcctgaaacagacccggacagacagacagacacagcctgaaacagacccggacagacagacagacacagcctgaaacagacccggacagacagacagacacagcctgaaacagacctagacagacagacacagattgaaacagacccagacaaacagacagacacagcctgaaacagacccagacacagcctgaaacagacccggacagacagacagacacagcctgaaacagacccagacagacagacagacacagcctgaaacagacccagacagacagaccgacgcagcctgaaagagacccagacagagagacaggcagacacagcctaaaacagacctggacagacaggcagacgtagtctgaaacagacctgaacagacagacagacgcacacacacaacagatgcgcagcaactccccgcccagggacccctcccggcctccctcgcacactgggaggaggaagccgccgagactgcagggagcctggccccggagccccgggtgcgccctggtctttggagcagccacggcccacaatcaccccccttttctaagactgcctgatccgaaataaagtattttgacagaa

SEQ ID NO: 50 - DNA, 2582 bp, Homo sapiens
  source 1..2582: mol_type = other DNA
gacagagccacagagggctgtgagcttgcccggccccaggtaacgctggcggtgggtgggcctccagcttggagcagagaccccccgaggcatctgcagacagaactggatggacccatgaatacggatttagctgctggaaagatggcttctgctgcctgctccatggaccccatcgacagctttgagctcctggatctcctgtttgaccggcaggacggcatcctgagacacgtggagctgggcgagggctggggtcacgtcaaggaccagcaggtcctgccaaaccccgactctgacgacttcctcagctccatcctgggctctggagactcactgcccagctccccactctggtcccccgaaggcagtgatagtggcatctccgaagacctcccctccgacccccaggacacccctccacgcagcggaccagccacctcccccgccggctgccatcctgcccagcctggcaaggggccctgcctctcctatcatcctggcaactcttgctccaccacaaccccagggccagtgatccaagtacctgaagcctctgtgaccatagacctggaaatgtggagcccaggaggaaggatctgtgctgagaagccggctgatccggtggacctgtccccacgatgcaatctcaccgtgaaaaacctcctcctttcgggcagcagtggggacctgcatcacctgggggcctcctacctcctgcgacctggggctgggcactgtcaggagctggtgctcaccgaggatgagaagaagctgctggctaaagaaggcatcaccctgcccactcagctgcccctcactaagtacgaggagcgagtgctgaaaaaaatccgccggaaaatccggaacaagcagtcggcgcaagaaagcaggaagaagaagaaggaatatatcgatggcctggagactcggatgtcagcttgcactgctcagaatcaggagttacagaggaaagtcttgcatctcgagaagcaaaacctgtccctcttggagcaactgaagaaactccaggccattgtggtgcagtccaccagcaagtcagcccagacaggcacctgtgtcgcagtcctgttgctgtcctttgccctcatcatcctcccctccatcagcccttttggccccaacaaaaccgagagccctggggactttgcgcctgtacgagtgttctccagaactttgcacaacgatgctgcctcccgcgtggctgctgatgctgtgccaggctccgaggccccaggaccccgacccgaggctgacacaacccgagaagagtctccaggaagccccggggcagactggggcttccaggacaccgcgaacctgaccaattcgacggaggagctggacaacgccaccctggtcctgaggaatgcaacagaggggctgggccaggtcgccctgctggactgggtggcgcctgggccgagcactggctcaggacgtgcagggctggaggcggcgggagacgagctgtgagccccgccaggactatgctcccaggcccctctgcccaggggtgccttggggatgctgcactgggcagctacccacctggggatgggacgtgaggccaagaccccagcagagatgccagaatgggggaggcacagctcatagccacacacccagggcctgactgaggcccacgcaggaaccgacactcagacacaaggcaaagagggccacaggacccgggaaatacacacagagccaggagcagaagcaaagagcagacacacatacagcctgaaacagacctggacagacagacacagcctgaaacagacccggacagacagacacagcctgaaacagacccagacaaacagacagacagacacagcctgaaacagacccagacagacagacagacagcctgaaacagacccagacacagcctgaaacagatccggacagacagacagaaacagcctgaaacagacccagacagacagacagacacagcctgaaacagacccggacagacagacagacacagcctgaaacagacccggacagacagacagacacagcctgaaacagacccggacagacagacagacacagcctgaaacagacctagacagacagacacagattgaaacagacccagacaaacagacagacacagcctgaaacagacccagacacagcctgaaacagacccggacagacagacagacacagcctgaaacagacccagacagacagacagacacagcctgaaacagacccagacagacagaccgacgcagcctgaaagagacccagacagagagacaggcagacacagcctaaaacagacctggacagacaggcagacgtagtctgaaacagacctgaacagacagacagacgcacacacacaacagatgcgcagcaactccccgcccagggacccctcccggcctccctcgcacactgggaggaggaagccgccgagactgcagggagcctggccccggagccccgggtgcgccctggtctttggagcagccacggcccacaatcaccccccttttctaagactgcctgatccgaaataaagtattttgacagaa

SEQ ID NO: 51 - DNA, 2481 bp, Homo sapiens
  source 1..2481: mol_type = other DNA
gacagagccacagagggctgtgagcttgcccggccccaggtaacgctggcggtgggtgggcctccagcttggagcagagaccccccgaggcatctgcagacagaactggatggacccatgaatacggatttagctgctggaaagatggcttctgctgcctgctccatggaccccatcgacagctttgagctcctggatctcctgtttgaccggcaggacggcatcctgagacacgtggagctgggcgagggctggggtcacgtcaaggaccagcaggtcctgccaaaccccgactctgacgacttcctcagctccatcctgggctctggagactcactgcccagctccccactctggtcccccgaaggcagtgatagtggcatctccgaagacctcccctccgacccccaggacacccctccacgcagcggaccagccacctcccccgccggctgccatcctgcccagcctggcaaggggccctgcctctcctatcatcctggcaactcttgctccaccacaaccccagggccagtgatccaagtacctgaagcctctgtgaccatagacctggaaatgtggagcccaggaggaaggatctgtgctgagaagccggctgatccggtggacctgtccccacgatgcaatctcaccgtgaaaaacctcctcctttcgggcagcagtggggacctgcaacagcatcacctgggggcctcctacctcctgcgacctggggctgggcactgtcaggagctggtgctcaccgaggatgagaagaagctgctggctaaagaaggcatcaccctgcccactcagctgcccctcactaaggatgtcagcttgcactgctcagaatcaggagttacagaggaaagtcttgcatctcgagaagcaaaacctgtccctcttggagcaactgaagaaactccaggccattgtggtgcagtccaccagcaagtcagcccagacaggcacctgtgtcgcagtcctgttgctgtcctttgccctcatcatcctcccctccatcagcccttttggccccaacaaaaccgagagccctggggactttgcgcctgtacgagtgttctccagaactttgcacaacgatgctgcctcccgcgtggctgctgatgctgtgccaggctccgaggccccaggaccccgacccgaggctgacacaacccgagaagagtctccaggaagccccggggcagactggggcttccaggacaccgcgaacctgaccaattcgacggaggagctggacaacgccaccctggtcctgaggaatgcaacagaggggctgggccaggtcgccctgctggactgggtggcgcctgggccgagcactggctcaggacgtgcagggctggaggcggcgggagacgagctgtgagccccgccaggactatgctcccaggcccctctgcccaggggtgccttggggatgctgcactgggcagctacccacctggggatgggacgtgaggccaagaccccagcagagatgccagaatgggggaggcacagctcatagccacacacccagggcctgactgaggcccacgcaggaaccgacactcagacacaaggcaaagagggccacaggacccgggaaatacacacagagccaggagcagaagcaaagagcagacacacatacagcctgaaacagacctggacagacagacacagcctgaaacagacccggacagacagacacagcctgaaacagacccagacaaacagacagacagacacagcctgaaacagacccagacagacagacagacagcctgaaacagacccagacacagcctgaaacagatccggacagacagacagaaacagcctgaaacagacccagacagacagacagacacagcctgaaacagacccggacagacagacagacacagcctgaaacagacccggacagacagacagacacagcctgaaacagacccggacagacagacagacacagcctgaaacagacctagacagacagacacagattgaaacagacccagacaaacagacagacacagcctgaaacagacccagacacagcctgaaacagacccggacagacagacagacacagcctgaaacagacccagacagacagacagacacagcctgaaacagacccagacagacagaccgacgcagcctgaaagagacccagacagagagacaggcagacacagcctaaaacagacctggacagacaggcagacgtagtctgaaacagacctgaacagacagacagacgcacacacacaacagatgcgcagcaactccccgcccagggacccctcccggcctccctcgcacactgggaggaggaagccgccgagactgcagggagcctggccccggagccccgggtgcgccctggtctttggagcagccacggcccacaatcaccccccttttctaagactgcctgatccgaaataaagtattttgacagaa

SEQ ID NO: 52 - DNA, 1505 bp, Homo sapiens
  source 1..1505: mol_type = other DNA
gtgacagagccacagagggctgtgagcttgcccagccccaggtaacgctggcggtgggtgggcctccagcttggagcagagaccccccgaggcatctgcagacagaactggatggacccatgaatacggatttagctgctggaaagatggcttctgctgcctgctccatggaccccatcgacagctttgagctcctggatctcctgtttgaccggcaggacggcatcctgagacacgtggagctgggcgagggctggggtcacgtcaaggaccagcaggtcctgccaaaccccgactctgacgacttcctcagctccatcctgggctctggagactcactgcccagctccccactctggtcccccgaaggcagtgatagtggcatctccgaagacctcccctccgacccccaggacacccctccacgcagcggaccagccacctcccccgccggctgccatcctgcccagcctggcaaggggccctgcctctcctatcatcctggcaactcttgctccaccacaaccccagggccagtgatccaagtacctgaagcctctgtgaccatagacctggaaatgtggagcccaggaggaaggatctgtgctgagaagccggctgatccggtggacctgtccccacgatgcaatctcaccgtgaaaaacctcctcctttcgggcagcagtggggacctgcaacagcatcacctgggggcctcctacctcctgcgacctggggctgggcactgtcaggagctggtgctcaccgaggatgagaagaagctgctggctaaagaaggcatcaccctgcccactcagctgcccctcactaagtacgaggagcgagtgctgaaaaaaatccgccggaaaatccggaacaagcagtcggcgcaagaaagcaggaagaagaagaaggaatatatcgatggcctggagactcggatgtcagcttgcactgctcagaatcaggagttacagaggaaagtcttgcacctcgagaagcaaaacctgtccctcttggagcaactgaagaaactccaggccattgtggtgcagtccaccagcaagtcagcccagacaggcacctgtgtcgcagtcctgttgctgtcctttgccctcatcatcctcccctccatcagcccttttggccccaacaaaaccgagagccctggggactttgcgcctgtacgagtgttctccagaactttgcacaacgatgctgcctcccgcgtggctgctgatgctgtgccaggctccgaggccccaggaccccgacccgaggctgacacaacccgagaagagtctccaggaagccccggggcagactggggcttccaggacaccgcgaacctgaccaattcgacggaggagctggacaacgccaccctggtcctgaggaatgcaacagaggggctgggccaggtcgccctgctggactgggtggcgcctgggccgagcactggctcaggacgtgcagggctggaggcggcgggagacgagctgtga

SEQ ID NO: 53 - DNA, 2206 bp, Homo sapiens
  source 1..2206: mol_type = other DNA
acagagccacagagggctgtgagcttgcccagccccaggtaacgctggcggtgggtgggccaccagcttggagcagagaccccccgaggcatctgcagacagaactggatggacccatgaatacggatttagctgctggaaagatggcttctgctgcctgctccatggaccccatcgacagctttgagctcctggatctcctgtttgaccggcaggacggcatcctgagacacgtggagctgggcgagggctggggtcacgtcaaggaccagcaggtcctgccaaaccccgactctgacgacttcctcagctccatcctgggctctggagactcactgcccagctccccactctggtcccccgaaggcagtgatagtggcatctccgaagacctcccctccgacccccaggacacccctccacgcagcggaccagccacctcccccgccggctgccatcctgcccagcctggcaaggggccctgcctctcctatcatcctggcaactcttgctccaccacaaccccagggccagtgatccaagtacctgaagcctctgtgaccatagacctggaaatgtggagcccaggaggaaggatctgtgctgagaagccggctgatccggtggacctgtccccacgatgcaatctcaccgtgaaaaacctcctcctttcgggcagcagtggggacctgcaacagcatcacctgggggcctcctacctcctgcgacctggggctgggcactgtcaggagctggtgctcaccgaggatgagaagaagctgctggctaaagaaggcatcaccctgcccactcagctgcccctcactaagtacgaggagcgagtgctgaaaaaaatccgccggaaaatccggaacaagcagtcggcgcaagaaagcaggaagaagaagaaggccattgtggtgcagtccaccagcaagtcagcccagacaggcacctgtgtcgcagtcctgttgctgtcctttgccctcatcatcctcccctccatcagcccttttggccccaacaaaaccgagagccctggggactttgcgcctgtacgagtgttctccagaactttgcacaacgatgctgcctcccgcgtggctgctgatgctgtgccaggctccgaggccccaggaccccgacccgaggctgacacaacccgagaagagtctccaggaagccccggggcagactggggcttccaggacaccgcgaacctgaccaattcgacggaggagctggacaacgccaccctggtcctgaggaatgcaacagaggggctgggccaggtcgccccgctggactgggtggcgcctgggcccgagcactggctcaggacgtgcagggctggaggcggcgggagacgagcttgtgagccccgccaggactatgctcccaggcccctctgcccaggggtgccttggggatgctgcactgggcagctacccacctggggatgggacgtgaggccaagaccccagcagagatgccagaatgggggaggcacagctcatagccacacacccagggcctgactgaggcccacgcaggaaccgacactcagacacaaggcaaagagggccacaggacccgggaaatacacacagagccaggagcagaagcaaagagcagacacacatacagcctgaaacagacctggacagacagacacagcctgaaacagacccggacagacagacacagcctgaaacagacccagacaaacagacagacagacacagcctgaaacagacccagacagacagacagacagcctgaaacagacccagacacagcctgaaacagatccggacagacagacagaaacagcctgaaacagacccagacagacagacagacacagcctgaaacagacccggacagacagacagacacagcctgaaagagacccagacagagagacaggcagacacagcctaaaacagacctggacagacaggcagacgtagtctgaaacagacctgaacagacagacagacgcacacacacaacagatgcgcagcaactccccgcccagggacccctcccggcctccctcgcacactgggaggaggaagccgccgagactgcagggagcctggccccggagccccgggtgcgccctggtctttggagcagccacggcccacaatcaccccccttttctaagactgcctgatccgaaataaagtattttgacaaa

SEQ ID NO: 54 - DNA, 2582 bp, Homo sapiens
  source 1..2582: mol_type = other DNA
acagagggctgtgagcttgcccggccccaggtaacgctggcggtgggtgggcctccagcttggagcagagaccccccgaggcatctgcagacagaactggatggacccatgaatacggatttagctgctggaaagatggcttctgctgcctgctccatggaccccatcgacagctttgagctcctggatctcctgtttgaccggcaggacggcatcctgagacacgtggagctgggcgagggctggggtcacgtcaaggaccaggtcctgccaaaccccgactctgacgacttcctcagctccatcctgggctctggagactcactgcccagctccccactctggtcccccgaaggcagtgatagtggcatctccgaagacctcccctccgacccccaggacacccctccacgcagcggaccagccacctcccccgccggctgccatcctgcccagcctggcaaggggccctgcctctcctatcatcctggcaactcttgctccaccacaaccccagggccagtgatccaagtacctgaagcctctgtgaccatagacctggaaatgtggagcccaggaggaaggatctgtgctgagaagccggctgatccggtggacctgtccccacgatgcaatctcaccgtgaaaaacctcctcctttcgggcagcagtggggacctgcaacagcatcacctgggggcctcctacctcctgcgacctggggctgggcactgtcaggagctggtgctcaccgaggatgagaagaagctgctggctaaagaaggcatcaccctgcccactcagctgcccctcactaagtacgaggagcgagtgctgaaaaaaatccgccggaaaatccggaacaagcagtcggcgcaagaaagcaggaagaagaagaaggaatatatcgatggcctggagactcggatgtcagcttgcactgctcagaatcaggagttacagaggaaagtcttgcatctcgagaagcaaaacctgtccctcttggagcaactgaagaaactccaggccattgtggtgcagtccaccagcaagtcagcccagacaggcacctgtgtcgcagtcctgttgctgtcctttgccctcatcatcctcccctccatcagcccttttggccccaacaaaaccgagagccctggggactttgcgcctgtacgagtgttctccagaactttgcacaacgatgctgcctcccgcgtggctgctgatgctgtgccaggctccgaggccccaggaccccgacccgaggctgacacaacccgagaagagtctccaggaagccccggggcagactggggcttccaggacaccgcgaacctgaccaattcgacggaggagctggacaacgccaccctggtcctgaggaatgcaacagaggggctgggccaggtcgccctgctggactgggtggcgcctgggccgagcactggctcaggacgtgcagggctggaggcggcgggagacgagctgtgagccccgccaggactatgctcccaggcccctctgcccaggggtgccttggggatgctgcactgggcagctacccacctggggatgggacgtgaggccaagaccccagcagagatgccagaatgggggaggcacagctcatagccacacacccagggcctgactgaggcccacgcaggaaccgacactcagacacaaggcaaagagggccacaggacccgggaaatacacacagagccaggagcagaagcaaagagcagacacacatacagcctgaaacagacctggacagacagacacagcctgaaacagacccggacagacagacacagcctgaaacagacccagacaaacagacagacagacacagcctgaaacagacccagacagacagacagacagcctgaaacagacccagacacagcctgaaacagatccggacagacagacagaaacagcctgaaacagacccagacagacagacagacacagcctgaaacagacccggacagacagacagacacagcctgaaacagacccggacagacagacagacacagcctgaaacagacccggacagacagacagacacagcctgaaacagacctagacagacagacacagattgaaacagacccagacaaacagacagacacagcctgaaacagacccagacacagcctgaaacagacccggacagacagacagacacagcctgaaacagacccagacagacagacagacacagcctgaaacagacccagacagacagaccgacgcagcctgaaagagacccagacagagagacaggcagacacagcctaaaacagacctggacagacaggcagacgtagtctgaaacagacctgaacagacagacagacgcacacacacaacagatgcgcagcaactccccgcccagggacccctcccggcctccctcgcacactgggaggaggaagccgccgagactgcagggagcctggccccggagccccgggtgcgccctggtctttggagcagccacggcccacaatcaccccccttttctaagactgcctgatccgaaataaagtattttgacagaaaaaaaa

SEQ ID NO: 55 - DNA, 1489 bp, Homo sapiens
  source 1..1489: mol_type = other DNA
aggtaacgctggcggtgggtgggcctccagcttggagcagagaccccccgaggcatctgcagacagaactggatggacccatgaatacggatttagctgctggaaagatggcttctgctgcctgctccatggaccccatcgacagctttgagctcctggatctcctgtttgaccggcaggacggcatcctgagacacgtggagctgggcgagggctggggtcacgtcaaggaccagcaggtcctgccaaaccccgactctgacgacttcctcagctccatcctgggctctggagactcactgcccagctccccactctggtcccccgaaggcagtgatagtggcatctccgaagacctcccctccgacccccaggacacccctccacgcagcggaccagccacctcccccgccggctgccatcctgcccagcctggcaaggggccctgcctctcctatcatcctggcaactcttgctccaccacaaccccagggccagtgatccaagtacctgaagcctctgtgaccatagacctggaaatgtggagcccaggaggaaggatctgtgctgagaagccggctgatccggtggacctgtccccacgatgcaatctcaccgtgaaaaacctcctcctttcgggcagcagtggggacctgcaacagcatcacctgggggcctcctacctcctgcgacctggggctgggcactgtcaggagctggtgctcaccgaggatgagaagaagctgctggctaaagaaggcatcaccctgcccactcagctgcccctcactaagtacgaggagcgagtgctgaaaaaaatccgccggaaaatccggaacaagcagtcggcgcaagaaagcaggaagaagaagaaggaatatatcgatggcctggagactcggatgtcagcttgcactgctcagaatcaggagttacagaggaaagtcttgcatctcgagaagcaaaacctgtccctcttggagcaactgaagaaactccaggccattgtggtgcagtccaccagcaagtcagcccagacaggcacctgtgtcgcagtcctgttgctgtcctttgccctcatcatcctcccctccatcagcccttttggccccaacaaaaccgagagccctggggactttgcgcctgtacgagtgttctccagaactttgcacaacgatgctgcctcccgcgtggctgctgatgctgtgccaggctccgaggccccaggaccccgacccgaggctgacacaacccgagaagagtctccaggaagccccggggcagactggggcttccaggacaccgcgaacctgaccaattcgacggaggagctggacaacgccaccctggtcctgaggaatgcaacagaggggctgggccaggtcgccctgctggactgggtggcgcctgggccgagcactggctcaggacgtgcagggctggaggcggcgggagacgagctgtgagccccgccaggactatgctccca

SEQ ID NO: 56 - DNA, 2618 bp, Homo sapiens
  source 1..2618: mol_type = other DNA
gtgaccgcttcagcatcttttgggagtggtgacagagccacagagggctgtgagcttgcccggccccaggtaacgctggcggtgggtgggcctccagcttggagcagagaccccccgaggcatctgcagacagaactggatggacccatgaatacggatttagctgctggaaagatggcttctgctgcctgctccatggaccccatcgacagctttgagctcctggatctcctgtttgaccggcaggacggcatcctgagacacgtggagctgggcgagggctggggtcacgtcaaggaccagcaggtcctgccaaaccccgactctgacgacttcctcagctccatcctgggctctggagactcactgcccagctccccactctggtcccccgaaggcagtgatagtggcatctccgaagacctcccctccgacccccaggacacccctccacgcagcggaccagccacctcccccgccggctgccatcctgcccagcctggcaaggggccctgcctctcctatcatcctggcaactcttgctccaccacaaccccagggccagtgatccaagtacctgaagcctctgtgaccatagacctggaaatgtggagcccaggaggaaggatctgtgctgagaagccggctgatccggtggacctgtccccacgatgcaatctcaccgtgaaaaacctcctcctttcgggcagcagtggggacctgcaacagcatcacctgggggcctcctacctcctgcgacctggggctgggcactgtcaggagctggtgctcaccgaggatgagaagaagctgctggctaaagaaggcatcaccctgcccactcagctgcccctcactaagtacgaggagcgagtgctgaaaaaaatccgccggaaaatccggaacaagcagtcggcgcaagaaagcaggaagaagaagaaggaatatatcgatggcctggagactcggatgtcagcttgcactgctcagaatcaggagttacagaggaaagtcttgcatctcgagaagcaaaacctgtccctcttggagcaactgaagaaactccaggccattgtggtgcagtccaccagcaagtcagcccagacaggcacctgtgtcgcagtcctgttgctgtcctttgccctcatcatcctcccctccatcagcccttttggccccaacaaaaccgagagccctggggactttgcgcctgtacgagtgttctccagaactttgcacaacgatgctgcctcccgcgtggctgctgatgctgtgccaggctccgaggccccaggaccccgacccgaggctgacacaacccgagaagagtctccaggaagccccggggcagactggggcttccaggacaccgcgaacctgaccaattcgacggaggagctggacaacgccaccctggtcctgaggaatgcaacagaggggctgggccaggtcgccctgctggactgggtggcgcctgggccgagcactggctcaggacgtgcagggctggaggcggcgggagacgagctgtgagccccgccaggactatgctcccaggcccctctgcccaggggtgccttggggatgctgcactgggcagctacccacctggggatgggacgtgaggccaagaccccagcagagatgccagaatgggggaggcacagctcatagccacacacccagggcctgactgaggcccacgcaggaaccgacactcagacacaaggcaaagagggccacaggacccgggaaatacacacagagccaggagcagaagcaaagagcagacacacatacagcctgaaacagacctggacagacagacacagcctgaaacagacccggacagacagacacagcctgaaacagacccagacaaacagacagacagacacagcctgaaacagacccagacagacagacagacagcctgaaacagacccagacacagcctgaaacagatccggacagacagacagaaacagcctgaaacagacccagacagacagacagacacagcctgaaacagacccggacagacagacagacacagcctgaaacagacccggacagacagacagacacagcctgaaacagacccggacagacagacagacacagcctgaaacagacctagacagacagacacagattgaaacagacccagacaaacagacagacacagcctgaaacagacccagacacagcctgaaacagacccggacagacagacagacacagcctgaaacagacccagacagacagacagacacagcctgaaacagacccagacagacagaccgacgcagcctgaaagagacccagacagagagacaggcagacacagcctaaaacagacctggacagacaggcagacgtagtctgaaacagacctgaacagacagacagacgcacacacacaacagatgcgcagcaactccccgcccagggacccctcccggcctccctcgcacactgggaggaggaagccgccgagactgcagggagcctggccccggagccccgggtgcgccctggtctttggagcagccacggcccacaatcaccccccttttctaagactgcctgatccgaaataaagtattttgacagaa

SEQ ID NO: 57 - DNA, 2586 bp, Homo sapiens
  source 1..2586: mol_type = other DNA
acagagccacagagggctgtgagcttgcccagccccaggtaacgctggcggtgggtgggcctccagcttggagcagagaccccccgaggcatctgcagacagaactggatggacccatgaatacggatttagctgctggaaagatggcttctgctgcctgctccatggaccccatcgacagctttgagctcctggatctcctgtttgaccggcaggacggcatcctgagacacgtggagctgggcgagggctggggtcacgtcaaggaccagcaggtcctgccaaaccccgactctgacgacttcctcagctccatcctgggctctggagactcactgcccagctccccactctggtcccccgaaggcagtgatagtggcatctccgaagacctcccctccgacccccaggacacccctccacgcagcggaccagccacctcccccgccggctgccatcctgcccagcctggcaaggggccctgcctctcctatcatcctggcaactcttgctccaccacaaccccagggccagtgatccaagtacctgaagcctctgtgaccatagacctggaaatgtggagcccaggaggaaggatctgtgctgagaagccggctgatccggtggacctgtccccacgatgcaatctcaccgtgaaaaacctcctcctttcgggcagcagtggggacctgcaacagcatcacctgggggcctcctacctcctgcgacctggggctgggcactgtcaggagctggtgctcaccgaggatgagaagaagctgctggctaaagaaggcatcaccctgcccactcagctgcccctcactaagtacgaggagcgagtgctgaaaaaaatccgccggaaaatccggaacaagcagtcggcgcaagaaagcaggaagaagaagaaggaatatatcgatggcctggagactcggatgtcagcttgcactgctcagaatcaggagttacagaggaaagtcttgcatctcgagaagcaaaacctgtccctcttggagcaactgaagaaactccaggccattgtggtgcagtccaccagcaagtcagcccagacaggcacctgtgtcgcagtcctgttgctgtcctttgccctcatcatcctcccctccatcagcccttttggccccaacaaaaccgagagccctggggactttgcgcctgtacgagtgttctccagaactttgcacaacgatgctgcctcccgcgtggctgctgatgctgtgccaggctccgaggccccaggaccccgacccgaggctgacacaacccgagaagagtctccaggaagccccggggcagactggggcttccaggacaccgcgaacctgaccaattcgacggaggagctggacaacgccaccctggtcctgaggaatgcaacagaggggctgggccaggtcgccctgctggactgggtggcgcctgggccgagcactggctcaggacgtgcagggctggaggcggcgggagacgagctgtgagccccgccaggactatgctcccaggcccctctgcccaggggtgccttggggatgctgcactgggcagctacccacctggggatgggacgtgaggccaagaccccagcagagatgccagaatgggggaggcacagctcatagccacacacccagggcctgactgaggcccacgcaggaaccgacactcagacacaaggcaaagagggccacaggacccgggaaatacacacagagccaggagcagaagcaaagagcagacacacatacagcctgaaacagacctggacagacagacacagcctgaaacagacccggacagacagacacagcctgaaacagacccagacaaacagacagacagacacagcctgaaacagacccagacagacagacagacagcctgaaacagacccagacacagcctgaaacagatccggacagacagacagaaacagcctgaaacagacccagacagacagacagacacagcctgaaacagacccggacagacagacagacacagcctgaaacagacccggacagacagacagacacagcctgaaacagacccggacagacagacagacacagcctgaaacagacctagacagacagacacagattgaaacagacccagacaaacagacagacacagcctgaaacagacccagacacagcctgaaacagacccggacagacagacagacacagcctgaaacagacccagacagacagacagacacagcctgaaacagacccagacagacagaccgacgcagcctgaaagagacccagacagagagacaggcagacacagcctaaaacagacctggacagacaggcagacgtagtctgaaacagacctgaacagacagacagacgcacacacacaacagatgcgcagcaactccccgcccagggacccctcccggcctccctcgcacactgggaggaggaagccgccgagactgcagggagcctggccccggagccccgggtgcgccctggtctttggagcagccacggcccacaatcaccccccttttctaagactgcctgatccgaaataaagtattttgacaaa

SEQ ID NO: 58 - DNA, 1485 bp, Homo sapiens
  source 1..1485: mol_type = other DNA
aacgctggcggtgggtgggcctccagcttggagcagagaccccccgaggcatctgcagacagaactggatggacccatgaatacggatttagctgctggaaagatggcttctgctgcctgctccatggaccccatcgacagctttgagctcctggatctcctgtttgaccggcaggacggcatcctgagacacgtggagctgggcgagggctggggtcacgtcaaggaccagcaggtcctgccaaaccccgactctgacgacttcctcagctccatcctgggctctggagactcactgcccagctccccactctggtcccccgaaggcagtgatagtggcatctccgaagacctcccctccgacccccaggacacccctccacgcagcggaccagccacctcccccgccggctgccatcctgcccagcctggcaaggggccctgcctctcctatcatcctggcaactcttgctccaccacaaccccagggccagtgatccaagtacctgaagcctctgtgaccatagacctggaaatgtggagcccaggaggaaggatctgtgctgagaagccggctgatccggtggacctgtccccacgatgcaatctcaccgtgaaaaacctcctcctttcgggcagcagtggggacctgcaacagcatcacctgggggcctcctacctcctgcgacctggggctgggcactgtcaggagctggtgctcaccgaggatgagaagaagctgctggctaaagaaggcatcaccctgcccactcagctgcccctcactaagtacgaggagcgagtgctgaaaaaaatccgccggaaaatccggaacaagcagtcggcgcaagaaagcaggaagaagaagaaggaatatatcgatggcctggagactcggatgtcagcttgcactgctcagaatcaggagttacagaggaaagtcttgcatctcgagaagcaaaacctgtccctcttggagcaactgaagaaactccaggccattgtggtgcagtccaccagcaagtcagcccagacaggcacctgtgtcgcagtcctgttgctgtcctttgccctcatcatcctcccctccatcagcccttttggccccaacaaaaccgagagccctggggactttgcgcctgtacgagtgttctccagaactttgcacaacgatgctgcctcccgcgtggctgctgatgctgtgccaggctccgaggccccaggaccccgacccgaggctgacacaacccgagaagagtctccaggaagccccggggcagactggggcttccaggacaccgcgaacctgaccaattcgacggaggagctggacaacgccaccctggtcctgaggaatgcaacagaggggctgggccaggtcgccctgctggactgggtggcgcctgggccgagcactggctcaggacgtgcagggctggaggcggcgggagacgagctgtgagccccgccaggactatgctccca

SEQ ID NO: 59 - protein, 461 aa, Homo sapiens
MNTDLAAGKMASAACSMDPIDSFELLDLLFDRQDGILRHVELGEGWGHVKDQQVLPNPDSDDFLSSILGSGDSLPSSPLWSPEGSDSGISEDLPSDPQDTPPRSGPATSPAGCHPAQPGKGPCLSYHPGNSCSTTTPGPVIQVPEASVTIDLEMWSPGGRICAEKPADPVDLSPRCNLTVKDLLLSGSSGDLQQHHLGASYLLRPGAGHCQELVLTEDEKKLLAKEGITLPTQLPLTKYEERVLKKIRRKIRNKQSAQESRKKKKEYIDGLETRMSACTAQNQELQRKVLHLEKQNLSLLEQLKKLQAIVVQSTSKSAQTGTCVAVLLLSFALIILPSISPFGPNKTESPGDFAPVRVFSRTLHNDAASRVAADAVPGSEAPGPRPEADTTREESPGSPGADWGFQDTANLTNSTEELDNATLVLRNATEGLGQVALLDWVAPGPSTGSGRAGLEAAGDEL

SEQ ID NO: 60 - protein, 459 aa, Homo sapiens
MNTDLAAGKMASAACSMDPIDSFELLDLLFDRQDGILRHVELGEGWGHVKDQQVLPNPDSDDFLSSILGSGDSLPSSPLWSPEGSDSGISEDLPSDPQDTPPRSGPATSPAGCHPAQPGKGPCLSYHPGNSCSTTTPGPVIQVPEASVTIDLEMWSPGGRICAEKPADPVDLSPRCNLTVKDLLLSGSSGDLHHLGASYLLRPGAGHCQELVLTEDEKKLLAKEGITLPTQLPLTKYEERVLKKIRRKIRNKQSAQESRKKKKEYIDGLETRMSACTAQNQELQRKVLHLEKQNLSLLEQLKKLQAIVVQSTSKSAQTGTCVAVLLLSFALIILPSISPFGPNKTESPGDFAPVRVFSRTLHNDAASRVAADAVPGSEAPGPRPEADTTREESPGSPGADWGFQDTANLTNSTEELDNATLVLRNATEGLGQVALLDWVAPGPSTGSGRAGLEAAGDEL

SEQ ID NO: 61 - protein, 337 aa, Homo sapiens
MNTDLAAGKMASAACSMDPIDSFELLDLLFDRQDGILRHVELGEGWGHVKDQQVLPNPDSDDFLSSILGSGDSLPSSPLWSPEGSDSGISEDLPSDPQDTPPRSGPATSPAGCHPAQPGKGPCLSYHPGNSCSTTTPGPVIQVPEASVTIDLEMWSPGGRICAEKPADPVDLSPRCNLTVKDLLLSGSSGDLQQHHLGASYLLRPGAGHCQELVLTEDEKKLLAKEGITLPTQLPLTKDVSLHCSESGVTEESLASREAKPVPLGATEETPGHCGAVHQQVSPDRHLCRSPVAVLCPHHPPLHQPFWPQQNREPWGLCACTSVLQNFAQRCCLPRGC

SEQ ID NO: 62 - protein, 473 aa, Homo sapiens [sequence omitted]

SEQ ID NO: 63 - protein, 460 aa, Homo sapiens
MNTDLAAGKMASAACSMDPIDSFELLDLLFDRQDGILRHVELGEGWGHVKDQVLPNPDSDDFLSSILGSGDSLPSSPLWSPEGSDSGISEDLPSDPQDTPPRSGPATSPAGCHPAQPGKGPCLSYHPGNSCSTTTPGPVIQVPEASVTIDLEMWSPGGRICAEKPADPVDLSPRCNLTVKDLLLSGSSGDLQQHHLGASYLLRPGAGHCQELVLTEDEKKLLAKEGITLPTQLPLTKYEERVLKKIRRKIRNKQSAQESRKKKKEYIDGLETRMSACTAQNQELQRKVLHLEKQNLSLLEQLKKLQAIVVQSTSKSAQTGTCVAVLLLSFALIILPSISPFGPNKTESPGDFAPVRVFSRTLHNDAASRVAADAVPGSEAPGPRPEADTTREESPGSPGADWGFQDTANLTNSTEELDNATLVLRNATEGLGQVALLDWVAPGPSTGSGRAGLEAAGDEL

SEQ ID NO: 64 - protein, 461 aa, Homo sapiens
MNTDLAAGKMASAACSMDPIDSFELLDLLFDRQDGILRHVELGEGWGHVKDQQVLPNPDSDDFLSSILGSGDSLPSSPLWSPEGSDSGISEDLPSDPQDTPPRSGPATSPAGCHPAQPGKGPCLSYHPGNSCSTTTPGPVIQVPEASVTIDLEMWSPGGRICAEKPADPVDLSPRCNLTVKNLLLSGSSGDLQQHHLGASYLLRPGAGHCQELVLTEDEKKLLAKEGITLPTQLPLTKYEERVLKKIRRKIRNKQSAQESRKKKKEYIDGLETRMSACTAQNQELQRKVLHLEKQNLSLLEQLKKLQAIVVQSTSKSAQTGTCVAVLLLSFALIILPSISPFGPNKTESPGDFAPVRVFSRTLHNDAASRVAADAVPGSEAPGPRPEADTTREESPGSPGADWGFQDTANLTNSTEELDNATLVLRNATEGLGQVALLDWVAPGPSTGSGRAGLEAAGDEL

SEQ ID NO: 65 - protein, 459 aa, Homo sapiens
MNTDLAAGKMASAACSMDPIDSFELLDLLFDRQDGILRHVELGEGWGHVKDQQVLPNPDSDDFLSSILGSGDSLPSSPLWSPEGSDSGISEDLPSDPQDTPPRSGPATSPAGCHPAQPGKGPCLSYHPGNSCSTTTPGPVIQVPEASVTIDLEMWSPGGRICAEKPADPVDLSPRCNLTVKNLLLSGSSGDLHHLGASYLLRPGAGHCQELVLTEDEKKLLAKEGITLPTQLPLTKYEERVLKKIRRKIRNKQSAQESRKKKKEYIDGLETRMSACTAQNQELQRKVLHLEKQNLSLLEQLKKLQAIVVQSTSKSAQTGTCVAVLLLSFALIILPSISPFGPNKTESPGDFAPVRVFSRTLHNDAASRVAADAVPGSEAPGPRPEADTTREESPGSPGADWGFQDTANLTNSTEELDNATLVLRNATEGLGQVALLDWVAPGPSTGSGRAGLEAAGDEL

SEQ ID NO: 66 - protein, 337 aa, Homo sapiens
MNTDLAAGKMASAACSMDPIDSFELLDLLFDRQDGILRHVELGEGWGHVKDQQVLPNPDSDDFLSSILGSGDSLPSSPLWSPEGSDSGISEDLPSDPQDTPPRSGPATSPAGCHPAQPGKGPCLSYHPGNSCSTTTPGPVIQVPEASVTIDLEMWSPGGRICAEKPADPVDLSPRCNLTVKNLLLSGSSGDLQQHHLGASYLLRPGAGHCQELVLTEDEKKLLAKEGITLPTQLPLTKDVSLHCSESGVTEESLASREAKPVPLGATEETPGHCGAVHQQVSPDRHLCRSPVAVLCPHHPPLHQPFWPQQNREPWGLCACTSVLQNFAQRCCLPRGC

SEQ ID NO: 67 - protein, 473 aa, Homo sapiens
MNTDLAAGKMASAACSMDPIDSFELLDLLFDRQDGILRHVELGEGWGHVKDQQVLPNPDSDDFLSSILGSGDSLPSSPLWSPEGSDSGISEDLPSDPQDTPPRSGPATSPAGCHPAQPGKGPCLSYHPGNSCSTTTPGPVIQVPEASVTIDLEMWSPGGRICAEKPADPVDLSPRCNLTVKNLLLSGSSGDLQQHHLGASYLLRPGAGHCQELVLTEDEKKLLAKEGITLPTQLPLTKYEERVLKKIRRKIRNKQSAQESRKKKKAIVVQSTSKSAQTGTCVAVLLLSFALIILPSISPFGPNKTESPGDFAPVRVFSRTLHNDAASRVAADAVPGSEAPGPRPEADTTREESPGSPGADWGFQDTANLTNSTEELDNATLVLRNATEGLGQVAPLDWVAPGPEHWLRTCRAGGGGRRACEPRQDYAPRPLCPGVPWGCCTGQLPTWGWDVRPRPQQRCQNGGGTAHSHTPRA

SEQ ID NO: 68 - protein, 460 aa, Homo sapiens
MNTDLAAGKMASAACSMDPIDSFELLDLLFDRQDGILRHVELGEGWGHVKDQVLPNPDSDDFLSSILGSGDSLPSSPLWSPEGSDSGISEDLPSDPQDTPPRSGPATSPAGCHPAQPGKGPCLSYHPGNSCSTTTPGPVIQVPEASVTIDLEMWSPGGRICAEKPADPVDLSPRCNLTVKNLLLSGSSGDLQQHHLGASYLLRPGAGHCQELVLTEDEKKLLAKEGITLPTQLPLTKYEERVLKKIRRKIRNKQSAQESRKKKKEYIDGLETRMSACTAQNQELQRKVLHLEKQNLSLLEQLKKLQAIVVQSTSKSAQTGTCVAVLLLSFALIILPSISPFGPNKTESPGDFAPVRVFSRTLHNDAASRVAADAVPGSEAPGPRPEADTTREESPGSPGADWGFQDTANLTNSTEELDNATLVLRNATEGLGQVALLDWVAPGPSTGSGRAGLEAAGDEL

SEQ ID NO: 69 - DNA, 20 bp, synthetic construct
  misc_feature 1..20: note = Synthetic sequence; gRNA Recognition Sequence
  source 1..20: mol_type = other DNA
ttcacggtgagattgcatcg

SEQ ID NO: 70 - DNA, 20 bp, synthetic construct
  misc_feature 1..20: note = Synthetic sequence; gRNA Recognition Sequence
  source 1..20: mol_type = other DNA
agttgccaggatgataggag

SEQ ID NO: 71 - DNA, 20 bp, synthetic construct
  misc_feature 1..20: note = Synthetic sequence; gRNA Recognition Sequence
  source 1..20: mol_type = other DNA
gagtgctgaaaaaaatccgc

SEQ ID NO: 72 - DNA, 20 bp, synthetic construct
  misc_feature 1..20: note = Synthetic sequence; gRNA Recognition Sequence
  source 1..20: mol_type = other DNA
aagaagaaggaatatatcga

SEQ ID NO: 73 - DNA, 20 bp, synthetic construct
  misc_feature 1..20: note = Synthetic sequence; gRNA Recognition Sequence
  source 1..20: mol_type = other DNA
tgaggaagtcgtcagagtcg

SEQ ID NO: 74 - DNA, 20 bp, synthetic construct
  misc_feature 1..20: note = Synthetic sequence; gRNA Recognition Sequence
  source 1..20: mol_type = other DNA
gaaaatccggaacaagcagt

SEQ ID NO: 75 - DNA, 20 bp, synthetic construct
  misc_feature 1..20: note = Synthetic sequence; gRNA Recognition Sequence
  source 1..20: mol_type = other DNA
gcctctgtgaccatagacct

SEQ ID NO: 76 - DNA, 20 bp, synthetic construct
  misc_feature 1..20: note = Synthetic sequence; gRNA Recognition Sequence
  source 1..20: mol_type = other DNA
cacggtgagattgcatcgtg

SEQ ID NO: 77 - DNA, 20 bp, synthetic construct
  misc_feature 1..20: note = Synthetic sequence; gRNA Recognition Sequence
  source 1..20: mol_type = other DNA
acccaggtctatggtcacag

SEQ ID NO: 78 - DNA, 20 bp, synthetic construct
  misc_feature 1..20: note = Synthetic sequence; gRNA Recognition Sequence
  source 1..20: mol_type = other DNA
cgatggcctggagactcggt

SEQ ID NO: 79 - DNA, 20 bp, synthetic construct
  misc_feature 1..20: note = Synthetic sequence; gRNA Recognition Sequence
  source 1..20: mol_type = other DNA
tgccactatcactgccttcg

SEQ ID NO: 80 - DNA, 20 bp, synthetic construct
  misc_feature 1..20: note = Synthetic sequence; gRNA Recognition Sequence
  source 1..20: mol_type = other DNA
ccatttcacttggcagtacg

SEQ ID NO: 81 - DNA, 20 bp, synthetic construct
  misc_feature 1..20: note = Synthetic sequence; gRNA Recognition Sequence
  source 1..20: mol_type = other DNA
tcctggatctcctgtttgac

SEQ ID NO: 82 - DNA, 20 bp, synthetic construct
  misc_feature 1..20: note = Synthetic sequence; gRNA Recognition Sequence
  source 1..20: mol_type = other DNA
ctcctgtttgaccggcagga

SEQ ID NO: 83 - DNA, 20 bp, synthetic construct
  misc_feature 1..20: note = Synthetic sequence; gRNA Recognition Sequence
  source 1..20: mol_type = other DNA
cccagactcaccttagtgag

SEQ ID NO: 84 - DNA, 20 bp, synthetic construct
  misc_feature 1..20: note = Synthetic sequence; gRNA Recognition Sequence
  source 1..20: mol_type = other DNA
ccaggatgataggagaggca

SEQ ID NO: 85 - DNA, 20 bp, synthetic construct
  misc_feature 1..20: note = Synthetic sequence; gRNA Recognition Sequence
  source 1..20: mol_type = other DNA
tcacggtgagattgcatcgt

SEQ ID NO: 86 - DNA, 20 bp, synthetic construct
  misc_feature 1..20: note = Synthetic sequence; gRNA Recognition Sequence
  source 1..20: mol_type = other DNA
ggacggcatcctgagacacg

SEQ ID NO: 87 - DNA, 20 bp, synthetic construct
  misc_feature 1..20: note = Synthetic sequence; gRNA Recognition Sequence
  source 1..20: mol_type = other DNA
agccccaggtcgcaggaggt

SEQ ID NO: 88 - DNA, 20 bp, synthetic construct
  misc_feature 1..20: note = Synthetic sequence; gRNA Recognition Sequence
  source 1..20: mol_type = other DNA
gtttcttcagttgctccaag

(19) United States
(12) Patent Application Publication
(10) Pub. No.: US 2023/0160012 A1
(43) Pub. Date: May 25, 2023

(54) Treatment of Liver Diseases With CAMP Responsive Element Binding Protein 3 Like 3 (CREB3L3) Inhibitors

(71) Applicant: Regeneron Pharmaceuticals, Inc., Tarrytown, NY (US)

(72) Inventors: Niek Verweij, Tarrytown, NY (US); Luca Andrea Lotta, Tarrytown, NY (US); Aris Baras, Tarrytown, NY (US)

(21) Appl. No.: 17/823,057

(22) Filed: Aug. 29, 2022

Related U.S. Application Data
(60) Provisional application No. 63/239,304, filed on Aug. 31, 2021.

(51) Int. Cl. C12Q 1/6883

(52) U.S. Cl. CPC C12Q 1/6883; C12Q 1/6869

(57) ABSTRACT

The present disclosure provides methods of treating subjects having a liver disease with a CAMP Responsive Element Binding Protein 3 Like 3 (CREB3L3) inhibitor, and methods of identifying subjects having an increased risk of developing a liver disease.

### REFERENCE TO SEQUENCE LISTING

This application includes a Sequence Listing submitted electronically as an XML file named 381203557SEQ, created on Aug. 23, 2022, with a size of 242 kilobytes. The Sequence Listing is incorporated herein by reference.

### FIELD

The present disclosure relates generally to the treatment of subjects having a liver disease with CAMP Responsive Element Binding Protein 3 Like 3 (CREB3L3) inhibitors, and methods of identifying subjects having an increased risk of developing a liver disease.

### BACKGROUND

Chronic liver disease and cirrhosis are leading causes of morbidity and mortality in the United States, accounting for 38,170 deaths (1.5% of total deaths) in 2014 (Kochanek et al., Nat’l. Vital Stat. Rep., 2016, 65,1-122). The most common etiologies of cirrhosis in the U.S. are alcoholic liver disease, chronic hepatitis C, and nonalcoholic fatty liver disease (NAFLD), together accounting for about 80% of patients awaiting liver transplant between 2004 and 2013 (Wong et al., Gastroenterology, 2015, 148, 547-555). The estimated prevalence of NAFLD in the U.S. is between 19 and 46 percent (Browning et al., Hepatology, 2004, 40, 1387-1395; Lazo et al., Am. J. Epidemiol., 2013, 178, 38-45; and Williams et al., Gastroenterology, 2011, 140, 124-131) and has been rising over time (Younossi et al., Clin. Gastroenterol. Hepatol., 2011, 9, 524-530), likely in conjunction with increased prevalence of obesity, which is one of its primary risk factors (Cohen et al., Science, 2011, 332, 1519-1523). While significant advances have been made in the treatment of hepatitis C, there are currently no evidence-based treatments for alcoholic or nonalcoholic liver disease or cirrhosis. Identifying naturally occurring genetic variants that protect from liver damage and liver disease outcomes can be a pathway to identify novel therapeutic targets for liver disease (Abul-Husn et al. N. Engl. J. Med., 2018, 378,1096-106).

CREB3L3 is a member of the basic-leucine zipper family and the AMP-dependent transcription factor family. CREB3L3 is localized to the endoplasmic reticulum and acts in response to cAMP stimulation during endoplasmic reticulum stress by activating unfolded protein response target genes’ transcription through box-B element. In vitro CREB3L3 binds the cyclic AMP response element (CRE) and the box-B element and has been linked to acute inflammatory response, hepatocellular carcinoma, triglyceride metabolism, and hepcidin expression.

### SUMMARY

The present disclosure provides methods of treating a subject having a liver disease, the methods comprising administering a CREB3L3 inhibitor to the subject.

The present disclosure also provides methods of treating a subject having parenchymal liver disease, the methods comprising administering a CREB3L3 inhibitor to the subject.

The present disclosure also provides methods of treating a subject having liver fibrosis, the methods comprising administering a CREB3L3 inhibitor to the subject.

The present disclosure also provides methods of treating a subject having liver cirrhosis, the methods comprising administering a CREB3L3 inhibitor to the subject.

The present disclosure also provides methods of treating a subject having non-alcoholic fatty liver disease (NAFLD), the methods comprising administering a CREB3L3 inhibitor to the subject.

The present disclosure also provides methods of treating a subject with a therapeutic agent that treats or inhibits a liver disease, wherein the subject has a liver disease, the methods comprising: determining whether the subject has a CREB3L3 variant nucleic acid molecule encoding a CREB3L3 predicted loss-of-function polypeptide by: obtaining or having obtained a biological sample from the subject; and performing or having performed a sequence analysis on the biological sample to determine if the subject has a genotype comprising the CREB3L3 variant nucleic acid molecule encoding the CREB3L3 predicted loss-of-function polypeptide; and administering or continuing to administer the therapeutic agent that treats or inhibits the liver disease in a standard dosage amount to a subject that is CREB3L3 reference, and administering a CREB3L3 inhibitor to the subject; and administering or continuing to administer the therapeutic agent that treats or inhibits the liver disease in an amount that is the same as or less than a standard dosage amount to a subject that is heterozygous for the CREB3L3 variant nucleic acid molecule, and administering a CREB3L3 inhibitor to the subject; wherein the presence of a genotype having the CREB3L3 variant nucleic acid molecule encoding the CREB3L3 predicted loss-of-function polypeptide indicates the subject has a reduced risk of developing the liver disease.

The present disclosure also provides methods of identifying a subject having an increased risk of developing a liver disease, the methods comprising: determining or having determined the presence or absence of a CREB3L3 variant nucleic acid molecule encoding a CREB3L3 predicted loss-of-function polypeptide in a biological sample obtained from the subject; wherein: when the subject is CREB3L3 reference, then the subject has an increased risk of developing the liver disease; and when the subject is heterozygous or homozygous for a CREB3L3 variant nucleic acid molecule encoding the CREB3L3 predicted loss-of-function polypeptide, then the subject has a decreased risk of developing the liver disease.

The present disclosure also provides therapeutic agents that treat or inhibit a liver disease for use in the treatment of a liver disease in a subject identified as having: a genomic nucleic acid molecule encoding a CREB3L3 predicted loss-of-function polypeptide, or the complement thereof, wherein the genomic nucleic acid molecule has a nucleotide sequence comprising an adenine at a position corresponding to position 6,120 according to SEQ ID NO:2, or the complement thereof; an mRNA molecule encoding a CREB3L3 predicted loss-of-function polypeptide, or the complement thereof, wherein the mRNA molecule has a nucleotide sequence comprising an adenine at a position corresponding to: position 661 according to SEQ ID NO:17, or the complement thereof; position 649 according to SEQ ID NO:18, or the complement thereof; position 624 according to SEQ ID NO:19, or the complement thereof; position 624 according to SEQ ID NO:20, or the complement thereof; position 658 according to SEQ ID NO:21, or the complement thereof; position 661 according to SEQ ID NO:22, or the complement thereof; position 661 according to SEQ ID NO:23, or the complement thereof; position 663 according to SEQ ID NO:24, or the complement thereof; position 660 according to SEQ ID NO:25, or the complement thereof; position 649 according to SEQ ID NO:26, or the complement thereof; position 624 according to SEQ ID NO:27, or the complement thereof; or position 691 according to SEQ ID NO:28, or the complement thereof; or a cDNA molecule encoding a CREB3L3 predicted loss-of-function polypeptide, or the complement thereof, wherein the cDNA molecule has a nucleotide sequence comprising an adenine at a position corresponding to: position 661 according to SEQ ID NO:45, or the complement thereof; position 649 according to SEQ ID NO:46, or the complement thereof; position 624 according to SEQ ID NO:47, or the complement thereof; position 624 according to SEQ ID NO:48, or the complement thereof; position 658 according to SEQ ID NO:49, or the complement thereof; position 661 according to SEQ ID NO:50, or the complement thereof; position 661 according to SEQ ID NO:51, or the complement thereof; position 663 according to SEQ ID NO:52, or the complement thereof; position 660 according to SEQ ID NO:53, or the complement thereof; position 649 according to SEQ ID NO:54, or the complement thereof; position 624 according to SEQ ID NO:55, or the complement thereof; or position 691 according to SEQ ID NO:56, or the complement thereof.

The present disclosure also provides CREB3L3 inhibitors for use in the treatment of a liver disease in a subject that: a) is reference for a CREB3L3 genomic nucleic acid molecule, a CREB3L3 mRNA molecule, or a CREB3L3 cDNA molecule; or b) is heterozygous for: i) a genomic nucleic acid molecule encoding a CREB3L3 predicted loss-of-function polypeptide, or the complement thereof, wherein the genomic nucleic acid molecule has a nucleotide sequence comprising an adenine at a position corresponding to position 6,120 according to SEQ ID NO:2, or the complement thereof; ii) an mRNA molecule encoding a CREB3L3 predicted loss-of-function polypeptide, or the complement thereof, wherein the mRNA molecule has a nucleotide sequence comprising an adenine at a position corresponding to: position 661 according to SEQ ID NO:17, or the complement thereof; position 649 according to SEQ ID NO:18, or the complement thereof; position 624 according to SEQ ID NO:19, or the complement thereof; position 624 according to SEQ ID NO:20, or the complement thereof; position 658 according to SEQ ID NO:21, or the complement thereof; position 661 according to SEQ ID NO:22, or the complement thereof; position 661 according to SEQ ID NO:23, or the complement thereof; position 663 according to SEQ ID NO:24, or the complement thereof; position 660 according to SEQ ID NO:25, or the complement thereof; position 649 according to SEQ ID NO:26, or the complement thereof; position 624 according to SEQ ID NO:27, or the complement thereof; or position 691 according to SEQ ID NO:28, or the complement thereof; or iii) a cDNA molecule encoding a CREB3L3 predicted loss-of-function polypeptide, or the complement thereof, wherein the cDNA molecule has a nucleotide sequence comprising an adenine at a position corresponding to: position 661 according to SEQ ID NO:45, or the complement thereof; position 649 according to SEQ ID NO:46, or the complement thereof; position 624 according to SEQ ID NO:47, or the complement thereof; position 624 according to SEQ ID NO:48, or the complement thereof; position 658 according to SEQ ID NO:49, or the complement thereof; position 661 according to SEQ ID NO:50, or the complement thereof; position 661 according to SEQ ID NO:51, or the complement thereof; position 663 according to SEQ ID NO:52, or the complement thereof; position 660 according to SEQ ID NO:53, or the complement thereof; position 649 according to SEQ ID NO:54, or the complement thereof; position 624 according to SEQ ID NO:55, or the complement thereof; or position 691 according to SEQ ID NO:56, or the complement thereof.

### DESCRIPTION

Various terms relating to aspects of the present disclosure are used throughout the specification and claims. Such terms are to be given their ordinary meaning in the art, unless otherwise indicated. Other specifically defined terms are to be construed in a manner consistent with the definitions provided herein.

Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred, in any respect. This holds for any possible non-expressed basis for interpretation, including matters of logic with respect to arrangement of steps or operational flow, plain meaning derived from grammatical organization or punctuation, or the number or type of aspects described in the specification.

As used herein, the singular forms “a,” “an” and “the” include plural referents unless the context clearly dictates otherwise.

As used herein, the term “about” means that the recited numerical value is approximate and small variations would not significantly affect the practice of the disclosed embodiments. Where a numerical value is used, unless indicated otherwise by the context, the term “about” means the numerical value can vary by ±10% and remain within the scope of the disclosed embodiments.

As used herein, the term “comprising” may be replaced with “consisting” or “consisting essentially of” in particular embodiments as desired.

As used herein, the term “isolated”, in regard to a nucleic acid molecule or a polypeptide, means that the nucleic acid molecule or polypeptide is in a condition other than its native environment, such as apart from blood and/or animal tissue. In some embodiments, an isolated nucleic acid molecule or polypeptide is substantially free of other nucleic acid molecules or other polypeptides, particularly other nucleic acid molecules or polypeptides of animal origin. In some embodiments, the nucleic acid molecule or polypeptide can be in a highly purified form, i.e., greater than 95% pure or greater than 99% pure. When used in this context, the term “isolated” does not exclude the presence of the same nucleic acid molecule or polypeptide in alternative physical forms, such as dimers or alternatively phosphorylated or derivatized forms.

As used herein, the terms “nucleic acid”, “nucleic acid molecule”, “nucleic acid sequence”, “polynucleotide”, or “oligonucleotide” can comprise a polymeric form of nucleotides of any length, can comprise DNA and/or RNA, and can be single-stranded, double-stranded, or multiple stranded. One strand of a nucleic acid also refers to its complement.

As used herein, the term “subject” includes any animal, including mammals. Mammals include, but are not limited to, farm animals (such as, for example, horse, cow, pig), companion animals (such as, for example, dog, cat), laboratory animals (such as, for example, mouse, rat, rabbits), and non-human primates (such as, for example, apes and monkeys). In some embodiments, the subject is a human. In some embodiments, the subject is a patient under the care of a physician.

Partial loss of function of the CREB3L3 gene is associated with a decreased risk of developing a liver disease in humans has been identified in accordance with the present disclosure. For example, a genetic alteration that changes the guanine at position 6,120 in the CREB3L3 reference genomic nucleic acid molecule (see, SEQ ID NO:1) to an adenine has been observed to indicate that the subject having such an alteration may have a decreased risk of developing a liver disease. It is believed that no variants of the CREB3L3 gene or protein have any known association with a liver disease in humans. Altogether, the genetic analyses described herein surprisingly indicate that the CREB3L3 gene and, in particular, a variant in the CREB3L3 gene, associates with a decreased risk of developing a liver disease. Moreover, the identification by the present disclosure of the association between additional variants and gene burden masks indicates that CREB3L3 itself (rather than linkage disequilibrium with variants in another gene) is responsible for a protective effect in liver diseases. Therefore, subjects that are CREB3L3 reference that have an increased risk of developing a liver disease, such as parenchymal liver disease, liver fibrosis, liver cirrhosis, or NAFLD, may be treated such that the liver disease is prevented, the symptoms thereof are reduced, and/or development of symptoms is repressed. Accordingly, the present disclosure provides methods of leveraging the identification of such variants in subjects to identify or stratify risk in such subjects of developing a liver disease, such as parenchymal liver disease, liver fibrosis, liver cirrhosis, or NAFLD, or to diagnose subjects as having an increased risk of developing a liver disease, such as parenchymal liver disease, liver fibrosis, liver cirrhosis, or NAFLD, such that subjects at risk or subjects with active disease may be treated accordingly.

For purposes of the present disclosure, any particular subject can be categorized as having one of three CREB3L3 genotypes: i) CREB3L3 reference; ii) heterozygous for a CREB3L3 variant nucleic acid molecule encoding a CREB3L3 predicted loss-of-function polypeptide; or iii) homozygous for a CREB3L3 variant nucleic acid molecule encoding a CREB3L3 predicted loss-of-function polypeptide. A subject is CREB3L3 reference when the subject does not have a copy of a CREB3L3 variant nucleic acid molecule encoding a CREB3L3 predicted loss-of-function polypeptide. A subject is heterozygous for a CREB3L3 variant nucleic acid molecule encoding a CREB3L3 predicted loss-of-function polypeptide when the subject has a single copy of a CREB3L3 variant nucleic acid molecule encoding a CREB3L3 predicted loss-of-function polypeptide. As used herein, a CREB3L3 variant nucleic acid molecule is any CREB3L3 nucleic acid molecule (such as, a genomic nucleic acid molecule, an mRNA molecule, or a cDNA molecule) encoding a CREB3L3 polypeptide having a partial loss-of-function, a complete loss-of-function, a predicted partial loss-of-function, or a predicted complete loss-of-function. A subject who has a CREB3L3 variant nucleic acid molecule encoding a CREB3L3 predicted loss-of-function polypeptide having a partial loss-of-function (or predicted partial loss-of-function) is hypomorphic for CREB3L3. The CREB3L3 variant nucleic acid molecule encoding a CREB3L3 predicted loss-of-function polypeptide can be any nucleic acid molecule encoding a CREB3L3 Asp182Asn-A, Asp182Asn-B, Asp182Asn-C, Asp182Asn-D, or Asp181Asn. In some embodiments, the CREB3L3 variant nucleic acid molecule encodes a CREB3L3 Asp182Asn-A, Asp182Asn-B, Asp182Asn-C, or Asp182Asn-D. A subject is homozygous for a CREB3L3 variant nucleic acid molecule encoding a CREB3L3 predicted loss-of-function polypeptide when the subject has two copies of a CREB3L3 variant nucleic acid molecule encoding a CREB3L3 predicted loss-of-function polypeptide.

For subjects that are genotyped or determined to be CREB3L3 reference, such subjects have an increased risk of developing a liver disease, such as parenchymal liver disease, liver fibrosis, liver cirrhosis, or NAFLD. For subjects that are genotyped or determined to be either CREB3L3 reference or heterozygous for a CREB3L3 variant nucleic acid molecule encoding a CREB3L3 predicted loss-of-function polypeptide, such subjects can be treated with a CREB3L3 inhibitor.

In any of the embodiments described throughout the present disclosure, the CREB3L3 variant nucleic acid molecule encoding a CREB3L3 predicted loss-of-function polypeptide can be any CREB3L3 nucleic acid molecule (such as, for example, genomic nucleic acid molecule, mRNA molecule, or cDNA molecule) encoding a CREB3L3 polypeptide having a partial loss-of-function, a complete loss-of-function, a predicted partial loss-of-function, or a predicted complete loss-of-function. For example, the CREB3L3 variant nucleic acid molecule can be any nucleic acid molecule encoding CREB3L3 Asp182Asn-A, Asp182Asn-B, Asp182Asn-C, Asp182Asn-D, or Asp181Asn. In some embodiments, the CREB3L3 variant nucleic acid molecule encodes CREB3L3 Asp182Asn-A, Asp182Asn-B, Asp182Asn-C, or Asp182Asn-D.

In any of the embodiments described throughout the present disclosure, the CREB3L3 predicted loss-of-function polypeptide can be any CREB3L3 polypeptide having a partial loss-of-function, a complete loss-of-function, a predicted partial loss-of-function, or a predicted complete loss-of-function. In any of the embodiments described throughout the present disclosure, the CREB3L3 predicted loss-of-function polypeptide can be any of the CREB3L3 polypeptides described herein including, for example, CREB3L3 Asp182Asn-A, Asp182Asn-B, Asp182Asn-C, Asp182Asn-D, or Asp181Asn. In some embodiments, the CREB3L3 predicted loss-of-function polypeptide is CREB3L3 Asp182Asn-A, Asp182Asn-B, Asp182Asn-C, or Asp182Asn-D.

In any of the embodiments described throughout the present disclosure, the liver disease a fatty liver disease (such as, for example, alcoholic fatty liver disease (AFLD), NAFLD or non-alcoholic steatohepatitis (NASH)), liver cirrhosis, liver fibrosis, an increased liver enzyme (such as, for example, alanine transaminase (ALT) or aspartate transaminase (AST)), simple steatosis, steatohepatitis, parenchymal liver disease, viral hepatitis, or hepatocellular carcinoma, or any of the complications of such conditions (including, but not limited to, heart or metabolic disease related to NASH or NAFLD, portal vein hypertension or thrombosis, esophageal or gastric varices or bleeding from those varices, and other liver-disease related co-morbidities). In some embodiments, the liver disease is a fatty liver disease. In some embodiments, the liver disease is AFLD. In some embodiments, the liver disease is NAFLD. In some embodiments, the liver disease is NASH. In some embodiments, the liver disease is liver cirrhosis. In some embodiments, the liver disease is liver fibrosis. In some embodiments, the liver disease is an increased liver enzyme. In some embodiments, the liver disease is increased ALT. In some embodiments, the liver disease is increased AST. In some embodiments, the liver disease is simple steatosis. In some embodiments, the liver disease is steatohepatitis. In some embodiments, the liver disease is parenchymal liver disease. In some embodiments, the liver disease is viral hepatitis. In some embodiments, the liver disease is hepatocellular carcinoma. In some embodiments, the liver disease is liver damage quantified by a liver biomarker (e.g., liver transaminase), a change in a liver biomarker, or by liver imaging.

Symptoms of liver disease include, but are not limited to, enlarged liver, fatigue, pain in the upper right abdomen, abdominal swelling (ascites), enlarged blood vessels just beneath the skin’s surface, enlarged breasts in men, enlarged spleen, red palms, and yellowing of the skin and eyes (jaundice), pruritus, dark urine color, pale stool color nausea or vomiting, loss of appetite, and tendency to bruise easily. Testing for liver diseases can involve blood tests, imaging of the liver, and biopsy of the liver. An individual is at increased risk of developing a liver disease if the subject has at least one known risk-factor (e.g., genetic factor such as a disease-causing mutation) placing individuals with that risk factor at a statistically significant greater risk of developing the disease than individuals without the risk factor. Risk factors for liver diseases include, for example, excessive alcohol use, obesity, high cholesterol, high levels of triglycerides in the blood, polycystic ovary syndrome, sleep apnea, type 2 diabetes, underactive thyroid (hypothyroidism), underactive pituitary gland (hypopituitarism), and metabolic syndromes including raised blood lipids.

The present disclosure provides methods of treating a subject having a liver disease, the methods comprising administering a CREB3L3 inhibitor to the subject.

The present disclosure also provides methods of treating a subject having parenchymal liver disease, the methods comprising administering a CREB3L3 inhibitor to the subject.

The present disclosure also provides methods of treating a subject having liver fibrosis, the methods comprising administering a CREB3L3 inhibitor to the subject.

The present disclosure also provides methods of treating a subject having liver cirrhosis, the methods comprising administering a CREB3L3 inhibitor to the subject.

The present disclosure also provides methods of treating a subject having NAFLD, the methods comprising administering a CREB3L3 inhibitor to the subject.

In some embodiments, the CREB3L3 inhibitor comprises an inhibitory nucleic acid molecule. In some embodiments, the inhibitory nucleic acid molecule comprises an antisense molecule, a small interfering RNA (siRNA) molecule, or a short hairpin RNA (shRNA) molecule. In some embodiments, the inhibitory nucleic acid molecule comprises an antisense molecule. In some embodiments, the inhibitory nucleic acid molecule comprises an siRNA molecule. In some embodiments, the inhibitory nucleic acid molecule comprises an shRNA molecule. Such inhibitory nucleic acid molecules can be designed to target any region of a CREB3L3 nucleic acid molecule, such as an mRNA molecule. In some embodiments, the inhibitory nucleic acid molecule hybridizes to a sequence within a CREB3L3 genomic nucleic acid molecule or mRNA molecule and decreases expression of the CREB3L3 polypeptide in a cell in the subject. In some embodiments, the CREB3L3 inhibitor comprises an antisense molecule that hybridizes to a CREB3L3 genomic nucleic acid molecule or mRNA molecule and decreases expression of the CREB3L3 polypeptide in a cell in the subject. In some embodiments, the CREB3L3 inhibitor comprises an siRNA that hybridizes to a CREB3L3 genomic nucleic acid molecule or mRNA molecule and decreases expression of the CREB3L3 polypeptide in a cell in the subject. In some embodiments, the CREB3L3 inhibitor comprises an shRNA that hybridizes to a CREB3L3 genomic nucleic acid molecule or mRNA molecule and decreases expression of the CREB3L3 polypeptide in a cell in the subject.

In some embodiments, the CREB3L3 inhibitor comprises a nuclease agent that induces one or more nicks or double-strand breaks at a recognition sequence(s) or a DNA-binding protein that binds to a recognition sequence within a CREB3L3 genomic nucleic acid molecule. The recognition sequence can be located within a coding region of the CREB3L3 gene, or within regulatory regions that influence the expression of the gene. A recognition sequence of the DNA-binding protein or nuclease agent can be located in an intron, an exon, a promoter, an enhancer, a regulatory region, or any non-protein coding region. The recognition sequence can include or be proximate to the start codon of the CREB3L3 gene. For example, the recognition sequence can be located about 10, about 20, about 30, about 40, about 50, about 100, about 200, about 300, about 400, about 500, or about 1,000 nucleotides from the start codon. As another example, two or more nuclease agents can be used, each targeting a nuclease recognition sequence including or proximate to the start codon. As another example, two nuclease agents can be used, one targeting a nuclease recognition sequence including or proximate to the start codon, and one targeting a nuclease recognition sequence including or proximate to the stop codon, wherein cleavage by the nuclease agents can result in deletion of the coding region between the two nuclease recognition sequences. Any nuclease agent that induces a nick or double-strand break into a desired recognition sequence can be used in the methods and compositions disclosed herein. Any DNA-binding protein that binds to a desired recognition sequence can be used in the methods and compositions disclosed herein.

Suitable nuclease agents and DNA-binding proteins for use herein include, but are not limited to, zinc finger protein or zinc finger nuclease (ZFN) pair, Transcription Activator-Like Effector (TALE) protein or Transcription Activator-Like Effector Nuclease (TALEN), or Clustered Regularly Interspersed Short Palindromic Repeats (CRISPR)/CRISPR-associated (Cas) systems. The length of the recognition sequence can vary, and includes, for example, recognition sequences that are about 30-36 bp for a zinc finger protein or ZFN pair, about 15-18 bp for each ZFN, about 36 bp for a TALE protein or TALEN, and about 20 bp for a CRISPR/Cas guide RNA.

In some embodiments, CRISPR/Cas systems can be used to modify a CREB3L3 genomic nucleic acid molecule within a cell. The methods and compositions disclosed herein can employ CRISPR-Cas systems by utilizing CRISPR complexes (comprising a guide RNA (gRNA) complexed with a Cas protein) for site-directed cleavage of CREB3L3 nucleic acid molecules.

Cas proteins generally comprise at least one RNA recognition or binding domain that can interact with gRNAs. Cas proteins can also comprise nuclease domains (such as, for example, DNase or RNase domains), DNA binding domains, helicase domains, protein-protein interaction domains, dimerization domains, and other domains. Suitable Cas proteins include, for example, a wild type Cas9 protein and a wild type Cpf1 protein (such as, for example, FnCpf1). A Cas protein can have full cleavage activity to create a double-strand break in a CREB3L3 genomic nucleic acid molecule or it can be a nickase that creates a single-strand break in a CREB3L3 genomic nucleic acid molecule. Additional examples of Cas proteins include, but are not limited to, Cas1, Cas1B, Cas2, Cas3, Cas4, Cas5, Cas5e (CasD), Cas6, Cas6e, Cas6f, Cas7, Cas8a1, Cas8a2, Cas8b, Cas8c, Cas9 (Csn1 or Cs×12), Cas10, Cas10d, CasF, CasG, CasH, Csy1, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmr1 , Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cu1966, and homologs or modified versions thereof. Cas proteins can also be operably linked to heterologous polypeptides as fusion proteins. For example, a Cas protein can be fused to a cleavage domain, an epigenetic modification domain, a transcriptional activation domain, or a transcriptional repressor domain. Cas proteins can be provided in any form. For example, a Cas protein can be provided in the form of a protein, such as a Cas protein complexed with a gRNA. Alternately, a Cas protein can be provided in the form of a nucleic acid molecule encoding the Cas protein, such as an RNA or DNA.

In some embodiments, targeted genetic modifications of a CREB3L3 genomic nucleic acid molecules can be generated by contacting a cell with a Cas protein and one or more gRNAs that hybridize to one or more gRNA recognition sequences within a target genomic locus in the CREB3L3 genomic nucleic acid molecule. For example, a gRNA recognition sequence can be located within a region of SEQ ID NO:1. The gRNA recognition sequence can also include or be proximate to a position corresponding to position 6,120 according to SEQ ID NO:1. For example, the gRNA recognition sequence can be located from about 1000, from about 500, from about 400, from about 300, from about 200, from about 100, from about 50, from about 45, from about 40, from about 35, from about 30, from about 25, from about 20, from about 15, from about 10, or from about 5 nucleotides of a position corresponding to position 6,120 according to SEQ ID NO:1. The gRNA recognition sequence can include or be proximate to the start codon of a CREB3L3 genomic nucleic acid molecule or the stop codon of a CREB3L3 genomic nucleic acid molecule. For example, the gRNA recognition sequence can be located from about 10, from about 20, from about 30, from about 40, from about 50, from about 100, from about 200, from about 300, from about 400, from about 500, or from about 1,000 nucleotides of the start codon or the stop codon.

The gRNA recognition sequences within a target genomic locus in a CREB3L3 genomic nucleic acid molecule are located near a Protospacer Adjacent Motif (PAM) sequence, which is a 2-6 base pair DNA sequence immediately following the DNA sequence targeted by the Cas9 nuclease. The canonical PAM is the sequence 5′-NGG-3′ where “N” is any nucleobase followed by two guanine (“G”) nucleobases. gRNAs can transport Cas9 to anywhere in the genome for gene editing, but no editing can occur at any site other than one at which Cas9 recognizes PAM. In addition, 5′-NGA-3′ can be a highly efficient non-canonical PAM for human cells. Generally, the PAM is about 2 to about 6 nucleotides downstream of the DNA sequence targeted by the gRNA. The PAM can flank the gRNA recognition sequence. In some embodiments, the gRNA recognition sequence can be flanked on the 3′ end by the PAM. In some embodiments, the gRNA recognition sequence can be flanked on the 5′ end by the PAM. For example, the cleavage site of Cas proteins can be about 1 to about 10 base pairs, about 2 to about 5 base pairs, or 3 base pairs upstream or downstream of the PAM sequence. In some embodiments (such as when Cas9 from S. pyogenes or a closely related Cas9 is used), the PAM sequence of the non-complementary strand can be 5′-NGG-3′, where N is any DNA nucleotide and is immediately 3′ of the gRNA recognition sequence of the non-complementary strand of the target DNA. As such, the PAM sequence of the complementary strand would be 5′-CCN-3′, where N is any DNA nucleotide and is immediately 5′ of the gRNA recognition sequence of the complementary strand of the target DNA.

A gRNA is an RNA molecule that binds to a Cas protein and targets the Cas protein to a specific location within a CREB3L3 genomic nucleic acid molecule. An exemplary gRNA is a gRNA effective to direct a Cas enzyme to bind to or cleave a CREB3L3 genomic nucleic acid molecule, wherein the gRNA comprises a DNA-targeting segment that hybridizes to a gRNA recognition sequence within the CREB3L3 genomic nucleic acid molecule that includes or is proximate to a position corresponding to position 6,120 according to SEQ ID NO:1. For example, a gRNA can be selected such that it hybridizes to a gRNA recognition sequence that is located about 5, about 10, about 15, about 20, about 25, about 30, about 35, about 40, about 45, about 50, about 100, about 200, about 300, about 400, about 500, or about 1,000 nucleotides from a position corresponding to position 6,120 according to SEQ ID NO:1. Other exemplary gRNAs comprise a DNA-targeting segment that hybridizes to a gRNA recognition sequence present within a CREB3L3 genomic nucleic acid molecule that includes or is proximate to the start codon or the stop codon. For example, a gRNA can be selected such that it hybridizes to a gRNA recognition sequence that is located about 5, about 10, about 15, about 20, about 25, about 30, about 35, about 40, about 45, about 50, about 100, about 200, about 300, about 400, about 500, or about 1,000 nucleotides of the start codon or located about 5, about 10, about 15, about 20, about 25, about 30, about 35, about 40, about 45, about 50, about 100, about 200, about 300, about 400, about 500, or about 1,000 nucleotides of the stop codon. Suitable gRNAs can comprise from about 17 to about 25 nucleotides, from about 17 to about 23 nucleotides, from about 18 to about 22 nucleotides, or from about 19 to about 21 nucleotides. In some embodiments, the gRNAs can comprise 20 nucleotides.

Examples of suitable gRNA recognition sequences located within the CREB3L3 reference gene are set forth in Table 1 as SEQ ID NOs:69-88.

TABLE 1

Guide RNA Recognition Sequences Near CREB3L3 Variation

| Strand | gRNA Recognition Sequence | SEQ ID NO: |
|---|---|---|
| - | TTCACGGTGAGATTGCATCG | 69 |
| - | AGTTGCCAGGATGATAGGAG | 70 |
| + | GAGTGCTGAAAAAAATCCGC | 71 |
| + | AAGAAGAAGGAATATATCGA | 72 |
| - | TGAGGAAGTCGTCAGAGTCG | 73 |
| + | GAAAATCCGGAACAAGCAGT | 74 |
| + | GCCTCTGTGACCATAGACCT | 75 |
| - | CACGGTGAGATTGCATCGTG | 76 |
| - | ACCCAGGTCTATGGTCACAG | 77 |
| + | CGATGGCCTGGAGACTCGGT | 78 |
| - | TGCCACTATCACTGCCTTCG | 79 |
| + | CCATTTCACTTGGCAGTACG | 80 |
| + | TCCTGGATCTCCTGTTTGAC | 81 |
| + | CTCCTGTTTGACCGGCAGGA | 82 |
| - | CCCAGACTCACCTTAGTGAG | 83 |
| - | CCAGGATGATAGGAGAGGCA | 84 |
| - | TCACGGTGAGATTGCATCGT | 85 |
| + | GGACGGCATCCTGAGACACG | 86 |
| - | AGCCCCAGGTCGCAGGAGGT | 87 |
| - | GTTTCTTCAGTTGCTCCAAG | 88 |

The Cas protein and the gRNA form a complex, and the Cas protein cleaves the target CREB3L3 genomic nucleic acid molecule. The Cas protein can cleave the nucleic acid molecule at a site within or outside of the nucleic acid sequence present in the target CREB3L3 genomic nucleic acid molecule to which the DNA-targeting segment of a gRNA will bind. For example, formation of a CRISPR complex (comprising a gRNA hybridized to a gRNA recognition sequence and complexed with a Cas protein) can result in cleavage of one or both strands in or near (such as, for example, within 1, 2, 3, 4, 5, 6, 7, 8, 9, 10, 20, 50, or more base pairs from) the nucleic acid sequence present in the CREB3L3 genomic nucleic acid molecule to which a DNA-targeting segment of a gRNA will bind.

Such methods can result, for example, in a CREB3L3 genomic nucleic acid molecule in which a region of SEQ ID NO:1 is disrupted, the start codon is disrupted, the stop codon is disrupted, or the coding sequence is disrupted or deleted. Optionally, the cell can be further contacted with one or more additional gRNAs that hybridize to additional gRNA recognition sequences within the target genomic locus in the CREB3L3 genomic nucleic acid molecule. By contacting the cell with one or more additional gRNAs (such as, for example, a second gRNA that hybridizes to a second gRNA recognition sequence), cleavage by the Cas protein can create two or more double-strand breaks or two or more single-strand breaks.

In some embodiments, the CREB3L3 inhibitor comprises a small molecule.

In some embodiments, the methods of treatment further comprise detecting the presence or absence of a CREB3L3 variant nucleic acid molecule encoding a CREB3L3 predicted loss-of-function polypeptide in a biological sample obtained from the subject. As used throughout the present disclosure, “a CREB3L3 variant nucleic acid molecule” is any CREB3L3 nucleic acid molecule (such as, for example, genomic nucleic acid molecule, mRNA molecule, or cDNA molecule) encoding a CREB3L3 polypeptide having a partial loss-of-function, a complete loss-of-function, a predicted partial loss-of-function, or a predicted complete loss-of-function.

The present disclosure also provides methods of treating a subject with a therapeutic agent that treats or inhibits a liver disease. In some embodiments, the subject has a liver disease. In some embodiments, the methods comprise determining whether the subject has a CREB3L3 variant nucleic acid molecule encoding a CREB3L3 predicted loss-of-function polypeptide by obtaining or having obtained a biological sample from the subject, and performing or having performed a sequence analysis on the biological sample to determine if the subject has a genotype comprising the CREB3L3 variant nucleic acid molecule. When the subject is CREB3L3 reference, the therapeutic agent that treats or inhibits the liver disease is administered or continued to be administered to the subject in a standard dosage amount, and a CREB3L3 inhibitor is administered to the subject. When the subject is heterozygous for a CREB3L3 variant nucleic acid molecule, the therapeutic agent that treats or inhibits the liver disease is administered or continued to be administered to the subject in an amount that is the same as or less than a standard dosage amount, and a CREB3L3 inhibitor is administered to the subject. The presence of a genotype having the CREB3L3 variant nucleic acid molecule encoding a CREB3L3 predicted loss-of-function polypeptide indicates the subject has a decreased risk of developing a liver disease. In some embodiments, the subject is CREB3L3 reference. In some embodiments, the subject is heterozygous for the CREB3L3 variant nucleic acid molecule encoding a CREB3L3 predicted loss-of-function polypeptide.

For subjects that are genotyped or determined to be either CREB3L3 reference or heterozygous for the CREB3L3 variant nucleic acid molecule encoding a CREB3L3 predicted loss-of-function polypeptide, such subjects can be treated with a CREB3L3 inhibitor, as described herein.

Detecting the presence or absence of a CREB3L3 variant nucleic acid molecule encoding a CREB3L3 predicted loss-of-function polypeptide in a biological sample from a subject and/or determining whether a subject has a CREB3L3 variant nucleic acid molecule encoding a CREB3L3 predicted loss-of-function polypeptide can be carried out by any of the methods described herein. In some embodiments, these methods can be carried out in vitro. In some embodiments, these methods can be carried out in situ. In some embodiments, these methods can be carried out in vivo. In any of these embodiments, the CREB3L3 variant nucleic acid molecule encoding a CREB3L3 predicted loss-of-function polypeptide can be present within a cell obtained from the subject.

In some embodiments, when the subject is CREB3L3 reference, the subject is also administered a therapeutic agent that treats or inhibits a liver disease in a standard dosage amount. In some embodiments, when the subject is heterozygous for a CREB3L3 variant nucleic acid molecule encoding a CREB3L3 predicted loss-of-function polypeptide, the subject is also administered a therapeutic agent that treats or inhibits a liver disease in a dosage amount that is the same as or less than a standard dosage amount.

In some embodiments, the treatment methods further comprise detecting the presence or absence of a CREB3L3 predicted loss-of-function polypeptide in a biological sample from the subject. In some embodiments, when the subject does not have a CREB3L3 predicted loss-of-function polypeptide, the subject is also administered a therapeutic agent that treats or inhibits a liver disease in a standard dosage amount. In some embodiments, when the subject has a CREB3L3 predicted loss-of-function polypeptide, the subject is also administered a therapeutic agent that treats or inhibits a liver disease in a dosage amount that is the same as or less than a standard dosage amount.

The present disclosure also provides methods of treating a subject with a therapeutic agent that treats or inhibits a liver disease. In some embodiments, the subject has a liver disease. In some embodiments, the method comprises determining whether the subject has a CREB3L3 predicted loss-of-function polypeptide by obtaining or having obtained a biological sample from the subject, and performing or having performed an assay on the biological sample to determine if the subject has a CREB3L3 predicted loss-of-function polypeptide. When the subject does not have a CREB3L3 predicted loss-of-function polypeptide, the therapeutic agent that treats or inhibits the liver disease is administered or continued to be administered to the subject in a standard dosage amount, and a CREB3L3 inhibitor is administered to the subject. When the subject has a CREB3L3 predicted loss-of-function polypeptide, the therapeutic agent that treats or inhibits the liver disease is administered or continued to be administered to the subject in an amount that is the same as or less than a standard dosage amount, and a CREB3L3 inhibitor is administered to the subject. The presence of a CREB3L3 predicted loss-of-function polypeptide indicates the subject has a decreased risk of developing a liver disease. In some embodiments, the subject has a CREB3L3 predicted loss-of-function polypeptide. In some embodiments, the subject does not have a CREB3L3 predicted loss-of-function polypeptide.

Detecting the presence or absence of a CREB3L3 predicted loss-of-function polypeptide in a biological sample from a subject and/or determining whether a subject has a CREB3L3 predicted loss-of-function polypeptide can be carried out by any of the methods described herein. In some embodiments, these methods can be carried out in vitro. In some embodiments, these methods can be carried out in situ. In some embodiments, these methods can be carried out in vivo. In any of these embodiments, the CREB3L3 predicted loss-of-function polypeptide can be present within a cell obtained from the subject.

Examples of therapeutic agents that treat or inhibit liver disease include, but are not limited to: disulfiram, naltrexone, acamprosate, prednisone, azathioprine, penicillamine, trientine, deferoxamine, ciprofloxacin, norofloxacin, ceftriaxone, ofloxacin, amoxicillin-clavulanate, phytonadione, bumetanide, furosemide, hydrochlorothiazide, chlorothiazide, amiloride, triamterene, spironolactone, octreotide, atenolol, metoprolol, nadolol, propranolol, timolol, and carvedilol, or any combination thereof.

Additional examples of liver disease therapeutic agents (e.g., for use in chronic hepatitis C treatment) include, but are not limited to, ribavirin, paritaprevir, OLYSIO® (simeprevir), grazoprevir, ledipasvir, ombitasvir, elbasvir, DAKLINZA® (daclatasvir), dasabuvir, ritonavir, sofosbuvir, velpatasvir, voxilaprevir, glecaprevir, pibrentasvir, peginterferon alfa-2a, peginterferon alfa-2b, and interferon alfa-2b.

Additional examples of liver disease therapeutic agents (e.g., for use in NFLD) include, but are not limited to, weight loss inducing agents such as orlistat or sibutramine; insulin sensitizing agents such as thiazolidinediones (TZDs), metformin, and meglitinides; lipid lowering agents such as statins, fibrates, and omega-3 fatty acids; atioxidants such as, vitamin E, betaine, N-Acetyl-cysteine, lecithin, silymarin, and beta-carotene; anti TNF agents such as pentoxifylline; probiotics, such as VSL#3; and cytoprotective agents such as ursodeoxycholic acid (UDCA). Other suitable treatments include ACE inhibitors/ARBs, oligofructose, and Incretin analogs.

Additional examples of liver disease therapeutic agents (e.g., for use in NASH) include, but are not limited to, OCALIVA® (obeticholic acid), selonsertib, elafibranor, cenicriviroc, GR_MD_02, MGL_3196, IMM124E, ARAMCHOL™ (arachidyl amido cholanoic acid), GS0976, emricasan, volixibat, NGM282, GS9674, tropifexor, MN_001, LMB763, BI_1467335, MSDC_0602, PF_05221304, DF102, saroglitazar, BMS986036, lanifibranor, semaglutide, nitazoxanide, GRI_0621, EYP001, VK2809, nalmefene, LIK066, MT_3995, elobixibat, namodenoson, foralumab, SAR425899, sotagliflozin, EDP_305, isosabutate, gemcabene, TERN_101, KBP_042, PF_06865571, DUR928, PF_06835919, NGM313, BMS_986171, namacizumab, CER_209, ND_L02_s0201, RTU_1096, DRX_065, IONIS_DGAT2Rx, INT_767, NC_001, seladepar, PXL770, TERN_201, NV556, AZD2693, SP_1373, VK0214, hepastem, TGFTX4, RLBN1127, GKT_137831, RYI_018, CB4209-CB4211, and JH_0920.

In some embodiments, the dose of the therapeutic agents that treat or inhibit a liver disease can be reduced by about 10%, by about 20%, by about 30%, by about 40%, by about 50%, by about 60%, by about 70%, by about 80%, or by about 90% for subjects that are heterozygous for a CREB3L3 variant nucleic acid molecule encoding a CREB3L3 predicted loss-of-function polypeptide (i.e., a less than the standard dosage amount) compared to subjects that are CREB3L3 reference (who may receive a standard dosage amount). In some embodiments, the dose of the therapeutic agents that treat or inhibit a liver disease can be reduced by about 10%, by about 20%, by about 30%, by about 40%, or by about 50%. In addition, the dose of therapeutic agents that treat or inhibit a liver disease in subjects that are heterozygous for a CREB3L3 variant nucleic acid molecule encoding a CREB3L3 predicted loss-of-function polypeptide can be administered less frequently compared to subjects that are CREB3L3 reference.

Administration of the therapeutic agents that treat or inhibit a liver disease and/or CREB3L3 inhibitors can be repeated, for example, after one day, two days, three days, five days, one week, two weeks, three weeks, one month, five weeks, six weeks, seven weeks, eight weeks, two months, or three months. The repeated administration can be at the same dose or at a different dose. The administration can be repeated once, twice, three times, four times, five times, six times, seven times, eight times, nine times, ten times, or more. For example, according to certain dosage regimens a subject can receive therapy for a prolonged period of time such as, for example, 6 months, 1 year, or more. In addition, the therapeutic agents that treat or inhibit a liver disease and/or CREB3L3 inhibitors can be administered sequentially or at the same time. In addition, the therapeutic agents that treat or inhibit a liver disease and/or CREB3L3 inhibitors can be administered in separate compositions or can be administered together in the same composition.

Administration of the therapeutic agents that treat or inhibit a liver disease and/or CREB3L3 inhibitors can occur by any suitable route including, but not limited to, parenteral, intravenous, oral, subcutaneous, intra-arterial, intracranial, intrathecal, intraperitoneal, topical, intranasal, or intramuscular. Pharmaceutical compositions for administration are desirably sterile and substantially isotonic and manufactured under GMP conditions. Pharmaceutical compositions can be provided in unit dosage form (i.e., the dosage for a single administration). Pharmaceutical compositions can be formulated using one or more physiologically and pharmaceutically acceptable carriers, diluents, excipients or auxiliaries. The formulation depends on the route of administration chosen. The term “pharmaceutically acceptable” means that the carrier, diluent, excipient, or auxiliary is compatible with the other ingredients of the formulation and not substantially deleterious to the recipient thereof.

The terms “treat”, “treating”, and “treatment” and “prevent”, “preventing”, and “prevention” as used herein, refer to eliciting the desired biological response, such as a therapeutic and prophylactic effect, respectively. In some embodiments, a therapeutic effect comprises one or more of a decrease/reduction in a liver disease, a decrease/reduction in the severity of a liver disease (such as, for example, a reduction or inhibition of development of a liver disease), a decrease/reduction in symptoms and liver disease-related effects, delaying the onset of symptoms and liver disease-related effects, reducing the severity of symptoms of liver disease-related effects, reducing the severity of an acute episode, reducing the number of symptoms and liver disease-related effects, reducing the latency of symptoms and liver disease-related effects, an amelioration of symptoms and liver disease-related effects, reducing secondary symptoms, reducing secondary infections, preventing relapse to a liver disease, decreasing the number or frequency of relapse episodes, increasing latency between symptomatic episodes, increasing time to sustained progression, expediting remission, inducing remission, augmenting remission, speeding recovery, or increasing efficacy of or decreasing resistance to alternative therapeutics, and/or an increased survival time of the affected host animal, following administration of the agent or composition comprising the agent. A prophylactic effect may comprise a complete or partial avoidance/inhibition or a delay of liver disease development/progression (such as, for example, a complete or partial avoidance/inhibition or a delay), and an increased survival time of the affected host animal, following administration of a therapeutic protocol. Treatment of a liver disease encompasses the treatment of subjects already diagnosed as having any form of a liver disease at any clinical stage or manifestation, the delay of the onset or evolution or aggravation or deterioration of the symptoms or signs of a liver disease, and/or preventing and/or reducing the severity of a liver disease.

The present disclosure also provides methods of identifying a subject having an increased risk of developing a liver disease. In some embodiments, the methods comprise determining or having determined the presence or absence of a CREB3L3 variant nucleic acid molecule (such as a genomic nucleic acid molecule, mRNA molecule, and/or cDNA molecule) encoding a CREB3L3 predicted loss-of-function polypeptide in a biological sample obtained from the subject. When the subject lacks a CREB3L3 variant nucleic acid molecule encoding a CREB3L3 predicted loss-of-function polypeptide (i.e., the subject is genotypically categorized as CREB3L3 reference), then the subject has an increased risk of developing a liver disease. When the subject has a CREB3L3 variant nucleic acid molecule encoding a CREB3L3 predicted loss-of-function polypeptide (i.e., the subject is heterozygous or homozygous for a CREB3L3 variant nucleic acid molecule), then the subject has a decreased risk of developing a liver disease compared to a subject that is CREB3L3 reference.

Having a single copy of a CREB3L3 variant nucleic acid molecule encoding a CREB3L3 predicted loss-of-function polypeptide is more protective of a subject from developing a liver disease than having no copies of a CREB3L3 variant nucleic acid molecule encoding a CREB3L3 predicted loss-of-function polypeptide. Without intending to be limited to any particular theory or mechanism of action, it is believed that a single copy of a CREB3L3 variant nucleic acid molecule encoding a CREB3L3 predicted loss-of-function polypeptide (i.e., heterozygous for a CREB3L3 variant nucleic acid molecule) is protective of a subject from developing a liver disease, and it is also believed that having two copies of a CREB3L3 variant nucleic acid molecule encoding a CREB3L3 predicted loss-of-function polypeptide (i.e., homozygous for a CREB3L3 variant nucleic acid molecule) may be more protective of a subject from developing a liver disease, relative to a subject with a single copy. Thus, in some embodiments, a single copy of a CREB3L3 variant nucleic acid molecule encoding a CREB3L3 predicted loss-of-function polypeptide may not be completely protective, but instead, may be partially or incompletely protective of a subject from developing a liver disease. While not desiring to be bound by any particular theory, there may be additional factors or molecules involved in the development of a liver disease that are still present in a subject having a single copy of a CREB3L3 variant nucleic acid molecule encoding a CREB3L3 predicted loss-of-function polypeptide, thus resulting in less than complete protection from the development of a liver disease.

Detecting the presence or absence of a CREB3L3 variant nucleic acid molecule encoding a CREB3L3 predicted loss-of-function polypeptide in a biological sample from the subject and/or determining whether a subject has a CREB3L3 variant nucleic acid molecule encoding a CREB3L3 predicted loss-of-function polypeptide can be carried out by any of the methods described herein. In some embodiments, these methods can be carried out in vitro. In some embodiments, these methods can be carried out in situ. In some embodiments, these methods can be carried out in vivo. In any of these embodiments, the CREB3L3 variant nucleic acid molecule encoding a CREB3L3 predicted loss-of-function polypeptide can be present within a cell obtained from the subject.

In some embodiments, when a subject is identified as having an increased risk of developing a liver disease, the subject is further treated with a therapeutic agent that treats or inhibits a liver disease and/or a CREB3L3 inhibitor, as described herein. For example, when the subject is CREB3L3 reference, and therefore has an increased risk of developing a liver disease, the subject is administered a CREB3L3 inhibitor. In some embodiments, such a subject is also administered a therapeutic agent that treats or inhibits a liver disease. In some embodiments, when the subject is heterozygous for a CREB3L3 variant nucleic acid molecule encoding a CREB3L3 predicted loss-of-function polypeptide, the subject is administered the therapeutic agent that treats or inhibits a liver disease in a dosage amount that is the same as or less than a standard dosage amount, and is also administered a CREB3L3 inhibitor. In some embodiments, the subject is CREB3L3 reference. In some embodiments, the subject is heterozygous for a CREB3L3 variant nucleic acid molecule encoding a CREB3L3 predicted loss-of-function polypeptide.

The present disclosure also provides methods of detecting the presence or absence of a CREB3L3 variant genomic nucleic acid molecule encoding a CREB3L3 predicted loss-of-function polypeptide in a biological sample obtained from a subject, and/or a CREB3L3 variant mRNA molecule encoding a CREB3L3 predicted loss-of-function polypeptide in a biological sample obtained from a subject, and/or a CREB3L3 variant cDNA molecule encoding a CREB3L3 predicted loss-of-function polypeptide produced from an mRNA molecule in a biological sample obtained from a subject. It is understood that gene sequences within a population and mRNA molecules encoded by such genes can vary due to polymorphisms such as single-nucleotide polymorphisms. The sequences provided herein for the CREB3L3 variant genomic nucleic acid molecule, CREB3L3 variant mRNA molecule, and CREB3L3 variant cDNA molecule are only exemplary sequences. Other sequences for the CREB3L3 variant genomic nucleic acid molecule, variant mRNA molecule, and variant cDNA molecule are also possible.

The biological sample can be derived from any cell, tissue, or biological fluid from the subject. The biological sample may comprise any clinically relevant tissue such as, for example, a bone marrow sample, a tumor biopsy, a fine needle aspirate, or a sample of bodily fluid, such as blood, gingival crevicular fluid, plasma, serum, lymph, ascitic fluid, cystic fluid, or urine. In some embodiments, the biological sample comprises a buccal swab. The biological sample used in the methods disclosed herein can vary based on the assay format, nature of the detection method, and the tissues, cells, or extracts that are used as the sample. A biological sample can be processed differently depending on the assay being employed. For example, when detecting any CREB3L3 variant nucleic acid molecule, preliminary processing designed to isolate or enrich the biological sample for the CREB3L3 variant nucleic acid molecule can be employed. A variety of techniques may be used for this purpose. When detecting the level of any CREB3L3 variant mRNA molecule, different techniques can be used enrich the biological sample with mRNA molecules. Various methods to detect the presence or level of an mRNA molecule or the presence of a particular variant genomic DNA locus can be used.

The present disclosure also provides methods of detecting a CREB3L3 variant nucleic acid molecule, or the complement thereof, encoding a CREB3L3 predicted loss-of-function polypeptide in a subject. The methods comprise assaying a biological sample obtained from the subject to determine whether a nucleic acid molecule in the biological sample is a CREB3L3 variant nucleic acid molecule encoding a CREB3L3 predicted loss-of-function polypeptide.

In some embodiments, the CREB3L3 variant nucleic acid molecule encoding the CREB3L3 predicted loss-of-function polypeptide, or the complement thereof, is a genomic nucleic acid molecule having a nucleotide sequence comprising an adenine at a position corresponding to position 6,120 according to SEQ ID NO:2, or the complement thereof.

In some embodiments, the CREB3L3 variant nucleic acid molecule encoding the CREB3L3 predicted loss-of-function polypeptide, or the complement thereof, is an mRNA molecule having a nucleotide sequence comprising an adenine at a position corresponding to: position 661 according to SEQ ID NO:17, or the complement thereof; position 649 according to SEQ ID NO:18, or the complement thereof; position 624 according to SEQ ID NO:19, or the complement thereof; position 624 according to SEQ ID NO:20, or the complement thereof; position 658 according to SEQ ID NO:21, or the complement thereof; position 661 according to SEQ ID NO:22, or the complement thereof; position 661 according to SEQ ID NO:23, or the complement thereof; position 663 according to SEQ ID NO:24, or the complement thereof; position 660 according to SEQ ID NO:25, or the complement thereof; position 649 according to SEQ ID NO:26, or the complement thereof; position 624 according to SEQ ID NO:27, or the complement thereof; or position 691 according to SEQ ID NO:28, or the complement thereof.

In some embodiments, the CREB3L3 variant nucleic acid molecule encoding the CREB3L3 predicted loss-of-function polypeptide, or the complement thereof, is a cDNA molecule produced from an mRNA molecule in the biological sample having a nucleotide sequence comprising an adenine at a position corresponding to: position 661 according to SEQ ID NO:45, or the complement thereof; position 649 according to SEQ ID NO:46, or the complement thereof; position 624 according to SEQ ID NO:47, or the complement thereof; position 624 according to SEQ ID NO:48, or the complement thereof; position 658 according to SEQ ID NO:49, or the complement thereof; position 661 according to SEQ ID NO:50, or the complement thereof; position 661 according to SEQ ID NO:51, or the complement thereof; position 663 according to SEQ ID NO:52, or the complement thereof; position 660 according to SEQ ID NO:53, or the complement thereof; position 649 according to SEQ ID NO:54, or the complement thereof; position 624 according to SEQ ID NO:55, or the complement thereof; or position 691 according to SEQ ID NO:56, or the complement thereof.

In some embodiments, the CREB3L3 variant nucleic acid molecule has a nucleotide sequence comprising: i) an adenine at a position corresponding to position 6,120 according to SEQ ID NO:2 (for genomic nucleic acid molecules); ii) an adenine at a position corresponding to: position 661 according to SEQ ID NO:17, position 649 according to SEQ ID NO:18, position 624 according to SEQ ID NO:19, position 624 according to SEQ ID NO:20, position 658 according to SEQ ID NO:21, position 661 according to SEQ ID NO:22, position 661 according to SEQ ID NO:23, position 663 according to SEQ ID NO:24, position 660 according to SEQ ID NO:25, position 649 according to SEQ ID NO:26, position 624 according to SEQ ID NO:27, or position 691 according to SEQ ID NO:28 (for mRNA molecules); or iii) an adenine at a position corresponding to: position 661 according to SEQ ID NO:45, position 649 according to SEQ ID NO:46, position 624 according to SEQ ID NO:47, position 624 according to SEQ ID NO:48, position 658 according to SEQ ID NO:49, position 661 according to SEQ ID NO:50, position 661 according to SEQ ID NO:51, position 663 according to SEQ ID NO:52, position 660 according to SEQ ID NO:53, position 649 according to SEQ ID NO:54, position 624 according to SEQ ID NO:55, or position 691 according to SEQ ID NO:56 (for cDNA molecules obtained from mRNA molecules).

In some embodiments, the biological sample comprises a cell or cell lysate. Such methods can further comprise, for example, obtaining a biological sample from the subject comprising a CREB3L3 genomic nucleic acid molecule or mRNA molecule, and if mRNA, optionally reverse transcribing the mRNA into cDNA. Such assays can comprise, for example determining the identity of these positions of the particular CREB3L3 nucleic acid molecule. In some embodiments, the method is an in vitro method.

In some embodiments, the determining step, detecting step, or sequence analysis comprises sequencing at least a portion of the nucleotide sequence of the CREB3L3 genomic nucleic acid molecule, the CREB3L3 mRNA molecule, or the CREB3L3 cDNA molecule produced from the mRNA molecule in the biological sample, wherein the sequenced portion comprises one or more variations that cause a loss-of-function (partial or complete) or are predicted to cause a loss-of-function (partial or complete).

In some embodiments, the determining step, detecting step, or sequence analysis comprises sequencing at least a portion of: i) the nucleotide sequence of the CREB3L3 genomic nucleic acid molecule in the biological sample, wherein the sequenced portion comprises a position corresponding to position 6,120 according to SEQ ID NO:2, or the complement thereof; ii) the nucleotide sequence of the CREB3L3 mRNA molecule in the biological sample, wherein the sequenced portion comprises a position corresponding to: position 661 according to SEQ ID NO:17, or the complement thereof; position 649 according to SEQ ID NO:18, or the complement thereof; position 624 according to SEQ ID NO:19, or the complement thereof; position 624 according to SEQ ID NO:20, or the complement thereof; position 658 according to SEQ ID NO:21, or the complement thereof; position 661 according to SEQ ID NO:22, or the complement thereof; position 661 according to SEQ ID NO:23, or the complement thereof; position 663 according to SEQ ID NO:24, or the complement thereof; position 660 according to SEQ ID NO:25, or the complement thereof; position 649 according to SEQ ID NO:26, or the complement thereof; position 624 according to SEQ ID NO:27, or the complement thereof; or position 691 according to SEQ ID NO:28, or the complement thereof; and/or iii) the nucleotide sequence of the CREB3L3 cDNA molecule produced from the mRNA in the biological sample, wherein the sequenced portion comprises a position corresponding to: position 661 according to SEQ ID NO:45, or the complement thereof; position 649 according to SEQ ID NO:46, or the complement thereof; position 624 according to SEQ ID NO:47, or the complement thereof; position 624 according to SEQ ID NO:48, or the complement thereof; position 658 according to SEQ ID NO:49, or the complement thereof; position 661 according to SEQ ID NO:50, or the complement thereof; position 661 according to SEQ ID NO:51, or the complement thereof; position 663 according to SEQ ID NO:52, or the complement thereof; position 660 according to SEQ ID NO:53, or the complement thereof; position 649 according to SEQ ID NO:54, or the complement thereof; position 624 according to SEQ ID NO:55, or the complement thereof; or position 691 according to SEQ ID NO:56, or the complement thereof. When the sequenced portion of the CREB3L3 nucleic acid molecule in the biological sample comprises: an adenine at a position corresponding to position 6,120 according to SEQ ID NO:2; an adenine at a position corresponding to: position 661 according to SEQ ID NO:17, position 649 according to SEQ ID NO:18, position 624 according to SEQ ID NO:19, position 624 according to SEQ ID NO:20, position 658 according to SEQ ID NO:21, position 661 according to SEQ ID NO:22, position 661 according to SEQ ID NO:23, position 663 according to SEQ ID NO:24, position 660 according to SEQ ID NO:25, position 649 according to SEQ ID NO:26, position 624 according to SEQ ID NO:27, or position 691 according to SEQ ID NO:28; or an adenine at a position corresponding to: position 661 according to SEQ ID NO:45, position 649 according to SEQ ID NO:46, position 624 according to SEQ ID NO:47, position 624 according to SEQ ID NO:48, position 658 according to SEQ ID NO:49, position 661 according to SEQ ID NO:50, position 661 according to SEQ ID NO:51, position 663 according to SEQ ID NO:52, position 660 according to SEQ ID NO:53, position 649 according to SEQ ID NO:54, position 624 according to SEQ ID NO:55, or position 691 according to SEQ ID NO:56, then the CREB3L3 nucleic acid molecule in the biological sample is a CREB3L3 variant nucleic acid molecule encoding a CREB3L3 predicted loss-of-function polypeptide.

In some embodiments, the determining step, detecting step, or sequence analysis comprises sequencing at least a portion of the nucleotide sequence of the CREB3L3 genomic nucleic acid molecule in the biological sample, wherein the sequenced portion comprises a position corresponding to position 6,120 according to SEQ ID NO:2, or the complement thereof. When the sequenced portion of the CREB3L3 nucleic acid molecule in the biological sample comprises an adenine at a position corresponding to position 6,120 according to SEQ ID NO:2, then the CREB3L3 nucleic acid molecule in the biological sample is a CREB3L3 variant genomic nucleic acid molecule encoding a CREB3L3 predicted loss-of-function polypeptide.

In some embodiments, the determining step, detecting step, or sequence analysis comprises sequencing at least a portion of the nucleotide sequence of the CREB3L3 mRNA molecule in the biological sample, wherein the sequenced portion comprises a position corresponding to: position 661 according to SEQ ID NO:17, or the complement thereof; position 649 according to SEQ ID NO:18, or the complement thereof; position 624 according to SEQ ID NO:19, or the complement thereof; position 624 according to SEQ ID NO:20, or the complement thereof; position 658 according to SEQ ID NO:21, or the complement thereof; position 661 according to SEQ ID NO:22, or the complement thereof; position 661 according to SEQ ID NO:23, or the complement thereof; position 663 according to SEQ ID NO:24, or the complement thereof; position 660 according to SEQ ID NO:25, or the complement thereof; position 649 according to SEQ ID NO:26, or the complement thereof; position 624 according to SEQ ID NO:27, or the complement thereof; or position 691 according to SEQ ID NO:28, or the complement thereof. When the sequenced portion of the CREB3L3 mRNA molecule in the biological sample comprises an adenine at a position corresponding to: position 661 according to SEQ ID NO:17, position 649 according to SEQ ID NO:18, position 624 according to SEQ ID NO:19, position 624 according to SEQ ID NO:20, position 658 according to SEQ ID NO:21, position 661 according to SEQ ID NO:22, position 661 according to SEQ ID NO:23, position 663 according to SEQ ID NO:24, position 660 according to SEQ ID NO:25, position 649 according to SEQ ID NO:26, position 624 according to SEQ ID NO:27, or position 691 according to SEQ ID NO:28, then the CREB3L3 nucleic acid molecule in the biological sample is a CREB3L3 variant mRNA molecule encoding a CREB3L3 predicted loss-of-function polypeptide.

In some embodiments, the determining step, detecting step, or sequence analysis comprises sequencing at least a portion of the nucleotide sequence of the CREB3L3 cDNA molecule produced from the mRNA molecule in the biological sample, wherein the sequenced portion comprises a position corresponding to: position 661 according to SEQ ID NO:45, or the complement thereof; position 649 according to SEQ ID NO:46, or the complement thereof; position 624 according to SEQ ID NO:47, or the complement thereof; position 624 according to SEQ ID NO:48, or the complement thereof; position 658 according to SEQ ID NO:49, or the complement thereof; position 661 according to SEQ ID NO:50, or the complement thereof; position 661 according to SEQ ID NO:51, or the complement thereof; position 663 according to SEQ ID NO:52, or the complement thereof; position 660 according to SEQ ID NO:53, or the complement thereof; position 649 according to SEQ ID NO:54, or the complement thereof; position 624 according to SEQ ID NO:55, or the complement thereof; position 691 according to SEQ ID NO:56, or the complement thereof. When the sequenced portion of the CREB3L3 cDNA molecule in the biological sample comprises an adenine at a position corresponding to: position 661 according to SEQ ID NO:45, position 649 according to SEQ ID NO:46, position 624 according to SEQ ID NO:47, position 624 according to SEQ ID NO:48, position 658 according to SEQ ID NO:49, position 661 according to SEQ ID NO:50, position 661 according to SEQ ID NO:51, position 663 according to SEQ ID NO:52, position 660 according to SEQ ID NO:53, position 649 according to SEQ ID NO:54, position 624 according to SEQ ID NO:55, or position 691 according to SEQ ID NO:56, then the CREB3L3 nucleic acid molecule in the biological sample is a CREB3L3 variant cDNA molecule encoding a CREB3L3 predicted loss-of-function polypeptide.

In some embodiments, the determining step, detecting step, or sequence analysis comprises: a) contacting the biological sample with a primer hybridizing to a portion of the nucleotide sequence of the CREB3L3: i) genomic nucleic acid molecule, or the complement thereof, that is proximate to a position corresponding to position 6,120 according to SEQ ID NO:2, or the complement thereof; ii) mRNA molecule, or the complement thereof, that is proximate to a position corresponding to: position 661 according to SEQ ID NO:17, or the complement thereof; position 649 according to SEQ ID NO:18, or the complement thereof; position 624 according to SEQ ID NO:19, or the complement thereof; position 624 according to SEQ ID NO:20, or the complement thereof; position 658 according to SEQ ID NO:21, or the complement thereof; position 661 according to SEQ ID NO:22, or the complement thereof; position 661 according to SEQ ID NO:23, or the complement thereof; position 663 according to SEQ ID NO:24, or the complement thereof; position 660 according to SEQ ID NO:25, or the complement thereof; position 649 according to SEQ ID NO:26, or the complement thereof; position 624 according to SEQ ID NO:27, or the complement thereof; or position 691 according to SEQ ID NO:28; and/or iii) cDNA molecule, or the complement thereof, that is proximate to a position corresponding to: position 661 according to SEQ ID NO:45, or the complement thereof; position 649 according to SEQ ID NO:46, or the complement thereof; position 624 according to SEQ ID NO:47, or the complement thereof; position 624 according to SEQ ID NO:48, or the complement thereof; position 658 according to SEQ ID NO:49, or the complement thereof; position 661 according to SEQ ID NO:50, or the complement thereof; position 661 according to SEQ ID NO:51, or the complement thereof; position 663 according to SEQ ID NO:52, or the complement thereof; position 660 according to SEQ ID NO:53, or the complement thereof; position 649 according to SEQ ID NO:54, or the complement thereof; or position 624 according to SEQ ID NO:55, or the complement thereof; position 691 according to SEQ ID NO:56; b) extending the primer at least through the position of the nucleotide sequence of the CREB3L3: i) genomic nucleic acid molecule, or the complement thereof, corresponding to position 6,120 according to SEQ ID NO:2, or the complement thereof; ii) mRNA molecule, or the complement thereof, corresponding to: position 661 according to SEQ ID NO:17, or the complement thereof; position 649 according to SEQ ID NO:18, or the complement thereof; position 624 according to SEQ ID NO:19, or the complement thereof; position 624 according to SEQ ID NO:20, or the complement thereof; position 658 according to SEQ ID NO:21, or the complement thereof; position 661 according to SEQ ID NO:22, or the complement thereof; position 661 according to SEQ ID NO:23, or the complement thereof; position 663 according to SEQ ID NO:24, or the complement thereof; position 660 according to SEQ ID NO:25, or the complement thereof; position 649 according to SEQ ID NO:26, or the complement thereof; position 624 according to SEQ ID NO:27, or the complement thereof; or position 691 according to SEQ ID NO:28, or the complement thereof; and/or iii) cDNA molecule, or the complement thereof, corresponding to: position 661 according to SEQ ID NO:45, or the complement thereof; position 649 according to SEQ ID NO:46, or the complement thereof; position 624 according to SEQ ID NO:47, or the complement thereof; position 624 according to SEQ ID NO:48, or the complement thereof; position 658 according to SEQ ID NO:49, or the complement thereof; position 661 according to SEQ ID NO:50, or the complement thereof; position 661 according to SEQ ID NO:51, or the complement thereof; position 663 according to SEQ ID NO:52, or the complement thereof; position 660 according to SEQ ID NO:53, or the complement thereof; position 649 according to SEQ ID NO:54, or the complement thereof; position 624 according to SEQ ID NO:55, or the complement thereof; or position 691 according to SEQ ID NO:56; and c) determining whether the extension product of the primer comprises: an adenine at a position corresponding to position 6,120 according to SEQ ID NO:2, or the complement thereof; an adenine at a position corresponding to: position 661 according to SEQ ID NO:17, or the complement thereof; position 649 according to SEQ ID NO:18, or the complement thereof; position 624 according to SEQ ID NO:19, or the complement thereof; position 624 according to SEQ ID NO:20, or the complement thereof; position 658 according to SEQ ID NO:21, or the complement thereof; position 661 according to SEQ ID NO:22, or the complement thereof; position 661 according to SEQ ID NO:23, or the complement thereof; position 663 according to SEQ ID NO:24, or the complement thereof; position 660 according to SEQ ID NO:25, or the complement thereof; position 649 according to SEQ ID NO:26, or the complement thereof; position 624 according to SEQ ID NO:27, or the complement thereof; or position 691 according to SEQ ID NO:28, or the complement thereof; or an adenine at a position corresponding to: position 661 according to SEQ ID NO:45, or the complement thereof; position 649 according to SEQ ID NO:46, or the complement thereof; position 624 according to SEQ ID NO:47, or the complement thereof; position 624 according to SEQ ID NO:48, or the complement thereof; position 658 according to SEQ ID NO:49, or the complement thereof; position 661 according to SEQ ID NO:50, or the complement thereof; position 661 according to SEQ ID NO:51, or the complement thereof; position 663 according to SEQ ID NO:52, or the complement thereof; position 660 according to SEQ ID NO:53, or the complement thereof; position 649 according to SEQ ID NO:54, or the complement thereof; position 624 according to SEQ ID NO:55, or the complement thereof; or position 691 according to SEQ ID NO:56, or the complement thereof.

In some embodiments, the determining step, detecting step, or sequence analysis comprises: a) contacting the biological sample with a primer hybridizing to a portion of the nucleotide sequence of the CREB3L3 genomic nucleic acid molecule, or the complement thereof, that is proximate to a position corresponding to position 6,120 according to SEQ ID NO:2, or the complement thereof; b) extending the primer at least through the position of the nucleotide sequence of the CREB3L3 genomic nucleic acid molecule, or the complement thereof, corresponding to position 6,120 according to SEQ ID NO:2, or the complement thereof; and c) determining whether the extension product of the primer comprises an adenine at a position corresponding to position 6,120 according to SEQ ID NO:2, or the complement thereof.

In some embodiments, the determining step, detecting step, or sequence analysis comprises: a) contacting the biological sample with a primer hybridizing to a portion of the nucleotide sequence of the CREB3L3 mRNA molecule, or the complement thereof, that is proximate to a position corresponding to: position 661 according to SEQ ID NO:17, or the complement thereof; position 649 according to SEQ ID NO:18, or the complement thereof; position 624 according to SEQ ID NO:19, or the complement thereof; position 624 according to SEQ ID NO:20, or the complement thereof; position 658 according to SEQ ID NO:21, or the complement thereof; position 661 according to SEQ ID NO:22, or the complement thereof; position 661 according to SEQ ID NO:23, or the complement thereof; position 663 according to SEQ ID NO:24, or the complement thereof; position 660 according to SEQ ID NO:25, or the complement thereof; position 649 according to SEQ ID NO:26, or the complement thereof; position 624 according to SEQ ID NO:27, or the complement thereof; or position 691 according to SEQ ID NO:28, or the complement thereof; b) extending the primer at least through the position of the nucleotide sequence of the CREB3L3 mRNA molecule corresponding to: position 661 according to SEQ ID NO:17, or the complement thereof; position 649 according to SEQ ID NO:18, or the complement thereof; position 624 according to SEQ ID NO:19, or the complement thereof; position 624 according to SEQ ID NO:20, or the complement thereof; position 658 according to SEQ ID NO:21, or the complement thereof; position 661 according to SEQ ID NO:22, or the complement thereof; position 661 according to SEQ ID NO:23, or the complement thereof; position 663 according to SEQ ID NO:24, or the complement thereof; position 660 according to SEQ ID NO:25, or the complement thereof; position 649 according to SEQ ID NO:26, or the complement thereof; position 624 according to SEQ ID NO:27, or the complement thereof; or position 691 according to SEQ ID NO:28, or the complement thereof; and c) determining whether the extension product of the primer comprises an adenine at a position corresponding to: position 661 according to SEQ ID NO:17, or the complement thereof; position 649 according to SEQ ID NO:18, or the complement thereof; position 624 according to SEQ ID NO:19, or the complement thereof; position 624 according to SEQ ID NO:20, or the complement thereof; position 658 according to SEQ ID NO:21, or the complement thereof; position 661 according to SEQ ID NO:22, or the complement thereof; position 661 according to SEQ ID NO:23, or the complement thereof; position 663 according to SEQ ID NO:24, or the complement thereof; position 660 according to SEQ ID NO:25, or the complement thereof; position 649 according to SEQ ID NO:26, or the complement thereof; position 624 according to SEQ ID NO:27, or the complement thereof; or position 691 according to SEQ ID NO:28, or the complement thereof.

In some embodiments, the determining step, detecting step, or sequence analysis comprises: a) contacting the biological sample with a primer hybridizing to a portion of the nucleotide sequence of the CREB3L3 cDNA molecule, or the complement thereof, that is proximate to a position corresponding to: position 661 according to SEQ ID NO:45, or the complement thereof; position 649 according to SEQ ID NO:46, or the complement thereof; position 624 according to SEQ ID NO:47, or the complement thereof; position 624 according to SEQ ID NO:48, or the complement thereof; position 658 according to SEQ ID NO:49, or the complement thereof; position 661 according to SEQ ID NO:50, or the complement thereof; position 661 according to SEQ ID NO:51, or the complement thereof; position 663 according to SEQ ID NO:52, or the complement thereof; position 660 according to SEQ ID NO:53, or the complement thereof; position 649 according to SEQ ID NO:54, or the complement thereof; position 624 according to SEQ ID NO:55, or the complement thereof; or position 691 according to SEQ ID NO:56, or the complement thereof; b) extending the primer at least through the position of the nucleotide sequence of the CREB3L3 cDNA molecule corresponding to: position 661 according to SEQ ID NO:45, or the complement thereof; position 649 according to SEQ ID NO:46, or the complement thereof; position 624 according to SEQ ID NO:47, or the complement thereof; position 624 according to SEQ ID NO:48, or the complement thereof; position 658 according to SEQ ID NO:49, or the complement thereof; position 661 according to SEQ ID NO:50, or the complement thereof; position 661 according to SEQ ID NO:51, or the complement thereof; position 663 according to SEQ ID NO:52, or the complement thereof; position 660 according to SEQ ID NO:53, or the complement thereof; position 649 according to SEQ ID NO:54, or the complement thereof; position 624 according to SEQ ID NO:55, or the complement thereof; or position 691 according to SEQ ID NO:56, or the complement thereof; and c) determining whether the extension product of the primer comprises an adenine at a position corresponding to: position 661 according to SEQ ID NO:45, or the complement thereof; position 649 according to SEQ ID NO:46, or the complement thereof; position 624 according to SEQ ID NO:47, or the complement thereof; position 624 according to SEQ ID NO:48, or the complement thereof; position 658 according to SEQ ID NO:49, or the complement thereof; position 661 according to SEQ ID NO:50, or the complement thereof; position 661 according to SEQ ID NO:51, or the complement thereof; position 663 according to SEQ ID NO:52, or the complement thereof; position 660 according to SEQ ID NO:53, or the complement thereof; position 649 according to SEQ ID NO:54, or the complement thereof; position 624 according to SEQ ID NO:55, or the complement thereof; or position 691 according to SEQ ID NO:56, or the complement thereof.

In some embodiments, the entire nucleic acid molecule is sequenced. In some embodiments, only a CREB3L3 genomic nucleic acid molecule is analyzed. In some embodiments, only a CREB3L3 mRNA is analyzed. In some embodiments, only a CREB3L3 cDNA obtained from CREB3L3 mRNA is analyzed.

In some embodiments, the determining step, detecting step, or sequence analysis comprises: a) amplifying at least a portion of the CREB3L3 nucleic acid molecule, or the complement thereof, in the biological sample, wherein the amplified portion comprises an adenine at a position corresponding to position 6,120 according to SEQ ID NO:2, or the complement thereof; an adenine at a position corresponding to: position 661 according to SEQ ID NO:17, or the complement thereof; position 649 according to SEQ ID NO:18, or the complement thereof; position 624 according to SEQ ID NO:19, or the complement thereof; position 624 according to SEQ ID NO:20, or the complement thereof; position 658 according to SEQ ID NO:21, or the complement thereof; position 661 according to SEQ ID NO:22, or the complement thereof; position 661 according to SEQ ID NO:23, or the complement thereof; position 663 according to SEQ ID NO:24, or the complement thereof; position 660 according to SEQ ID NO:25, or the complement thereof; position 649 according to SEQ ID NO:26, or the complement thereof; position 624 according to SEQ ID NO:27, or the complement thereof; or position 691 according to SEQ ID NO:28, or the complement thereof; or an adenine at a position corresponding to: position 661 according to SEQ ID NO:45, or the complement thereof; position 649 according to SEQ ID NO:46, or the complement thereof; position 624 according to SEQ ID NO:47, or the complement thereof; position 624 according to SEQ ID NO:48, or the complement thereof; position 658 according to SEQ ID NO:49, or the complement thereof; position 661 according to SEQ ID NO:50, or the complement thereof; position 661 according to SEQ ID NO:51, or the complement thereof; position 663 according to SEQ ID NO:52, or the complement thereof; position 660 according to SEQ ID NO:53, or the complement thereof; position 649 according to SEQ ID NO:54, or the complement thereof; position 624 according to SEQ ID NO:55, or the complement thereof; or position 691 according to SEQ ID NO:56, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising an adenine at a position corresponding to position 6,120 according to SEQ ID NO:2, or the complement thereof; an adenine at a position corresponding to: position 661 according to SEQ ID NO:17, or the complement thereof; position 649 according to SEQ ID NO:18, or the complement thereof; position 624 according to SEQ ID NO:19, or the complement thereof; position 624 according to SEQ ID NO:20, or the complement thereof; position 658 according to SEQ ID NO:21, or the complement thereof; position 661 according to SEQ ID NO:22, or the complement thereof; position 661 according to SEQ ID NO:23, or the complement thereof; position 663 according to SEQ ID NO:24, or the complement thereof; position 660 according to SEQ ID NO:25, or the complement thereof; position 649 according to SEQ ID NO:26, or the complement thereof; position 624 according to SEQ ID NO:27, or the complement thereof; or position 691 according to SEQ ID NO:28, or the complement thereof; or an adenine at a position corresponding to: position 661 according to SEQ ID NO:45, or the complement thereof; position 649 according to SEQ ID NO:46, or the complement thereof; position 624 according to SEQ ID NO:47, or the complement thereof; position 624 according to SEQ ID NO:48, or the complement thereof; position 658 according to SEQ ID NO:49, or the complement thereof; position 661 according to SEQ ID NO:50, or the complement thereof; position 661 according to SEQ ID NO:51, or the complement thereof; position 663 according to SEQ ID NO:52, or the complement thereof; position 660 according to SEQ ID NO:53, or the complement thereof; position 649 according to SEQ ID NO:54, or the complement thereof; position 624 according to SEQ ID NO:55, or the complement thereof; or position 691 according to SEQ ID NO:56, or the complement thereof; and d) detecting the detectable label.

In some embodiments, the determining step, detecting step, or sequence analysis comprises: a) amplifying at least a portion of the CREB3L3 genomic nucleic acid molecule, or the complement thereof, in the biological sample, wherein the portion comprises an adenine at a position corresponding to position 6,120 according to SEQ ID NO:2, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising an adenine at a position corresponding to position 6,120 according to SEQ ID NO:2, or the complement thereof; and d) detecting the detectable label.

In some embodiments, the determining step, detecting step, or sequence analysis comprises: a) amplifying at least a portion of the CREB3L3 mRNA molecule, or the complement thereof, in the biological sample, wherein the portion comprises an adenine at a position corresponding to: position 661 according to SEQ ID NO:17, or the complement thereof; position 649 according to SEQ ID NO:18, or the complement thereof; position 624 according to SEQ ID NO:19, or the complement thereof; position 624 according to SEQ ID NO:20, or the complement thereof; position 658 according to SEQ ID NO:21, or the complement thereof; position 661 according to SEQ ID NO:22, or the complement thereof; position 661 according to SEQ ID NO:23, or the complement thereof; position 663 according to SEQ ID NO:24, or the complement thereof; position 660 according to SEQ ID NO:25, or the complement thereof; position 649 according to SEQ ID NO:26, or the complement thereof; position 624 according to SEQ ID NO:27, or the complement thereof; or position 691 according to SEQ ID NO:28, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising an adenine at a position corresponding to: position 661 according to SEQ ID NO:17, or the complement thereof; position 649 according to SEQ ID NO:18, or the complement thereof; position 624 according to SEQ ID NO:19, or the complement thereof; position 624 according to SEQ ID NO:20, or the complement thereof; position 658 according to SEQ ID NO:21, or the complement thereof; position 661 according to SEQ ID NO:22, or the complement thereof; position 661 according to SEQ ID NO:23, or the complement thereof; position 663 according to SEQ ID NO:24, or the complement thereof; position 660 according to SEQ ID NO:25, or the complement thereof; position 649 according to SEQ ID NO:26, or the complement thereof; position 624 according to SEQ ID NO:27, or the complement thereof; or position 691 according to SEQ ID NO:28, or the complement thereof; and d) detecting the detectable label.

In some embodiments, the determining step, detecting step, or sequence analysis comprises: a) amplifying at least a portion of the CREB3L3 cDNA molecule, or the complement thereof, in the biological sample, wherein the portion comprises an adenine at a position corresponding to: position 661 according to SEQ ID NO:45, or the complement thereof; position 649 according to SEQ ID NO:46, or the complement thereof; position 624 according to SEQ ID NO:47, or the complement thereof; position 624 according to SEQ ID NO:48, or the complement thereof; position 658 according to SEQ ID NO:49, or the complement thereof; position 661 according to SEQ ID NO:50, or the complement thereof; position 661 according to SEQ ID NO:51, or the complement thereof; position 663 according to SEQ ID NO:52, or the complement thereof; position 660 according to SEQ ID NO:53, or the complement thereof; position 649 according to SEQ ID NO:54, or the complement thereof; position 624 according to SEQ ID NO:55, or the complement thereof; or position 691 according to SEQ ID NO:56, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising an adenine at a position corresponding to: position 661 according to SEQ ID NO:45, or the complement thereof; position 649 according to SEQ ID NO:46, or the complement thereof; position 624 according to SEQ ID NO:47, or the complement thereof; position 624 according to SEQ ID NO:48, or the complement thereof; position 658 according to SEQ ID NO:49, or the complement thereof; position 661 according to SEQ ID NO:50, or the complement thereof; position 661 according to SEQ ID NO:51, or the complement thereof; position 663 according to SEQ ID NO:52, or the complement thereof; position 660 according to SEQ ID NO:53, or the complement thereof; position 649 according to SEQ ID NO:54, or the complement thereof; position 624 according to SEQ ID NO:55, or the complement thereof; or position 691 according to SEQ ID NO:56, or the complement thereof; and d) detecting the detectable label.

In some embodiments, the nucleic acid molecule is mRNA and the determining step further comprises reverse-transcribing the mRNA into a cDNA prior to the amplifying step.

In some embodiments, the determining step, detecting step, or sequence analysis comprises: contacting the CREB3L3 nucleic acid molecule, or the complement thereof, in the biological sample with an alteration-specific probe comprising a detectable label, wherein the alteration-specific probe comprises a nucleotide sequence which hybridizes under stringent conditions to the nucleotide sequence of the CREB3L3 nucleic acid molecule, or the complement thereof, comprising: an adenine at a position corresponding to position 6,120 according to SEQ ID NO:2, or the complement thereof; an adenine at a position corresponding to: position 661 according to SEQ ID NO:17, or the complement thereof; position 649 according to SEQ ID NO:18, or the complement thereof; position 624 according to SEQ ID NO:19, or the complement thereof; position 624 according to SEQ ID NO:20, or the complement thereof; position 658 according to SEQ ID NO:21, or the complement thereof; position 661 according to SEQ ID NO:22, or the complement thereof; position 661 according to SEQ ID NO:23, or the complement thereof; position 663 according to SEQ ID NO:24, or the complement thereof; position 660 according to SEQ ID NO:25, or the complement thereof; position 649 according to SEQ ID NO:26, or the complement thereof; position 624 according to SEQ ID NO:27, or the complement thereof; or position 691 according to SEQ ID NO:28, or the complement thereof; or an adenine at a position corresponding to: position 661 according to SEQ ID NO:45, or the complement thereof; position 649 according to SEQ ID NO:46, or the complement thereof; position 624 according to SEQ ID NO:47, or the complement thereof; position 624 according to SEQ ID NO:48, or the complement thereof; position 658 according to SEQ ID NO:49, or the complement thereof; position 661 according to SEQ ID NO:50, or the complement thereof; position 661 according to SEQ ID NO:51, or the complement thereof; position 663 according to SEQ ID NO:52, or the complement thereof; position 660 according to SEQ ID NO:53, or the complement thereof; position 649 according to SEQ ID NO:54, or the complement thereof; position 624 according to SEQ ID NO:55, or the complement thereof; or position 691 according to SEQ ID NO:56, or the complement thereof; and detecting the detectable label.

In some embodiments, the determining step, detecting step, or sequence analysis comprises: contacting the CREB3L3 genomic nucleic acid molecule, or the complement thereof, in the biological sample with an alteration-specific probe comprising a detectable label, wherein the alteration-specific probe comprises a nucleotide sequence which hybridizes under stringent conditions to the nucleotide sequence of the CREB3L3 genomic nucleic acid molecule, or the complement thereof, comprising an adenine at a position corresponding to position 6,120 according to SEQ ID NO:2, or the complement thereof; and detecting the detectable label.

In some embodiments, the determining step, detecting step, or sequence analysis comprises: contacting the CREB3L3 mRNA molecule, or the complement thereof, in the biological sample with an alteration-specific probe comprising a detectable label, wherein the alteration-specific probe comprises a nucleotide sequence which hybridizes under stringent conditions to the nucleotide sequence of the CREB3L3 mRNA molecule, or the complement thereof, comprising an adenine at a position corresponding to: position 661 according to SEQ ID NO:17, or the complement thereof; position 649 according to SEQ ID NO:18, or the complement thereof; position 624 according to SEQ ID NO:19, or the complement thereof; position 624 according to SEQ ID NO:20, or the complement thereof; position 658 according to SEQ ID NO:21, or the complement thereof; position 661 according to SEQ ID NO:22, or the complement thereof; position 661 according to SEQ ID NO:23, or the complement thereof; position 663 according to SEQ ID NO:24, or the complement thereof; position 660 according to SEQ ID NO:25, or the complement thereof; position 649 according to SEQ ID NO:26, or the complement thereof; position 624 according to SEQ ID NO:27, or the complement thereof; or position 691 according to SEQ ID NO:28, or the complement thereof; and detecting the detectable label.

In some embodiments, the determining step, detecting step, or sequence analysis comprises: contacting the CREB3L3 cDNA molecule, or the complement thereof, produced from an mRNA molecule in the biological sample with an alteration-specific probe comprising a detectable label, wherein the alteration-specific probe comprises a nucleotide sequence which hybridizes under stringent conditions to the nucleotide sequence of the CREB3L3 cDNA molecule, or the complement thereof, comprising an adenine at a position corresponding to: position 661 according to SEQ ID NO:45, or the complement thereof; position 649 according to SEQ ID NO:46, or the complement thereof; position 624 according to SEQ ID NO:47, or the complement thereof; position 624 according to SEQ ID NO:48, or the complement thereof; position 658 according to SEQ ID NO:49, or the complement thereof; position 661 according to SEQ ID NO:50, or the complement thereof; position 661 according to SEQ ID NO:51, or the complement thereof; position 663 according to SEQ ID NO:52, or the complement thereof; position 660 according to SEQ ID NO:53, or the complement thereof; position 649 according to SEQ ID NO:54, or the complement thereof; position 624 according to SEQ ID NO:55, or the complement thereof; or position 691 according to SEQ ID NO:56, or the complement thereof; and detecting the detectable label.

In some embodiments, the CREB3L3 nucleic acid molecule is present within a cell obtained from the subject.

Alteration-specific polymerase chain reaction techniques can be used to detect mutations such as SNPs in a nucleic acid sequence. Alteration-specific primers can be used because the DNA polymerase will not extend when a mismatch with the template is present.

In some embodiments, the determining step, detecting step, or sequence analysis comprises contacting the biological sample with a primer or probe, such as an alteration-specific primer or alteration-specific probe, that specifically hybridizes to a CREB3L3 variant genomic sequence, variant mRNA sequence, or variant cDNA sequence and not the corresponding CREB3L3 reference sequence under stringent conditions, and determining whether hybridization has occurred.

In some embodiments, the assay comprises RNA sequencing (RNA-Seq). In some embodiments, the assays also comprise reverse transcribing mRNA into cDNA, such as by the reverse transcriptase polymerase chain reaction (RT-PCR).

In some embodiments, the methods utilize probes and primers of sufficient nucleotide length to bind to the target nucleotide sequence and specifically detect and/or identify a polynucleotide comprising a CREB3L3 variant genomic nucleic acid molecule, variant mRNA molecule, or variant cDNA molecule. The hybridization conditions or reaction conditions can be determined by the operator to achieve this result. The nucleotide length may be any length that is sufficient for use in a detection method of choice, including any assay described or exemplified herein. Such probes and primers can hybridize specifically to a target nucleotide sequence under high stringency hybridization conditions. Probes and primers may have complete nucleotide sequence identity of contiguous nucleotides within the target nucleotide sequence, although probes differing from the target nucleotide sequence and that retain the ability to specifically detect and/or identify a target nucleotide sequence may be designed by conventional methods. Probes and primers can have about 80%, about 85%, about 90%, about 91%, about 92%, about 93%, about 94%, about 95%, about 96%, about 97%, about 98%, about 99%, or 100% sequence identity or complementarity with the nucleotide sequence of the target nucleic acid molecule.

In some embodiments, to determine whether a CREB3L3 nucleic acid molecule (genomic nucleic acid molecule, mRNA molecule, or cDNA molecule), or complement thereof, within a biological sample comprises a nucleotide sequence comprising: an adenine at a position corresponding to position 6,120 according to SEQ ID NO:2; an adenine at a position corresponding to: position 661 according to SEQ ID NO:17, position 649 according to SEQ ID NO:18, position 624 according to SEQ ID NO:19, position 624 according to SEQ ID NO:20, position 658 according to SEQ ID NO:21, position 661 according to SEQ ID NO:22, position 661 according to SEQ ID NO:23, position 663 according to SEQ ID NO:24, position 660 according to SEQ ID NO:25, position 649 according to SEQ ID NO:26, position 624 according to SEQ ID NO:27, or position 691 according to SEQ ID NO:28; or an adenine at a position corresponding to: position 661 according to SEQ ID NO:45, position 649 according to SEQ ID NO:46, position 624 according to SEQ ID NO:47, position 624 according to SEQ ID NO:48, position 658 according to SEQ ID NO:49, position 661 according to SEQ ID NO:50, position 661 according to SEQ ID NO:51, position 663 according to SEQ ID NO:52, position 660 according to SEQ ID NO:53, position 649 according to SEQ ID NO:54, position 624 according to SEQ ID NO:55, or position 691 according to SEQ ID NO:56, the biological sample can be subjected to an amplification method using a primer pair that includes a first primer derived from the 5′ flanking sequence adjacent to: an adenine at a position corresponding to position 6,120 according to SEQ ID NO:2; an adenine at a position corresponding to: position 661 according to SEQ ID NO:17, position 649 according to SEQ ID NO:18, position 624 according to SEQ ID NO:19, position 624 according to SEQ ID NO:20, position 658 according to SEQ ID NO:21, position 661 according to SEQ ID NO:22, position 661 according to SEQ ID NO:23, position 663 according to SEQ ID NO:24, position 660 according to SEQ ID NO:25, position 649 according to SEQ ID NO:26, position 624 according to SEQ ID NO:27, or position 691 according to SEQ ID NO:28; or an adenine at a position corresponding to: position 661 according to SEQ ID NO:45, position 649 according to SEQ ID NO:46, position 624 according to SEQ ID NO:47, position 624 according to SEQ ID NO:48, position 658 according to SEQ ID NO:49, position 661 according to SEQ ID NO:50, position 661 according to SEQ ID NO:51, position 663 according to SEQ ID NO:52, position 660 according to SEQ ID NO:53, position 649 according to SEQ ID NO:54, position 624 according to SEQ ID NO:55, or position 691 according to SEQ ID NO:56, and a second primer derived from the 3′ flanking sequence adjacent to: an adenine at a position corresponding to position 6,120 according to SEQ ID NO:2; an adenine at a position corresponding to: position 661 according to SEQ ID NO:17, position 649 according to SEQ ID NO:18, position 624 according to SEQ ID NO:19, position 624 according to SEQ ID NO:20, position 658 according to SEQ ID NO:21, position 661 according to SEQ ID NO:22, position 661 according to SEQ ID NO:23, position 663 according to SEQ ID NO:24, position 660 according to SEQ ID NO:25, position 649 according to SEQ ID NO:26, position 624 according to SEQ ID NO:27, or position 691 according to SEQ ID NO:28; or an adenine at a position corresponding to: position 661 according to SEQ ID NO:45, position 649 according to SEQ ID NO:46, position 624 according to SEQ ID NO:47, position 624 according to SEQ ID NO:48, position 658 according to SEQ ID NO:49, position 661 according to SEQ ID NO:50, position 661 according to SEQ ID NO:51, position 663 according to SEQ ID NO:52, position 660 according to SEQ ID NO:53, position 649 according to SEQ ID NO:54, position 624 according to SEQ ID NO:55, or position 691 according to SEQ ID NO:56 to produce an amplicon that is indicative of the presence of the SNP at positions encoding: an adenine at a position corresponding to position 6,120 according to SEQ ID NO:2; an adenine at a position corresponding to: position 661 according to SEQ ID NO:17, position 649 according to SEQ ID NO:18, position 624 according to SEQ ID NO:19, position 624 according to SEQ ID NO:20, position 658 according to SEQ ID NO:21, position 661 according to SEQ ID NO:22, position 661 according to SEQ ID NO:23, position 663 according to SEQ ID NO:24, position 660 according to SEQ ID NO:25, position 649 according to SEQ ID NO:26, position 624 according to SEQ ID NO:27, or position 691 according to SEQ ID NO:28; or an adenine at a position corresponding to: position 661 according to SEQ ID NO:45, position 649 according to SEQ ID NO:46, position 624 according to SEQ ID NO:47, position 624 according to SEQ ID NO:48, position 658 according to SEQ ID NO:49, position 661 according to SEQ ID NO:50, position 661 according to SEQ ID NO:51, position 663 according to SEQ ID NO:52, position 660 according to SEQ ID NO:53, position 649 according to SEQ ID NO:54, position 624 according to SEQ ID NO:55, or position 691 according to SEQ ID NO:56. In some embodiments, the amplicon may range in length from the combined length of the primer pairs plus one nucleotide base pair to any length of amplicon producible by a DNA amplification protocol. This distance can range from one nucleotide base pair up to the limits of the amplification reaction, or about twenty thousand nucleotide base pairs. Optionally, the primer pair flanks a region including positions comprising: an adenine at a position corresponding to position 6,120 according to SEQ ID NO:2; an adenine at a position corresponding to: position 661 according to SEQ ID NO:17, position 649 according to SEQ ID NO:18, position 624 according to SEQ ID NO:19, position 624 according to SEQ ID NO:20, position 658 according to SEQ ID NO:21, position 661 according to SEQ ID NO:22, position 661 according to SEQ ID NO:23, position 663 according to SEQ ID NO:24, position 660 according to SEQ ID NO:25, position 649 according to SEQ ID NO:26, position 624 according to SEQ ID NO:27, or position 691 according to SEQ ID NO:28; or an adenine at a position corresponding to: position 661 according to SEQ ID NO:45, position 649 according to SEQ ID NO:46, position 624 according to SEQ ID NO:47, position 624 according to SEQ ID NO:48, position 658 according to SEQ ID NO:49, position 661 according to SEQ ID NO:50, position 661 according to SEQ ID NO:51, position 663 according to SEQ ID NO:52, position 660 according to SEQ ID NO:53, position 649 according to SEQ ID NO:54, position 624 according to SEQ ID NO:55, or position 691 according to SEQ ID NO:56 and at least 1, 2, 3, 4, 5, 6, 7, 8, 9, 10, or more nucleotides on each side of positions comprising: an adenine at a position corresponding to position 6,120 according to SEQ ID NO:2; an adenine at a position corresponding to: position 661 according to SEQ ID NO:17, position 649 according to SEQ ID NO:18, position 624 according to SEQ ID NO:19, position 624 according to SEQ ID NO:20, position 658 according to SEQ ID NO:21, position 661 according to SEQ ID NO:22, position 661 according to SEQ ID NO:23, position 663 according to SEQ ID NO:24, position 660 according to SEQ ID NO:25, position 649 according to SEQ ID NO:26, position 624 according to SEQ ID NO:27, or position 691 according to SEQ ID NO:28; or an adenine at a position corresponding to: position 661 according to SEQ ID NO:45, position 649 according to SEQ ID NO:46, position 624 according to SEQ ID NO:47, position 624 according to SEQ ID NO:48, position 658 according to SEQ ID NO:49, position 661 according to SEQ ID NO:50, position 661 according to SEQ ID NO:51, position 663 according to SEQ ID NO:52, position 660 according to SEQ ID NO:53, position 649 according to SEQ ID NO:54, position 624 according to SEQ ID NO:55, or position 691 according to SEQ ID NO:56.

Similar amplicons can be generated from the mRNA and/or cDNA sequences. PCR primer pairs can be derived from a known sequence, for example, by using computer programs intended for that purpose, such as the PCR primer analysis tool in Vector NTI version 10 (Informax Inc., Bethesda Md.); PrimerSelect (DNASTAR Inc., Madison, Wis.); and Primer3 (Version 0.4.0.COPYRGT., 1991, Whitehead Institute for Biomedical Research, Cambridge, Mass.). Additionally, the sequence can be visually scanned and primers manually identified using known guidelines.

Illustrative examples of nucleic acid sequencing techniques include, but are not limited to, chain terminator (Sanger) sequencing and dye terminator sequencing. Other methods involve nucleic acid hybridization methods other than sequencing, including using labeled primers or probes directed against purified DNA, amplified DNA, and fixed cell preparations (fluorescence in situ hybridization (FISH)). In some methods, a target nucleic acid molecule may be amplified prior to or simultaneous with detection. Illustrative examples of nucleic acid amplification techniques include, but are not limited to, polymerase chain reaction (PCR), ligase chain reaction (LCR), strand displacement amplification (SDA), and nucleic acid sequence based amplification (NASBA). Other methods include, but are not limited to, ligase chain reaction, strand displacement amplification, and thermophilic SDA (tSDA).

In hybridization techniques, stringent conditions can be employed such that a probe or primer will specifically hybridize to its target. In some embodiments, a polynucleotide primer or probe under stringent conditions will hybridize to its target sequence to a detectably greater degree than to other non-target sequences, such as, at least 2-fold, at least 3-fold, at least 4-fold, or more over background, including over 10-fold over background. In some embodiments, a polynucleotide primer or probe under stringent conditions will hybridize to its target nucleotide sequence to a detectably greater degree than to other nucleotide sequences by at least 2-fold. In some embodiments, a polynucleotide primer or probe under stringent conditions will hybridize to its target nucleotide sequence to a detectably greater degree than to other nucleotide sequences by at least 3-fold. In some embodiments, a polynucleotide primer or probe under stringent conditions will hybridize to its target nucleotide sequence to a detectably greater degree than to other nucleotide sequences by at least 4-fold. In some embodiments, a polynucleotide primer or probe under stringent conditions will hybridize to its target nucleotide sequence to a detectably greater degree than to other nucleotide sequences by over 10-fold over background. Stringent conditions are sequence-dependent and will be different in different circumstances.

Appropriate stringency conditions which promote DNA hybridization, for example, 6X sodium chloride/sodium citrate (SSC) at about 45° C., followed by a wash of 2X SSC at 50° C., are known or can be found in Current Protocols in Molecular Biology, John Wiley & Sons, N.Y. (1989), 6.3.1-6.3.6. Typically, stringent conditions for hybridization and detection will be those in which the salt concentration is less than about 1.5 M Na⁺ ion, typically about 0.01 to 1.0 M Na⁺ ion concentration (or other salts) at pH 7.0 to 8.3 and the temperature is at least about 30° C. for short probes (such as, for example, 10 to 50 nucleotides) and at least about 60° C. for longer probes (such as, for example, greater than 50 nucleotides). Stringent conditions may also be achieved with the addition of destabilizing agents such as formamide. Optionally, wash buffers may comprise about 0.1% to about 1% SDS. Duration of hybridization is generally less than about 24 hours, usually about 4 to about 12 hours. The duration of the wash time will be at least a length of time sufficient to reach equilibrium.

The present disclosure also provides methods of detecting the presence of a CREB3L3 predicted loss-of-function polypeptide comprising performing an assay on a biological sample obtained from the subject to determine whether a CREB3L3 polypeptide in the biological sample contains one or more variations that causes the polypeptide to have a loss-of-function (partial or complete) or predicted loss-of-function (partial or complete). The CREB3L3 predicted loss-of-function polypeptide can be any of the CREB3L3 predicted loss-of-function polypeptides described herein. In some embodiments, the methods detect the presence of CREB3L3 Asp182Asn-A, Asp182Asn-B, Asp182Asn-C, Asp182Asn-D, or Asp181Asn. In some embodiments, the methods detect the presence of CREB3L3 Asp182Asn-A, Asp182Asn-B, Asp182Asn-C, or Asp182Asn-D.

In some embodiments, the methods comprise performing an assay on a biological sample obtained from a subject to determine whether a CREB3L3 polypeptide in the biological sample comprises an asparagine at a position corresponding to position 182 according to SEQ ID NO:64, an asparagine at a position corresponding to position 182 according to SEQ ID NO:65, an asparagine at a position corresponding to position 182 according to SEQ ID NO:66, an asparagine at a position corresponding to position 182 according to SEQ ID NO:67, or an asparagine at a position corresponding to position 181 according to SEQ ID NO:68.

In some embodiments, the detecting step comprises sequencing at least a portion of the CREB3L3 polypeptide that comprises a position corresponding to: position 182 according to SEQ ID NO:64, position 182 according to SEQ ID NO:65, position 182 according to SEQ ID NO:66, position 182 according to SEQ ID NO:67, position 181 according to SEQ ID NO:68, SEQ ID NO:59, SEQ ID NO:60, SEQ ID NO:61, SEQ ID NO:62, or SEQ ID NO:63.

In some embodiments, the detecting step comprises an immunoassay for detecting the presence of a CREB3L3 polypeptide that comprises a position corresponding to: position 182 according to SEQ ID NO:64, position 182 according to SEQ ID NO:65, position 182 according to SEQ ID NO:66, or position 182 according to SEQ ID NO:67, or SEQ ID NO:59, SEQ ID NO:60, SEQ ID NO:61, SEQ ID NO:62, or SEQ ID NO:63.

In some embodiments, when the subject does not have a CREB3L3 predicted loss-of-function polypeptide, the subject has an increased risk of developing a liver disease or any of parenchymal liver disease, liver fibrosis, liver cirrhosis, or NAFLD. In some embodiments, when the subject has a CREB3L3 predicted loss-of-function polypeptide, the subject has a decreased risk of developing a liver disease or any of parenchymal liver disease, liver fibrosis, liver cirrhosis, or NAFLD.

The present disclosure also provides isolated nucleic acid molecules that hybridize to CREB3L3 variant genomic nucleic acid molecules, CREB3L3 variant mRNA molecules, and/or CREB3L3 variant cDNA molecules (such as any of the genomic variant nucleic acid molecules, mRNA variant molecules, and cDNA variant molecules disclosed herein). In some embodiments, such isolated nucleic acid molecules hybridize to CREB3L3 variant nucleic acid molecules under stringent conditions. Such nucleic acid molecules can be used, for example, as probes, primers, alteration-specific probes, or alteration-specific primers as described or exemplified herein.

In some embodiments, the isolated nucleic acid molecules hybridize to a portion of the CREB3L3 nucleic acid molecule that includes a position corresponding to: position 6,120 according to SEQ ID NO:2, position 661 according to SEQ ID NO:17, position 649 according to SEQ ID NO:18, position 624 according to SEQ ID NO:19, position 624 according to SEQ ID NO:20, position 658 according to SEQ ID NO:21, position 661 according to SEQ ID NO:22, position 661 according to SEQ ID NO:23, position 663 according to SEQ ID NO:24, position 660 according to SEQ ID NO:25, position 649 according to SEQ ID NO:26, position 624 according to SEQ ID NO:27, position 691 according to SEQ ID NO:28, position 661 according to SEQ ID NO:45, position 649 according to SEQ ID NO:46, position 624 according to SEQ ID NO:47, position 624 according to SEQ ID NO:48, position 658 according to SEQ ID NO:49, position 661 according to SEQ ID NO:50, position 661 according to SEQ ID NO:51, position 663 according to SEQ ID NO:52, position 660 according to SEQ ID NO:53, position 649 according to SEQ ID NO:54, position 624 according to SEQ ID NO:55, or position 691 according to SEQ ID NO:56.

In some embodiments, such isolated nucleic acid molecules comprise or consist of at least about 5, at least about 8, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, at least about 200, at least about 300, at least about 400, at least about 500, at least about 600, at least about 700, at least about 800, at least about 900, at least about 1000, at least about 2000, at least about 3000, at least about 4000, or at least about 5000 nucleotides. In some embodiments, such isolated nucleic acid molecules comprise or consist of at least about 5, at least about 8, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, or at least about 25 nucleotides. In some embodiments, the isolated nucleic acid molecules comprise or consist of at least about 18 nucleotides. In some embodiments, the isolated nucleic acid molecules comprise or consists of at least about 15 nucleotides. In some embodiments, the isolated nucleic acid molecules consist of or comprise from about 10 to about 35, from about 10 to about 30, from about 10 to about 25, from about 12 to about 30, from about 12 to about 28, from about 12 to about 24, from about 15 to about 30, from about 15 to about 25, from about 18 to about 30, from about 18 to about 25, from about 18 to about 24, or from about 18 to about 22 nucleotides. In some embodiments, the isolated nucleic acid molecules consist of or comprise from about 18 to about 30 nucleotides. In some embodiments, the isolated nucleic acid molecules comprise or consist of at least about 15 nucleotides to at least about 35 nucleotides.

In some embodiments, the isolated nucleic acid molecules hybridize to at least about 15 contiguous nucleotides of a nucleic acid molecule that is at least about 70%, at least about 75%, at least about 80%, at least about 85%, at least about 90%, at least about 95%, at least about 96%, at least about 97%, at least about 98%, at least about 99%, or 100% identical to CREB3L3 variant genomic nucleic acid molecules, CREB3L3 variant mRNA molecules, and/or CREB3L3 variant cDNA molecules. In some embodiments, the isolated nucleic acid molecules consist of or comprise from about 15 to about 100 nucleotides, or from about 15 to about 35 nucleotides. In some embodiments, the isolated nucleic acid molecules consist of or comprise from about 15 to about 100 nucleotides. In some embodiments, the isolated nucleic acid molecules consist of or comprise from about 15 to about 35 nucleotides.

In some embodiments, the isolated alteration-specific probes or alteration-specific primers comprise at least about 15 nucleotides, wherein the alteration-specific probe or alteration-specific primer comprises a nucleotide sequence which is complementary to the nucleotide sequence of a portion of a CREB3L3 nucleic acid molecule encoding a CREB3L3 predicted loss-of-function polypeptide, or the complement thereof. In some embodiments, the portion comprises a position corresponding to: position 6,120 according to SEQ ID NO:2, or the complement thereof; position 661 according to SEQ ID NO:17, or the complement thereof; position 649 according to SEQ ID NO:18, or the complement thereof; position 624 according to SEQ ID NO:19, or the complement thereof; position 624 according to SEQ ID NO:20, or the complement thereof; position 658 according to SEQ ID NO:21, or the complement thereof; position 661 according to SEQ ID NO:22, or the complement thereof; position 661 according to SEQ ID NO:23, or the complement thereof; position 663 according to SEQ ID NO:24, or the complement thereof; position 660 according to SEQ ID NO:25, or the complement thereof; position 649 according to SEQ ID NO:26, or the complement thereof; position 624 according to SEQ ID NO:27, or the complement thereof; position 691 according to SEQ ID NO:28, or the complement thereof; position 661 according to SEQ ID NO:45, or the complement thereof; position 649 according to SEQ ID NO:46, or the complement thereof; position 624 according to SEQ ID NO:47, or the complement thereof; position 624 according to SEQ ID NO:48, or the complement thereof; position 658 according to SEQ ID NO:49, or the complement thereof; position 661 according to SEQ ID NO:50, or the complement thereof; position 661 according to SEQ ID NO:51, or the complement thereof; position 663 according to SEQ ID NO:52, or the complement thereof; position 660 according to SEQ ID NO:53, or the complement thereof; position 649 according to SEQ ID NO:54, or the complement thereof; position 624 according to SEQ ID NO:55, or the complement thereof; or position 691 according to SEQ ID NO:56, or the complement thereof. In some embodiments, the portion comprises positions corresponding to: positions 6,120-6,122 according to SEQ ID NO:2, or the complement thereof; positions 661-663 according to SEQ ID NO:17, or the complement thereof; positions 649-651 according to SEQ ID NO:18, or the complement thereof; positions 624-626 according to SEQ ID NO:19, or the complement thereof; positions 624-626 according to SEQ ID NO:20, or the complement thereof; positions 658-660 according to SEQ ID NO:21, or the complement thereof; positions 661-663 according to SEQ ID NO:22, or the complement thereof; positions 661-663 according to SEQ ID NO:23, or the complement thereof; positions 663-665 according to SEQ ID NO:24, or the complement thereof; positions 660-662 according to SEQ ID NO:25, or the complement thereof; positions 649-651 according to SEQ ID NO:26, or the complement thereof; positions 624-626 according to SEQ ID NO:27, or the complement thereof; positions 691-693 according to SEQ ID NO:28, or the complement thereof; positions 661-663 according to SEQ ID NO:45, or the complement thereof; positions 649-651 according to SEQ ID NO:46, or the complement thereof; positions 624-626 according to SEQ ID NO:47, or the complement thereof; positions 624-626 according to SEQ ID NO:48, or the complement thereof; positions 658-660 according to SEQ ID NO:49, or the complement thereof; positions 661-663 according to SEQ ID NO:50, or the complement thereof; positions 661-663 according to SEQ ID NO:51, or the complement thereof; positions 663-665 according to SEQ ID NO:52, or the complement thereof; positions 660-662 according to SEQ ID NO:53, or the complement thereof; positions 649-651 according to SEQ ID NO:54, or the complement thereof; positions 624-626 according to SEQ ID NO:55, or the complement thereof; or positions 691-693 according to SEQ ID NO:56, or the complement thereof.

In some embodiments, the alteration-specific probes and alteration-specific primers comprise DNA. In some embodiments, the alteration-specific probes and alteration-specific primers comprise RNA.

In some embodiments, the probes and primers described herein (including alteration-specific probes and alteration-specific primers) have a nucleotide sequence that specifically hybridizes to any of the nucleic acid molecules disclosed herein, or the complement thereof. In some embodiments, the probes and primers specifically hybridize to any of the nucleic acid molecules disclosed herein under stringent conditions.

In some embodiments, the primers, including alteration-specific primers, can be used in second generation sequencing or high throughput sequencing. In some instances, the primers, including alteration-specific primers, can be modified. In particular, the primers can comprise various modifications that are used at different steps of, for example, Massive Parallel Signature Sequencing (MPSS), Polony sequencing, and 454 Pyrosequencing. Modified primers can be used at several steps of the process, including biotinylated primers in the cloning step and fluorescently labeled primers used at the bead loading step and detection step. Polony sequencing is generally performed using a paired-end tags library wherein each molecule of DNA template is about 135 bp in length. Biotinylated primers are used at the bead loading step and emulsion PCR. Fluorescently labeled degenerate nonamer oligonucleotides are used at the detection step. An adaptor can contain a 5′-biotin tag for immobilization of the DNA library onto streptavidin-coated beads.

The probes and primers described herein can be used to detect a nucleotide variation within any of the CREB3L3 variant genomic nucleic acid molecules, CREB3L3 variant mRNA molecules, and/or CREB3L3 variant cDNA molecules disclosed herein. The primers described herein can be used to amplify the CREB3L3 variant genomic nucleic acid molecules, CREB3L3 variant mRNA molecules, or CREB3L3 variant cDNA molecules, or a fragment thereof.

The present disclosure also provides pairs of primers comprising any of the primers described above. For example, if one of the primers′ 3′-ends hybridizes to a guanine at a position corresponding to position 6,120 according to SEQ ID NO:1 (rather than an adenine) in a particular CREB3L3 nucleic acid molecule, then the presence of the amplified fragment would indicate the presence of a CREB3L3 reference genomic nucleic acid molecule. Conversely, if one of the primers′ 3′-ends hybridizes to an adenine at a position corresponding to position 6,120 according to SEQ ID NO:2 (rather than a guanine) in a particular CREB3L3 nucleic acid molecule, then the presence of the amplified fragment would indicate the presence of the CREB3L3 variant genomic nucleic acid molecule. In some embodiments, the nucleotide of the primer complementary to the adenine at a position corresponding to position 6,120 according to SEQ ID NO:2 can be at the 3′ end of the primer. In addition, if one of the primers’ 3′-ends hybridizes to a guanine at a position corresponding to position 661 according to SEQ ID NO:3 (rather than an adenine) in a particular CREB3L3 nucleic acid molecule, then the presence of the amplified fragment would indicate the presence of a CREB3L3 reference mRNA molecule. Conversely, if one of the primers’ 3′-ends hybridizes to an adenine at a position corresponding to position 661 according to SEQ ID NO:17 (rather than a guanine) in a particular CREB3L3 mRNA molecule, then the presence of the amplified fragment would indicate the presence of the CREB3L3 variant mRNA molecule. In some embodiments, the nucleotide of the primer complementary to the adenine at a position corresponding to position 661 according to SEQ ID NO:17 can be at the 3′ end of the primer.

If one of the primers’ 3′-ends hybridizes to a guanine at a position corresponding to position 649 according to SEQ ID NO:4 (rather than an adenine) in a particular CREB3L3 nucleic acid molecule, then the presence of the amplified fragment would indicate the presence of a CREB3L3 reference mRNA molecule. Conversely, if one of the primers’ 3′-ends hybridizes to an adenine at a position corresponding to position 649 according to SEQ ID NO:18 (rather than a guanine) in a particular CREB3L3 mRNA molecule, then the presence of the amplified fragment would indicate the presence of the CREB3L3 variant mRNA molecule. In some embodiments, the nucleotide of the primer complementary to the adenine at a position corresponding to position 649 according to SEQ ID NO:18 can be at the 3′ end of the primer.

If one of the primers’ 3′-ends hybridizes to a guanine at a position corresponding to position 624 according to SEQ ID NO:5 (rather than an adenine) in a particular CREB3L3 nucleic acid molecule, then the presence of the amplified fragment would indicate the presence of a CREB3L3 reference mRNA molecule. Conversely, if one of the primers’ 3′-ends hybridizes to an adenine at a position corresponding to position 624 according to SEQ ID NO:19 (rather than a guanine) in a particular CREB3L3 mRNA molecule, then the presence of the amplified fragment would indicate the presence of the CREB3L3 variant mRNA molecule. In some embodiments, the nucleotide of the primer complementary to the adenine at a position corresponding to position 624 according to SEQ ID NO:19 can be at the 3′ end of the primer.

If one of the primers’ 3′-ends hybridizes to a guanine at a position corresponding to position 624 according to SEQ ID NO:6 (rather than an adenine) in a particular CREB3L3 nucleic acid molecule, then the presence of the amplified fragment would indicate the presence of a CREB3L3 reference mRNA molecule. Conversely, if one of the primers’ 3′-ends hybridizes to an adenine at a position corresponding to position 624 according to SEQ ID NO:20 (rather than a guanine) in a particular CREB3L3 mRNA molecule, then the presence of the amplified fragment would indicate the presence of the CREB3L3 variant mRNA molecule. In some embodiments, the nucleotide of the primer complementary to the adenine at a position corresponding to position 624 according to SEQ ID NO:20 can be at the 3′ end of the primer.

If one of the primers’ 3′-ends hybridizes to a guanine at a position corresponding to position 658 according to SEQ ID NO:7 (rather than an adenine) in a particular CREB3L3 nucleic acid molecule, then the presence of the amplified fragment would indicate the presence of a CREB3L3 reference mRNA molecule. Conversely, if one of the primers’ 3′-ends hybridizes to an adenine at a position corresponding to position 658 according to SEQ ID NO:21 (rather than a guanine) in a particular CREB3L3 mRNA molecule, then the presence of the amplified fragment would indicate the presence of the CREB3L3 variant mRNA molecule. In some embodiments, the nucleotide of the primer complementary to the adenine at a position corresponding to position 658 according to SEQ ID NO:21 can be at the 3′ end of the primer.

If one of the primers’ 3′-ends hybridizes to a guanine at a position corresponding to position 661 according to SEQ ID NO:8 (rather than an adenine) in a particular CREB3L3 nucleic acid molecule, then the presence of the amplified fragment would indicate the presence of a CREB3L3 reference mRNA molecule. Conversely, if one of the primers’ 3′-ends hybridizes to an adenine at a position corresponding to position 661 according to SEQ ID NO:22 (rather than a guanine) in a particular CREB3L3 mRNA molecule, then the presence of the amplified fragment would indicate the presence of the CREB3L3 variant mRNA molecule. In some embodiments, the nucleotide of the primer complementary to the adenine at a position corresponding to position 661 according to SEQ ID NO:22 can be at the 3′ end of the primer.

If one of the primers’ 3′-ends hybridizes to a guanine at a position corresponding to position 661 according to SEQ ID NO:9 (rather than an adenine) in a particular CREB3L3 nucleic acid molecule, then the presence of the amplified fragment would indicate the presence of a CREB3L3 reference mRNA molecule. Conversely, if one of the primers’ 3′-ends hybridizes to an adenine at a position corresponding to position 661 according to SEQ ID NO:23 (rather than a guanine) in a particular CREB3L3 mRNA molecule, then the presence of the amplified fragment would indicate the presence of the CREB3L3 variant mRNA molecule. In some embodiments, the nucleotide of the primer complementary to the adenine at a position corresponding to position 661 according to SEQ ID NO:23 can be at the 3′ end of the primer.

If one of the primers’ 3′-ends hybridizes to a guanine at a position corresponding to position 663 according to SEQ ID NO:10 (rather than an adenine) in a particular CREB3L3 nucleic acid molecule, then the presence of the amplified fragment would indicate the presence of a CREB3L3 reference mRNA molecule. Conversely, if one of the primers’ 3′-ends hybridizes to an adenine at a position corresponding to position 663 according to SEQ ID NO:24 (rather than a guanine) in a particular CREB3L3 mRNA molecule, then the presence of the amplified fragment would indicate the presence of the CREB3L3 variant mRNA molecule. In some embodiments, the nucleotide of the primer complementary to the adenine at a position corresponding to position 663 according to SEQ ID NO:24 can be at the 3′ end of the primer.

If one of the primers’ 3′-ends hybridizes to a guanine at a position corresponding to position 663 according to SEQ ID NO:11 (rather than an adenine) in a particular CREB3L3 nucleic acid molecule, then the presence of the amplified fragment would indicate the presence of a CREB3L3 reference mRNA molecule. Conversely, if one of the primers’ 3′-ends hybridizes to an adenine at a position corresponding to position 660 according to SEQ ID NO:25 (rather than a guanine) in a particular CREB3L3 mRNA molecule, then the presence of the amplified fragment would indicate the presence of the CREB3L3 variant mRNA molecule. In some embodiments, the nucleotide of the primer complementary to the adenine at a position corresponding to position 660 according to SEQ ID NO:25 can be at the 3′ end of the primer.

If one of the primers’ 3′-ends hybridizes to a guanine at a position corresponding to position 649 according to SEQ ID NO:12 (rather than an adenine) in a particular CREB3L3 nucleic acid molecule, then the presence of the amplified fragment would indicate the presence of a CREB3L3 reference mRNA molecule. Conversely, if one of the primers’ 3′-ends hybridizes to an adenine at a position corresponding to position 649 according to SEQ ID NO:26 (rather than a guanine) in a particular CREB3L3 mRNA molecule, then the presence of the amplified fragment would indicate the presence of the CREB3L3 variant mRNA molecule. In some embodiments, the nucleotide of the primer complementary to the adenine at a position corresponding to position 649 according to SEQ ID NO:26 can be at the 3′ end of the primer.

If one of the primers’ 3′-ends hybridizes to a guanine at a position corresponding to position 624 according to SEQ ID NO:13 (rather than an adenine) in a particular CREB3L3 nucleic acid molecule, then the presence of the amplified fragment would indicate the presence of a CREB3L3 reference mRNA molecule. Conversely, if one of the primers’ 3′-ends hybridizes to an adenine at a position corresponding to position 624 according to SEQ ID NO:27 (rather than a guanine) in a particular CREB3L3 mRNA molecule, then the presence of the amplified fragment would indicate the presence of the CREB3L3 variant mRNA molecule. In some embodiments, the nucleotide of the primer complementary to the adenine at a position corresponding to position 624 according to SEQ ID NO:27 can be at the 3′ end of the primer.

If one of the primers’ 3′-ends hybridizes to a guanine at a position corresponding to position 691 according to SEQ ID NO:14 (rather than an adenine) in a particular CREB3L3 nucleic acid molecule, then the presence of the amplified fragment would indicate the presence of a CREB3L3 reference mRNA molecule. Conversely, if one of the primers’ 3′-ends hybridizes to an adenine at a position corresponding to position 691 according to SEQ ID NO:28 (rather than a guanine) in a particular CREB3L3 mRNA molecule, then the presence of the amplified fragment would indicate the presence of the CREB3L3 variant mRNA molecule. In some embodiments, the nucleotide of the primer complementary to the adenine at a position corresponding to position 691 according to SEQ ID NO:28 can be at the 3′ end of the primer.

If one of the primers’ 3′-ends hybridizes to a guanine at a position corresponding to position 661 according to SEQ ID NO:31 (rather than an adenine) in a particular CREB3L3 nucleic acid molecule, then the presence of the amplified fragment would indicate the presence of a CREB3L3 reference cDNA molecule. Conversely, if one of the primers’ 3′-ends hybridizes to an adenine at a position corresponding to position 661 according to SEQ ID NO:45 (rather than a guanine) in a particular CREB3L3 cDNA molecule, then the presence of the amplified fragment would indicate the presence of the CREB3L3 variant cDNA molecule. In some embodiments, the nucleotide of the primer complementary to the adenine at a position corresponding to position 661 according to SEQ ID NO:45 can be at the 3′ end of the primer.

If one of the primers’ 3′-ends hybridizes to a guanine at a position corresponding to position 649 according to SEQ ID NO:32 (rather than an adenine) in a particular CREB3L3 nucleic acid molecule, then the presence of the amplified fragment would indicate the presence of a CREB3L3 reference cDNA molecule. Conversely, if one of the primers’ 3′-ends hybridizes to an adenine at a position corresponding to position 649 according to SEQ ID NO:46 (rather than a guanine) in a particular CREB3L3 cDNA molecule, then the presence of the amplified fragment would indicate the presence of the CREB3L3 variant cDNA molecule. In some embodiments, the nucleotide of the primer complementary to the adenine at a position corresponding to position 649 according to SEQ ID NO:46 can be at the 3′ end of the primer.

If one of the primers’ 3′-ends hybridizes to a guanine at a position corresponding to position 624 according to SEQ ID NO:33 (rather than an adenine) in a particular CREB3L3 nucleic acid molecule, then the presence of the amplified fragment would indicate the presence of a CREB3L3 reference cDNA molecule. Conversely, if one of the primers’ 3′-ends hybridizes to an adenine at a position corresponding to position 624 according to SEQ ID NO:47 (rather than a guanine) in a particular CREB3L3 cDNA molecule, then the presence of the amplified fragment would indicate the presence of the CREB3L3 variant cDNA molecule. In some embodiments, the nucleotide of the primer complementary to the adenine at a position corresponding to position 624 according to SEQ ID NO:47 can be at the 3′ end of the primer.

If one of the primers’ 3′-ends hybridizes to a guanine at a position corresponding to position 624 according to SEQ ID NO:31 (rather than an adenine) in a particular CREB3L3 nucleic acid molecule, then the presence of the amplified fragment would indicate the presence of a CREB3L3 reference cDNA molecule. Conversely, if one of the primers’ 3′-ends hybridizes to an adenine at a position corresponding to position 624 according to SEQ ID NO:48 (rather than a guanine) in a particular CREB3L3 cDNA molecule, then the presence of the amplified fragment would indicate the presence of the CREB3L3 variant cDNA molecule. In some embodiments, the nucleotide of the primer complementary to the adenine at a position corresponding to position 624 according to SEQ ID NO:48 can be at the 3′ end of the primer.

If one of the primers’ 3′-ends hybridizes to a guanine at a position corresponding to position 658 according to SEQ ID NO:35 (rather than an adenine) in a particular CREB3L3 nucleic acid molecule, then the presence of the amplified fragment would indicate the presence of a CREB3L3 reference cDNA molecule. Conversely, if one of the primers’ 3′-ends hybridizes to an adenine at a position corresponding to position 658 according to SEQ ID NO:49 (rather than a guanine) in a particular CREB3L3 cDNA molecule, then the presence of the amplified fragment would indicate the presence of the CREB3L3 variant cDNA molecule. In some embodiments, the nucleotide of the primer complementary to the adenine at a position corresponding to position 658 according to SEQ ID NO:49 can be at the 3′ end of the primer.

If one of the primers’ 3′-ends hybridizes to a guanine at a position corresponding to position 661 according to SEQ ID NO:36 (rather than an adenine) in a particular CREB3L3 nucleic acid molecule, then the presence of the amplified fragment would indicate the presence of a CREB3L3 reference cDNA molecule. Conversely, if one of the primers’ 3′-ends hybridizes to an adenine at a position corresponding to position 661 according to SEQ ID NO:50 (rather than a guanine) in a particular CREB3L3 cDNA molecule, then the presence of the amplified fragment would indicate the presence of the CREB3L3 variant cDNA molecule. In some embodiments, the nucleotide of the primer complementary to the adenine at a position corresponding to position 661 according to SEQ ID NO:50 can be at the 3′ end of the primer.

If one of the primers’ 3′-ends hybridizes to a guanine at a position corresponding to position 661 according to SEQ ID NO:37 (rather than an adenine) in a particular CREB3L3 nucleic acid molecule, then the presence of the amplified fragment would indicate the presence of a CREB3L3 reference cDNA molecule. Conversely, if one of the primers’ 3′-ends hybridizes to an adenine at a position corresponding to position 661 according to SEQ ID NO:51 (rather than a guanine) in a particular CREB3L3 cDNA molecule, then the presence of the amplified fragment would indicate the presence of the CREB3L3 variant cDNA molecule. In some embodiments, the nucleotide of the primer complementary to the adenine at a position corresponding to position 661 according to SEQ ID NO:51 can be at the 3′ end of the primer.

If one of the primers’ 3′-ends hybridizes to a guanine at a position corresponding to position 663 according to SEQ ID NO:38 (rather than an adenine) in a particular CREB3L3 nucleic acid molecule, then the presence of the amplified fragment would indicate the presence of a CREB3L3 reference cDNA molecule. Conversely, if one of the primers’ 3′-ends hybridizes to an adenine at a position corresponding to position 663 according to SEQ ID NO:52 (rather than a guanine) in a particular CREB3L3 cDNA molecule, then the presence of the amplified fragment would indicate the presence of the CREB3L3 variant cDNA molecule. In some embodiments, the nucleotide of the primer complementary to the adenine at a position corresponding to position 663 according to SEQ ID NO:52 can be at the 3′ end of the primer.

If one of the primers’ 3′-ends hybridizes to a guanine at a position corresponding to position 663 according to SEQ ID NO:39 (rather than an adenine) in a particular CREB3L3 nucleic acid molecule, then the presence of the amplified fragment would indicate the presence of a CREB3L3 reference cDNA molecule. Conversely, if one of the primers’ 3′-ends hybridizes to an adenine at a position corresponding to position 660 according to SEQ ID NO:53 (rather than a guanine) in a particular CREB3L3 cDNA molecule, then the presence of the amplified fragment would indicate the presence of the CREB3L3 variant cDNA molecule. In some embodiments, the nucleotide of the primer complementary to the adenine at a position corresponding to position 660 according to SEQ ID NO:53 can be at the 3′ end of the primer.

If one of the primers’ 3′-ends hybridizes to a guanine at a position corresponding to position 649 according to SEQ ID NO:40 (rather than an adenine) in a particular CREB3L3 nucleic acid molecule, then the presence of the amplified fragment would indicate the presence of a CREB3L3 reference cDNA molecule. Conversely, if one of the primers’ 3′-ends hybridizes to an adenine at a position corresponding to position 649 according to SEQ ID NO:54 (rather than a guanine) in a particular CREB3L3 cDNA molecule, then the presence of the amplified fragment would indicate the presence of the CREB3L3 variant cDNA molecule. In some embodiments, the nucleotide of the primer complementary to the adenine at a position corresponding to position 649 according to SEQ ID NO:54 can be at the 3′ end of the primer.

If one of the primers’ 3′-ends hybridizes to a guanine at a position corresponding to position 624 according to SEQ ID NO:41 (rather than an adenine) in a particular CREB3L3 nucleic acid molecule, then the presence of the amplified fragment would indicate the presence of a CREB3L3 reference cDNA molecule. Conversely, if one of the primers’ 3′-ends hybridizes to an adenine at a position corresponding to position 624 according to SEQ ID NO:55 (rather than a guanine) in a particular CREB3L3 cDNA molecule, then the presence of the amplified fragment would indicate the presence of the CREB3L3 variant cDNA molecule. In some embodiments, the nucleotide of the primer complementary to the adenine at a position corresponding to position 624 according to SEQ ID NO:55 can be at the 3′ end of the primer.

If one of the primers’ 3′-ends hybridizes to a guanine at a position corresponding to position 691 according to SEQ ID NO:42 (rather than an adenine) in a particular CREB3L3 nucleic acid molecule, then the presence of the amplified fragment would indicate the presence of a CREB3L3 reference cDNA molecule. Conversely, if one of the primers’ 3′-ends hybridizes to an adenine at a position corresponding to position 691 according to SEQ ID NO:56 (rather than a guanine) in a particular CREB3L3 cDNA molecule, then the presence of the amplified fragment would indicate the presence of the CREB3L3 variant cDNA molecule. In some embodiments, the nucleotide of the primer complementary to the adenine at a position corresponding to position 691 according to SEQ ID NO:56 can be at the 3′ end of the primer.

In the context of the present disclosure “specifically hybridizes” means that the probe or primer (such as, for example, the alteration-specific probe or alteration-specific primer) does not hybridize to a nucleic acid sequence encoding a CREB3L3 reference genomic nucleic acid molecule, a CREB3L3 reference mRNA molecule, and/or a CREB3L3 reference cDNA molecule.

In any of the embodiments described throughout the present disclosure, the probes (such as, for example, an alteration-specific probe) can comprise a label. In some embodiments, the label is a fluorescent label, a radiolabel, or biotin.

The present disclosure also provides supports comprising a substrate to which any one or more of the probes disclosed herein is attached. Solid supports are solid-state substrates or supports with which molecules, such as any of the probes disclosed herein, can be associated. A form of solid support is an array. Another form of solid support is an array detector. An array detector is a solid support to which multiple different probes have been coupled in an array, grid, or other organized pattern. A form for a solid-state substrate is a microtiter dish, such as a standard 96-well type. In some embodiments, a multiwell glass slide can be employed that normally contains one array per well. In some embodiments, the support is a microarray.

In some embodiments, any of the methods described herein can further comprise determining the subject’s gene burden of having a CREB3L3 variant nucleic acid molecule (such as, for example a CREB3L3 variant nucleic acid molecule (such as, for example a CREB3L3 variant nucleic acid molecule encoding a CREB3L3 predicted loss-of-function polypeptide), and/or a CREB3L3 predicted loss-of-function variant polypeptide associated with a decreased risk of developing a liver disease. The gene burden is the aggregate of all variants in the CREB3L3 gene, which can be carried out in an association analysis with a liver disease. In some embodiments, the subject is homozygous for one or more CREB3L3 variant nucleic acid molecules associated with a decreased risk of developing a liver disease. In some embodiments, the subject is heterozygous for one or more CREB3L3 variant nucleic acid molecules associated with a decreased risk of developing a liver disease. The result of the association analysis suggests that CREB3L3 variant nucleic acid molecules (such as, for example a CREB3L3 variant nucleic acid molecule (such as, for example a CREB3L3 variant nucleic acid molecule encoding a CREB3L3 predicted loss-of-function polypeptide) are associated with decreased risk of developing a liver disease. When the subject has a lower gene burden, the subject is at a higher risk of developing a liver disease and the subject is administered or continued to be administered the therapeutic agent that treats, prevents, or inhibits a liver disease in a standard dosage amount, and/or a CREB3L3 inhibitor. When the subject has a greater gene burden, the subject is at a decreased risk of developing a liver disease and the subject is administered or continued to be administered the therapeutic agent that treats, prevents, or inhibits a liver disease in an amount that is the same as or less than the standard dosage amount. The greater the gene burden, the lower the risk of developing a liver disease. Table 2 lists representative CREB3L3 variant nucleic acid molecules that can be used in the gene burden analysis. The associations between CREB3L3 and liver phenotypes were driven by multiple rare pLOF variants in the gene. In some embodiments, the gene burden analysis includes the variant 19:4159750:G:A (Asp182Asn).

TABLE 2

Predicted loss-of-function variants with alternate allele frequency < 1% in CREB3L3 identified by exome sequencing and included in gene burden association analyses

| | |
|---|---|
| Genomic coordinates for the genetic variant, C:P:R:A | transcriptlds |
| 19:4153747:C:CAT | ENST00000078445:ENST00000595923:ENST00000602257:ENST0 0000602147 |
| 19:4153748:A:G | ENST00000078445:ENST00000595923:ENST00000602257:ENST0 0000602147 |
| 19:4153749:T:C | ENST00000078445:ENST00000595923:ENST00000602257:ENST0 0000602147 |
| 19:4153749:T:G | ENST00000078445:ENST00000595923:ENST00000602257:ENST0 0000602147 |
| 19:4153775:G:GCCTAC | ENST00000078445:ENST00000595923:ENST00000602257:ENST0 0000602147 |
| 19:4153776:T:C | ENST00000078445:ENST00000595923:ENST00000602257:ENST0 0000602147 |
| 19:4154898:G:A | ENST00000078445:ENST00000595923:ENST00000602257:ENST0 0000602147 |
| 19:4154905:TC:T | ENST00000078445:ENST00000595923:ENST00000602257:ENST0 0000602147 |
| 19:4154965:CG:C | ENST00000078445:ENST00000595923:ENST00000602257:ENST0 0000602147 |
| 19:4154990:TG:T | ENST00000078445:ENST00000595923:ENST00000602257:ENST0 0000602147 |
| 19:4155001:G:T | ENST00000078445:ENST00000595923:ENST00000602257:ENST0 0000602147 |
| 19:4155007:TG:T | ENST00000078445:ENST00000595923:ENST00000602257:ENST0 0000602147 |
| 19:4155009:G:A | ENST00000078445:ENST00000595923:ENST00000602257:ENST0 0000602147 |
| 19:4155028:G:A | ENST00000078445:ENST00000595923:ENST00000602257:ENST0 0000602147 |
| 19:4156993:A:T | ENST00000078445:ENST00000602257:ENST00000602147 |
| 19:4156994:G:A | ENST00000078445:ENST00000602257:ENST00000602147 |
| 19:4156995:C:CA | ENST00000078445:ENST00000602257:ENST00000602147 |
| 19:4157078:GT:G | ENST00000078445:ENST00000595923:ENST00000602257:ENST0 0000602147 |
| 19:4157079:T:TC | ENST00000078445:ENST00000595923:ENST00000602257:ENST0 0000602147 |
| 19:4157079:TC:T | ENST00000078445:ENST00000595923:ENST00000602257:ENST0 0000602147 |
| 19:4157109:G:T | ENST00000078445:ENST00000595923:ENST00000602257:ENST0 0000602147 |
| 19:4157130:C:T | ENST00000078445:ENST00000595923:ENST00000602257:ENST0 0000602147 |
| 19:4157143:C:CA | ENST00000078445:ENST00000595923:ENST00000602257:ENST0 0000602147 |
| 19:4157187:C:T | ENST00000078445:ENST00000595923:ENST00000602257:ENST0 0000602147 |
| 19:4157192:TGGCAA:T | ENST00000078445:ENST00000595923:ENST00000602257:ENST0 0000602147 |
| 19:4157196:A:T | ENST00000078445:ENST00000595923:ENST00000602257:ENST0 0000602147 |
| 19:4157197:AG:A | ENST00000078445:ENST00000595923:ENST00000602257:ENST0 0000602147 |
| 19:4157201:G:GA | ENST00000078445:ENST00000595923:ENST00000602257:ENST0 0000602147 |
| 19:4157207:C:A | ENST00000078445:ENST00000595923:ENST00000602257:ENST0 0000602147 |
| 19:4157258:G:GC | ENST00000078445:ENST00000595923:ENST00000602257:ENST0 0000602147 |
| 19:4157295:G:T | ENST00000078445:ENST00000595923:ENST00000602257:ENST0 0000602147 |
| 19:4157296:G:A | ENST00000078445:ENST00000595923:ENST00000602257:ENST0 0000602147 |
| 19:4157297:T:C | ENST00000078445:ENST00000595923:ENST00000602257:ENST0 0000602147 |
| 19:4159662:A:G | ENST00000078445:ENST00000595923:ENST00000602257:ENST0 0000602147 |
| 19:4159669:TG:T | ENST00000078445:ENST00000595923:ENST00000602257:ENST0 0000602147 |
| 19:4159670:GGA:G | ENST00000078445:ENST00000595923:ENST00000602257:ENST0 0000602147 |
| 19:4159673:GC:G | ENST00000078445:ENST00000595923:ENST00000602257:ENST0 0000602147 |
| 19:4159679:G:GAGGA | ENST00000078445:ENST00000595923:ENST00000602257:ENST0 0000602147 |
| 19:4159679:GA:G | ENST00000078445:ENST00000595923:ENST00000602257:ENST0 0000602147 |
| 19:4159679:GAGGA:G | ENST00000078445:ENST00000595923:ENST00000602257:ENST0 0000602147 |
| 19:4159682:GA:G | ENST00000078445:ENST00000595923:ENST00000602257:ENST0 0000602147 |
| 19:4159692:T:A | ENST00000078445:ENST00000595923:ENST00000602257:ENST0 0000602147 |
| 19:4159695:TGA:T | ENST00000078445:ENST00000595923:ENST00000602257:ENST0 0000602147 |
| 19:4159715:TG:T | ENST00000078445:ENST00000595923:ENST00000602257:ENST0 0000602147 |
| 19:4159723:TC:T | ENST00000078445:ENST00000595923:ENST00000602257:ENST0 0000602147 |
| 19:4159723:TCC:T | ENST00000078445:ENST00000595923:ENST00000602257:ENST0 0000602147 |
| 19:4159729:C:T | ENST00000078445:ENST00000595923:ENST00000602257:ENST0 0000602147 |
| 19:4159733:GC:G | ENST00000078445:ENST00000595923:ENST00000602257:ENST0 0000602147 |
| 19:4159733:GCA:G | ENST00000078445:ENST00000595923:ENST00000602257:ENST0 0000602147 |
| 19:4159734:C:A | ENST00000078445:ENST00000595923:ENST00000602257:ENST0 0000602147 |
| 19:4159736:ATC:A | ENST00000078445:ENST00000595923:ENST00000602257:ENST0 0000602147 |
| 19:4159745:TG:T | ENST00000078445:ENST00000595923:ENST00000602257:ENST0 0000602147 |
| 19:4159746:GA:G | ENST00000078445:ENST00000595923:ENST00000602257:ENST0 0000602147 |
| 19:4159751:AC:A | ENST00000078445:ENST00000595923:ENST00000602257:ENST0 0000602147 |
| 19:4159754:TC:T | ENST00000078445:ENST00000595923:ENST00000602257:ENST0 0000602147 |
| 19:4159759:CT:C | ENST00000078445:ENST00000595923:ENST00000602257:ENST0 0000602147 |
| 19:4159759:CTT:C | ENST00000078445:ENST00000595923:ENST00000602257:ENST0 0000602147 |
| 19:4159763:C:A | ENST00000078445:ENST00000595923:ENST00000602257:ENST0 0000602147 |
| 19:4159763:CG:C | ENST00000078445:ENST00000595923:ENST00000602257:ENST0 0000602147 |
| 19:4159766:GC:G | ENST00000078445:ENST00000595923:ENST00000602257:ENST0 0000602147 |
| 19:4159773:TG:T | ENST00000078445:ENST00000595923:ENST00000602257:ENST0 0000602147 |
| 19:4159783:G:A | ENST00000078445:ENST00000595923:ENST00000602257:ENST0 0000602147 |
| 19:4159784:T:A | ENST00000078445:ENST00000595923:ENST00000602257:ENST0 0000602147 |
| 19:4164501:A:G | ENST00000078445:ENST00000595923:ENST00000602147 |
| 19:4164502:G:A | ENST00000078445:ENST00000595923:ENST00000602147 |
| 19:4164503:C:T | ENST00000078445:ENST00000595923:ENST00000602147 |
| 19:4164506:C:T | ENST00000078445:ENST00000595923:ENST00000602147 |
| 19:4164507:A:G | ENST00000602257 |
| 19:4164516:TG:T | ENST00000078445:ENST00000595923:ENST00000602257:ENST0 0000602147 |
| 19:4164529:C:A | ENST00000078445:ENST00000595923:ENST00000602257:ENST0 0000602147 |
| 19:4164536:C:T | ENST00000078445:ENST00000595923:ENST00000602257:ENST0 0000602147 |
| 19:4164560:G:T | ENST00000078445:ENST00000595923:ENST00000602257:ENST0 0000602147 |
| 19:4164575:G:T | ENST00000078445:ENST00000595923:ENST00000602257:ENST0 0000602147 |
| 19:4164586:GA:G | ENST00000078445:ENST00000595923:ENST00000602257:ENST0 0000602147 |
| 19:4164606:G:GC | ENST00000078445:ENST00000595923:ENST00000602257:ENST0 0000602147 |
| 19:4164641:G:A | ENST00000078445:ENST00000595923:ENST00000602257:ENST0 0000602147 |
| 19:4168350:G:C | ENST00000078445:ENST00000595923:ENST00000602257 |
| 19:4168353:C:G | ENST00000078445:ENST00000595923:ENST00000602257 |
| 19:4168359:GC:G | ENST00000078445:ENST00000595923:ENST00000602257 |
| 19:4168360:C:T | ENST00000078445:ENST00000595923:ENST00000602257 |
| 19:4168367:T:TG | ENST00000078445:ENST00000595923:ENST00000602257 |
| 19:4168368:G:GA | ENST00000078445:ENST00000595923:ENST00000602257 |
| 19:4168383:GA:G | ENST00000078445:ENST00000595923:ENST00000602257 |
| 19:4168403:C:A | ENST00000078445:ENST00000595923:ENST00000602257 |
| 19:4168407:GC:G | ENST00000078445:ENST00000595923:ENST00000602257 |
| 19:4168459:T:G | ENST00000078445:ENST00000595923:ENST00000602257 |
| 19:4170138:A:G | ENST00000078445:ENST00000595923:ENST00000602257:ENST0 0000602147 |
| 19:4170165:C:A | ENST00000602147 |
| 19:4170170:GT:G | ENST00000078445:ENST00000595923:ENST00000602257:ENST0 0000602147 |
| 19:4170174:C:T | ENST00000078445:ENST00000595923:ENST00000602257 |
| 19:4170194:C:T | ENST00000602147 |
| 19:4170195:G:T | ENST00000078445:ENST00000595923:ENST00000602257 |
| 19:4170199:AG:A | ENST00000078445:ENST00000595923:ENST00000602257:ENST0 0000602147 |
| 19:4170201:C:T | ENST00000078445:ENST00000595923:ENST00000602257 |
| 19:4170209:G:C | ENST00000078445:ENST00000595923:ENST00000602257:ENST0 0000602147 |
| 19:4170210:T:A | ENST00000078445:ENST00000595923:ENST00000602257:ENST0 0000602147 |
| 19:4171089:A:G | ENST00000078445:ENST00000595923:ENST00000602257:ENST0 0000602147 |
| 19:4171116:CT:C | ENST00000078445:ENST00000595923:ENST00000602257:ENST0 0000602147 |
| 19:4171134:C:T | ENST00000078445:ENST00000595923:ENST00000602257 |
| 19:4171142:C:T | ENST00000602147 |
| 19:4171150:C:A | ENST00000078445:ENST00000595923:ENST00000602257 |
| 19:4171402:C:A | ENST00000602147 |
| 19:4171439:CA:C | ENST00000078445:ENST00000595923:ENST00000602257:ENST0 0000602147 |
| 19:4171448:C:T | ENST00000602147 |
| 19:4171457:TG:T | ENST00000078445:ENST00000595923:ENST00000602257:ENST0 0000602147 |
| 19:4171476:C:T | ENST00000078445:ENST00000595923:ENST00000602257 |
| 19:4171654:A:G | ENST00000078445:ENST00000595923:ENST00000602257:ENST0 0000602147 |
| 19:4171655:G:T | ENST00000078445:ENST00000595923:ENST00000602257:ENST0 0000602147 |
| 19:4171668:CT:C | ENST00000078445:ENST00000595923:ENST00000602257:ENST0 0000602147 |
| 19:4171673:CA:C | ENST00000078445:ENST00000595923:ENST00000602257:ENST0 0000602147 |
| 19:4171678:C:T | ENST00000602147 |
| 19:4171688:TC:T | ENST00000078445:ENST00000595923:ENST00000602257:ENST0 0000602147 |
| 19:4171692:G:GCTGCT | ENST00000078445:ENST00000595923:ENST00000602257:ENST0 0000602147 |
| 19:4171702:T:C | ENST00000602147 |
| 19:4171704:A:C | ENST00000602147 |
| 19:4171736:C:T | ENST00000078445:ENST00000595923:ENST00000602257 |
| 19:4171757:C:T | ENST00000078445:ENST00000595923:ENST00000602257 |
| 19:4171757:CGAGAA:C | ENST00000078445:ENST00000595923:ENST00000602257 |
| 19:4171761:AAG:A | ENST00000078445:ENST00000595923:ENST00000602257 |
| 19:4171765:GTC:G | ENST00000078445:ENST00000595923:ENST00000602257 |
| 19:4171771:AG:A | ENST00000078445:ENST00000595923:ENST00000602257 |
| 19:4171791:G:A | ENST00000078445:ENST00000595923:ENST00000602257 |
| 19:4171811:AACCTG:A | ENST00000078445:ENST00000595923:ENST00000602257 |
| 19:4171824:C:A | ENST00000078445:ENST00000595923:ENST00000602257 |
| 19:4171827:C:CGGAG | ENST00000078445:ENST00000595923:ENST00000602257 |
| 19:4171832:G:T | ENST00000078445:ENST00000595923:ENST00000602257 |
| 19:4171869:CAG:C | ENST00000078445:ENST00000595923:ENST00000602257 |
| 19:4171883:C:T | ENST00000078445:ENST00000595923:ENST00000602257 |
| 19:4171903:G:A | ENST00000078445:ENST00000595923:ENST00000602257 |
| 19:4171942:G:GCTGGAGGC | ENST00000078445:ENST00000595923:ENST00000602257 |
| 19:4171946:G:T | ENST00000078445:ENST00000595923:ENST00000602257 |
| 19:4171948:GGC:G | ENST00000078445:ENST00000595923:ENST00000602257 |
| 19:4171955:G:T | ENST00000078445:ENST00000595923:ENST00000602257 |

In some embodiments, the subject’s gene burden of having any one or more CREB3L3 variant nucleic acid molecules (such as, for example a CREB3L3 variant nucleic acid molecule (such as, for example a CREB3L3 variant nucleic acid molecule encoding a CREB3L3 predicted loss-of-function polypeptide) represents a weighted aggregate of a plurality of any of the CREB3L3 variant nucleic acid molecules. In some embodiments, the gene burden is calculated using at least about 2, at least about 3, at least about 4, at least about 5, at least about 10, at least about 20, at least about 30, at least about 40, at least about 50, at least about 60, at least about 70, at least about 80, at least about 100, at least about 120, at least about 150, at least about 200, at least about 250, at least about 300, at least about 400, at least about 500, at least about 1,000, at least about 10,000, at least about 100,000, or at least about or more than 1,000,000 genetic variants present in or around (up to 10 Mb) the CREB3L3 gene where the gene burden is the number of alleles multiplied by the association estimate with a liver disease or related outcome for each allele (e.g., a weighted polygenic burden score). This can include any genetic variants, regardless of their genomic annotation, in proximity to the CREB3L3 gene (up to 10 Mb around the gene) that show a non-zero association with liver disease-related traits in a genetic association analysis. In some embodiments, when the subject has a gene burden above a desired threshold score, the subject has a decreased risk of developing a liver disease. In some embodiments, when the subject has a gene burden below a desired threshold score, the subject has an increased risk of developing a liver disease.

In some embodiments, the gene burden may be divided into quintiles, e.g., top quintile, intermediate quintile, and bottom quintile, wherein the top quintile of gene burden corresponds to the lowest risk group and the bottom quintile of gene burden corresponds to the highest risk group. In some embodiments, a subject having a greater gene burden comprises the highest weighted gene burdens, including, but not limited to the top 10%, top 20%, top 30%, top 40%, or top 50% of gene burdens from a subject population. In some embodiments, the genetic variants comprise the genetic variants having association with a liver disease in the top 10%, top 20%, top 30%, top 40%, or top 50% of p-value range for the association. In some embodiments, each of the identified genetic variants comprise the genetic variants having association with a liver disease with p-value of no more than about 10-2, about 10-3, about 10-4, about 10-5, about 10-6, about 10-7, about 10-8, about 10-9, about 10-10, about 10-11, about 10-12, about 10-13, about 10-14, about or 10-15. In some embodiments, the identified genetic variants comprise the genetic variants having association with a liver disease with p-value of less than 5 x 10-8. In some embodiments, the identified genetic variants comprise genetic variants having association with a liver disease in high-risk subjects as compared to the rest of the reference population with odds ratio (OR) about 1.5 or greater, about 1.75 or greater, about 2.0 or greater, or about 2.25 or greater for the top 20% of the distribution; or about 1.5 or greater, about 1.75 or greater, about 2.0 or greater, about 2.25 or greater, about 2.5 or greater, or about 2.75 or greater. In some embodiments, the odds ratio (OR) may range from about 1.0 to about 1.5, from about 1.5 to about 2.0, from about 2.0 to about 2.5, from about 2.5 to about 3.0, from about 3.0 to about 3.5, from about 3.5 to about 4.0, from about 4.0 to about 4.5, from about 4.5 to about 5.0, from about 5.0 to about 5.5, from about 5.5 to about 6.0, from about 6.0 to about 6.5, from about 6.5 to about 7.0, or greater than 7.0. In some embodiments, high-risk subjects comprise subjects having gene burdens in the bottom decile, quintile, or tertile in a reference population. The threshold of the gene burden is determined on the basis of the nature of the intended practical application and the risk difference that would be considered meaningful for that practical application.

In some embodiments, when a subject is identified as having an increased risk of developing a liver disease, the subject is further administered a therapeutic agent that treats, prevents, or inhibits a liver disease, and/or a CREB3L3 inhibitor, as described herein. For example, when the subject is CREB3L3 reference, and therefore has an increased risk of developing a liver disease, the subject is administered a CREB3L3 inhibitor. In some embodiments, such a subject is also administered a therapeutic agent that treats, prevents, or inhibits a liver disease. In some embodiments, when the subject is heterozygous for a CREB3L3 variant nucleic acid molecule (such as, for example a CREB3L3 variant nucleic acid molecule (such as, for example a CREB3L3 variant nucleic acid molecule encoding a CREB3L3 predicted loss-of-function polypeptide), the subject is administered the therapeutic agent that treats, prevents, or inhibits a liver disease in a dosage amount that is the same as or less than a standard dosage amount, and is also administered a CREB3L3 inhibitor. In some embodiments, the subject is CREB3L3 reference. In some embodiments, the subject is heterozygous for a CREB3L3 variant nucleic acid molecule. Furthermore, when the subject has a lower gene burden for having a CREB3L3 variant nucleic acid molecule (such as, for example a CREB3L3 variant nucleic acid molecule encoding a CREB3L3 predicted loss-of-function polypeptide), and therefore has an increased risk of developing a liver disease, the subject is administered a therapeutic agent that treats, prevents, or inhibits a liver disease. In some embodiments, when the subject has a lower gene burden for having a CREB3L3 variant nucleic acid molecule (such as, for example a CREB3L3 variant nucleic acid molecule encoding a CREB3L3 predicted loss-of-function polypeptide), the subject is administered the therapeutic agent that treats, prevents, or inhibits a liver disease in a dosage amount that is the same as or greater than the standard dosage amount administered to a subject who has a greater gene burden for having a CREB3L3 variant nucleic acid molecule (such as, for example a CREB3L3 variant nucleic acid molecule encoding a CREB3L3 predicted loss-of-function polypeptide).

The nucleotide sequence of a CREB3L3 reference genomic nucleic acid molecule is set forth in SEQ ID NO:1. Referring to SEQ ID NO:1, position 6,120 is a guanine.

A CREB3L3 variant genomic nucleic acid molecule exists, wherein the guanine at position 6,120 is replaced with an adenine. The nucleotide sequence of this CREB3L3 variant genomic nucleic acid molecule is set forth in SEQ ID NO:2.

The nucleotide sequence of a CREB3L3 reference mRNA molecule is set forth in SEQ ID NO:3. Referring to SEQ ID NO:3, position 661 is a guanine.

The nucleotide sequence of another CREB3L3 reference mRNA molecule is set forth in SEQ ID NO:4. Referring to SEQ ID NO:4, position 649 is a guanine.

The nucleotide sequence of another CREB3L3 reference mRNA molecule is set forth in SEQ ID NO:5. Referring to SEQ ID NO:5, position 624 is a guanine.

The nucleotide sequence of another CREB3L3 reference mRNA molecule is set forth in SEQ ID NO:6. Referring to SEQ ID NO:6, position 624 is a guanine.

The nucleotide sequence of another CREB3L3 reference mRNA molecule is set forth in SEQ ID NO:7. Referring to SEQ ID NO:7, position 658 is a guanine.

The nucleotide sequence of another CREB3L3 reference mRNA molecule is set forth in SEQ ID NO:8. Referring to SEQ ID NO:8, position 661 is a guanine.

The nucleotide sequence of another CREB3L3 reference mRNA molecule is set forth in SEQ ID NO:9. Referring to SEQ ID NO:9, position 661 is a guanine.

The nucleotide sequence of another CREB3L3 reference mRNA molecule is set forth in SEQ ID NO:10. Referring to SEQ ID NO:10, position 663 is a guanine.

The nucleotide sequence of another CREB3L3 reference mRNA molecule is set forth in SEQ ID NO:11. Referring to SEQ ID NO:11, position 663 is a guanine.

The nucleotide sequence of another CREB3L3 reference mRNA molecule is set forth in SEQ ID NO:12. Referring to SEQ ID NO:12, position 649 is a guanine.

The nucleotide sequence of another CREB3L3 reference mRNA molecule is set forth in SEQ ID NO:13. Referring to SEQ ID NO:13, position 624 is a guanine.

The nucleotide sequence of another CREB3L3 reference mRNA molecule is set forth in SEQ ID NO:14. Referring to SEQ ID NO:14, position 691 is a guanine.

The nucleotide sequence of another CREB3L3 reference mRNA molecule is set forth in SEQ ID NO:15. Referring to SEQ ID NO:15, position 660 is a guanine.

The nucleotide sequence of another CREB3L3 reference mRNA molecule is set forth in SEQ ID NO:16. Referring to SEQ ID NO:16, position 620 is a guanine.

A CREB3L3 variant mRNA molecule exists, wherein the guanine at position 661 is replaced with an adenine. The nucleotide sequence of this CREB3L3 variant mRNA molecule is set forth in SEQ ID NO:17.

Another CREB3L3 variant mRNA molecule exists, wherein the guanine at position 649 is replaced with an adenine. The nucleotide sequence of this CREB3L3 variant mRNA molecule is set forth in SEQ ID NO:18.

Another CREB3L3 variant mRNA molecule exists, wherein the guanine at position 624 is replaced with an adenine. The nucleotide sequence of this CREB3L3 variant mRNA molecule is set forth in SEQ ID NO:19.

Another CREB3L3 variant mRNA molecule exists, wherein the guanine at position 624 is replaced with an adenine. The nucleotide sequence of this CREB3L3 variant mRNA molecule is set forth in SEQ ID NO:20.

Another CREB3L3 variant mRNA molecule exists, wherein the guanine at position 658 is replaced with an adenine. The nucleotide sequence of this CREB3L3 variant mRNA molecule is set forth in SEQ ID NO:21.

Another CREB3L3 variant mRNA molecule exists, wherein the guanine at position 661 is replaced with an adenine. The nucleotide sequence of this CREB3L3 variant mRNA molecule is set forth in SEQ ID NO:22.

Another CREB3L3 variant mRNA molecule exists, wherein the guanine at position 661 is replaced with an adenine. The nucleotide sequence of this CREB3L3 variant mRNA molecule is set forth in SEQ ID NO:23.

Another CREB3L3 variant mRNA molecule exists, wherein the guanine at position 663 is replaced with an adenine. The nucleotide sequence of this CREB3L3 variant mRNA molecule is set forth in SEQ ID NO:24.

Another CREB3L3 variant mRNA molecule exists, wherein the guanine at position 663 is replaced with an adenine. The nucleotide sequence of this CREB3L3 variant mRNA molecule is set forth in SEQ ID NO:25.

Another CREB3L3 variant mRNA molecule exists, wherein the guanine at position 649 is replaced with an adenine. The nucleotide sequence of this CREB3L3 variant mRNA molecule is set forth in SEQ ID NO:26.

Another CREB3L3 variant mRNA molecule exists, wherein the guanine at position 624 is replaced with an adenine. The nucleotide sequence of this CREB3L3 variant mRNA molecule is set forth in SEQ ID NO:27.

Another CREB3L3 variant mRNA molecule exists, wherein the guanine at position 691 is replaced with an adenine. The nucleotide sequence of this CREB3L3 variant mRNA molecule is set forth in SEQ ID NO:28.

Another CREB3L3 variant mRNA molecule exists, wherein the guanine at position 660 is replaced with an adenine. The nucleotide sequence of this CREB3L3 variant mRNA molecule is set forth in SEQ ID NO:29.

Another CREB3L3 variant mRNA molecule exists, wherein the guanine at position 620 is replaced with an adenine. The nucleotide sequence of this CREB3L3 variant mRNA molecule is set forth in SEQ ID NO:30.

The nucleotide sequence of a CREB3L3 reference cDNA molecule is set forth in SEQ ID NO:31. Referring to SEQ ID NO:31, position 661 is a guanine.

The nucleotide sequence of another CREB3L3 reference cDNA molecule is set forth in SEQ ID NO:32. Referring to SEQ ID NO:32, position 649 is a guanine.

The nucleotide sequence of another CREB3L3 reference cDNA molecule is set forth in SEQ ID NO:33. Referring to SEQ ID NO:33, position 624 is a guanine.

The nucleotide sequence of another CREB3L3 reference cDNA molecule is set forth in SEQ ID NO:34. Referring to SEQ ID NO:34, position 624 is a guanine.

The nucleotide sequence of another CREB3L3 reference cDNA molecule is set forth in SEQ ID NO:35. Referring to SEQ ID NO:35, position 658 is a guanine.

The nucleotide sequence of another CREB3L3 reference cDNA molecule is set forth in SEQ ID NO:36. Referring to SEQ ID NO:36, position 661 is a guanine.

The nucleotide sequence of another CREB3L3 reference cDNA molecule is set forth in SEQ ID NO:37. Referring to SEQ ID NO:37, position 661 is a guanine.

The nucleotide sequence of another CREB3L3 reference cDNA molecule is set forth in SEQ ID NO:38. Referring to SEQ ID NO:38, position 663 is a guanine.

The nucleotide sequence of another CREB3L3 reference cDNA molecule is set forth in SEQ ID NO:39. Referring to SEQ ID NO:39, position 663 is a guanine.

The nucleotide sequence of another CREB3L3 reference cDNA molecule is set forth in SEQ ID NO:40. Referring to SEQ ID NO:40, position 649 is a guanine.

The nucleotide sequence of another CREB3L3 reference cDNA molecule is set forth in SEQ ID NO:41. Referring to SEQ ID NO:41, position 624 is a guanine.

The nucleotide sequence of another CREB3L3 reference cDNA molecule is set forth in SEQ ID NO:42. Referring to SEQ ID NO:42, position 691 is a guanine.

The nucleotide sequence of another CREB3L3 reference cDNA molecule is set forth in SEQ ID NO:43. Referring to SEQ ID NO:43, position 660 is a guanine.

The nucleotide sequence of another CREB3L3 reference cDNA molecule is set forth in SEQ ID NO:43. Referring to SEQ ID NO:43, position 620 is a guanine.

A CREB3L3 variant cDNA molecule exists, wherein the guanine at position 661 is replaced with an adenine. The nucleotide sequence of this CREB3L3 variant cDNA molecule is set forth in SEQ ID NO:45.

Another CREB3L3 variant cDNA molecule exists, wherein the guanine at position 649 is replaced with an adenine. The nucleotide sequence of this CREB3L3 variant cDNA molecule is set forth in SEQ ID NO:46.

Another CREB3L3 variant cDNA molecule exists, wherein the guanine at position 624 is replaced with an adenine. The nucleotide sequence of this CREB3L3 variant cDNA molecule is set forth in SEQ ID NO:47.

Another CREB3L3 variant cDNA molecule exists, wherein the guanine at position 624 is replaced with an adenine. The nucleotide sequence of this CREB3L3 variant cDNA molecule is set forth in SEQ ID NO:48.

Another CREB3L3 variant cDNA molecule exists, wherein the guanine at position 658 is replaced with an adenine. The nucleotide sequence of this CREB3L3 variant cDNA molecule is set forth in SEQ ID NO:49.

Another CREB3L3 variant cDNA molecule exists, wherein the guanine at position 661 is replaced with an adenine. The nucleotide sequence of this CREB3L3 variant cDNA molecule is set forth in SEQ ID NO:50.

Another CREB3L3 variant cDNA molecule exists, wherein the guanine at position 661 is replaced with an adenine. The nucleotide sequence of this CREB3L3 variant cDNA molecule is set forth in SEQ ID NO:51.

Another CREB3L3 variant cDNA molecule exists, wherein the guanine at position 663 is replaced with an adenine. The nucleotide sequence of this CREB3L3 variant cDNA molecule is set forth in SEQ ID NO:52.

Another CREB3L3 variant cDNA molecule exists, wherein the guanine at position 663 is replaced with an adenine. The nucleotide sequence of this CREB3L3 variant cDNA molecule is set forth in SEQ ID NO:53.

Another CREB3L3 variant cDNA molecule exists, wherein the guanine at position 649 is replaced with an adenine. The nucleotide sequence of this CREB3L3 variant cDNA molecule is set forth in SEQ ID NO:54.

Another CREB3L3 variant cDNA molecule exists, wherein the guanine at position 624 is replaced with an adenine. The nucleotide sequence of this CREB3L3 variant cDNA molecule is set forth in SEQ ID NO:55.

Another CREB3L3 variant cDNA molecule exists, wherein the guanine at position 691 is replaced with an adenine. The nucleotide sequence of this CREB3L3 variant cDNA molecule is set forth in SEQ ID NO:56.

Another CREB3L3 variant cDNA molecule exists, wherein the guanine at position 660 is replaced with an adenine. The nucleotide sequence of this CREB3L3 variant cDNA molecule is set forth in SEQ ID NO:57.

Another CREB3L3 variant cDNA molecule exists, wherein the guanine at position 620 is replaced with an adenine. The nucleotide sequence of this CREB3L3 variant cDNA molecule is set forth in SEQ ID NO:58.

The genomic nucleic acid molecules, mRNA molecules, and cDNA molecules can be from any organism. For example, the genomic nucleic acid molecules, mRNA molecules, and cDNA molecules can be human or an ortholog from another organism, such as a non-human mammal, a rodent, a mouse, or a rat. It is understood that gene sequences within a population can vary due to polymorphisms such as single-nucleotide polymorphisms. The examples provided herein are only exemplary sequences. Other sequences are also possible.

Also provided herein are functional polynucleotides that can interact with the disclosed nucleic acid molecules. Examples of functional polynucleotides include, but are not limited to, antisense molecules, aptamers, ribozymes, triplex forming molecules, and external guide sequences. The functional polynucleotides can act as effectors, inhibitors, modulators, and stimulators of a specific activity possessed by a target molecule, or the functional polynucleotides can possess a de novo activity independent of any other molecules.

The isolated nucleic acid molecules disclosed herein can comprise RNA, DNA, or both RNA and DNA. The isolated nucleic acid molecules can also be linked or fused to a heterologous nucleic acid sequence, such as in a vector, or a heterologous label. For example, the isolated nucleic acid molecules disclosed herein can be within a vector or as an exogenous donor sequence comprising the isolated nucleic acid molecule and a heterologous nucleic acid sequence. The isolated nucleic acid molecules can also be linked or fused to a heterologous label. The label can be directly detectable (such as, for example, fluorophore) or indirectly detectable (such as, for example, hapten, enzyme, or fluorophore quencher). Such labels can be detectable by spectroscopic, photochemical, biochemical, immunochemical, or chemical means. Such labels include, for example, radiolabels, pigments, dyes, chromogens, spin labels, and fluorescent labels. The label can also be, for example, a chemiluminescent substance; a metal-containing substance; or an enzyme, where there occurs an enzyme-dependent secondary generation of signal. The term “label” can also refer to a “tag” or hapten that can bind selectively to a conjugated molecule such that the conjugated molecule, when added subsequently along with a substrate, is used to generate a detectable signal. For example, biotin can be used as a tag along with an avidin or streptavidin conjugate of horseradish peroxidate (HRP) to bind to the tag, and examined using a calorimetric substrate (such as, for example, tetramethylbenzidine (TMB)) or a fluorogenic substrate to detect the presence of HRP. Exemplary labels that can be used as tags to facilitate purification include, but are not limited to, myc, HA, FLAG or 3XFLAG, 6XHis or polyhistidine, glutathione-S-transferase (GST), maltose binding protein, an epitope tag, or the Fc portion of immunoglobulin. Numerous labels include, for example, particles, fluorophores, haptens, enzymes and their calorimetric, fluorogenic and chemiluminescent substrates and other labels.

The isolated nucleic acid molecules, or the complement thereof, can also be present within a host cell. In some embodiments, the host cell can comprise the vector that comprises any of the nucleic acid molecules described herein, or the complement thereof. In some embodiments, the nucleic acid molecule is operably linked to a promoter active in the host cell. In some embodiments, the promoter is an exogenous promoter. In some embodiments, the promoter is an inducible promoter. In some embodiments, the host cell is a bacterial cell, a yeast cell, an insect cell, or a mammalian cell. In some embodiments, the host cell is a bacterial cell. In some embodiments, the host cell is a yeast cell. In some embodiments, the host cell is an insect cell. In some embodiments, the host cell is a mammalian cell.

The disclosed nucleic acid molecules can comprise, for example, nucleotides or non-natural or modified nucleotides, such as nucleotide analogs or nucleotide substitutes. Such nucleotides include a nucleotide that contains a modified base, sugar, or phosphate group, or that incorporates a non-natural moiety in its structure. Examples of non-natural nucleotides include, but are not limited to, dideoxynucleotides, biotinylated, aminated, deaminated, alkylated, benzylated, and fluorophor-labeled nucleotides.

The nucleic acid molecules disclosed herein can also comprise one or more nucleotide analogs or substitutions. A nucleotide analog is a nucleotide which contains a modification to either the base, sugar, or phosphate moieties. Modifications to the base moiety include, but are not limited to, natural and synthetic modifications of A, C, G, and T/U, as well as different purine or pyrimidine bases such as, for example, pseudouridine, uracil-5-yl, hypoxanthin-9-yl (I), and 2-aminoadenin-9-yl. Modified bases include, but are not limited to, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo (such as, for example, 5-bromo), 5-trifluoromethyl and other 5-substituted uracils and cytosines, 7-methylguanine, 7-methyladenine, 8-azaguanine, 8-azaadenine, 7-deazaguanine, 7-deazaadenine, 3-deazaguanine, and 3-deazaadenine.

Nucleotide analogs can also include modifications of the sugar moiety. Modifications to the sugar moiety include, but are not limited to, natural modifications of the ribose and deoxy ribose as well as synthetic modifications. Sugar modifications include, but are not limited to, the following modifications at the 2′ position: OH; F; O—, S—, or N-alkyl; O—, S—, or N-alkenyl; O—, S— or N-alkynyl; or O-alkyl-O-alkyl, wherein the alkyl, alkenyl, and alkynyl may be substituted or unsubstituted C₁₋₁₀alkyl or C₂₋₁₀alkenyl, and C₂₋₁₀alkynyl. Exemplary 2′ sugar modifications also include, but are not limited to, —O[(CH₂)ₙO]ₘCH₃, —O(CH₂)ₙOCH₃, —O(CH₂)ₙNH₂, —O(CH₂)ₙCH₃, —O(CH₂)ₙ—ONH₂, and —O(CH₂)ₙON[(CH₂)ₙCH₃)]₂, where n and m, independently, are from 1 to about 10. Other modifications at the 2′ position include, but are not limited to, C₁₋₁₀alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of 5′ terminal nucleotide. Modified sugars can also include those that contain modifications at the bridging ring oxygen, such as CH₂ and S. Nucleotide sugar analogs can also have sugar mimetics, such as cyclobutyl moieties in place of the pentofuranosyl sugar.

Nucleotide analogs can also be modified at the phosphate moiety. Modified phosphate moieties include, but are not limited to, those that can be modified so that the linkage between two nucleotides contains a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. These phosphate or modified phosphate linkage between two nucleotides can be through a 3′-5′ linkage or a 2′-5′ linkage, and the linkage can contain inverted polarity such as 3′-5′ to 5′-3′ or 2′-5′ to 5′-2′. Various salts, mixed salts, and free acid forms are also included. Nucleotide substitutes also include peptide nucleic acids (PNAs).

The present disclosure also provides vectors comprising any one or more of the nucleic acid molecules disclosed herein. In some embodiments, the vectors comprise any one or more of the nucleic acid molecules disclosed herein and a heterologous nucleic acid. The vectors can be viral or nonviral vectors capable of transporting a nucleic acid molecule. In some embodiments, the vector is a plasmid or cosmid (such as, for example, a circular double-stranded DNA into which additional DNA segments can be ligated). In some embodiments, the vector is a viral vector, wherein additional DNA segments can be ligated into the viral genome. Expression vectors include, but are not limited to, plasmids, cosmids, retroviruses, adenoviruses, adeno-associated viruses (AAV), plant viruses such as cauliflower mosaic virus and tobacco mosaic virus, yeast artificial chromosomes (YACs), Epstein-Barr (EBV)-derived episomes, and other expression vectors known in the art.

Desired regulatory sequences for mammalian host cell expression can include, for example, viral elements that direct high levels of polypeptide expression in mammalian cells, such as promoters and/or enhancers derived from retroviral LTRs, cytomegalovirus (CMV) (such as, for example, CMV promoter/enhancer), Simian Virus 40 (SV40) (such as, for example, SV40 promoter/enhancer), adenovirus, (such as, for example, the adenovirus major late promoter (AdMLP)), polyoma and strong mammalian promoters such as native immunoglobulin and actin promoters. Methods of expressing polypeptides in bacterial cells or fungal cells (such as, for example, yeast cells) are also well known. A promoter can be, for example, a constitutively active promoter, a conditional promoter, an inducible promoter, a temporally restricted promoter (such as, for example, a developmentally regulated promoter), or a spatially restricted promoter (such as, for example, a cell-specific or tissue-specific promoter).

Percent identity (or percent complementarity) between particular stretches of nucleotide sequences within nucleic acid molecules or amino acid sequences within polypeptides can be determined routinely using BLAST programs (basic local alignment search tools) and PowerBLAST programs (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489). Herein, if reference is made to percent sequence identity, the higher percentages of sequence identity are preferred over the lower ones.

The present disclosure also provides compositions comprising any one or more of the isolated nucleic acid molecules, genomic nucleic acid molecules, mRNA molecules, and/or cDNA molecules disclosed herein. In some embodiments, the composition is a pharmaceutical composition. In some embodiments, the compositions comprise a carrier and/or excipient. Examples of carriers include, but are not limited to, poly(lactic acid) (PLA) microspheres, poly(D,L-lactic-coglycolic-acid) (PLGA) microspheres, liposomes, micelles, inverse micelles, lipid cochleates, and lipid microtubules. A carrier may comprise a buffered salt solution such as PBS, HBSS, etc.

As used herein, the phrase “corresponding to” or grammatical variations thereof when used in the context of the numbering of a particular nucleotide or nucleotide sequence or position refers to the numbering of a specified reference sequence when the particular nucleotide or nucleotide sequence is compared to a reference sequence (such as, for example, SEQ ID NO:1, SEQ ID NO:3, or SEQ ID NO:31). In other words, the residue (such as, for example, nucleotide or amino acid) number or residue (such as, for example, nucleotide or amino acid) position of a particular polymer is designated with respect to the reference sequence rather than by the actual numerical position of the residue within the particular nucleotide or nucleotide sequence. For example, a particular nucleotide sequence can be aligned to a reference sequence by introducing gaps to optimize residue matches between the two sequences. In these cases, although the gaps are present, the numbering of the residue in the particular nucleotide or nucleotide sequence is made with respect to the reference sequence to which it has been aligned.

For example, a CREB3L3 nucleic acid molecule comprising a nucleotide sequence encoding a CREB3L3 predicted loss-of-function polypeptide, wherein the nucleotide sequence comprises an adenine at a position corresponding to position 6,120 according to SEQ ID NO:2 means that if the nucleotide sequence of the CREB3L3 genomic nucleic acid molecule is aligned to the sequence of SEQ ID NO:2, the CREB3L3 sequence has an adenine residue at the position that corresponds to position 6,120 of SEQ ID NO:2. The same applies for a CREB3L3 mRNA molecules comprising a nucleotide sequence encoding a CREB3L3 predicted loss-of-function polypeptide, wherein the nucleotide sequence comprises an adenine at a position corresponding to position 661 according to SEQ ID NO:17, and a CREB3L3 cDNA molecules comprising a nucleotide sequence encoding a CREB3L3 predicted loss-of-function polypeptide, wherein the nucleotide sequence comprises an adenine at a position corresponding to position 661 according to SEQ ID NO:45. In other words, these phrases refer to a nucleic acid molecule encoding a CREB3L3 polypeptide, wherein the genomic nucleic acid molecule has a nucleotide sequence that comprises an adenine residue that is homologous to the adenine residue at position 6,120 of SEQ ID NO:2 (or wherein the mRNA molecule has a nucleotide sequence that comprises an adenine residue that is homologous to the adenine residue at position 661 of SEQ ID NO:17, or wherein the cDNA molecule has a nucleotide sequence that comprises an adenine residue that is homologous to the adenine residue at position 661 of SEQ ID NO:45).

As described herein, a position within a CREB3L3 genomic nucleic acid molecule that corresponds to position 6,120 according to SEQ ID NO:2, for example, can be identified by performing a sequence alignment between the nucleotide sequence of a particular CREB3L3 nucleic acid molecule and the nucleotide sequence of SEQ ID NO:2. A variety of computational algorithms exist that can be used for performing a sequence alignment to identify a nucleotide position that corresponds to, for example, position 6,120 in SEQ ID NO:2. For example, by using the NCBI BLAST algorithm (Altschul et al., Nucleic Acids Res., 1997, 25, 3389-3402) or CLUSTALW software (Sievers and Higgins, Methods Mol. Biol., 2014, 1079, 105-116) sequence alignments may be performed. However, sequences can also be aligned manually.

The amino acid sequences of CREB3L3 reference polypeptides are set forth in SEQ ID NO:59 (Isoform 1), SEQ ID NO:60 (Isoform 2), SEQ ID NO:61 (Isoform 3), SEQ ID NO:62 (Isoform 4), and SEQ ID NO:63 (Isoform 5).

Referring to SEQ ID NO:59 (Isoform 1), the CREB3L3 reference polypeptide is 467 amino acids in length. Referring to SEQ ID NO:59, position 182 is an aspartic acid.

Referring to SEQ ID NO:60 (Isoform 2), the CREB3L3 reference polypeptide is 459 amino acids in length. Referring to SEQ ID NO:60, position 182 is an aspartic acid.

Referring to SEQ ID NO:61 (Isoform 3), the CREB3L3 reference polypeptide is 337 amino acids in length. Referring to SEQ ID NO:61, position 182 is an aspartic acid.

Referring to SEQ ID NO:62 (Isoform 4), the CREB3L3 reference polypeptide is 473 amino acids in length. Referring to SEQ ID NO:62, position 182 is an aspartic acid.

Referring to SEQ ID NO:63 (Isoform 5), the CREB3L3 reference polypeptide is 473 amino acids in length. Referring to SEQ ID NO:63, position 181 is an aspartic acid.

The amino acid sequences of CREB3L3 predicted loss-of-function polypeptides are set forth in SEQ ID NO:64 (Isoform 1), SEQ ID NO:65 (Isoform 2), SEQ ID NO:66 (Isoform 3), SEQ ID NO:67 (Isoform 4), and SEQ ID NO:68 (Isoform 5). Referring to SEQ ID NO:64, (Asp182Asn-A; Isoform 1), position 182 is an asparagine. Referring to SEQ ID NO:65, (Asp182Asn-B; Isoform 2), position 182 is an asparagine. Referring to SEQ ID NO:66, (Asp182Asn-C; Isoform 3), position 182 is an asparagine. Referring to SEQ ID NO:67, (Asp182Asn-D; Isoform 4), position 182 is an asparagine. Referring to SEQ ID NO:68, (Asp181Asn; Isoform 5), position 181 is an asparagine.

The nucleotide and amino acid sequences listed in the accompanying sequence listing are shown using standard letter abbreviations for nucleotide bases, and three-letter code for amino acids. The nucleotide sequences follow the standard convention of beginning at the 5′ end of the sequence and proceeding forward (i.e., from left to right in each line) to the 3′ end. Only one strand of each nucleotide sequence is shown, but the complementary strand is understood to be included by any reference to the displayed strand. The amino acid sequence follows the standard convention of beginning at the amino terminus of the sequence and proceeding forward (i.e., from left to right in each line) to the carboxy terminus.

The present disclosure also provides therapeutic agents that treat or inhibit a liver disease for use in the treatment of a liver disease (or for use in the preparation of a medicament for treating a liver disease) in a subject, wherein the subject has any of the CREB3L3 variant genomic nucleic acid molecules, variant mRNA molecules, and/or variant cDNA molecules encoding a CREB3L3 predicted loss-of-function polypeptide described herein. The therapeutic agents that treat or inhibit a liver disease can be any of the therapeutic agents that treat or inhibit a liver disease described herein.

In some embodiments, the subject is identified as having a genomic nucleic acid molecule encoding a CREB3L3 predicted loss-of-function polypeptide, wherein the genomic nucleic acid molecule has a nucleotide sequence comprising an adenine at a position corresponding to position 6,120 according to SEQ ID NO:2, or the complement thereof.

In some embodiments, the subject is identified as having an mRNA molecule encoding a CREB3L3 predicted loss-of-function polypeptide, wherein the mRNA molecule has a nucleotide sequence comprising an adenine at a position corresponding to: position 661 according to SEQ ID NO:17, or the complement thereof; position 649 according to SEQ ID NO:18, or the complement thereof; position 624 according to SEQ ID NO:19, or the complement thereof; position 624 according to SEQ ID NO:20, or the complement thereof; position 658 according to SEQ ID NO:21, or the complement thereof; position 661 according to SEQ ID NO:22, or the complement thereof; position 661 according to SEQ ID NO:23, or the complement thereof; position 663 according to SEQ ID NO:24, or the complement thereof; position 660 according to SEQ ID NO:25, or the complement thereof; position 649 according to SEQ ID NO:26, or the complement thereof; position 624 according to SEQ ID NO:27, or the complement thereof; or position 691 according to SEQ ID NO:28, or the complement thereof.

In some embodiments, the subject is identified as having a cDNA molecule encoding a CREB3L3 predicted loss-of-function polypeptide, wherein the cDNA molecule has a nucleotide sequence comprising an adenine at a position corresponding to: position 661 according to SEQ ID NO:45, or the complement thereof; position 649 according to SEQ ID NO:46, or the complement thereof; position 624 according to SEQ ID NO:47, or the complement thereof; position 624 according to SEQ ID NO:48, or the complement thereof; position 658 according to SEQ ID NO:49, or the complement thereof; position 661 according to SEQ ID NO:50, or the complement thereof; position 661 according to SEQ ID NO:51, or the complement thereof; position 663 according to SEQ ID NO:52, or the complement thereof; position 660 according to SEQ ID NO:53, or the complement thereof; position 649 according to SEQ ID NO:54, or the complement thereof; position 624 according to SEQ ID NO:55, or the complement thereof; or position 691 according to SEQ ID NO:56, or the complement thereof.

In some embodiments, the subject is identified as having: i) a genomic nucleic acid molecule having a nucleotide sequence encoding a CREB3L3 predicted loss-of-function polypeptide, wherein the nucleotide sequence comprises an adenine at a position corresponding to position 6,120 according to SEQ ID NO:2, or the complement thereof; ii) an mRNA molecule having a nucleotide sequence encoding a CREB3L3 predicted loss-of-function polypeptide, wherein the nucleotide sequence comprises an adenine at a position corresponding to: position 661 according to SEQ ID NO:17, or the complement thereof; position 649 according to SEQ ID NO:18, or the complement thereof; position 624 according to SEQ ID NO:19, or the complement thereof; position 624 according to SEQ ID NO:20, or the complement thereof; position 658 according to SEQ ID NO:21, or the complement thereof; position 661 according to SEQ ID NO:22, or the complement thereof; position 661 according to SEQ ID NO:23, or the complement thereof; position 663 according to SEQ ID NO:24, or the complement thereof; position 660 according to SEQ ID NO:25, or the complement thereof; position 649 according to SEQ ID NO:26, or the complement thereof; position 624 according to SEQ ID NO:27, or the complement thereof; or position 691 according to SEQ ID NO:28, or the complement thereof; or iii) a cDNA molecule having a nucleotide sequence encoding a CREB3L3 predicted loss-of-function polypeptide, wherein the nucleotide sequence comprises an adenine at a position corresponding to: position 661 according to SEQ ID NO:45, or the complement thereof; position 649 according to SEQ ID NO:46, or the complement thereof; position 624 according to SEQ ID NO:47, or the complement thereof; position 624 according to SEQ ID NO:48, or the complement thereof; position 658 according to SEQ ID NO:49, or the complement thereof; position 661 according to SEQ ID NO:50, or the complement thereof; position 661 according to SEQ ID NO:51, or the complement thereof; position 663 according to SEQ ID NO:52, or the complement thereof; position 660 according to SEQ ID NO:53, or the complement thereof; position 649 according to SEQ ID NO:54, or the complement thereof; position 624 according to SEQ ID NO:55, or the complement thereof; or position 691 according to SEQ ID NO:56, or the complement thereof.

In some embodiments, the subject is identified as having a genomic nucleic acid molecule having a nucleotide sequence encoding a CREB3L3 predicted loss-of-function polypeptide, wherein the nucleotide sequence comprises an adenine at a position corresponding to position 6,120 according to SEQ ID NO:2, or the complement thereof.

In some embodiments, the subject is identified as having an mRNA molecule having a nucleotide sequence encoding a CREB3L3 predicted loss-of-function polypeptide, wherein the nucleotide sequence comprises an adenine at a position corresponding to: position 661 according to SEQ ID NO:17, or the complement thereof; position 649 according to SEQ ID NO:18, or the complement thereof; position 624 according to SEQ ID NO:19, or the complement thereof; position 624 according to SEQ ID NO:20, or the complement thereof; position 658 according to SEQ ID NO:21, or the complement thereof; position 661 according to SEQ ID NO:22, or the complement thereof; position 661 according to SEQ ID NO:23, or the complement thereof; position 663 according to SEQ ID NO:24, or the complement thereof; position 660 according to SEQ ID NO:25, or the complement thereof; position 649 according to SEQ ID NO:26, or the complement thereof; position 624 according to SEQ ID NO:27, or the complement thereof; or position 691 according to SEQ ID NO:28, or the complement thereof.

In some embodiments, the subject is identified as having a cDNA molecule having a nucleotide sequence encoding a CREB3L3 predicted loss-of-function polypeptide, wherein the nucleotide sequence comprises an adenine at a position corresponding to: position 661 according to SEQ ID NO:45, or the complement thereof; position 649 according to SEQ ID NO:46, or the complement thereof; position 624 according to SEQ ID NO:47, or the complement thereof; position 624 according to SEQ ID NO:48, or the complement thereof; position 658 according to SEQ ID NO:49, or the complement thereof; position 661 according to SEQ ID NO:50, or the complement thereof; position 661 according to SEQ ID NO:51, or the complement thereof; position 663 according to SEQ ID NO:52, or the complement thereof; position 660 according to SEQ ID NO:53, or the complement thereof; position 649 according to SEQ ID NO:54, or the complement thereof; position 624 according to SEQ ID NO:55, or the complement thereof; or position 691 according to SEQ ID NO:56, or the complement thereof.

In some embodiments, the subject is identified as having a CREB3L3 predicted loss-of-function polypeptide that comprises an asparagine at a position corresponding to: position 182 according to SEQ ID NO:64, position 182 according to SEQ ID NO:65, position 182 according to SEQ ID NO:66, position 182 according to SEQ ID NO:67, or position 181 according to SEQ ID NO:68.

The present disclosure also provides CREB3L3 inhibitors for use in the treatment of a liver disease (or for use in the preparation of a medicament for treating a liver disease) in a subject, wherein the subject is heterozygous for any of the CREB3L3 variant genomic nucleic acid molecules, variant mRNA molecules, and/or variant cDNA molecules encoding a CREB3L3 predicted loss-of-function polypeptide described herein, or wherein the subject is reference for a CREB3L3 genomic nucleic acid molecule, mRNA molecule, or cDNA molecule. The CREB3L3 inhibitors can be any of the CREB3L3 inhibitors described herein.

In some embodiments, the subject is reference for a CREB3L3 genomic nucleic acid molecule, a CREB3L3 mRNA molecule, or a CREB3L3 cDNA molecule. In some embodiments, the subject is reference for a CREB3L3 genomic nucleic acid molecule. In some embodiments, the subject is reference for a CREB3L3 mRNA molecule. In some embodiments, the subject is reference for a CREB3L3 cDNA molecule.

In some embodiments, the subject is identified as being heterozygous for a genomic nucleic acid molecule encoding a CREB3L3 predicted loss-of-function polypeptide, wherein the genomic nucleic acid molecule has a nucleotide sequence comprising an adenine at a position corresponding to position 6,120 according to SEQ ID NO:2, or the complement thereof.

In some embodiments, the subject is identified as being heterozygous for an mRNA molecule encoding a CREB3L3 predicted loss-of-function polypeptide, wherein the mRNA molecule has a nucleotide sequence comprising an adenine at a position corresponding to: position 661 according to SEQ ID NO:17, or the complement thereof; position 649 according to SEQ ID NO:18, or the complement thereof; position 624 according to SEQ ID NO:19, or the complement thereof; position 624 according to SEQ ID NO:20, or the complement thereof; position 658 according to SEQ ID NO:21, or the complement thereof; position 661 according to SEQ ID NO:22, or the complement thereof; position 661 according to SEQ ID NO:23, or the complement thereof; position 663 according to SEQ ID NO:24, or the complement thereof; position 660 according to SEQ ID NO:25, or the complement thereof; position 649 according to SEQ ID NO:26, or the complement thereof; position 624 according to SEQ ID NO:27, or the complement thereof; or position 691 according to SEQ ID NO:28, or the complement thereof.

In some embodiments, the subject is identified as being heterozygous for a cDNA molecule encoding a CREB3L3 predicted loss-of-function polypeptide, wherein the cDNA molecule has a nucleotide sequence comprising an adenine at a position corresponding to: position 661 according to SEQ ID NO:45, or the complement thereof; position 649 according to SEQ ID NO:46, or the complement thereof; position 624 according to SEQ ID NO:47, or the complement thereof; position 624 according to SEQ ID NO:48, or the complement thereof; position 658 according to SEQ ID NO:49, or the complement thereof; position 661 according to SEQ ID NO:50, or the complement thereof; position 661 according to SEQ ID NO:51, or the complement thereof; position 663 according to SEQ ID NO:52, or the complement thereof; position 660 according to SEQ ID NO:53, or the complement thereof; position 649 according to SEQ ID NO:54, or the complement thereof; position 624 according to SEQ ID NO:55, or the complement thereof; or position 691 according to SEQ ID NO:56, or the complement thereof.

In some embodiments, the subject is identified as being heterozygous for: i) a genomic nucleic acid molecule having a nucleotide sequence encoding a CREB3L3 predicted loss-of-function polypeptide, wherein the nucleotide sequence comprises an adenine at a position corresponding to position 6,120 according to SEQ ID NO:2, or the complement thereof; ii) an mRNA molecule having a nucleotide sequence encoding a CREB3L3 predicted loss-of-function polypeptide, wherein the nucleotide sequence comprises an adenine at a position corresponding to: position 661 according to SEQ ID NO:17, or the complement thereof; position 649 according to SEQ ID NO:18, or the complement thereof; position 624 according to SEQ ID NO:19, or the complement thereof; position 624 according to SEQ ID NO:20, or the complement thereof; position 658 according to SEQ ID NO:21, or the complement thereof; position 661 according to SEQ ID NO:22, or the complement thereof; position 661 according to SEQ ID NO:23, or the complement thereof; position 663 according to SEQ ID NO:24, or the complement thereof; position 660 according to SEQ ID NO:25, or the complement thereof; position 649 according to SEQ ID NO:26, or the complement thereof; position 624 according to SEQ ID NO:27, or the complement thereof; or position 691 according to SEQ ID NO:28, or the complement thereof; or iii) a cDNA molecule having a nucleotide sequence encoding a CREB3L3 predicted loss-of-function polypeptide, wherein the nucleotide sequence comprises an adenine at a position corresponding to: position 661 according to SEQ ID NO:45, or the complement thereof; position 649 according to SEQ ID NO:46, or the complement thereof; position 624 according to SEQ ID NO:47, or the complement thereof; position 624 according to SEQ ID NO:48, or the complement thereof; position 658 according to SEQ ID NO:49, or the complement thereof; position 661 according to SEQ ID NO:50, or the complement thereof; position 661 according to SEQ ID NO:51, or the complement thereof; position 663 according to SEQ ID NO:52, or the complement thereof; position 660 according to SEQ ID NO:53, or the complement thereof; position 649 according to SEQ ID NO:54, or the complement thereof; position 624 according to SEQ ID NO:55, or the complement thereof; or position 691 according to SEQ ID NO:56, or the complement thereof.

In some embodiments, the subject is identified as being heterozygous for a genomic nucleic acid molecule having a nucleotide sequence encoding a CREB3L3 predicted loss-of-function polypeptide, wherein the nucleotide sequence comprises an adenine at a position corresponding to position 6,120 according to SEQ ID NO:2, or the complement thereof.

In some embodiments, the subject is identified as being heterozygous for an mRNA molecule having a nucleotide sequence encoding a CREB3L3 predicted loss-of-function polypeptide, wherein the nucleotide sequence comprises an adenine at a position corresponding to: position 661 according to SEQ ID NO:17, or the complement thereof; position 649 according to SEQ ID NO:18, or the complement thereof; position 624 according to SEQ ID NO:19, or the complement thereof; position 624 according to SEQ ID NO:20, or the complement thereof; position 658 according to SEQ ID NO:21, or the complement thereof; position 661 according to SEQ ID NO:22, or the complement thereof; position 661 according to SEQ ID NO:23, or the complement thereof; position 663 according to SEQ ID NO:24, or the complement thereof; position 660 according to SEQ ID NO:25, or the complement thereof; position 649 according to SEQ ID NO:26, or the complement thereof; position 624 according to SEQ ID NO:27, or the complement thereof; or position 691 according to SEQ ID NO:28, or the complement thereof.

In some embodiments, the subject is identified as being heterozygous for a cDNA molecule having a nucleotide sequence encoding a CREB3L3 predicted loss-of-function polypeptide, wherein the nucleotide sequence comprises an adenine at a position corresponding to: position 661 according to SEQ ID NO:45, or the complement thereof; position 649 according to SEQ ID NO:46, or the complement thereof; position 624 according to SEQ ID NO:47, or the complement thereof; position 624 according to SEQ ID NO:48, or the complement thereof; position 658 according to SEQ ID NO:49, or the complement thereof; position 661 according to SEQ ID NO:50, or the complement thereof; position 661 according to SEQ ID NO:51, or the complement thereof; position 663 according to SEQ ID NO:52, or the complement thereof; position 660 according to SEQ ID NO:53, or the complement thereof; position 649 according to SEQ ID NO:54, or the complement thereof; position 624 according to SEQ ID NO:55, or the complement thereof; or position 691 according to SEQ ID NO:56, or the complement thereof.

All patent documents, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the present disclosure can be used in combination with any other feature, step, element, embodiment, or aspect unless specifically indicated otherwise. Although the present disclosure has been described in some detail by way of illustration and example for purposes of clarity and understanding, it will be apparent that certain changes and modifications may be practiced within the scope of the appended claims.

The following examples are provided to describe the embodiments in greater detail. They are intended to illustrate, not to limit, the claimed embodiments. The following examples provide those of ordinary skill in the art with a disclosure and description of how the compounds, compositions, articles, devices and/or methods described herein are made and evaluated, and are intended to be purely exemplary and are not intended to limit the scope of any claims. Efforts have been made to ensure accuracy with respect to numbers (such as, for example, amounts, temperature, etc.), but some errors and deviations may be accounted for. Unless indicated otherwise, parts are parts by weight, temperature is in °C or is at ambient temperature, and pressure is at or near atmospheric.

### Examples

### Example 1: Loss-of-Function of CREB3L3 is Associated with Lower Liver Damage as Measured by Circulating Alanine Transferase Levels and Protection Against Liver Disease in Humans

To identify genetic factors contributing to predisposition for or protection against chronic liver disease, exome sequencing was performed in 567,237 participants of European ancestry from the UK Biobank cohort (UKB) and Geisinger Health System DiscovEHR study (GHS). For each rare genetic missense or pLOF variant in the genome, associations with alanine aminotransferase (ALT), a widely used biomarker of liver damage, were estimated for the burden of rare loss-of-function and missense variants identified by exome sequencing were estimated for each gene in the genome. Statistically significant findings were subsequently evaluated for their association with clinical diagnoses of chronic liver disease in UKB, GHS and the SINAI cohorts.

The exome-wide analysis identified rs140312652, a missense genetic variant encoding for a change in amino acid of CREB3L3 (Asp182Asn) to be strongly associated with lower ALT (-1.95 U/L, P=7.9x10⁻¹²) at the exome-wide level of statistical significance (p<5x10⁻⁸, Table 3).

TABLE 3

Asp182Asn in CREB3L3 is associated with lower liver damage as measured by circulating alanine transferase levels. The results are from an inverse variance weighted meta-analysis in the GHS and UKB cohorts

| Per allele beta (95% CI) in SD units of ALT | Per allele beta (95% CI) in U/L of ALT | p-value | Genotype counts, RR \| RA \| AA genotypes | AAF, fraction of 1 |
|---|---|---|---|---|
| -0.14 (-0.18,-0.10) | -1.95 (-2.51,-1.39) | 7.9x10⁻¹² | 538,682\|2,074\|6 | 0.00193 |

RR indicates the number of individuals carrying the alternate allele A of rs140312652 (19:4159750:G:A, human genome build 38) in CREB3L3 (homozygous non-carriers); RA indicates the number of individuals carrying rare missense or pLOF variants in a single CREB3L3 allele (heterozygous carriers); AA indicates the number of individuals carrying rare missense or pLOF variants in both CREB3L3alleles (homozygous carriers); AAF indicates the alternative allele frequency of pLOF or missense alleles in CREB3L3 included in the analysis; pLOF indicates predicted loss of function; U/L indicates units per liter; SD indicates standard deviation; CI indicates confidence interval.

The association of rare (alternative allele frequency < 1%) loss of function variants in pLOF was also associated with lower ALT (Table 4). Indicating that loss of CREB3L3 function is associated with lower liver damage as measured by ALT.

TABLE 4

Rare predicted loss-of-function variants in CREB3L3 are associated with lower liver damage as measured by circulating alanine transferase levels

| Per allele beta (95% CI) in SD units of ALT | Per allele beta (95% CI) In U/L of ALT | p-value | Genotype counts, RR \| RA \| AA genotypes | AAF, fraction of 1 |
|---|---|---|---|---|
| -0.05 (-0.09,-0.01) | -0.69 (-1.18,-0.20) | 0.0055 | 540,18112,72112 | 0.00251 |

RR indicates the number of individuals carrying no rare pLOF variants in CREB3L3 (homozygous non-carriers); RA indicates the number of individuals carrying rare pLOF variants in a single CREB3L3 allele (heterozygous carriers); AA indicates the number of individuals carrying rare pLOF variants in both CREB3L3 alleles (homozygous carriers); AAF indicates the alternative allele frequency of pLOF alleles in CREB3L3 included in the analysis; pLOF indicates predicted loss of function; U/L indicates units per liter; SD indicates standard deviation; CI indicates confidence interval.

In this analysis forTable 4, the burden of pLOF CREB3L3 variants with an AAF below 1% was the exposure variable, while ALT levels were the outcome variable. The results are from an inverse variance weighted meta-analysis in the GHS and UKB cohorts

The association of Asp182Asn in CREB3L3 with liver disease outcomes was estimated next. Asp182Asn in CREB3L3 was associated with protection against parenchymal liver disease and non-alcoholic liver disease (Table 5). Heterozygous carriers of these genetic variants had 25% lower odds of liver disease compared to non-carriers.

TABLE 5

Asp182Asn in CREB3L3 is associated with protection against liver disease across etiologies

| Outcome | Per allele odds ratio (95% CI) | p-value | Genotype counts, RR \| RA \| AA genotypes |
|---|---|---|---|
| Parenchymal liver disease | 0.76 (0.58, 0.99) | 0.039 | Cases:17,673 \|47\| 0 Controls: 444,219 \| 1,810 \| 5 |
| Non-alcoholic liver disease | 0.75 (0.56, 0.99) | 0.043 | Cases: 15,548\|39\|0 Controls: 450,543 \| 1,731 \| 5 |

RR indicates the number of individuals homozygous for the reference genotype (homozygous non-carriers); RA indicates the number of individuals heterozygous for the genetic exposure genotype (heterozygous carriers); AA indicates the number of individuals homozygous for the genetic exposure genotype (homozygous carriers); AAF indicates the alternative allele frequency of the genetic exposure genotype; CI indicates confidence interval.

In this analysis for Table 5, Asp182Asn in CREB3L3 was the exposure variable, while the liver disease were the outcome variables. The results are from an inverse variance weighted meta-analysis in the GHS, UKB, SINAI and UPENN-PMBB cohorts.

### Participating Cohorts

Genetic association studies were performed in the United Kingdom Biobank (UKB) cohort (Sudlow et al., PLoS Med, 2015, 12, e1001779) and the DiscoverEHR cohort from the Geisinger Health System (GHS) MyCode Community Health Initiative (Carey et al., Genet. Med., 2016, 18, 906-13). UKB is a population-based cohort study of people aged between 40 and 69 years recruited through 22 testing centers in the UK between 2006-2010. Over 430,000 European ancestry participants from UKB with available whole-exome sequencing and clinical phenotype data were included. The GHS MyCode study Community Health Initiative is a health system-based cohort of patients from Central and Eastern Pennsylvania (USA) recruited in 2007-2019. Over 130,000 European ancestry participants from GHS with available whole-exome sequencing and clinical phenotype data were included. The associations with liver outcomes also included the Mount Sinai BioMe Biobank cohort (SINAI, Cell, 2019, 177, 58-69), The University of Pennsylvania Penn Medicine BioBank (UPENN-PMBB; Park et al., 2020, doi:10.1038/s41436-019-0625-8).

### Phenotype Definitions

Clinical laboratory measurements for ALT and other biomarkers were extracted from electronic health records (EHRs) of participants from GHS. Median values were calculated for all participants with two or more measurements. In UKB, ALT and other biomarkers were measured by IFCC (International Federation of Clinical Chemistry) analysis on a Beckman Coulter AU5800 at the baseline visit of the study; Prior to genetic association analysis, continues phenotype values were transformed by the inverse standard normal function, applied within each ancestry group and separately in men and women.

Disease outcomes were defined according to the International Classification of Diseases Tenth Revision (ICD-10) and read codes stored in EHRs. Self-reported disease status was used when available. Office of Population Censuses and Surveys Classification of Interventions and Procedures version 4 (OPCS4) codes were used for medical procedures. Individuals with liver diseases were identified as described in Table 6, combining EHR records, self-reports, and ALT measurements.

TABLE 6

Definitions of liver disease and coronary disease outcomes in UKB, GHS, and SINAI cohort

| | | |
|---|---|---|
| Liver disease outcome | Case definition | Controls definition |
| Parenchymal liver disease | ICD10: K70,K71,K72,K73,K74,K753,K75 3,K752,K754,K758, K759,K760,K767,K7681 OPCS4: G10,G144,J01 UKB.f.20002: 1604,1158,1141 | See footnote* |
| Non-alcoholic liver disease | ICD10: K721,K740,K741,K742,K746,K75 8,K760 | See footnote* |

* Participants were excluded from the control population if they were diagnosed with the
“any liver disease” outcome codes (as defined in the table) or if they had elevated ALT >33 U/L for men and >25 U/L for women.

ICD10 indicates the 10th revision of the International Statistical Classification of Diseases and Related Health Problems; UKB.OPCS4 indicates Office of Population Censuses and Surveys (OPCS) Classification of Interventions and Procedures version 4 as used in the UK Biobank (UKB); UKB.f.20002 indicates self-reported non-cancer illness codes as used in UKB. UKB.f.20004 indicates self-reported medical procedures as used in UKB.

### Genotype Data

High coverage whole exome sequencing was performed as previously described (Science, 2016, 354:aaf6814.; and Nature, 2020, 586:749-756) and as summarized below. NimbleGen probes (VCRome; for part of the GHS cohort) or a modified version of the xGen design available from Integrated DNA Technologies (IDT; for the rest of GHS and other cohorts) were used for target sequence capture of the exome. A unique 6 base pair (bp) barcode (VCRome) or 10 bp barcode (IDT) was added to each DNA fragment during library preparation to facilitate multiplexed exome capture and sequencing. Equal amounts of sample were pooled prior to exome capture. Sequencing was performed using 75 bp paired-end reads on Illumina v4 HiSeq 2500 (for part of the GHS cohort) or NovaSeq (for the rest of GHS and other cohorts) instruments. Sequencing had a coverage depth (i.e., number of sequence-reads covering each nucleotide in the target areas of the genome) sufficient to provide greater than 20x coverage over 85% of targeted bases in 96% of VCRome samples and 20x coverage over 90% of targeted bases in 99% of IDT samples. Data processing steps included sample de-multiplexing using Illumina software, alignment to the GRCh38 Human Genome reference sequence including generation of binary alignment and mapping files (BAM), processing of BAM files (e.g., marking of duplicate reads and other read mapping evaluations). Variant calling was performed using the GLNexus system (DOI: 10.1101/343970). Variant mapping and annotation were based on the GRCh38 Human Genome reference sequence and Ensembl v85 gene definitions using the snpEff software. The snpEff predictions that involve protein-coding transcripts with an annotated start and stop were then combined into a single functional impact prediction by selecting the most deleterious functional effect class for each gene. The hierarchy (from most to least deleterious) for these annotations was frameshift, stop-gain, stop-loss, splice acceptor, splice donor, stop-lost, in-frame indel, missense, other annotations. Predicted LOF genetic variants included: a) insertions or deletions resulting in a frameshift, b) insertions, deletions or single nucleotide variants resulting in the introduction of a premature stop codon or in the loss of the transcription start site or stop site, and c) variants in donor or acceptor splice sites. Missense variants were classified for likely functional impact according to the number of in silico prediction algorithms that predicted deleteriousness using SIFT (Adzhubei et al., Nat. Methods, 2010, 7, 248-9) and Polyphen2_HVAR (Adzhubei et al., Nat. Methods, 2010, 7, 248-9), LRT (Chun et al., Genome Res., 2009, 19, 1553-61) and MutationTaster (Schwarz et al., Nat. Methods, 2010, 7, 575-6). For each gene, the alternative allele frequency (AAF) and functional annotation of each variant determined inclusion into these 7 gene burden exposures: 1) pLOF variants with AAF < 1%; 2) pLOF or missense variants predicted deleterious by 5/5 algorithms with AAF < 1%; 3) pLOF or missense variants predicted deleterious by 5/5 algorithms with AAF < 0.1%; 4) pLOF or missense variants predicted deleterious by at least 1/5 algorithms with AAF < 1%; 5) pLOF or missense variants predicted deleterious by at least 1/5 algorithms with AAF < 0.1%; 6) pLOF or any missense with AAF < 1%; 7) pLOF or any missense variants with AAF < 0.1%.

### Association Analysis of Gene Burden of Rare pLOF and Missense Variation in

Association between the burden of rare predicted loss-of-function or missense variants in a given gene and phenotype was tested by fitting a linear (for quantitative traits) or firth bias-corrected logistic (for binary traits) regression model adjusted for a polygenic score that approximates a genomic kinship matrix using REGENIE v1.0 (see, world wide web at “doi.org/10.1101/2020.06.19.162354”). Analyses were stratified by ancestry and adjusted for age, age², sex, age-by-sex and age²-by-sex interaction terms, experimental batch-related covariates, 10 common variant-derived principal components, and 20 rare variant-derived principal components. Results across cohorts for each variant-phenotype association were combined using fixed effects inverse variance weighted meta-analysis. In gene burden tests, all individuals are labeled as heterozygotes if they carry one or more qualifying rare variant (as described above based on frequency and functional annotation) and as homozygotes if they carry any qualifying variant in the homozygous state. This “composite genotype” is then used to test for association. Linear interaction models were fitted with the same analytical approach.

Various modifications of the described subject matter, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety and for all purposes.

1. A method of treating a subject having a liver disease, parenchymal liver disease, liver fibrosis, liver cirrhosis, and/or non-alcoholic fatty liver disease (NAFLD), the method comprising administering a CAMP Responsive Element Binding Protein 3 Like 3 (CREB3L3) inhibitor to the subject.

2-5. (canceled)

6. The method according to claim 1, wherein the CREB3L3 inhibitor comprises an inhibitory nucleic acid molecule.

7. The method according to claim 6, wherein the inhibitory nucleic acid molecule comprises an antisense nucleic acid molecule, a small interfering RNA (siRNA), or a short hairpin RNA (shRNA) that hybridizes to a CREB3L3 nucleic acid molecule.

8-14. (canceled)

15. The method according to claim 1, further comprising detecting the presence or absence of a CREB3L3 variant nucleic acid molecule encoding a CREB3L3 predicted loss-of-function polypeptide in a biological sample obtained from the subject.

16. The method according to claim 15, further comprising administering a therapeutic agent that treats or inhibits a liver disease in a standard dosage amount to a subject wherein the CREB3L3 variant nucleic acid molecule is absent from the biological sample.

17. The method according to claim 15, further comprising administering a therapeutic agent that treats or inhibits a liver disease in a dosage amount that is the same as or less than a standard dosage amount to a subject that is heterozygous for the CREB3L3 variant nucleic acid molecule.

18. The method according to claim 15, wherein the CREB3L3 variant nucleic acid molecule encodes Asp182Asn-A, Asp182Asn-B, Asp182Asn-C, Asp182Asn-D, or Asp181Asn.

19. The method according to claim 15, wherein the CREB3L3 variant nucleic acid molecule encodes Asp182Asn-A, Asp182Asn-B, Asp182Asn-C, or Asp182Asn-D.

20. The method according to claim 18, wherein the CREB3L3 variant nucleic acid molecule is:
a genomic nucleic acid molecule having a nucleotide sequence comprising an adenine at a position corresponding to position 6,120 according to SEQ ID NO:2;
an mRNA molecule having a nucleotide sequence comprising an adenine at a position corresponding to: position 661 according to SEQ ID NO: 17, position 649 according to SEQ ID NO: 18, position 624 according to SEQ ID NO:19, 624 according to SEQ ID NO:20, position 658 according to SEQ ID NO:21, position 661 according to SEQ ID NO:22, position 661 according to SEQ ID NO:23, position 663 according to SEQ ID NO:24, position 660 according to SEQ ID NO:25, position 649 according to SEQ ID NO:26, position 624 according to SEQ ID NO:27, or position 691 according to SEQ ID NO:28; or
a cDNA molecule having a nucleotide sequence comprising an adenine at a position corresponding to: position 661 according to SEQ ID NO:45, position 649 according to SEQ ID NO:46, position 624 according to SEQ ID NO:47, position 624 according to SEQ ID NO:48, position 658 according to SEQ ID NO:49, position 661 according to SEQ ID NO:50, position 661 according to SEQ ID NO:51, position 663 according to SEQ ID NO:52, position 660 according to SEQ ID NO:53, position 649 according to SEQ ID NO:54, position 624 according to SEQ ID NO:55, or position 691 according to SEQ ID NO:56.

21-35. (canceled)

36. A method of treating a subject with a therapeutic agent that treats or inhibits a liver disease, wherein the subject has a liver disease, the method comprising:
determining whether the subject has a CAMP Responsive Element Binding Protein 3 Like 3 (CREB3L3) variant nucleic acid molecule encoding a CREB3L3 predicted loss-of-function polypeptide by:
obtaining or having obtained a biological sample from the subject; and
performing or having performed a sequence analysis on the biological sample to determine if the subject has a genotype comprising the CREB3L3 variant nucleic acid molecule encoding the CREB3L3 predicted loss-of-function polypeptide; and
administering or continuing to administer the therapeutic agent that treats or inhibits the liver disease in a standard dosage amount to a subject that is CREB3L3 reference, and administering a CREB3L3 inhibitor to the subject; and
administering or continuing to administer the therapeutic agent that treats or inhibits the liver disease in an amount that is the same as or less than a standard dosage amount to a subject that is heterozygous for the CREB3L3 variant nucleic acid molecule, and administering a CREB3L3 inhibitor to the subject;
wherein the presence of a genotype having the CREB3L3 variant nucleic acid molecule encoding the CREB3L3 predicted loss-of-function polypeptide indicates the subject has a reduced risk of developing the liver disease.

37. The method according to claim 36, wherein the subject is CREB3L3 reference, and the subject is administered or continued to be administered the therapeutic agent that treats or inhibits the liver disease in a standard dosage amount, and is administered a CREB3L3 inhibitor.

38. The method according to claim 36, wherein the subject is heterozygous for a CREB3L3 variant nucleic acid molecule, and the subject is administered or continued to be administered the therapeutic agent that treats or inhibits the liver disease in an amount that is the same as or less than a standard dosage amount, and is administered a CREB3L3 inhibitor.

39. The method according to claim 36, wherein the CREB3L3 variant nucleic acid molecule encodes Asp182Asn-A, Asp182Asn-B, Asp182Asn-C, Asp182Asn-D, or Asp181Asn.

40. The method according to claim 36, wherein the CREB3L3 variant nucleic acid molecule encodes Asp182Asn-A, Asp182Asn-B, Asp182Asn-C, or Asp182Asn-D.

41. The method according to claim 39, wherein the CREB3L3 variant nucleic acid molecule is:
a genomic nucleic acid molecule having a nucleotide sequence comprising an adenine at a position corresponding to position 6,120 according to SEQ ID NO:2;
an mRNA molecule having a nucleotide sequence comprising an adenine at a position corresponding to: position 661 according to SEQ ID NO: 17, position 649 according to SEQ ID NO: 18, position 624 according to SEQ ID NO: 19, position 624 according to SEQ ID NO:20, position 658 according to SEQ ID NO:21, position 661 according to SEQ ID NO:22, position 661 according to SEQ ID NO:23, position 663 according to SEQ ID NO:24, position 660 according to SEQ ID NO:25, position 649 according to SEQ ID NO:26, position 624 according to SEQ ID NO:27, or position 691 according to SEQ ID NO:28; or
a cDNA molecule produced from an mRNA molecule, wherein the cDNA molecule has a nucleotide sequence comprising an adenine at a position corresponding to position: position 661 according to SEQ ID NO:45, position 649 according to SEQ ID NO:46, position 624 according to SEQ ID NO:47, position 624 according to SEQ ID NO:48, position 658 according to SEQ ID NO:49, position 661 according to SEQ ID NO:50, position 661 according to SEQ ID NO:51, position 663 according to SEQ ID NO:52, position 660 according to SEQ ID NO:53, position 649 according to SEQ ID NO:54, position 624 according to SEQ ID NO:55, or position 691 according to SEQ ID NO:56.

42-56. (canceled)

57. The method according to claim 36, wherein the CREB3L3 inhibitor comprises an inhibitory nucleic acid molecule.

58. The method according to claim 57, wherein the inhibitory nucleic acid molecule comprises an antisense nucleic acid molecule, a small interfering RNA (siRNA), or a short hairpin RNA (shRNA) that hybridizes to a CREB3L3 nucleic acid molecule.

59-97. (canceled)
